(19) United States
(12) Patent Application Publication
(10) Pub. No.: US 2021/0290539 A1
(43) Pub. Date: Sep. 23, 2021

(54) ENGINEERED HEMICHANNELS, ENGINEERED VESICLES, AND USES THEREOF

(71) Applicants: Robert G. GOURDIE, Roanoke, VA (US); VIRGINIA TECH INTELLECTUAL PROPERTIES, INC., BLACKSBURG, VA (US); VIRGINIA POLYTECHNIC INSTITUTE AND STATE UNIVERSITY, Blacksburg, VA (US)

(72) Inventors: Robert G. GOURDIE, Roanoke, VA (US); L. JANE JOURDAN, Roanoke, VA (US); Eda ROGERS, Roanoke, VA (US)

(21) Appl. No.: 17/264,793

(22) PCT Filed: Jul. 30, 2019
(86) PCT No.: PCT/US2019/044248

§ 371 Date: Jan. 29, 2021

Related U.S. Application Data
(60) Provisional application No. 62/712,067, filed on Jul. 30, 2018.
(60) Provisional application No. 62/865,895, filed on Jun. 24, 2019.
(60) Provisional application No. 62/823,471, filed on Mar. 25, 2019.
(60) Provisional application No. 62/823,457, filed on Mar. 25, 2019.

(51) Int. Cl. A61K 9/127; C07K 14/705; A61K 47/46; A61K 9/00; A61P 17/02; A61P 9/06; A61P 25/00; A61P 27/02; A23C 9/152

(52) U.S. Cl. CPC A61K 9/1278; C07K 14/705; A61K 9/1271; A61K 47/46; A61K 9/0056; A61K 38/00; A61P 9/06; A61P 25/00; A61P 27/02; A23C 9/1528; A23C 9/1526; A61P 17/02

(57) ABSTRACT

Described herein are engineered hemichannels, engineered vesicles that can contain the one or more of the engineered hemichannels, pharmaceutical formulations thereof, and uses thereof. In some aspects, the engineered vesicles can include one or more cargo molecules. Also described herein are methods of loading the engineered vesicles. In some aspects, loading of one or more cargo molecules engineered vesicles can be optionally via an engineered hemichannel contained in the engineered vesicle.

### CROSS-REFERENCE TO RELATED APPLICATIONS

This application claims the benefit of and priority to co-pending U.S. Provisional Patent Application No. 62/712,067 filed on Jul. 30, 2018, entitled “ENGINEERED HEMICHANNELS, ENGINEERED VESICLES, AND USES THEREOF,” the contents of which is incorporated by reference herein in its entirety.

This application also claims the benefit of and priority to co-pending U.S. Provisional Patent Application No. 62/823,457 filed on Mar. 25, 2019, entitled “METHODS FOR HEMICHANNEL LOADING OF EXOSOMAL DRUG DELIVERY VEHICLES WITH THERAPEUTIC MOLECULES,” the contents of which is incorporated by reference herein in its entirety.

This application also claims the benefit of and priority to co-pending U.S. Provisional Patent Application No. 62/823,471 filed on Mar. 25, 2019, entitled “TARGETING THE CX43 CARBOXYL TERMINAL H2 DOMAIN PRESERVES LEFT VENTRICULAR FUNCTION FOLLOWING ISCHEMIA-REPERFUSION INJURY,” the contents of which is incorporated by reference herein in its entirety.

This application also claims the benefit of and priority to co-pending U.S. Provisional Patent Application No. 62/865,895 filed on Jun. 24, 2019, entitled “ENGINEERED HEMICHANNELS, ENGINEERED VESICLES, AND USES THEREOF,” the contents of which is incorporated by reference herein in its entirety.

### STATEMENT REGARDING FEDERALLY SPONSORED RESEARCH

This invention was made with Government support HL56728 and HL141855 awarded by the National Institutes of Health. The Government has certain rights in the invention.

### SEQUENCE LISTING

This application contains a sequence listing filed in electronic form as an ASCII.txt file entitled VTIP_0170WP_ST25.txt, created on Jul. 30, 2019. The content of the sequence listing is incorporated herein in its entirety.

### TECHNICAL FIELD

The subject matter disclosed herein is generally directed to engineered vesicles and vesicle-mediated delivery of cargo compounds.

### BACKGROUND

Peptides and other small biologic compounds (e.g. polynucleotides) have a great potential to provide new therapies. Although initial results can be promising, they are difficult to translate into clinical therapies. Small biologic molecules are prone to rapid degradation and/or neutralization upon administration. As such, there exists a need for compositions and methods for delivery of small biologic and other compounds.

### SUMMARY

Described herein are aspects of an engineered hemichannel comprising: an engineered connexin 43 polypeptide comprising a non-functional c-terminus, wherein the engineered hemichannel is non-responsive to a change in pH. In aspects, the engineered hemichannel of is responsive to calcium concentration. In aspects, the engineered connexin 43 polypeptide has a modified c-terminal region as compared to SEQ ID NO: 1. In aspects, the modification in the c-terminal region renders the engineered hemichannel non-responsive to changes in pH. In aspects, the hemichannel is composed of 3-10 engineered connexin 43 polypeptides. In aspects, the change in pH is a change to an acidic pH. In aspects, the change in pH is a change to a pH less than 8.5.

Descried herein are aspects of an engineered polypeptide comprising: a modified connexin 43 polypeptide, wherein the modified connexin 43 polypeptide is modified as compared to SEQ ID NO: 1 and comprises one or more amino acid deletions, one or more amino acid insertions, one or more amino acid mutations, or any combination thereof in the c-terminal region of SEQ ID NO 1. In some aspects, the engineered polypeptide is an amino acid sequence according to any one of SEQ ID NOs: 3-12. In some aspects, engineered polypeptide is an amino acid sequence that is about 50-100 percent identical to amino acids 1-224 of SEQ ID NO: 1 and has amino acids 225 to 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, or 382 of SEQ ID NO: 1 deleted. In some aspects, the engineered polypeptide is an amino acid sequence that is about 50-100 percent identical to amino acids 1-224 of SEQ ID NO: 1 and has amino acids 382 to 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, or 381, of SEQ ID NO: 1 deleted. In some aspects, the engineered polypeptide is about 50 percent to about 100% identical to amino acids 1-224 of SEQ ID NO: 1 and has one or more of amino acids 225-382 of SEQ ID NO: 1 deleted. In some aspects, amino acids 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, or any combination thereof of SEQ ID NO: 1 is deleted. In some aspects, the engineered polypeptide is about 50-100 percent identical to amino acids 1-224 of SEQ ID NO: 1 and has one or more amino acids inserted between any two amino acids from amino acid residues 224-382 of SEQ ID NO: 1.

In some aspects, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids are inserted between any two amino acid residues in the c-terminus region ranging from amino acid residues 224 and 382 of SEQ ID NO: 1. In some aspects, at least two insertions are present in the engineered polypeptide. In some aspects, the insertions are the same amino acid, peptide, or polypeptide. In some aspects, at least two of the insertions can be different from each other. In some aspects, the insertion is A, I, L, M, V, F, W, Y, N, C, Q, S, T, D, E, R, H, K, G, P or any combination thereof. In some aspects, the engineered polypeptide can include one or more amino acid mutations in the c-terminal region as compared to SEQ ID NO: 1. In some aspects, any one or more of the amino acids residues 225-382 can be substituted with any one of amino acids A, I, L, M, V, F, W, Y, N, C, Q, S, T, D, E, R, H, K, G, P that is not the same as the amino acid that it is being substituted for. In some aspects, the mutation is selected from the group consisting of: S368A, S368D, S365A, S365D, S373A, S373A D379A, E381A, S364P, C298A, E381A, D379A, D378A, S325A, S328A, S330A, and any combination thereof.

Described herein are aspects of a polynucleotide comprising: a polynucleotide configured to encode an engineered polypeptide as described herein, such as any of those above.

Described herein are aspects of a vector comprising: a polynucleotide as described herein, such as above, and a regulatory polynucleotide, wherein the regulatory polynucleotide is operably linked to the polynucleotide configured to encode the engineered polypeptide.

Described herein are aspects of a cell comprising a vector as described herein, such as above.

Described herein are aspects of a cell comprising a polynucleotide as described herein, such as above.

Described herein are aspects of a cell comprising an engineered hemichannel as described herein, such as above, one or more polypeptides as described herein, such as above, or both.

Described herein are aspects of an engineered hemichannel comprising: an engineered polypeptide as described herein, such as above. In some aspects, the engineered hemichannel has 3 to 10 engineered polypeptides as described herein, such as above. In some aspects, the engineered polypeptides are all the same. In some aspects, at least two of the engineered polypeptides are different. In some aspects, all of the engineered polypeptides are different.

Described herein are aspects of an engineered vesicle comprising: a lipid bilayer; and an engineered hemichannel as described elsewhere herein, an engineered polypeptide as described elsewhere herein, or both, wherein the engineered polypeptide is integrated in the lipid bilayer.

Described herein are aspects of an engineered vesicle comprising: a lipid bilayer; and a plurality of engineered polypeptides, wherein each engineered polypeptide of the plurality of engineered polypeptides is as described elsewhere herein wherein the engineered polypeptides are integrated in the lipid bilayer. In some aspects, the plurality of engineered polypeptides forms a hemichannel. In some aspects, the engineered vesicle, further comprises a cargo compound, wherein the cargo compound is contained within the engineered vesicle.

Described herein are aspects of an engineered vesicle comprising: a lipid bilayer; and an engineered hemichannel as described elsewhere herein. In some aspects, the engineered vesicle further comprises a cargo compound, wherein the cargo compound is contained within the engineered vesicle.

In some aspects, the engineered vesicle described herein is substantially spherical and has a diameter of about 1 nm to about 200 nm.

In some aspects, the engineered vesicle described herein is a milk-based engineered vesicle.

Described herein are aspects of an engineered vesicle comprising: a milk exosome; and a peptide cargo molecule contained within the milk exosome, wherein the peptide compound is selected from the group of: SEQ ID NOS: 13-47, 49-114, and 133 and combinations thereof. In some aspects, the milk exosome is a natural milk exosome. In some aspects, the engineered vesicle further comprises an esterase.

Described herein are aspects of a cell, wherein the cell is capable of producing an engineered vesicle as described elsewhere herein. In some aspects, the cell is capable of secreting the engineered vesicles. In some aspects, the cell comprises an engineered vesicle as described elsewhere herein.

Described herein are aspects of a cell that includes an engineered vesicle as described elsewhere herein.

Described herein are aspects of a method of loading a cargo compound in an engineered vesicle as described elsewhere herein, the method comprising: exposing an engineered vesicle to a solution comprising a low concentration of calcium and a cargo compound, wherein the low concentration of calcium opens the engineered hemichannel of the engineered vesicle, allowing the cargo compound to enter the engineered vesicle through the open engineered hemichannel, and closing the engineered hemichannel by exposing the engineered vesicle to a solution comprising a high concentration of calcium. In some aspects, the solution comprising a low concentration of calcium further comprises EDTA. In some aspects, the low concentration of calcium ranges from 0 mM to about 0.2 mM. In some aspects, the high concentration of calcium ranges from 0 mM to about 2 mM. In some aspects, the cargo compound comprises one or more cleavable ester groups. In some aspects, one or more of the one or more cleavable ester groups is cleaved by an esterase present in the engineered vesicle.

Described herein are aspects of a method that can include the step of opening an engineered hemichannel as describe elsewhere herein, by contacting the engineered hemichannel with a solution comprising a low concentration of Ca²⁺, wherein the low concentration of Ca²⁺ is capable of stimulating opening of the engineered hemichannel. In some aspects, the solution further comprises a cargo compound, wherein the concentration of the cargo compound in solution is such that it drives movement of the agent through the engineered hemichannel. In some aspects, the engineered hemichannel is integrated in a lipid bilayer of a vesicle. In some aspects, the method further includes the step of closing the engineered hemichannel by removing the engineered hemichannel from contact with the solution comprising a low concentration of calcium. In some aspects, the step of closing the engineered hemichannel is carried out by raising the concentration of calcium in the solution. In some aspects, the cargo compound comprises one or more cleavable ester bond-linked groups. In some aspects, cleavable ester bond-linked group is cleaved by an esterase or via other ester bond breaking acitivty present in the engineered vesicle.

Described herein are aspects of a method of loading a cargo compound into a vesicle, comprising: exposing a vesicle or component thereof to a cargo compound, allowing the cargo compound to enter the vesicle, be encapsulated by the vesicle, or both, wherein the vesicle comprises an esterase and wherein the cargo compound comprises one or more cleavable groups, wherein each cleavable group is linked by an ester bond to the cargo compound. In some aspects, the vesicle is an engineered vesicle as described elsewhere herein. In some aspects, the vesicle is a milk exosome as described elsewhere herein. In some aspects, the vesicle and cargo compound are exposed to a pH gradient formed between the inside of the vesicle and the outside of the vesicle during the step of exposing the vesicle or component thereof to the cargo compound, allowing the cargo compound to enter the vesicle, or both. In some aspects, the vesicle is exposed to an acidic pH. In some aspects, the vesicle is exposed to a basic pH. In some aspects, the vesicle is exposed to a pH of 8.5 or greater. In some aspects, the steps of exposing and allowing occur for at least 1 hour. In some aspects, the cargo compound is negatively charged. In some aspects, the cargo compound is positively charged. In some aspects, the cargo compound is neutrally charged. In some aspects, the cargo compound further comprises one or more charge modifying groups capable of shielding a charged group, adding a charged group, or both to the compound and modifying the charge of the cargo compound.

Described herein are aspects of a method comprising: administering an amount of an engineered vesicle as described herein or a cell as described herein to a subject. In some aspects, the subject has a disease, disorder, or condition. In some aspects, the subject has a chronic wound. In some aspects, subject has a diabetic ulcer. In some aspects the engineered vesicle comprises a cargo compound. In some aspects, the cargo compound is a peptide compound. In some aspects, the peptide compound is selected from the group of: SEQ ID NOS: 13-47, 49-114, 133, and combinations thereof. In some aspects, the cargo compound comprises one or more cleavable ester groups. In some aspects, one or more of the one or more cleavable ester groups is cleaved by an esterase present in the engineered vesicle.

Described herein are aspects of a method of treating a disease in a subject in need thereof, the method comprising: administering an engineered vesicle containing a cargo compound as described herein, wherein the cargo compound is capable of treating and/or preventing a disease or a symptom thereof in the subject. In some aspects, the disease is a skin wound, a chronic wound, myocardial infarction, heart failure, neural stroke, lung injury, macular degeneration, and radiation injury. In some aspects, the disease is a diabetic ulcer. In some aspects, the cargo compound comprises one or more cleavable ester groups. In some aspects, one or more of the one or more cleavable ester groups is cleaved by an esterase present in the engineered vesicle.

Described herein are aspects of an engineered polypeptide comprising: a peptide, wherein the peptide consists of a plurality of amino acids having a sequence identical to SEQ ID NO: 14 or 112. In some aspects, the engineered polypeptide further comprises a second polypeptide, wherein the second polypeptide is capable of performing a function different from the peptide consisting of a plurality of amino acids having a sequence identical to SEQ ID NO: 14 or 112. In some aspects, wherein the second polypeptide is a selectable marker.

Described herein are aspects of an engineered polypeptide comprising: a peptide, wherein the peptide consists of a plurality of amino acids having a sequence identical to SEQ ID NO: 14 or 112.

Described herein are aspects of an engineered peptide consisting of: a peptide having a sequence identical to SEQ ID NO: 14 or 112.

Described herein are aspects of a pharmaceutical formulation comprising: an engineered polypeptide of any one of claims 87-90 or an engineered peptide of claim 91; and a pharmaceutically acceptable carrier.

Described herein are aspects of a method comprising: administering an engineered polypeptide as described herein or an engineered peptide as described herein or a pharmaceutical formulation as described herein to a subject. In some aspects, the subject has or is suspected of having a disease.

Described herein are aspects of a method of treating a subject in need thereof, the method comprising: administering an engineered polypeptide as described elsewhere herein or an engineered peptide as described elsewhere herein or a pharmaceutical formulation as described elsewhere herein to the subject in need thereof.

Described herein are aspects of a pharmaceutical formulation comprising: an engineered vesicle as described herein; and a pharmaceutically acceptable carrier. In some aspects, the pharmaceutically acceptable carrier is milk or a milk product. Described herein are aspects of a method comprising: administering the pharmaceutical formulation where the pharmaceutically acceptable carrier is milk or a milk product as described to a subject in need thereof.

### BRIEF DESCRIPTION OF THE DRAWINGS

Further aspects of the present disclosure will be readily appreciated upon review of the detailed description of its various aspects, described below, when taken in conjunction with the accompanying drawings.

FIGS. 1A-1D. alpha CT1 interacts with Zonula Occludens-1 (ZO-1) PDZ2 and the Connexin 43 (Cx43) Carboxyl Terminus (CT). FIG. 1A) Schematics of full length Cx43 and alpha CT1 peptide. FIG. 1B) alpha CT1 interaction with ZO-1 PDZ domains as indicated by EDC zero-length cross-linking to GST fusion PDZ1, PDZ2 and PDZ3 polypeptides and neutravidin labeling of biotin-tagged peptide at concentrations of 5, 25 and 50 μM. The deletion of deletion of the CT Isoleucine (I) in alpha CT1-I renders this peptide incompetent to interact with the ZO-1 PDZ2 domain. FIG. 1C) Coomassie blue gel of EDC cross-linked products of kinase reaction mixtures containing GST-Cx43 CT and PKC-ε, with (alpha CT1) and without (Vehicle) alpha CT1. The fainter band above GST-Cx43 bands (indicated by lines) in the alpha CT1 lanes were cut from gels and analyzed by Tandem Mass Spectrometry (MS/MS). The boxes to right of gel show Cx43 CT peptides identified by MS/MS as being cross-linked to alpha CT1. FIG. 1D) Tandem mass spectrum of a quintuply charged crosslinked peptide (m/z: 674.1) between Cx43 345-366 (a-chain) and alpha CT1 peptide through Cx43 K346 and E8 in alpha CT1 (b-chain). Only the b- and y-sequence specific ions are labeled. Arrow indicates ion (ba52+) consistent with cross-linkage between Cx43 CT lysine K346 and the glutamic acid (E) residue of alpha CT1 at position −1.

FIGS. 2A-2D. Molecular modeling of the alpha CT1 and Cx43 CT complex. FIG. 2A) Schematics of Cx43 and the secondary structure of Cx43 CT from amino acid residues Glycine252 (G252) through to Isoleucine 382 (1382). The depiction of secondary structure in FIG. 2A has been modified from a diagram originally provided by Sosinsky and co-workers 30. FIG. 2B) ZDOCK and FIG. 2C) Schrodinger molecular modeling software analysis of the structure of a proposed alpha CT1-Cx43 CT complex. The protonated structure of alpha CT1 peptide and Cx43 CT (PDB:1r5s), constrained by a salt-bridge interaction between K346 in the Cx43 CT and the glutamic acid (E) at position −1 of alpha CT1. The alpha CT1-Cx43 interaction shown represents that based on the lowest energy minimization score determined in the model. FIG. 2D) Schrodinger molecular modeling software, a 2D map of alpha CT1-Cx43 CT in anti-parallel orientation showing location of amino acids predicted to bond to each other and the type of bond that is predicted to occur.

FIGS. 3A-3F. alpha CT1 variants with alanine substitutions of negatively charged amino acids show abrogated ability to bind Cx43 CT (FIGS. 3A-F). SPR was used to analyze interactions of biotin-alpha CT1 and biotin-alpha CT1 variant peptides, immobilized to streptavidin-coated chips, with the Cx43 CT (Cx43-CT: amino acids 255 to 382) and Cx43 CT-KK/QQ as analytes, respectively. The mean of three runs is plotted for each analyte concentration. The exposure of the sensor chip to the specific analyte is indicated by the gray area. Sensorgrams obtained for: A) Cx43 CT and biotin-alpha CT1. B) Cx43 CT-KK/QQ and biotin-alpha CT1. FIG. 3C) Cx43 CT and biotin-M1 AALAI. FIG. 3D) Cx43 CT-KK/QQ and biotin-M1 AALAI. FIG. 3E) Cx43 CT and biotin-M3 DDLAI. FIG. 3F) Cx43 CT-KK/QQ and biotin-M3 DDLAI.

FIGS. 4A-4C. alpha CT1 interaction stabilizes PDZ2 and destabilizes Cx43 CT secondary structure. FIG. 4A) Melt curves (top) and first derivative of melt curves (bottom) for ZO-1 PDZ2 at 500 μg/mL in combination alpha CT1 at concentrations of 25, 50 and 100 μM. FIG. 4B) Temperature maxima (Tm) from Boltzman curves from left-to-right of Cx43 CT (Cx43-CT: amino acids 255 to 382) alone, Cx43 CT in combination with alpha CT1, and the alpha CT1 variants including: M1 (AALAI), M2 (AALEI), M3 (DDLAI), M4 scrambled, alpha CT-I and alpha CT11. alpha CT1, alpha CT1-I and alpha CT11 show similar abilities to destabilize (i.e., significantly decrease the Tm of) Cx43 CT. **p<0.01, ***p<0.002, N=6. FIG. 4C) Temperature maxima (Tm) from Boltzman curves from left-to-right of PDZ2 alone, and PDZ2 in combination with alpha CT1 (also referred to herein by the acronyms aCT1, αCT1, ACT1) and alpha CT1 variants including alpha CT1 variants including: M1 (AALAI), M2 (AALEI), M3 (DDLAI), M4 scrambled, alpha CT-I and alpha CT11. M3 (DDLAI), alpha CT1, and alpha CT11 show similar abilities to stabilize (i.e., significantly increase the Tm of) PDZ2. **p<0.01, ***p<0.002, N=6

FIGS. 5A-5C. Cx43 mimetic peptides that retain Cx43-binding capability are able to induce phosphorylation of Cx43-CT at serine 368 (S368). FIG. 5A) Blots of Cx43-pS368 (top) and total Cx43 (bottom) in kinase reactions mixtures including no-kinase controls with substrate (Cx43-CT: amino acids 255 to 382), but no PKC-ε (PKC-minus); Cx43-CT substrate with PKC-ε (PKC-plus); and mixtures containing PKC-ε, Cx43 CT, and biotin-tagged alpha CT1, biotin-tagged alpha CT1 mutant peptides with alanine substitutions (M1 AALAI, M2 AALEI, M3 DDLAI) and biotin-tagged M4 scrambled. Peptides are at 20 μM. FIG. 5B) Blots of Cx43-pS368 (top) and total Cx43 (bottom) in kinase reactions mixtures including no-kinase controls with Cx43 CT substrate, but no PKC-ε (PKC-minus); Cx43-CT substrate with PKC-c (PKC-plus); and mixtures containing PKC-ε, Cx43 CT, and biotin-alpha CT1, biotin-alpha CT1-I or biotin-alpha CT11 with no antennapedia sequence at peptide NT) and biotin-M4 scrambled peptide. Peptides are at 20 μM. FIG. 5C) Chart showing that the ability of unmodified alpha CT1 and the Cx43 CT interaction-competent peptides biotin-alpha CT1-I or biotin-alpha CT11 to induce S368 phosphorylation was 3-5 fold greater than that of non-Cx43 CT interacting peptides. *p<0.05, **p<0.01, ***p<0.002, N=5 alpha CT1 and M4, other peptides N=3.

FIGS. 6A-6B. Pre-Ischemia treatment with peptides competent to interact with Cx43 CT protect hearts from ischemia-reperfusion (I/R) injury. Langendorff I/R protocols were performed on adult mouse hearts instrumented to monitor LV function (protocol in FIG. 9). Representative pressure traces from hearts from: (FIG. 6A) Vehicle control and (FIG. 6B) 10 μM alpha CT1 infused hearts. Note that the alpha CT1 treatment results in notable recovery of LV function during reperfusion.

FIGS. 7A-7H. Pre-Ischemic treatment with peptides interacting with Cx43 CT protect hearts from ischemia-reperfusion injury in association with increased pS368 in LV myocardium. Langendorff ischemia-reperfusion (I/R) injury protocols were performed on adult mouse hearts instrumented to monitor LV contractility (protocol in FIG. 9). LV Systolic responses are shown in FIGS. 7A-7C: (FIG. 7A) Plots of left ventricular (LV) systolic developed pressure against balloon volume; (FIG. 7B) LV maximal rate of tension development (+dP/dt) against balloon volume; (FIG. 7C) Maximal systolic elastance (Emax)—i.e., the slope from (FIG. 7A); (FIG. 7D) Plots of LV end diastolic pressure (EDP) against balloon volume; (FIG. 7E) Maximal rate of relaxation (−dP/dt) against balloon volume; (FIG. 7F) Stiffness, the reciprocal of the slope from (FIG. 7D); (FIG. 7G) Percentage of LV contractile function recovery post-ischemia relative to baseline level. Data shown are mean±S.E. N=4-8. *p<0.05, ***p<0.001, N=4-8 hearts/group. H) Blots of Cx43-pS368 (top) and total Cx43 (bottom) of LV samples infused with peptide for 20 minutes according to the protocol in FIG. 9. For hearts used in Western blots, the protocol did not proceed to the ischemia and reperfusion phases, being terminated after the peptide infusion step. Only those peptides competent to interact with Cx43 CT increase pS368 levels relative to total Cx43 above vehicle control.

FIGS. 8A-8H. Pre- and Post-Ischemic treatment with alpha CT11 protect hearts from ischemia-reperfusion injury. Langendorff I/R protocols were performed on adult mouse hearts instrumented to monitor LV contractility. Protocol in FIG. 9, except that a 20-minute peptide infusion was begun after ischemic injury at the initiation of reperfusion. (FIG. 8A) Plots of left ventricular (LV) developed pressure against balloon volume; (FIG. 8B) Maximal systolic elastance (Emax), the slope from (FIG. 8A); (FIG. 8C) Maximal rate of tension development (+dP/dt) against balloon volume; (FIG. 8D) Plots of end diastolic pressure (EDP) against balloon volume; (FIG. 8E) Stiffness, the reciprocal of the slope from (FIG. 8D); (FIG. 8F) Maximal rate of relaxation (−dP/dt) against balloon volume. *p<0.05, ***p<0.001, N=4-8. G) Laser scanning confocal microscopic fields from sections of Vehicle control, alpha CT1, and alpha CT11 group hearts stained for Cx43 (green), nuclei (DAPI-blue), and Alexa647-conjugated streptavidin (red). H) Average intensities of biotinylated peptide (indicated by streptavidin Alexa647 fluorescence intensity level relative to background) in Vehicle control, alpha CT1, and alpha CT11 groups. **p<0.05; not significant (ns) N=5 hearts/group. Scale bar=5 μm.

FIG. 9. Ischemia reperfusion injury model/protocol. The protocol involved a 20-minute period of no flow ischemia period followed by 40 minutes of reperfusion, LV contractile function was monitored throughout the whole process. For treatment, peptides were infused into hearts over a 20-minute period just prior to the ischemic episode. Expanded representative pressure traces for each of these phases are shown below.

FIG. 10. Blots of EDC cross-linked products of kinase reaction mixtures containing GST-Cx43 CT, GST-Cx43 CT QQ/KK in which the lysine (K) residues were mutated to neutral glutamines (Q), PKC-ε and alpha CT1 (at 5, 10 and 25 μM) and a scrambled alpha CT1 (M4 scr) variant at the same concentrations. Alpha CT1 was observed to be covalently linked by EDC to Cx43 CT in a concentration-dependent manner.

FIGS. 11A-11B. The alpha CT1 variant peptide M2 AALEI shows limited ability to bind Cx43 CT. SPR was used to analyze interactions of biotin-M2 AALEI with the Cx43 CT (FIG. 11A) and Cx43 CT-KK/QQ (FIG. 11B) as respective analytes. The mean of three runs is plotted for each analyte concentration. The exposure of the sensor chip to the specific analyte is indicated by the gray area.

FIG. 12 shows Connexin 43 hemichannels are competent to take up alphaCT11 (aCT11) (RPRPDDLEI MW—1110.22 daltons (SEQ ID NO: 13)) and that this uptake was prevented by Cx43 hemichannel blockers. Media containing 0.1 mM Ca²⁺ was used to open Cx43 hemichannels in the presence of 50 μM alphaCT11 peptide and/or the hemichannel blockers; Gap19 (200 μM) and carbenoxolone (50 μM). Hemichannel opening by reduced external Ca²⁺ was associated with high levels of alphaCT11 uptake. Dramatically lower levels of peptide uptake were observed in the 0.1 mM Ca²⁺ solution also containing the Cx43 hemichannel blockers Gap19 and carbenoxolone. When the external solution contained 1.8 mM Ca²⁺, alphaCT11 take up was not observed consistent with hemichannels being closed.

FIGS. 13A-13E. Short peptides based on the Carboxyl-Terminus (CT) of the gap junction protein connexin 43 (Cx43) provide high levels of protection against ischemia reperfusion injury to the heart. Contractile function of the left ventricle (LV) of isolated beating mouse hearts was continuously recorded (FIG. 13A) during ex vivo perfusion (FIG. 13B) in a model simulating ischemia-reperfusion (I/R) injury to the heart. To induce an ischemic injury, hearts were subjected to a no flow ischemic injury for 20 minutes (indicated by loss of pressure recording on (FIG. 13A) and subsequently reperfused with oxygenated buffer solution for about 40 minutes. This was observed to result in about a 80-90% loss of LV contractile function in control hearts (FIG. 13C) By contrast, hearts treated for 20 minutes with either the Cx43 CT-based peptide RPRPDDLE (8 amino acids) (SEQ ID NO: 14, also referred to alpha CT11-I) or RPRPDDLEI (9 amino acids) (SEQ ID NO: 13, also referred to as alpha CT11) both showed striking levels (p<0.001) of cardioprotection, with recovery of LV contractile function 5-6 times higher than that of hearts subject to vehicle control or inactive peptide control perfusions (FIG. 13C). To confirm cardioprotection, staining of hearts after measurement of contractile function was performed using 2,3,4-triphenyltetrazolium chloride (TTC) to indicate sectors of dead (white staining) and live (red staining) heart muscle. Treatment with therapeutic peptide resulted in dramatic improvements in preservation of live heart muscle (FIG. 13D), with treated hearts having about 57% (p<0.05) more muscle than control hearts subject to the I/R injury protocol (FIG. 13E).

FIGS. 14A-14D HeLa cell exosomes retain Calcein AM dye. (FIG. 14A) HeLa cells engineered to express Cx43-GFP-inset shows Cx43GFP gap junctions (GJs). (FIG. 14B) Nanosight size distribution of Cx43GFP+ exosomes from HeLa cells. (FIG. 14C) Laser scanning confocal microscopy (LSCM) image of Cx43GFP+ exosomes loaded with Calcein red dye. (FIG. 14D) Significant co-localization of exosomal Cx43GFP+ with Calcein red measured at time points >60 minutes. This co-localization confirms exosomal retention of Calcein, indicating that the ester bond had been cleaved and the dye was now trapped in the exosome. Calcein AM includes acetoxymethyl (AM) groups, which facilitate the movement of the molecule across membranes. Once inside cells, the ester bonds linking these groups are cleaved by intracellular ester bond breaking activity, such as esterases, trapping the molecule. We have determined that exosomes contain ester bond breaking activities, and thus can be loaded with Calcein, and other molecules with ester-linked moieties that promote movement across the exosomal membrane. For chemically modified amino acids, peptides and polypeptides with chemical groups attached to D and E residues and/or the original terminal carboxyl group by ester bonds, esterase cleavage can restore COOH groups at these sites and thus the chemical structure of the peptide found in nature. e.g. FIG. 15. Scale bars: A=100 μm, C=5 μm.

FIG. 15 shows a schematic that can demonstrate exosomal loading of an esterified cargo compound to increase loading efficiency of the exosome with the cargo molecule.

FIG. 16 shows a fluorescent microscopic image that can demonstrate that milk exosomes retain Calcein dye. Exosomes were isolated from unpasteurized milk and incubated with Calcein AM dye. Milk exosomes retained dye, indicating that they contain esterase activity needed for ester bond cleavage, and hence dye and/or peptide retention used in aspects described herein.

FIG. 17 shows a schematic demonstrating suggested mechanisms of action for alpha CT11 activity and interaction with connexin43 and Connexin43 hemichannels and loading of an engineered exosome as described herein with an exemplary cargo (e.g. alpha CT11) compound, and delivery of a cargo compound. FIG. 17 shows on mechanism of cargo compound delivery that involves gap junction channel formation between connexins on the exosome and the cell to which the cargo can be delivered. In FIG. 17, this is connexon43 on both the exosome and cell. It will be appreciated other delivery methods are possible and described herein.

FIGS. 18A-18E can demonstrate post-ischemic alpha CT11 results in dramatic preservation of LV contractile function in isolated, perfused hearts in association with alpha CT11 permeance into myocytes.

FIGS. 19A-19B can demonstrate the Cx43 Gap Junction perinexus, which is a specialized zone of myocyte interaction at the edge of GJs. FIG. 19A shows an electron micrograph of GJ and adjacent perinexal cleft. FIG. 19B shows STORM super resolution image of a Cx43 GJ, with adjacent clusters of Nav1.5 VGSCs in the adjacent perinexus (Peri).

FIGS. 20A-20B can demonstrate that post-MI treatment with alpha CT11 can reduce infarct size by about 48% in a mouse in vivo myocardial infarction model. This post-infraction treatment can significantly improve ventricular ejection fraction, indicating that the treatment preserves heart ventricular function.

FIG. 21 can demonstrate that alpha CT11 can suppress discordant alterans in wedge preparations of ventricular tissue during ischemia. Discordant alternans of action potential duration (APD) is a phenomenon where different regions of cardiac tissue exhibit an alternating sequence of APD that are out-of-phase. Discordant alternans is highly arrhythmogenic since it can induce spatial heterogeneity of refractoriness, which can cause wavebreak and reentry. Thus, alpha CT11 can have powerful anti-arrhythmic benefits in this setting.

FIGS. 22A-22H can demonstrate that HC-mediated alpha CT11 uptake into the cytoplasm of MDCK Cx43 cells and LV myocytes in perfused mouse hearts.

FIG. 23 shows mass spectrometry results that can demonstrate that alpha CT11 can be degraded after about 30 minutes in blood serum.

FIGS. 24A-24E can demonstrate isolation, cargo loading, and uptake of exosomes expressing Cx43GFP. (FIG. 24A) HeLa cells engineered to express Cx43GFP-show GFP+ GJs between cells. (FIG. 24B) Nanosight size and concentration of Cx43GFP exosomes.

(FIG. 24C) Cx43GFP exosomes loaded with hemichannel (HC) permeant dye Atto-565 by increasing alkalinity of buffer. (FIG. 24D) Cellular uptake of exosomes. (FIG. 24E) Co-localization analysis can confirm hemichannel switch can allow for cargo compound loading (as demonstrated via dye loading) Scale A=100 μm, C, D=10 μm.

FIG. 25 can demonstrate uptake of exosomes in I/R injured heart by an oral and/or IP delivery route.

FIG. 26 shows a graph that can demonstrate that a calcium switch (e.g. calcium concentration) can be used to allow RPRPDDLEI (SEQ ID NO: 13) to permeate *p<0.05, **p<0.001.

FIGS. 27A-27D. HeLa cell exosomes retain Calcein dye: (FIG. 27A) HeLa cells engineered to express Cx43-GFP-inset shows Cx43-GFP gap junctions (GJs). (FIG. 27B) Nanosight size distribution of Cx43GFP+ exosomes from HeLa cells. (FIG. 27C) Laser scanning confocal microscopy (LCSM) image of Cx43GFP+ exosomes loaded with Calcein red dye. (FIG. 27D) Significant colocalization of exosomal Cx43GFP+ with Calcein red measured at time points >60 minutes. This co-localization confirms exosomal retention of Calcein, indicating that the dye's ester bonds have been cleaved and the dye is now trapped in the exosome. Scalre bars: A=100 microns, C=5 microns.

FIGS. 28A-28D. (FIG. 28A) shows a cartoon depiction of the two alpha helical regions of the Connexin 43 (Cx43) carboxyl terminus (CT), H1 and H2. (FIG. 28B) Schematic representation of the Cx43 Y313-A348 peptide synthesized for a binding surface surrogate with linkable cysteine (Cys) on the amino terminus and CT. (FIG. 28C) Single letter amino acid sequence of Cx43 Y313-A348 peptide with predicted helix secondary structure underlined. (FIG. 28D) Surface Plasmon Resonance (SPR) analysis of substrate captured aCT1 (700-1000 RUs) binding recombinant Cx43 CT (100 μM, light grey), unlinked Cx43 Y313-A348 peptide (25 μM, black), and disulfide linked Cx43 Y313-A348 (25 μM, dark grey). SPR indicates that non-disulfide linked Cx43 Y313-A348 peptide shows levels of interaction with aCT1 comparable to the full Cx43 CT polypeptide sequence (about 150 amino acids). Disulfide cross-linking Cx43 Y313-A348 into a looped conformation results in a loss of aCT1 binding, thus aCT1 interaction with this peptide requires a degree conformational flexibility. Cx43 Y313-A348 peptide can provide an assay for screening for novel Cx43 interacting drugs.

FIGS. 29A-29B. (FIG. 29A) (Top) Fluorescently tagged RhodamineB aCT11 peptide (RPRPDDLEI (SEQ ID NO: 13)); Bottom—acid-stable allyl protecting groups linked by ester bonds to peptide at aspartic (D) and glutamic (E) acid residues of aCT11. (FIG. 29B) Mass spectra (MALDI) of RhodamineB aCT11 peptide (TOP) and RhodamineB aCT11 peptide with each of it D and E residues and terminal carboxylic acid group converted with ester bond linked protecting groups (Bottom). The peaks show molecular masses that correspond to the expected structure (non-methylated ‘VT’—TOP) and all 4 groups methylated (VT Me—Bottom) for the methylated version. The 2 peaks in each of the spectra shown correspond to the mass+hydrogen and mass+sodium.

FIGS. 30A-30B show microscopic and SEM images of (FIG. 30A)—EVs isolated from cow milk loaded with neutral non-fluorescent Calcein AM (10 μM) for 48 hours at 37 C in PBS buffer at pH 8.5. Scale=5 μm. This protocol resulted in efficient loading and retention of dye in the EVs—owing to esterase activity that cleaved ester bonded shielding groups from Calcein AM converting it to negatively charged fluorescent Calcein. Calcein AM uptake into milk EVs was respectively inhibited and blocked by 0.1 and 1 μM PMSF an inhibitor of carboxylesterases. (FIG. 30B) show negative stain electron micrograph of an exosome isolated from cow milk. Scale bar=50 nm. We have adapted our methods of isolation from milk to obtain high yields of EV, taking particular care not to cause rapid and/or massive precipitation of milk casein, as well as in centrifugation steps, which can reduce EV yields from milk.

FIGS. 31A-31C. Milk EVs incubated with Calcein AM showing time (FIG. 31A), pH (FIG. 31B) and concentration dependent effects on uptake of Calcein by EVs (FIG. 31C). Scale bars=5 μm. Methyl groups linked by ester bonds to Calcein shield negatively charged moieties. Cleavage of these groups by ester bond breaking activities within EVs results in Calcein becoming negatively charged, fluorescent and retained within the EV. FIG. 31A. EVs were incubated for 1, 2 or 3 hours in PBS at 37 C at pH 7.4 with Calcein AM (5 μM). Increasing numbers of EVs show Calcein fluorescence with increasing time—indicating time dependent uptake. FIG. 31B. EVs were incubated at pH 6.6, 7.4 and 8.5 in PBS buffer at 37 C with Calcein AM (5 μM). Increasing numbers of EVs show Calcein fluorescence with increasing alkalinity of the buffer—indicating pH dependent uptake. Without being bound by theory, the mechanism driving EV uptake can be a pH gradient between the outside (less acidic) and inside (more acidic) that favors that accumulation of neutral to weakly basic Calcein inside the EV. FIG. 31C Increasing numbers of EVs show Calcein fluorescence with increasing concentration of the dye—indicating concentration dependent uptake during incubation in 37 C PBS at pH 8.5.

FIG. 32 shows a panel of microscopic images that can demonstrate the effect of carge shielding groups and on upatake of a cargo molecule. Milk EVs incubated with fluorescent-tagged RhodamineB-aCT11 with charge shielding allyl groups linked by ester bonds at aspartic (D) and glutamic (E) acid residues, as well as its carboxyl terminus—RhodB-aCT11-Est. Scale bar=25 μm. The EVs have been incubated for 1, 2, 4 or 24 hours in PBS at 37 degrees C. with RhodB-aCT11-Est (1 mM) with the pH of PBS buffer solutions at pH 6.6, 7.4 and 8.5. Peptide uptake in to EVs occurs in a time and pH dependent manner, with the highest levels of uptake occurring in EVs incubated for 4 or 24 hours at pH 6.6. With its chemical groups shielding negatively charged COOH groups, RhodB-aCT11-Est has a positive charge. Fluor-tagged RhodamineB-aCT11 with no charge shielding groups showed little evidence of uptake by milk EVs. Without being bound by theory, the mechanism driving EV uptake can be a pH gradient between outside (more acidic) and inside (less acidic) of the EV that favors that accumulation of positively charged RhodB-aCT11-Est inside the EV.

FIGS. 33A-33F. (FIG. 33A) Monolayer of HeLa cells. Scale bar=400 μm. (FIG. 33B) Fluorescently tagged RhodamineB aCT11 peptide (RhodB-aCT11). RhodB-aCT11 peptide does not have the acid-stable allyl protecting groups linked by ester bonds to peptide at aspartic (D) and glutamic (E) acid residues, as well as the carboxyl terminus, of aCT11 referred to in this figure as RhodB-aCT11-Est. HeLa cell monolayer incubated with RhodB-aCT11 peptide at 500 μM in culture media for 90 minutes at 37 C. Scale bar=80 μm. Little evidence for uptake of RhodB-aCT11 is resolved at this magnification following treatment. (FIGS. 33C-33F). By contrast to RhodB-aCT11, RhodB-aCT11-Est (the peptide with allyl protecting groups) is detectable as diffuse fluorescent signal within cultured cells incubated with different concentrations of the peptide between 500 and 2000 μM. This result indicates that RhodB-aCT11-Est is cell permeant and stably accumulates inside cells following esterase cleavage of the allyl groups. The concentration dependent uptake of RhodB-aCT11-Est can be used in methods wherein exosome producing cells are incubated with the peptide. Cells can take up the peptide, cytoplasmic esterases will cleave the allyl groups converting the peptide to RhodB-aCT11. RhodB-aCT11-Est, or any chemically modified drug molecule designed for cell uptake using ester bonded groups or similar chemical modifications, can be packaged as cargo into EVs and exported by the cell into the media. EVs loaded with cargo molecules by this method can then be isolated using standard protocols and used in the treatment and other methods detailed herein.

FIGS. 34A-34B. (FIG. 34A) Monolayers of HeLa cells incubated with fluorescent-tagged RhodamineB-aCT11, a cell-permeant peptide with allyl groups linked by ester bonds at aspartic (D) and glutamic (E) acid residues, as well as its carboxyl terminus (A) or Rhodamine B aCT11 peptide not having ester bonded groups (B). Scale bars=400 μm. The cells have been incubated for 30 or 90 minutes with different concentrations of the peptides between 200 and 2000 μM. Only cells incubated with the cell-permeant peptides show peptide uptake, which is seen to occur in a time and concentration dependent manner. Cellular uptake in FIG. 34A is particularly evident following 90 minutes at the higher peptide concentrations. The uniform fluorescence in the 2000 mM incubations in B result from general fluorescence of concentrated peptide dissolved in the media i.e., it does not indicate uptake. RhodB-aCT11-Est taken up in this manner by cells can be packaged as cargo into EVs and following isolation can be used in treatment and other methods detailed herein.

### DETAILED DESCRIPTION

Before the present disclosure is described in greater detail, it is to be understood that this disclosure is not limited to particular aspects described, and as such may, of course, vary. It is also to be understood that the terminology used herein is for the purpose of describing particular aspects only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used in the specification and the appended claims, the singular forms “a,” “an,” and “the” include plural referents unless the context clearly dictates otherwise.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As will be apparent to those of skill in the art upon reading this disclosure, each of the individual aspects described and illustrated herein has discrete components and features which may be readily separated from or combined with the features of any of the other several aspects without departing from the scope or spirit of the present disclosure. Any recited method can be carried out in the order of events recited or in any other order that is logically possible.

Aspects of the present disclosure will employ, unless otherwise indicated, techniques of molecular biology, microbiology, organic chemistry, biochemistry, physiology, cell biology, cancer biology, and the like, which are within the skill of the art. Such techniques are explained fully in the literature.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible unless the context clearly dictates otherwise.

### Definitions

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “additive effect” refers to an effect arising between two or more molecules, compounds, substances, factors, or compositions that is equal to or the same as the sum of their individual effects.

As used herein, “administering” refers to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratym panic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, intracardiac, epidural, intratracheal, intranasal, and intracranial injections or infusion techniques

As used herein, “agent” refers to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “amphiphilic” refers to a molecule combining hydrophilic and lipophilic (hydrophobic) properties.

As used herein, “antibody” refers to a glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen.

As used herein, “anti-infective” refers to compounds or molecules that can either kill an infectious agent or inhibit it from spreading. Anti-infectives include, but are not limited to, antibiotics, antibacterials, antifungals, antivirals, and antiprotozoans.

As used herein, “aptamer” refers to single-stranded DNA or RNA molecules that can bind to pre-selected targets including proteins with high affinity and specificity. Their specificity and characteristics are not directly determined by their primary sequence, but instead by their tertiary structure.

As used herein “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basal cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, glioma, glioblastoma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sezary syndrome, skin cancer, small intestine cancer, large intestine cancer (colon cancer), soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, ovarian cancer and Wilms Tumor.

As used herein, “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, “chemotherapeutic agent” or “chemotherapeutic” refers to a therapeutic agent utilized to prevent or treat cancer.

As used herein, “concentrated” refers to a molecule or population thereof, including but not limited to a polynucleotide, peptide, polypeptide, protein, antibody, or fragments thereof, that is distinguishable from its naturally occurring counterpart in that the concentration or number of molecules per volume is greater than that of its naturally occurring counterpart.

As used herein, “control” refers to an alternative subject or sample used in an experiment for comparison purpose and included to minimize or distinguish the effect of variables other than an independent variable.

As used herein with reference to the relationship between DNA, cDNA, cRNA, RNA, protein/peptides, and the like “corresponding to” refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “culturing” refers to maintaining cells under conditions in which they can proliferate and avoid senescence as a group of cells. “Culturing” can also include conditions in which the cells also or alternatively differentiate.

As used herein, “deoxyribonucleic acid (DNA)” and “ribonucleic acid (RNA)” generally refers to any polyribonucleotide or polydeoxribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), Long non-coding RNA (LncRNA) or coding mRNA (messenger RNA).

As used herein, “DNA molecule” can include nucleic acids/polynucleotides that are made of DNA.

As used herein, “dose,” “unit dose,” or “dosage” refers to physically discrete units suitable for use in a subject, each unit containing a predetermined quantity of the engineered vesicles described herein and/or a pharmaceutical formulation thereof calculated to produce the desired response or responses in association with its administration.

As used herein, “effective amount” refers to the amount of a compound provided herein that is sufficient to effect beneficial or desired biological, emotional, medical, or clinical response of a cell, tissue, system, animal, or human. An effective amount can be administered in one or more administrations, applications, or dosages. The term can also include, within its scope, amounts effective to enhance or restore to substantially normal physiological function. The “effective amount” can refer to the amount of an engineered vesicle described herein that can treat or prevent a disease or disorder or a symptom thereof in a subject to which it is administered.

As used herein, the term “encode” refers to the principle that DNA can be transcribed into RNA, which can then be translated into amino acid sequences that can form proteins.

As used herein, “extracellular vesicle” refers to a membrane-vesicle that can be formed in cells by e.g. endocytosis of the plasma membrane. Extracellular vesicles can be formed intracellularly and can contain a lipid bilayer that surrounds an internal phase, which is typically aqueous and composed of intracellular contents. After formation, the extracellular vesicle can be secreted by the cell. The term “extracellular vesicle” can include nanovesicles, exosomes and microvesicles. Extracellular vesicles can be secreted by cells and can be circulated in body fluids and/or be associated with cells, tissues and/or extracellular matrix. Extracellular vesicles can range in size from about 20 nm to about 3,000 or more nm. Exosomes can form via the endocytic pathway. Cobelli et al. 2017. Ann NY Acad. Sci. 1410(1):57-67). Macrovesicles can form from outward budding of the plasma membrane. See also Raposo and Stoorvogel. 2013 J. Cell Biol. 200(4):373. Extracellular vesicles can be synthetically produced as described elsewhere herein.

As used herein, the terms “Fc portion,” “Fc region,” and the like are used interchangeably herein and can refer to the fragment crystallizable region of an antibody that interacts with cell surface receptors called Fc receptors and some proteins of the complement system. The IgG Fc region is composed of two identical protein fragments that are derived from the second and third constant domains of the IgG antibody's two heavy chains.

The term “hydrophilic”, as used herein, refers to substances that have strongly polar groups that are readily soluble in water.

The term “hydrophobic”, as used herein, refers to substances that lack an affinity for water; tending to repel and not absorb water as well as not dissolve in or mix with water.

As used herein, ““inflammation”, “inflammatory response” or “immune response” refers to the reaction of living tissues to injury, infection or irritation characterized by redness, warmth, swelling, pain, and loss of function, produced as the result of increased blood flow and an influx of immune cells and secretions. Inflammation is the body's reaction to invading infectious microorganisms and results in an increase in blood flow to the affected area, the release of chemicals that draw white blood cells, an increased flow of plasma, and the arrival of monocytes (or astrocytes in the case of the brain) to clean up the debris. Anything that stimulates the inflammatory response can be considered inflammatory.

As used herein, “identity,” refers to a relationship between two or more nucleotide or polypeptide sequences, as determined by comparing the sequences. In the art, “identity” can also refer to the degree of sequence relatedness between nucleotide or polypeptide sequences as determined by the match between strings of such sequences. “Identity” can be readily calculated by known methods, including, but not limited to, those described in (Computational Molecular Biology, Lesk, A, M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J. Applied Math. 1988, 48: 1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48: 443-453) algorithm (e.g., NBLAST, and XBLAST). The default parameters are used to determine the identity for the polypeptides of the present disclosure, unless stated otherwise.

As used herein, “immunomodulator,” refers to an agent, such as a therapeutic agent, which is capable of modulating or regulating one or more immune function or response.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein “leukemia” refers to broadly progressive, malignant diseases of the blood-forming organs and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “lipophilic”, as used herein, refers to compounds having an affinity for lipids.

As used herein, “liposome” refers to lipid vesicles comprising one or more natural and/or synthetic lipid bilayers surrounding an internal compartment(s). The number of compartments depends on the number of bilayers present. The internal compartment(s) between the lipid bilayers can be aqueous. Liposomes can be substantially spherical. Liposomes can be prepared according to standard techniques known to those skilled in the art. For example, without limitation, suspending a suitable lipid, e.g., phosphatidyl choline, in an aqueous medium followed by sonication of the mixture will result in the formation of liposomes. Alternatively, rapidly mixing a solution of lipid in ethanol-water, for example, by injecting a lipid through a needle into an agitated ethanol-water solution can form lipid vesicles. Liposomes can also be composed of other amphiphilic substances, e.g., sphingomyelin, phosphatidylcholine, phosphatidylethanolamine, phosphatidylserine, and cholesterol or lipids containing poly(ethylene glycol) (PEG).

As used herein, “mammal,” for the purposes of treatments, refers to any animal classified as a mammal, including human, domestic and farm animals, nonhuman primates, and zoo, sports, or pet animals, such as, but not limited to, dogs, horses, cats, and cows.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “melanoma” refers to a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo malignant melanoma, malignant melanoma, nodular melanoma subungal melanoma, and superficial spreading melanoma.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid,” “nucleotide sequence,” and “polynucleotide” can be used interchangeably herein and generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompass such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used interchangeably herein, “operatively linked” and “operably linked” in the context of recombinant or engineered polynucleotide molecules (e.g. DNA and RNA) vectors, and the like refers to the regulatory and other sequences useful for expression, stabilization, replication, and the like of the coding and transcribed non-coding sequences of a nucleic acid that are placed in the nucleic acid molecule in the appropriate positions relative to the coding sequence so as to drive and/or effect expression or other characteristic of the coding sequence or transcribed non-coding sequence. This same term can be applied to the arrangement of coding sequences, non-coding and/or transcription control elements (e.g. promoters, enhancers, and termination elements), and/or selectable markers in an expression vector. “Operatively linked” can also refer to an indirect attachment (i.e. not a direct fusion) of two or more polynucleotide sequences or polypeptides to each other via a linking molecule (also referred to herein as a linker).

As used herein, “organism”, “host”, and “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein, “patient” refers to an organism, host, or subject in need of treatment.

As used herein, “peptide” refers to chains of at least 2 amino acids that are short, relative to a protein or polypeptide.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “plasmid” as used herein refers to a non-chromosomal double-stranded DNA sequence including an intact “replicon” such that the plasmid is replicated in a host cell.

As used herein, “positive control” refers to a “control” that is designed to produce the desired result, provided that all reagents are functioning properly and that the experiment is properly conducted.

As used herein, “preventative” and “prevent” refers to hindering or stopping a disease or condition before it occurs, even if undiagnosed, or while the disease or condition is still in the sub-clinical phase.

As used herein, “polypeptides” or “proteins” refer to amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be required for the structure, function, and regulation of the body's cells, tissues, and organs.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that there are numerous amino acid and peptide analogs which can be incorporated into the disclosed compositions. The opposite stereoisomers of naturally occurring peptides are disclosed, as well as the stereoisomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site-specific way (Thorson et al., Methods in Molec. Biol. 77:43-73 (1991), Zoller, Current Opinion in Biotechnology, 3:348-354 (1992); Ibba, Biotechnology & Genetic Engineering Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994), all of which are herein incorporated by reference at least for material related to amino acid analogs).

Molecules can be produced that resemble polypeptides, but which are not connected via a natural peptide linkage. For example, linkages for amino acids or amino acid analogs can include CH₂NH—, —CH₂S—, —CH₂—CH₂—, —CH═CH—(cis and trans), COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. It is understood that peptide analogs can have more than one atom between the bond atoms, such as b-alanine, g-aminobutyric acid, and the like.

Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, greater ability to cross biological barriers (e.g., gut, blood vessels, blood-brain-barrier), and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference). As used herein, “promoter” can include all sequences capable of driving transcription of a coding or a non-coding sequence. In particular, the term “promoter” as used herein refers to a DNA sequence generally described as the 5′ regulator region of a gene, located proximal to the start codon. The transcription of an adjacent coding sequence(s) is initiated at the promoter region. The term “promoter” also includes fragments of a promoter that are functional in initiating transcription of the gene.

As used herein, “purified” or “purify” are used in reference to a nucleic acid sequence, peptide, or polypeptide that has increased purity relative to the natural environment. A purified compound, compounds, molecules, or other substance can have enhanced, improved, and/or substantially different properties and/or effects as compared to the compound(s) and/or molecules in its natural state.

As used herein, the term “recombinant” or “engineered” generally refer to a non-naturally occurring nucleic acid, nucleic acid construct, or polypeptide. Such non-naturally occurring nucleic acids may include natural nucleic acids that have been modified, for example that have deletions, substitutions, inversions, insertions, etc., and/or combinations of nucleic acid sequences of different origin that are joined using molecular biology technologies (e.g., a nucleic acid sequences encoding a fusion protein (e.g., a protein or polypeptide formed from the combination of two different proteins or protein fragments), the combination of a nucleic acid encoding a polypeptide to a promoter sequence, where the coding sequence and promoter sequence are from different sources or otherwise do not typically occur together naturally (e.g., a nucleic acid and a constitutive promoter), etc. Recombinant or engineered can also refer to the polypeptide encoded by the recombinant nucleic acid. Non-naturally occurring nucleic acids or polypeptides include nucleic acids and polypeptides modified by man.

As used herein, “regeneration” refers to the renewal, re-growth, or restoration of a body or a bodily part, tissue, or substance after injury or as a normal bodily process. In contrast to scarring, tissue regeneration involves the restoration of the tissue to its original structural, functional, and physiological condition. This can also be referred to herein as tissue “complexity”. The restoration can be partial or complete, meaning 10, 20, 30, 40, 50, 60, 70, 80, 90, 100% restoration, or any amount of restoration in between as compared to native or control levels. As an example, in the case of a skin injury, tissue regeneration can involve the restoration of hair follicles, glandular structures, blood vessels, muscle, or fat. In the case of a brain injury, tissue regeneration can involve maintenance or restoration of neurons. As an example, in the case of skin injury, an improvement in tissue regeneration can be assessed by measurements of the volume of fibrous scar tissue to normal regenerated skin as a ratio. As another example, counts can be made of discrete regenerating structures such as regenerating skin glands normalized to the volume of the wound area. As another example, counts of the density of cardiomyocytes can be made in the area of heart normally comprised of scar tissue following the healing of a myocardial infarction. Echocardiography can be used to measure the amount of recovery of cardiac function resulting from the regeneration of muscle cell in this scar tissue. Tissue regeneration can involve the recruitment and differentiation of stem cells and/or progenitor cells to replace the damaged cells. These stem cells can be generated from the exogenous stem cells comprising the tissue engineered composition or be endogenous prompted by the composition to join, fuse or otherwise combine in the regenerative repair process.

As used herein, “sarcoma” refers to a tumor which is made up of a substance like the embryonic connective tissue and is generally composed of closely packed cells embedded in a fibrillar or homogeneous substance. Sarcomas include, for example, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

As used herein, “scar tissue” refers to the fibrous (fibrotic) connective tissue that forms at the site of injury or disease in any tissue of the body, caused by the overproduction of disorganized collagen and other connective tissue proteins, which acts to patch the break in the tissue. Scar tissue may replace injured skin and underlying muscle, damaged heart muscle, or diseased areas of internal organs such as the liver. Dense and thick, it is usually paler than the surrounding tissue because it is poorly supplied with blood, and although it structurally replaces destroyed tissue, it cannot perform the functions of the missing tissue. It is composed of collagenous fibers, which will often restrict normal elasticity in the tissue involved. Scar tissue can limit the range of muscle movement or prevent proper circulation of fluids when affecting the lymphatic or circulatory system. Glial scar tissue following injury to the brain or spinal cord is one of the main obstacles to restoration of neural function following damage to the central nervous system.

As used herein, “separated” refers to the state of being physically divided from the original source or population such that the separated compound, agent, particle, or molecule can no longer be considered part of the original source or population.

As used herein, the term “specific binding” refers to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less. 10⁻⁴ M or less. 10⁻⁵ M or less, 10⁻⁶ M or less, 10⁻⁷ M or less, 10⁻⁸M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some aspects, specific binding can be accomplished by a plurality of weaker interactions (e.g., a plurality of individual interactions, wherein each individual interaction is characterized by a Kd of greater than 10⁻³ M). In some aspects, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, a “stem cell” refers to an undifferentiated cell found among differentiated cells in a tissue or organ, or introduced as part of the tissue engineered composition as described elsewhere herein. The primary roles of stem cells in a living organism are to maintain and repair the tissue in which they are found. It is also recognized that stem cells can exist as cancer stem cells, which can be self-renewing population of transformed cells that can give rise to new tumors and metastases, in cancers that include multiple myeloma and those of the brain, breast, colon, skin, pancreas, lung, prostate and ovaries.

As used herein, “stem cell differentiation” refers to the process whereby an unspecialized cell (e.g., stem cell) acquires the features of a specialized cell such as a skin, neural, heart, liver, or muscle cell.

As used interchangeably herein, “subject,” “individual,” or “patient” refers to a vertebrate organism, such as a mammal (e.g. human). “Subject” can also refer to a cell, a population of cells, a tissue, an organ, or an organism, preferably to human and constituents thereof.

As used herein, “substantially pure” means that an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” refer to an amount (e.g. mass, volume, dosage, concentration, and/or time period) needed to achieve one or more desired result(s). For example, a therapeutically effective amount refers to an amount needed to achieve one or more therapeutic effects.

As used herein, “therapeutic” refers to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating” and “treatment” refer generally to obtaining a desired pharmacological and/or physiological effect. The effect can be, but does not necessarily have to be, prophylactic in terms of preventing or partially preventing a disease, symptom or condition thereof, such as a disease, disorder, condition described in the present application. The effect can be therapeutic in terms of a partial or complete cure of a disease, condition, symptom or adverse effect attributed to the disease, disorder, or condition. The term “treatment” as used herein covers any treatment of a disease or disorder described herein in a subject, particularly a human, and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the term “vector” or “vector system” used in reference to a vehicle used to introduce an exogenous nucleic acid sequence into a cell. A vector may include a DNA molecule, linear or circular (e.g. plasmids), which includes a segment encoding a polypeptide of interest operatively linked to additional segments that provide for its transcription and translation upon introduction into a host cell or host cell organelles. Such additional segments may include promoter and terminator sequences, and may also include one or more origins of replication, one or more selectable markers, an enhancer, a polyadenylation signal, etc. Expression vectors are generally derived from yeast or bacterial genomic or plasmid DNA, or viral DNA, and can contain elements of both. Vector systems can contain one or more vectors or other components.

### Discussion

Non-selective or controllable delivery of therapeutics can result in undesirable or untolerated side effects that prevent the use of many compounds or their use at doses that are greater than desired. Further, some types of compounds are difficult to deliver because the induce immune responses in the subject or are broken down prior to reaching their target cells. An example of such a compound are protein and peptide compounds. These compounds can stimulate an aberrant and undesirable immune reaction, as well as be broken down by endogenous proteases and peptidases. As such, there exists at least these needs for improved delivery compositions and strategies.

With that said, described herein are engineered hemichannels, where the engineered hemichannels can include at least one modified connexin 43 polypeptide that lacks a functional c-terminus and can be opened and/or closed in a selective and/or controlled manner. The engineered hemichannels can be incorporated into vesicles, including but not limited to endosomal vesicles. The endosomal vesicles can be loaded with a cargo compound and/or other agent. The endosomal vesicles containing the engineered hemichannel can be administered to a subject and can be used to deliver a cargo compound and/or other agent to the subject Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Engineered Hemichannels

Described herein are engineered hemichannels. The engineered hemichannels can be composed of a plurality of engineered hemichannel polypeptides. In some aspects, the hemichannel polypeptides can be engineered connexin polypeptides, a family of proteins which are encoded by some different 21 genes in humans and numerous other related connexin, innexin, and pannexin molecules found in humans and other animal species (Sanchez et al., 2019 PMID: 31109150). Thus, in other aspects, engineered hemichannels can comprise connexin, pannexin and innexin hemichannels. Where the hemichannel is composed of engineered connexin polypeptides, the hemichannel can also be referred to as an engineered connexon. The engineered connexin polypeptide can be an engineered connexin 43 polypeptide. The engineered connexin 43 polypeptide can have a non-functional c-terminal region as compared to a wild-type connexin 43 polypeptide (e.g. SEQ ID NO: 1). A functional c-terminal region of a wild-type connexin 43 polypeptide can be responsive to c-terminal regulatory cues, such as oxidative and metabolic stress, voltage, redox potential changes, pH and reactive oxygen species. Loss of a functional c-terminal region of a wild-type connexin 43 polypeptide can also alter channel selectively to the chemical and physical properties of molecules transiting the pore including to properties such as molecular charge, shape, and hydrophobicity.

Hemichannels that are composed of wild-type connexin 43 polypeptides are thus responsive to environmental and other regulatory cues that act on or through the c-terminus of the connexin 43 polypeptide. The engineered hemichannels that contain an engineered connexin 43 polypeptide can be less responsive and/or completely unresponsive to one or more c-terminal regulatory cues. As discussed in greater detail elsewhere herein, the reduced and/or lack of responsiveness to c-terminal regulatory cues, such as pH, can be advantageous and can allow for selective and/or controlled and/or selective passage of a cargo compound and/or other agent through the engineered hemichannel. In some aspects, the engineered connexin 43 polypeptide can have reduced or lack responsiveness to acidic pHs. In some aspects, the engineered connexin 43 polypeptide can have reduced or lack responsiveness to a pH less than 8.5. Thus, in some aspects, the connex 43 polypeptide can have reduced responsiveness or lack of responsiveness to a change in pH to an acidic pH or a pH of less than 8.5. The engineered connexin 43 polypeptide and engineered connexons thereof can be responsive to calcium (e.g. Ca²⁺).

Structurally, the engineered connexin 43 polypeptide can contain a primary amino acid sequence modification (e.g. mutation, insertion, deletion, or combination thereof) that can result in an alteration in the function of the connexin 43 polypeptide. Such modifications are described elsewhere herein in some aspects, the primary amino acid sequence modification occurs such that the engineered connexin 43 polypeptide contains a non-function c-terminal portion as compared to a wild-type connexin 43 polypeptide. Objective assays are described elsewhere herein and are known in the art that can be employed to test if any particular modification to the primary amino acid sequence of a wild-type connexin 43 polypeptide, including but not limited to those described herein, results in an engineered connexin 43 polypeptide that contains a non-functional c-terminal portion and thus are fully described and enabled by this disclosure.

Engineered connexin 43 polypeptides can be generated by any insertion(s), deletion(s) and/or substitution(s) of amino acids within the primary sequence of a wild-type connexin 43 polypeptide (e.g. SEQ ID NO: 1) and can be incorporated into the engineered hemichannels as described elsewhere herein. In a non-limiting example and as detailed elsewhere herein, a serine at position 368 (3368) can be substituted with alanine to render the channel less sensitive pH. D379A, S364P and/or C298A substitutions of a wild-type connexin 43 polypeptide can also form hemichannels in the provided compositions. In other examples, deletions or mutations of a wild-type connexin 43 L2 (SEQ ID NO: 97), JM 1 (SEQ ID NO: 54), JM2 (SEQ ID NO: 55), Src (SEQ ID NO: 88), H2 (SEQ ID NO: 93), and aCT sequences (SEQ ID NOs: 13-47, 49-53, 111, 112, and 133) can also provide hemichannels with the provided properties. Other examples include sequences in the connexin that interact with the C-terminal (CT) such as the N-terminal (NT) or cytoplasmic loop domains (e.g., the L2 domain).

The engineered hemichannels described herein can also be generated by swapping desirable domains between connexins and between connexins and other proteins. For example, a chimeric Cx43 (connexin 43) protein can made be made by substituting Cx26 extracellular loop domains (E-loop) E1 and E2) (underlined and bolded in SEQ ID NO: 2) with the E-loop sequences of Cx43 (underlined and bolded in SEQ ID NO: 1), and can provide an engineered hemichannel with the regulatory properties of Cx26 (SEQ ID NO: 2), but the hemichannel docking specificity of hemichannels composed of wild-type connexin 43.

Engineered Connexin 43 Polypeptides

The engineered hemichannels described herein can be composed of a plurality of engineered connexin 43 polypeptides that can be modified such that the responsiveness of the c-terminal region is altered as compared to a wild-type connexin 43. The engineered hemichannel can be composed of one or more engineered connexin 43 polypeptides that have a c-terminus with altered or modified functionality. In other words, the engineered hemichannel can be composed of one or more engineered connexin 43 polypeptides that have a c-terminus with altered or modified responsiveness to a C-terminal regulatory cues as compared to a wild-type connexin 43 polypeptide as previously discussed. In some aspects, the engineered hemichannels can be composed of one or more engineered connexin 43 polypeptides that lack a functional c-terminus. Stated differently, the engineered hemichannels can be composed of one or more engineered connexin 43 polypeptides that contain a non-functional c-terminus. This is described in greater detail elsewhere herein.

For reference, wild-type connexin 43 polypeptide is composed of four alpha-helical transmembrane domains connected by two extracellular loops and one cytoplasmic loop. Wild-type connexin 43 polypeptide contains an intracellular N- and C-terminus. Wild-type connexin 43 polypeptide has a molecular weight of about 43 kDa. A wild-type connexon can be formed from six connexin 43 polypeptides that form a hemichannel that can be in an open or closed state. The wild-type connexons can form gap junctions between cells when a connexon from one cell adjoins a connexon of an adjacent cell. SEQ ID NO: 1 is an example sequence of a wild-type human connexin 43 polypeptide. Wild-type sequences from other species will instantly be appreciated by one of ordinary skill in the art based on this disclosure.

As described in greater detail below, an engineered connexin 43 polypeptide can include a modified c-terminal region as compared to a wild-type connexin 43. For reference, the sequences provided are made with reference to human sequences, but it will be appreciated by those of ordinary skill in the art that the equivalent sequences encoded by the Gja1/GJA1 gene are expressed in other species (e.g. mouse, rat, monkey, birds, reptiles, amphibians, and fish etc.) and can also be used with the same or equivalent modifications to those described herein.

C-Terminal Modifications

The engineered connexin 43 polypeptides described herein can be modified connexin 43 polypeptides in that they can contain a c-terminus with altered responsiveness to regulatory cues as compared to wild-type connexin 43 as previously described. In some aspects, the engineered connexin 43 polypeptide can contain a non-functional c-terminus. As used herein a “non-functional c-terminus” of a connexin 43 polypeptide can a c-terminus of a connexin 43 polypeptide that has a changed, altered, and/or otherwise modified response to one or more c-terminal regulatory cues as compared to the responsiveness of a wild-type connexin 43. The non-functional c-terminus can have reduced or eliminated response to one or more c-terminal regulatory cue as compared to the responsiveness of the wild-type connexin 43 to the same regulatory cue(s). It is noted that the change in responsiveness to the regulatory cue(s) can be observed when the engineered connexin 43 polypeptide is not oligomerized into an engineered connexon and/or when the engineered connexin 43 polypeptide is oligomerized into an engineered connexon.

The engineered connexin 43 polypeptide can retain the calcium responsive domain (which is not part of the c-terminus region) and thus can be responsive to calcium (e.g. Ca²⁺). Thus, engineered connexons that are composed of engineered connexin 43 polypeptides can be responsive to calcium. In some aspects, the calcium responsiveness can be substantially the same as a wild-type connexin 43 connexon. In some aspects, the calcium responsiveness can be increased as compared to a wild-type connexin 43 connexon. In some aspects, the calcium responsiveness can be reduced as compared to a wild-type connexin 43 connexon.

With reference to SEQ ID NO: 1, the c-terminal region of the wild-type polypeptide can refer to residues 225 through 382. The engineered connexin 43 polypeptides can be generated by deleting one or more of the amino acids in the c-terminal region of the wild-type connexin 43 polypeptide. When two or more amino acids are deleted, the deleted amino acids can be contiguous, be discontiguous, or a combination thereof (some deleted amino acids are contiguous and some are not). The engineered connexin 43 polypeptides can be generated by inserting one or more of the amino acids in the c-terminal region of the wild-type connexin 43 polypeptide. When two or more amino acids are inserted, the inserted amino acids can be contiguous, be discontiguous, or a combination thereof (some inserted amino acids are contiguous and some are not). The engineered connexin 43 polypeptide can be generated by mutating one or more amino acids in the c-terminal region of the wild-type connexin 43 polypeptide. When two or more amino acids are mutated, the mutated amino acids can be contiguous, be discontiguous, or a combination thereof (some inserted amino acids are contiguous and some are not). In some aspects, the engineered connexin 43 can have an amino acid sequence about 50-100% identical to any one of SEQ ID NOs: 3-12.

Deletions

The engineered connexin 43 polypeptide can have an amino acid sequence that can be about 50, 55, 60, 65, 70, 75, 80, 85, 90, 92, 93, 94, 95, 96, 97, 98, 99-100 percent identical to amino acids 1-224 of SEQ ID NO: 1 and have contiguous amino acids 225 to 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, or 382 of SEQ ID NO: 1 deleted.

The engineered connexin 43 polypeptide can have an amino acid sequence that can be about 50, 55, 60, 65, 70, 75, 80, 85, 90, 92, 93, 94, 95, 96, 97, 98, 99-100 percent identical to amino acids 1-224 of SEQ ID NO: 1 and have contiguous amino acids 382 to 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, or 381, of SEQ ID NO: 1 deleted.

The engineered connexin 43 polypeptide can have an amino acid sequence that can be about 50 percent to about 100% identical to amino acids 1-224 of SEQ ID NO: 1 and can include a deletion of any one or more of contiguous or non-contiguous amino acids 225-382 of SEQ ID NO: 1. In some aspects, amino acid residue(s) 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, or any combination thereof of SEQ ID NO: 1 can be deleted in the engineered connexin 43 polypeptide.

In some aspects, the deletions can result in the generation of a peptidase cleavage site in the C-terminus of the engineered connexin 43 polypeptide and form a pro-protein that can be cleaved by a peptidase to result in the final and/or active engineered connexin 43 polypeptide.

Insertions

The engineered connexin 43 polypeptide can have an amino acid sequence that can be about 50-100 percent identical to amino acids 1-224 of SEQ ID NO: 1 and have one or more amino acids inserted between any two amino acids from amino acid residues 225-382 of SEQ ID NO: 1. In some aspects, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more additional amino acids can be inserted between any two amino acid residues in the c-terminus region ranging from amino acid residues 224 and 382 of SEQ ID NO: 1. It is noted that residue 224 is discussed here, but is not necessarily considered part of the c-terminus and included to reference an insertion that can occur between amino acid residue 224 and 225 of SEQ ID NO: 1.

In some aspects, more than one different insertion of one or more amino acids between any two amino acid residues 225-382 of SEQ ID NO: 1 can be made. For illustration, a first insertion can be made between amino acids 228 and 229 and a second can be made between two other amino acid residues (e.g. 301 and 302). The number of different insertions can range from 1 to 50 or more. Where multiple insertions are included, the insertions can be the same. In other words, the same additional amino acid(s) are inserted just at different positions. In other aspects where multiple insertions are included, at least two of the insertions can be different from each other. In other aspects where multiple insertions are included, all insertions are different from each other.

In some aspects, an insertion can be A, I, L, M, V, F, W, Y, N, C, Q, S, T, D, E, R, H, K, G, P or any combination thereof. In some aspects, the insertion(s) can result in the generation of a peptidase cleavage site in the c-terminus of the engineered connexin 43 polypeptide and form a pro-protein that can be cleaved by a peptidase to result in the final and/or active engineered connexin 43 polypeptide.

Mutations

As discussed above, the engineered connexin 43 polypeptide can contain one or more amino acid mutations in the c-terminal region as compared to the wild-type (e.g. SEQ ID NO: 1) connexin 43 polypeptide. Any one or more of the amino acids residues 225-382 can be substituted with any one of amino acids A, I, L, M, V, F, W, Y, N, C, Q, S, T, D, E, R, H, K, G, P that is not the same as the amino acid that it is being substituted for. For example, amino acid 226 can be substituted with any one of A, L, M, V, F, W, Y, N, C, Q, S, T, D, E, R, H, K, G, P but not I. The mutation(s) can render the engineered connexin 43 polypeptide more or less responsive to a c-terminal regulatory cue as previously described.

In some aspects, Serine 368 (S368) can be substituted in the engineered connexin 43 polypeptide with alanine. In some aspects, D379 can be substituted in the engineered connexin 43 polypeptide with alanine. In some aspects, 5365 can be substituted in the engineered connexin 43 polypeptide with proline. In some aspects, C298 can be substituted in the engineered connexin 43 polypeptide with alanine. These substitutions can render the engineered connexin 43 polypeptide (or an engineered connexon containing the engineered connexin 43 polypeptide) less responsive or not responsive to pH, and other connexin 43 C-terminal regulatory cues, as compared to a wild-type connexin 43 polypeptide (or wild-type connexon). In some aspects, the engineered connexin 43 polypeptide can include a 5368A, D379A, E381A, S364P, C298A mutation or any combination thereof.

In some aspects, the mutations can result in the generation of a peptidase cleavage site in the c-terminus of the engineered connexin 43 polypeptide and form a pro-protein that can be cleaved by a peptidase to result in the final and/or active engineered connexin 43 polypeptide.

Post-Translational Modifications

Previously discussed modifications of the wild-type connexin 43 polypeptide included modifications of the polypeptide sequence. The c-terminal region can also or alternatively be modified with a post-translational modification. Sites that often undergo post-translational modification are those that have a functional group that can serve as a nucleophile in the reaction: the hydroxyl groups of serine, threonine, and tyrosine; the amine forms of lysine, arginine, and histidine; the thiolate anion of cysteine; the carboxylates of aspartate and glutamate; and the N- and C-termini. The resulting engineered connexin 43 polypeptide with a post-translational can have reduced or eliminated responsiveness to c-terminal regulatory cues. The post-translational can be phosphorylation of one or more serine, tyrosine, and/or threonine residues in the c-terminal region. Other post-translational modifications resulting in reduced or eliminated responsiveness to c-terminal regulatory cues include amidation, biotinylation, cysteinylation, deamidation, farnesylation, formylation, geranylgeranylation, glutathionylation, glycation, glycosylation, hydroxylation, methylation, mono-ADP-ribosylation, myristoylation, oxidation, palmitoylation, poly(ADP-ribosyl)ation, stearoylation, or sulfation. In another aspect the connexin 43 polypeptide can be subject to proteolytic cleavage by peptidases. For example, peptidases that the connexin 43 polypeptide can be cleaved by include calpains, serine proteases, and MMPs. Site for such peptide cleavage events include locations on Cx43 cleaved by MMP2, MMP7 and MMP9 at between P277 and L278, A357 and 1358 and D379 and L380, as well as multiple calpain cleavage sites between P355 and P375.

Other Polypeptide Region Modifications

As described above, the engineered connexin 43 polypeptide can contain one or more modifications to the c-terminal region, which can in some aspects, alter the responsiveness of the engineered connexin 43 polypeptide (or engineered connexon thereof) to one or more c-terminal regulatory cues. Additionally, the engineered connexin 43 polypeptide can contain one or more modifications to the non-c-terminal region of the polypeptide (e.g. the amino acids equivalent to 1-225 of the wild-type connexin 43 polypeptide (SEQ ID NO: 1). These modifications are discussed here and can be coupled with any of the c-terminal modifications previously discussed.

In some aspects, one or more of the extracellular loop domains can also be substituted in the engineered connexin 43 polypeptide with an extracellular loop domain from another connexin polypeptide. In some aspects, one or more of the extracellular domains of the engineered connexin 43 polypeptide can be substituted with an extracellular domain from a connexin 26 (SEQ ID NO: 2).

Additional Modifications to the Engineered Connexin 43 Polypeptides

The engineered connexin 43 polypeptides can further include one or more additional modifications. The engineered connexin 43 polypeptide can further include one or more reporter proteins (also referred to as selectable markers) operatively linked to an engineered connexin 43 polypeptide described elsewhere herein. Exemplary reporter proteins include but are not limited to β-galactosidase, GUS; fluorescent proteins such as green fluorescent protein (GFP), cyan (CFP), yellow (YFP), red (RFP), luciferase, cell surface proteins and, epitope tags such as but not limited to, e.g. FLAG- and His-tags. The reporter protein can be fused directly to or be linked indirectly via a linking amino acid or peptide to the C- and/or N-terminus of the engineered connexin 43 polypeptide. Other additional polypeptides can include but are not limited to BAD, VSVG, HA, myc, and V5.

Polynucleotides and Vectors

Also described herein are polynucleotides that can, inter alia, encode one or more of the engineered connexin polypeptides described herein. The polynucleotides can be recombinant polynucleotides. The polynucleotides and/or vectors described herein can be generated by any suitable technique such as recombinant polynucleotide techniques and de novo nucleic acid synthesis techniques. The polynucleotides can further include one or more selectable marker (or reporter) genes.

In some aspects, non-coding nucleotides can be placed at the 5′ and/or 3′ end of the polynucleotides encoding an engineered connexin 43 polypeptide as described elsewhere herein without affecting the functional properties of the molecule. A polyadenylation region at the 3′-end of the coding region of a polynucleotide can be included. The polyadenylation region can be derived from an endogenous gene, from a variety of bacterial, animal (e.g. mammalian), and/or plant genes, from T-DNA, or through chemical synthesis. In further aspects, the nucleotides encoding an engineered connexin 43 polypeptide can be conjugated to a nucleic acid encoding a signal or transit (or leader) sequence at the N-terminal end (for example) of the engineered connexin 43 polypeptide that can co-translationally or post-translationally directs transfer of the engineered connexin 43 polypeptide. The polynucleotide sequence can also be altered so that the engineered connexin 43 polypeptide is conjugated or operatively linked to a linker, selectable marker, or other sequence for, post-translational modification, folding, synthesis, purification, and/or identification of the resulting engineered connexin 43 polypeptide. In one aspect, the recombinant polynucleotide sequence can include at least one regulatory sequence operatively linked to the polynucleotide that can encode a connexin 43 polypeptide described herein.

Methods of expressing polypeptides from polynucleotides are generally known in the art. Further, an appropriate or desired nucleotide sequence corresponding to a polypeptide disclosed herein, will be appreciated by those of skill in the art in view of the generally available tools and techniques known in the art to determine appropriate nucleotide sequences to express polypeptides. Such tools include various software and web-based programs and tools capable of generating nucleotides sequences that correspond to or otherwise encode a given polypeptide.

Also provided herein are vectors that can contain one or more of the polynucleotides or described herein. In aspects, the vector can contain one or more polynucleotides that can encode an engineered connexin 43 polypeptide. The vectors can be useful in producing bacterial, fungal, yeast, plant cells (including but not limited to grapefruit cells), animal cells, and transgenic animals that can express an engineered connexin polypeptide and/or engineered connexon thereof. Within the scope of this disclosure are vectors containing one or more of the polynucleotide sequences described herein.

The polynucleotide can be codon optimized for expression in a specific cell-type and/or subject type. An example of a codon optimized sequence, is in this instance a sequence optimized for expression in a eukaryote, e.g., humans (i.e. being optimized for expression in a human or human cell), or for another eukaryote, animal or mammal as herein discussed is within the ambit of the skilled artisan. It will be appreciated that other examples are possible and codon optimization for a host species other than human, or for codon optimization for specific organs is known. In some embodiments, an enzyme coding sequence encoding a hem ichannel (or a peptide cargo compound) is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in cod on usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

Regulatory Elements

In aspects, the polynucleotides described herein can include one or more regulatory elements that can be operatively linked to the polynucleotide that can encode a polypeptide capable of allosterically interaction with a polypeptide upon sequence-specific recognition of a target sequence that are described elsewhere herein. The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1a promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., engineered connexin polypeptides, proteins, enzymes, mutant forms thereof, fusion proteins thereof, etc.). With regards to regulatory sequences, mention is made of U.S. patent application Ser. No. 10/491,026, the contents of which are incorporated by reference herein in their entirety. With regards to promoters, mention is made of PCT publication WO 2011/028929 and U.S. application Ser. No. 12/511,940, the contents of which are incorporated by reference herein in their entirety. In an embodiment of the vector for delivering an effector protein, the minimal promoter is the Mecp2 promoter, tRNA promoter, or U6. In a further embodiment, the minimal promoter is tissue specific.

To express a polynucleotide that encodes an engineered connexin 43 polypeptide in a cell, the polynucleotide can be combined (e.g., in a vector) with transcriptional and/or translational initiation regulatory sequences, e.g. promoters, that direct the transcription of the gene and/or translation of the encoded protein in a cell. In some aspects a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In other aspects, tissue (or cell)-specific promoters or inducible/conditional promoters may be employed to direct expression of the polynucleotide in a specific cell type, under certain environmental conditions, and/or during a specific state of development. Suitable tissue specific promoters can include, but are not limited to, liver specific promoters (e.g. APOA2, SERPIN A1 (hAAT), CYP3A4, and MIR122), pancreatic cell promoters (e.g. INS, IRS2, Pdx1, Alx3, Ppy), cardiac specific promoters (e.g. Myh6 (alpha MHC), MYL2 (MLC-2v), TN13 (cTnl), NPPA (ANF), Slc8a1 (Ncx1)), central nervous system cell promoters (SYN1, GFAP, INA, NES, MOBP, MBP, TH, FOXA2 (HNF3 beta)), skin cell specific promoters (e.g. FLG, K14, TGM3), immune cell specific promoters, (e.g. ITGAM, CD43 promoter, CD14 promoter, CD45 promoter, CD68 promoter), urogenital cell specific promoters (e.g. Pbsn, Upk2, Sbp, Fer1I4), endothelial cell specific promoters (e.g. ENG), pluripotent and embryonic germ layer cell specific promoters (e.g. Oct4, NANOG, Synthetic Oct4, T brachyury, NES, SOX17, FOXA2, MIR122), and muscle cell specific promoter (e.g. Desmin). Other tissue and/or cell specific promoters are generally known in the art and are within the scope of this disclosure.

Inducible/conditional promoters can be positively inducible/conditional promoters (e.g. a promoter that activates transcription of the polynucleotide upon appropriate interaction with an activated activator, or an inducer (compound, environmental condition, or other stimulus) or a negative/conditional inducible promoter (e.g. a promoter that is repressed (e.g. bound by a repressor) until the repressor condition of the promotor is removed (e.g. inducer binds a repressor bound to the promoter stimulating release of the promoter by the repressor or removal of a chemical repressor from the promoter environment). The inducer can be a compound, compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

In order to ensure appropriate expression in a plant cell, the components of the CRISPR-Cas system described herein are typically placed under control of a plant promoter, i.e. a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. “Regulated promoter” refers to promoters that direct gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In particular embodiments, one or more of the engineered connexins are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed.

Examples of promoters that are inducible and that allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc), or light inducible systems (Phytochrome, LOV domains, or cryptochrome)., such as a Light Inducible Transcriptional Effector (LITE) that direct changes in transcriptional activity in a sequence-specific manner. The components of a light inducible system may include an engineered connexin, a light-responsive cytochrome heterodimer (e.g. from Arabidopsis thaliana), and a transcriptional activation/repression domain.

In particular embodiments, transient or inducible expression can be achieved by using, for example, chemical-regulated promotors, i.e. whereby the application of an exogenous chemical induces gene expression. Modulating of gene expression can also be obtained by a chemical-repressible promoter, where application of the chemical represses gene expression. Chemical-inducible promoters include, but are not limited to, the maize 1n2-2 promoter, activated by benzene sulfonamide herbicide safeners (De Veylder et al., (1997) Plant Cell Physiol 38:568-77), the maize GST promoter (GST-II-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

The expression system can include elements for translocation to and/or expression in a specific plant organelle.

Selectable markers and Tags

One or more of the polypeptides can be operably linked, fused to, or otherwise modified to include (such inserted between two amino acids between the N- and C-terminus of the polypeptide) a selectable marker, affinity, or other protein tag. It will be appreciated that the polynucleotide encoding such selectable markers or tags can be incorporated into a polynucleotide encoding one or more of the engineered connexins or other polypeptides described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure. Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; fluorescence tags, such as GFP and mCherry; protein tags that may allow specific enzymatic modification (such as biotinylation by biotin ligase) or chemical modification (such as reaction with FlAsH-EDT2 for fluorescence imaging). Selectable markers and tags can be operably linked to one or more components of the engineered connexins or other polypeptides described herein via suitable linker, such as a glycine or glycine serine linkers as short as GS or GG up to (GGGGG)₃ or (GGGGS)₃. Other suitable linkers are described elsewhere herein.

Examples of additional selectable markers include, but are not limited to, DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); the generation of new primer sites for PCR (e.g., the juxtaposition of two DNA sequences not previously juxtaposed), the inclusion of DNA sequences not acted upon or acted upon by a restriction endonuclease or other DNA modifying enzyme, chemical, etc.; epitope tags (e.g. GFP, FLAG- and His-tags), and, the inclusion of a DNA sequences required for a specific modification (e.g., methylation) that allows its identification. Other suitable markers will be appreciated by those of skill in the art.

Vectors and Vector Systems

In general, and throughout this specification, the term “vector” refers to a nucleic acid molecule capable of transporting another nucleic acid to which it has been linked. It is a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” and “operatively-linked are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells.

With regards to recombination and cloning methods, mention is made of U.S. patent application Ser. No. 10/815,730, published Sep. 2, 2004 as US 2004-0171156 A1, the contents of which are herein incorporated by reference in their entirety.

Advantageous vectors include lentiviruses and adeno-associated viruses, and types of such vectors can also be selected for targeting particular types of cells.

In particular embodiments, use is made of bicistronic vectors for cargo compounds and hem ichannel polypeptide. In some aspects, expression of the cargo compound and/or hemichannel polypeptide driven by the CBh promoter. The RNA may preferably be driven by a Pol III promoter, such as a U6 promoter. In some aspects, the two are combined.

Vectors can be designed for expression of cargo compound and/or hemichannel transcripts (e.g. nucleic acid transcripts, proteins, or enzymes) in prokaryotic or eukaryotic cells. For example, cargo compound and/or hem ichannel can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89). In some embodiments, a vector is a yeast expression vector. Examples of vectors for expression in yeast Saccharomyces cerivisae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors may contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2p plasmids, yeast integrative plasmids, yeast replicative plasmids, shuttle vectors, and episomal plasmids.

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneiji, et al., 1983. Ce/133: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other aspects can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of a cargo compound and/or hemichannel so as to drive expression of the one or more elements of the cargo compound and/or hemichannel.

In some embodiments, one or more vectors driving expression of one or more elements of a cargo compound and/or hem ichannel are introduced into a host cell such that expression of the elements of the cargo compound and/or hemichannel direct formation of a cargo compound and/or hemichannel. For example, cargo compound and/or hemichannel could each be operably linked to separate regulatory elements on separate vectors. RNA(s) of the cargo compound and/or hemichannel can be delivered to an animal or mammal, e.g., an animal or mammal that constitutively or inducibly or conditionally expresses cargo compound and/or hemichannel or an exosome that incorporates one or both; or an animal or mammal that is otherwise expressing cargo compound and/or hemichannel or has cells and/or exosomes containing cargo compound and/or hemichannel(s), such as by way of prior administration thereto of a vector or vectors that code for and express in vivo cargo compound and/or hemichannel(s). Alternatively, two or more of the elements expressed from the same or different regulatory elements, may be combined in a single vector, with one or more additional vectors providing any components of the system not included in the first vector. Cargo compounds and/or hemichannels that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding cargo compound and/or hem ichannel, embedded within one or more intron sequences (e.g., each in a different intron, two or more in at least one intron, or all in a single intron). In some embodiments, the cargo compound and/or hem ichannel can be operably linked to and expressed from the same promoter.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors.

In some aspects, a vector capable of expressing a cargo compound and/or hemichannel polynucleotide in a cell can be composed of or contain a minimal promoter operably linked to a polynucleotide sequence encoding the cargo compound and/or hem ichannel and a second minimal promoter operably linked to a polynucleotide sequence encoding at least one engineered conexin polynucleotide, and optionally a cargo molecule polynucleotide, wherein the length of the vector sequence comprising the minimal promoters and polynucleotide sequences is less than 4.4 Kb. In an embodiment, the vector can be a viral vector. In aspects, the viral vector is an is an adeno-associated virus (AAV) or an adenovirus vector.

Viral Vectors

In aspects, the one or more of the polynucleotides described herein can be incorporated into a viral vector. Viral vectors and systems thereof can be useful for producing viral particles for delivery of and/or expression of one or more components of the engineered vesicle system described herein. The viral vector can be part of a viral vector system involving multiple vectors to increase the safety of these systems. The viral vectors can be retro viral vectors. The viral vectors can be lentiviral vectors. Other aspects of viral vectors and viral particles produce therefrom are described elsewhere herein. In some aspects, the viral vectors are configured to produce replication incompetent viral particles for improved safety of these systems.

Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the expression of the engineered connexins described and/or cargo molecules described herein can include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. A retrovirus can also be engineered to allow for conditional expression of the inserted transgene, such that only certain cell types are infected by the lentivirus.

Adeno Associated Virus Vectors

One or more cargo compound and/or hemichannel polynucleotides can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue or cell of interest.

In terms of in vivo delivery, AAV is advantageous over other viral vectors for a couple of reasons such as low toxicity (this may be due to the purification method not requiring ultra-centrifugation of cell particles that can activate the immune response) and a low probability of causing insertional mutagenesis because it doesn't integrate into the host genome.

rAAV vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

As to AAV, the AAV can be AAV1, AAV2, AAV5 or any combination thereof. One can select the AAV of the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008).

Lentiviral Vectors

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types. Advantages of using a lentiviral approach can include the ability to transduce or infect non-dividing cells and can typically produce high viral titers, which can increase efficiency or efficacy of production and delivery.

In some embodiments, an HIV-based lentiviral vector system can be used. In some embodiments, a FIV-based lentiviral vector system can be used.

In embodiments, minimal non-primate lentiviral vectors based on the equine infectious anemia virus (EIAV) are also contemplated (see, e.g., Balagaan, J Gene Med 2006; 8: 275-285). In another embodiment, RetinoStat®, an equine infectious anemia virus-based lentiviral gene therapy vector that expresses angiostatic proteins endostatin and angiostatin that is delivered via a subretinal injection for the treatment of the web form of age-related macular degeneration is also contemplated (see, e.g., Binley et al., HUMAN GENE THERAPY 23:980-991 (September 2012)) and this vector may be modified for the hemichannel/exosome system described herein.

In another embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the engineered vesicle system and/or cargo molecules described herein.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, U520090111106 and U.S. Pat. No. 7,259,015. Any of these systems or a variant thereof can be used to deliver a cargo polynucleotide and/or hemichannel polynucleotide to a cell. Other adaptations of lentiviral vectors for delivery of a cargo polynucleotide and/or hemichannel polynucleotide to a cell are generally known in the art.

Cells and Transgenic Plants and Animals

Also described herein are cells that can be transformed with one or more polynucleotides (including vectors) described herein. The cells that are transformed with one or more polynucleotides described can express one or more engineered connexon 43 polypeptides described herein. The cells can be bacterial, yeast, fungi, insect, plant, or mammalian. Suitable mammalian cells include, but are not limited to, HeLa, MEFs, CHOs, HEK-293, N2A, MDCK, and variant cells, BHK-21 cells, myeloma cells, iPS or other pluripotent stem cells (which can be autologous or heterologous), mesenchymal stem cells, liver stem cells, mammary stem cells, pancreatic stem cells, neuronal stem cells, cancer stem cells, embryonic stem cells. The cells can be totipotent, pluripotent, multipotent, or oligopotent. In some aspects the mammalian cells can produce a native connexin 43 and/or connexon thereof. In some aspects the mammalian cells can do not produce a native connexin 43 and/or connexon. In some aspects, the cells can be those that have specific or select abilities or characteristics, such as penetration into certain tissues, such as skin, eye, brain, liver, heart, muscle, intestine, and pancreas. As discussed elsewhere herein engineered vesicles that can be produced from these cells can also have the specific or select ability or characteristic of the cell from which they are generated. Such cells include, but are not limited to, human umbilical cord blood mesenchymal stem cells (can permeate unbroken skin), tumor cells that have metastasized to the brain (e.g. those that metastasize from breast cancer) (which can pass the blood brain barrier), uveal melanomas (can permeate the blood eye barrier) Other suitable mammalian cells are generally known in the art. Techniques for transforming cells are generally known in the art and can include, but are not limited to, transfection, electroporation, gene gun, and virus and/or viral vector mediated transduction. The cells can be useful in the production of the recombinant polypeptides described herein. The cells can be used for the production of engineered vesicles, such as engineered extracellular vesicles, that can express an engineered connexon that can include one or more connexin 43 polypeptides described herein. Discussion of vesicle production is discussed elsewhere herein.

Other exogenous proteins can be co-expressed with the one or more connexin 43 polypeptides described herein. Other proteins include, but are not limited to various proteases, kinases, phosphatases, glycosylases, and methylases. In some aspects, co-expression of a protein, such as a protease or kinase, can facilitate production of the engineered connexin 43 polypeptide.

Any suitable methods for nucleic acid delivery for transformation of a cell, as described herein or as would be known to one of ordinary skill in the art. In addition to those described elsewhere herein, such methods can include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) can be stably or transiently transformed.

Also provided herein are transgenic animals, including but not limited to mice, chickens, bovine, ovine, goats, pigs, and other mammals that express one or more polypeptides and/or engineered connexons described herein. Methods for producing transgenic animals that can express recombinant polypeptides are generally known in the art and will be appreciated by those of skill in the art.

The polynucleotide sequences and vectors described above can be used to produce transgenic plants that can express an engineered connexin polypeptide and/or engineered hem ichannel described herein. The present disclosure includes transgenic plants having one or more cells where the one or more cells contain any of the recombinant polynucleotides or vectors previously described that have DNA sequences encoding an engineered connexin polypeptide and/or engineered hemichannel described herein. The transgenic plant can be made from any suitable plant species or variety including, but not limited to Arabidopsis, rice, wheat, corn, maize, tobacco, soybean, Brassicas, tomato, potato, alfalfa, sugarcane, and/or sorghum.

Techniques for transforming a wide variety of plant cells with vectors or naked nucleic acids are well known in the art and described in the technical and scientific literature. See, for example, Weising et al. Ann. Rev. Genet. 1988, 22:421-477. For example, the vector or naked nucleic acid may be introduced directly into the genomic DNA of a plant cell using techniques such as, but not limited to, electroporation and microinjection of plant cell protoplasts, or the recombinant nucleic acid can be introduced directly to plant tissue using ballistic methods, such as DNA particle bombardment.

Microinjection techniques are known in the art and well described in the scientific and patent literature. The introduction of a recombinant nucleic acid using polyethylene glycol precipitation is described in Paszkowski et al. EMBO J. 1984, 3:2717-2722. Electroporation techniques are described in Fromm et al. Proc. Natl. Acad. Sci. USA. 1985, 82:5824. Ballistic transformation techniques are described in Klein et al. Nature. 1987, 327:70-73. The recombinant nucleic acid may also be combined with suitable T-DNA flanking regions and introduced into a conventional Agrobacterium tumefaciens host vector, or other suitable vector. The virulence functions of the Agrobacterium tumefaciens host will direct the insertion of the recombinant nucleic acid including the exogenous nucleic acid and adjacent marker into the plant cell DNA when the cell is infected by the bacteria. Agrobacterium tumefaciens-mediated transformation techniques, including disarming and use of binary vectors, are known to those of skill in the art and are well described in the scientific literature. See, for example, Horsch et al. Science. 1984, 233:496-498; Fraley et al. Proc. Natl. Acad. Sci. USA. 1983, 80:4803; and Gene Transfer to Plants, Potrykus, ed., Springer-Verlag, Berlin, 1995.

A further method for introduction of the vector or recombinant nucleic acid into a plant cell is by transformation of plant cell protoplasts (stable or transient). Plant protoplasts are enclosed only by a plasma membrane and will therefore more readily take up macromolecules like exogenous DNA. These engineered protoplasts can be capable of regenerating whole plants. Suitable methods for introducing exogenous DNA into plant cell protoplasts include electroporation and polyethylene glycol (PEG) transformation. Following electroporation, transformed cells are identified by growth on appropriate medium containing a selective agent.

The presence and copy number of the exogenous nucleic acid in a transgenic plant can be determined using methods well known in the art, e.g., Southern blotting analysis. Expression of the exogenous root PV phytase nucleic acid or antisense nucleic acid in a transgenic plant may be confirmed by detecting an increase or decrease of mRNA or the root PV phytase polypeptide in the transgenic plant. Methods for detecting and quantifying mRNA or proteins are well known in the art.

Transformed plant cells that are derived by any of the above transformation techniques, or other techniques now known or later developed, can be cultured to regenerate a whole plant. In aspects, such regeneration techniques may rely on manipulation of certain phytohormones in a tissue culture growth medium, typically relying on a biocide or herbicide selectable marker that has been introduced together with the exogenous nucleic acid. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. Plant Phys. 1987, 38:467-486.

Once the engineered connexin polypeptide and/or engineered hemichannel described herein. has been confirmed to be stably incorporated in the genome of a transgenic plant, it can be introduced into other plants by sexual crossing. Any of a number of standard breeding techniques can be used, depending upon the species to be crossed.

Methods of Making the Engineered Connexin 43 Polypeptides

The engineered connexin polypeptides described herein can be made by any suitable method. Suitable methods include, but are not limited to, various recombinant polynucleotide and protein expression techniques, which will be appreciated by those of ordinary skill in the art, de novo peptide, polypeptide techniques. In some aspects, an engineered connexin 43 polypeptide can be generated by cleaving a wild-type connexin 43 polypeptide using a suitable enzyme to truncate all or a portion of the c-terminal region. The suitable enzyme can be a protease. The protease can be a peptidase. Suitable enzymes include, but are not limited to, MMP2, MMP7, MMP9, serine proteases, and calpains. In other aspects, cells that generate endosomal vesicles that can contain a wild-type connexin 43 connexon and/or wild-type connexin can be exposed to specific conditions (e.g. ischemia, hypoxia, glucose deprivation, exposure to a compound or chemical) that can result in production of a connexin 43 having a modified (e.g. truncated, phosphorylated, or other chemical modification of the wild-type connexin 43) c-terminal region or truncate (or otherwise modify) an already produced connexin 43 in the c-terminal region).

In some aspects, the engineered connexin 43 polypeptide can include a c-terminus (CT) deletion as compared to a wild-type connexin 43 polypeptide (e.g. SEQ ID NO: 1) that can be achieved by activation or use of endogenous or exogenous peptidases or other chemical means that enable controlled removal of the connexin CT. For example, normal non-mutated Cx43 contains numerous consensus sites for peptidase cleavage including those mediated by MMP2, MMP7, MM9 (PMID: 16769909; PMID: 26424967), serine proteases (PMID: 4009696) and calpains (PMID:28065778). The provided composition can also be generated by exposing cells or tissues producing EVs to certain conditions, including for example ischemia, hypoxia, glucose deprivation, drug or chemical treatment resulting in desired modification to hemichannel activity, including for example the cleavage of the connexin CT, phosphorylation of serine, tyrosine, and threonine residues and other chemical modifications.

Deletion or chemical modification of the connexin may be achieved in any stage prior to or during extracellular or engineered vesical (EV) (e.g. an endosomal vesicle) biogenesis, such that the provided EVs can be loaded with and deliver a cargo in the desired controlled manner as is discussed in greater detail elsewhere herein. In one non-limiting example, a wild-type connexin 43 c-terminus can be cleaved by direct provision or activation of exogenous or endogenous peptidases to generate an engineered connexin 43 polypeptide. In another non-limiting example, cells can be engineered to co-express a specific peptidase that is capable of mediating cleavage of a wild-type connexin 43 c-terminus that can be turned on or off using a genetic control mechanism (e.g., a Tet-on promoter), a drug, other compound, and/or other stimulus. A new peptidase cleavage sequence not present in wild-type connexin 43 can be also be genetically introduced into the sequence of the connexin to enable control over the specificity and timing of the connexin deletion event.

EVs containing one or more engineered hemichannels described herein can be used to control and optimize uptake, transport, and/or delivery of the cargo molecules (e.g. therapeutic molecules). This is discussed in greater detail elsewhere herein.

Engineered Hemichannels Containing a Connexin 43 Polypeptide

Described herein are engineered hemichannels that can be composed of one or more engineered connexins described herein. In some aspects the engineered hemichannels can include one or more engineered connexin 43 polypeptides. As previously discussed, the engineered connexin 43 polypeptides can form and be included in an engineered connexon. The engineered connexon can contain 6 engineered connexin 43 polypeptides as described herein. In some aspects, the engineered hemichannel can contain 3, 4, 5, 6, 7, 8, 9, 10, 11, 12, 13, 14, 15, 16, 17, 18, 19, 20, or more engineered connexin 43 polypeptides as described elsewhere herein. In some aspects, the engineered connexin 43 polypeptides are the same engineered connexin 43 polypeptides. In some aspects, at least two of the engineered connexin 43 polypeptides are different from each other. In some aspects, each of the engineered connexin 43 polypeptides in the engineered connexon can be different from each other. In another aspect, the engineered connexon can be heteromers and homomers of Cx43 (connexin 43) and/or other connexins including but not limited to Cx40 (encoded by Gja5/GJA5), Cx45 (encoded by Gja7/GJA7), Cx37 (encoded by Gja4/GJA4), Cx30 (Gjb6/GJB6), Cx36 (encoded by Gja9/GJA9), Cx46 (encoded by Gja4/GJA4), Cx47(Gjc2/GJC2), Cx50 (encoded by Gja8/GJA8), Cx32 (encoded by Gjb1/GJB1), and Cx26 (encoded by Gjb2/GJB2) or variants of Cx43 or these connexins, as a non-limiting example, Cx43 and Cx43 fused to GFP. The ratios of these connexins in the subunit can be varied. In some aspects, the first connexin to second connexin type can range from 1:5 to 5:1. By way of a non-limiting example, in some aspects the ratios of the connexins can be varied from 5 connexin 43 polypeptide to 5 connexin 43-GFP polypeptides, to 1 connexin 43 polypeptide to 6 connexin 43-GFP polypeptides, 5 connexin 43 polypeptide to 5 Cx40 polypeptides, 5 connexin 43 polypeptides to 1 connexin 40-GFP polypeptide and so one—different heteromeric Cx43-containing connexons having different desirable properties.

The connexin 43 polypeptides can form an engineered connexon that can be incorporated into cell-produced vesicles (such as an EV) by cell machinery (e.g. endoplasmic reticulum) during vesicle production via a cell. As described in greater detail elsewhere herein, a cell can be engineered to express one or more of the engineered connexin 43 polypeptides, which can be incorporated into a cell-produced vesicle (including, but not limited to an extracellular vesicle). In other aspects, synthetic membrane vesicles can be produced absent a cell that can spontaneously form under appropriate conditions and can incorporate engineered connexin 43 polypeptides into the membrane of the vesicles as engineered connexons that can span the membrane of the synthetic vesicles. Thus, the engineered hemichannels described herein can be embedded in exosomes (e.g., exosomes isolated from milk) or exosome-mimicking lipid bilayers via cell-free synthesis using translation of plasmids encoding a connexin (e.g., Cx43), innexins or pannexins in the presence of exosomal or exosome like particles. The integration of such denovo synthesized hemichannel-comprising molecules can result in integrated and functionally active HCs in exosomes. This is discussed in greater detail elsewhere herein.

The engineered connexon containing engineered connexin 43 polypeptides can be controllably and selectively responsive to a c-regulatory cue. In some aspects, engineered connexon containing the engineered connexin 43 polypeptides has reduced or no responsiveness to pH, voltage, oxidative and metabolic stress, redox potential changes, pH and reactive oxygen species, as well as the chemical and physical properties of molecules transiting the pore, as compared to a wild-type connexon composed of wild-type connexin 43 polypeptides.

The engineered hemichannels or connexons containing one or more engineered connexin 43 polypeptides can be responsive to calcium. In some aspects, the engineered hemichannels or connexons containing one or more engineered connexin 43 polypeptides can be responsive to environmental calcium concentrations. In some aspects, the response to calcium of the engineered hemichannels or connexons containing one or more engineered connexin 43 polypeptides can be substantially the same as compared to wild-type connexon 43 (a wild-type connexon composed of six wild-type connexon 43 polypeptides). In some aspects, the response of the engineered hemichannels or connexons containing one or more engineered connexin 43 polypeptides to calcium can be increased as compared to wild-type connexon 43. In some aspects, the response to calcium of the engineered hemichannels or connexons containing one or more engineered connexin 43 polypeptides can be present but reduced as compared to wild-type connexon 43. As previously discussed, the engineered hemichannels or connexons containing one or more engineered connexin polypeptides can have an altered response to a c-terminal regulatory signal.

Engineered Vesicles

As discussed elsewhere herein, the engineered connexin 43 polypeptides can form engineered connexons. The engineered connexons can be incorporated into a membrane of a vesicle to form an engineered vesicle. Engineered vesicle is also abbreviated as “EV” herein. In some aspects, the engineered vesicle can be isolated from milk or be made from milk or a milk product (also referred to herein as “milk-based EVs”. In some aspects of milk-based EVs, the milk-based EV can include one or more engineered connexin 43 polypeptides and/or connexons thereof. In other aspects, of the milk-based EVs do not contain any engineered connexin 43 polypeptides. The membrane can be a lipid bilayer. The engineered vesicle can be an engineered liposome. In some aspects the engineered vesicle can be a polymer some. Polymersomes can be vesicles that can be composed of polymers, such as amphiphilic polymers (such as block copolymers). Polymersomes can be of any suitable dimension such as those stated elsewhere herein. The engineered vesicle can be an engineered extracellular vesicle. The engineered extracellular vesicle can be an engineered exosome. The engineered vesicle can be an engineered microvesicle. The engineered connexon that can contain engineered connexin 43 polypeptides can be integrated with the engineered vesicle membrane. The engineered connexon can span the engineered vesicle membrane such that when open, the engineered connexon forms a pore in the engineered vesicle membrane. The engineered connexon can also exist as in a closed state and not form a pore.

In some aspects, the engineered vesicle can be a milk-based exosome. As previously discussed, the milk-based exosome can optionally include one or more engineered connexin 43 polypeptides described elsewhere herein. Milk based-exosomes are exosomes produced by mammary tissue or cells from mammals and excreted in milk. They can be isolated using centrifugation methods, discussed and demonstrated elsewhere herein. In some aspects, in preparation of milk exosomes care, must be taken with the other constituents of milk. For example, casein can be caused to precipitate out of solution, aggregating to form a dense and insoluble product that can enmesh EVs and prevent their efficient isolation. Thus, in some aspects care must be taken to remove casein with care to prevent EV loss using methods known to those skilled in the art. The prompts of such precipitation include acidity, temperature, calcium concentration, exposure to solutions such as ethanol and so on. In some aspects, they are produced from a transgenic animal engineered to express a cargo compound and/or hemichannel as described elsewhere herein from their mammary tissue under control of a mammary specific promoter. Thus, in some aspects, milk-based engineered exosomes can be produced by transgenic animals that can include one or more engineered hemichannels. In short, the transgenic animal can be a mammal engineered to express the engineered connexon(s) and produce the engineered connexon in a cell, e.g. a mammary cell, capable of producing a milk-EV that integrates the one or more of the engineered connexon(s) described herein. Any suitable method of making a transgenic animal (e.g. a mammal) can be used. Methods of making transgenic mammals are generally known in the art.

In some aspects, the milk-based engineered exosomes can be produced via a cell-free method that can include inclusion of exosomal or other vesical membrane components as well as engineered connexon(s) described herein, and optimally, milk-based connexon(s) also described elsewhere herein. The engineered exosome or vesicle can self assemble from the components and integrate the engineered connexon(s) and optionally the milk-based connexon(s) into the vesicle membrane.

In some aspects, the engineered vesicles produced can also contain one or more cargo peptide and/or polynucleotides. The engineered exosomes can then be harvested from milk using an appropriate method (e.g. a centrifugation based-method). In other aspects, isolated and/or engineered EVs can be added to milk or a milk product to afford the benefits that EVs can derive from suspension in this media during storage, loading, drug formulation or delivery to a patient. Such benefits can include association and protection by casein and its byproducts during milk EV transit and uptake from the gut.

The pore permeability can be dependent on the number of engineered connexin polypeptides in the engineered connexon. The pore can be varied depending on the exact engineered connexin polypeptides incorporated in the engineered connexon. The pore can also vary depending on stimulus and the specific responsiveness of the engineered connexon to that stimulus. An engineered connexon can assume one open configuration in response to a first stimulus and assume a different open configuration in response to a second stimulus. Thus, the engineered connexon can have a first permeability that is associated with the response to the first stimulus and can have a second permeability that is associated with the response to the second stimulus. It will be appreciated that this can be the same for additional stimuli. The permeability can be designed by specific configuration and design of the engineered connexon and/or configuration and design of the engineered connexin polypeptides that are included in the engineered connexon. In aspects, unitary permeability can range from about 0 (which is also referred to herein as the closed position) to about 10⁻⁴ cm²s⁻¹. The engineered connexin polypeptides in the engineered connexon may also assume different conductance substrates that may vary between unitary conductances of between 0 and 400 pS.

Engineered vesicles can contain any number of engineered hemichannels or connexons described herein, such as engineered connexons. In some aspects, the engineered vesicles can contain wild-type or natural connexons or other natural hemichannels in addition to an engineered connexon. The type of engineered connexons present in the vesicle membrane can be the same. In some aspects the vesicle membrane can incorporate 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more of engineered hemichannels.

The engineered vesicle can be substantially spherical. The diameter of the engineered vesicle can range from about 10 nm to about 5 μm or more. The diameter of the engineered vesicle can range from about 10 nm to about 20, 30, 40, 50, 60, 70, 80, 90, 100, 110, 120, 130, 140, 150, 160, 170, 180, 190, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 525, 550, 575, 600 625, 650, 675, 700, 725, 750, 775, 800, 900, 925, 950, 975, 1000, 1050, 1100, 1150, 1200, 1250, 1300, 1350, 1400, 1450, 1500, 1550, 1600, 1650, 1700, 1750, 1800, 1850, 1900, 1950, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900 to about 50000 nm.

The engineered vesicle can include one or more targeting moiety. The targeting moiety can be attached or otherwise integrated with the outer surface or membrane of the engineered vesicle. Suitable targeting moieties can be, without limitation, an antibody or fragment thereof, an aptamer, a cell surface receptor or other ligand, and connexins or connexons. In some aspects, the targeting moiety can be a connexon (natural or engineered connexon) present in the engineered vesicle, which can be capable of forming specific homotypic and heterotypic interactions with the extracellular docking motifs of certain other connexins and/or connexons present on the cell surface of a target cell. In some aspects, the targeting molecule comprises an antibody or fragment thereof, a polypeptide, a dendrimer, an aptamer, an oligomer or a small molecule. In particular aspects, the targeting moiety can have an affinity for a receptor expressed in cancer cells. For example, the targeting moiety can bind to human epidermal growth factor receptor (EGFR), vascular endothelial growth factor receptor, folic acid receptor, melanocyte stimulating hormone receptor, integrin avb3, integrin avb5, transferrin receptor, interleukin receptors, lectins, insulin-like growth factor receptor, hepatocyte growth factor receptor or basic fibroblast growth factor receptor. In some aspects, the antibody fragment is an EGFR single-domain antibody fragment. Other suitable targeting moieties are known in the art. See also, Senter, et al., Bioconjugate Chem., 2:447-451, (1991); Bagshawe, K. D., Br. J. Cancer, 60:275-281, (1989); Bagshawe, et al., Br, J, Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)).

The targeting moiety can exploit receptor-mediated, magnetic directing, and cell-mediated drug delivery systems. For example, receptor mediated targeting may be exploited through the ligands for the transferrin receptor (see Tortorella S, The Significance of Transferrin Receptors in Oncology: the Development of Functional Nano-Based Drug Delivery Systems, Curr Drug Deliv. 2014 Jan. 5), the folate receptor (see Saul, J M, Controlled targeting of liposomal doxorubicin via the folate receptor, in vitro, Journal of Controlled Release 92 (2003) 49-67), IL-13 receptor, the epidermal growth factor receptor (EGF-R), the choline receptor (see Li J, Choline transporter-targeting and co-delivery system for glioma therapy, Biomaterials, 2013 December; 34(36):9142-8) to name a few. Cell surface receptors for malignant glioma have been characterized and are known in the art (see Li Y M, Cell surface receptors in malignant glioma, Neurosurgery. 2011 October; 69(4):980-94).

The engineered vesicles can be immune tolerable, which can refer to their ability to not induce a significant immune response in a subject to which they are administered. This can reduce any antigenicity of any cargo compound and, in some instances, allow some cargo compounds that normally can induce an aberrant immune response in a subject, to be tolerated by the subject because the immune response can be reduced or eliminated completely. In short, when a potentially immune-reactive therapeutic molecule is cloaked within the engineered vesicle described herein, the immune-reactive therapeutic molecule can be shielded from the patient's immune system until it is delivered via gap junction channels (or other method) into the interior of the target cell—a space that is also shielded from immune surveillance.

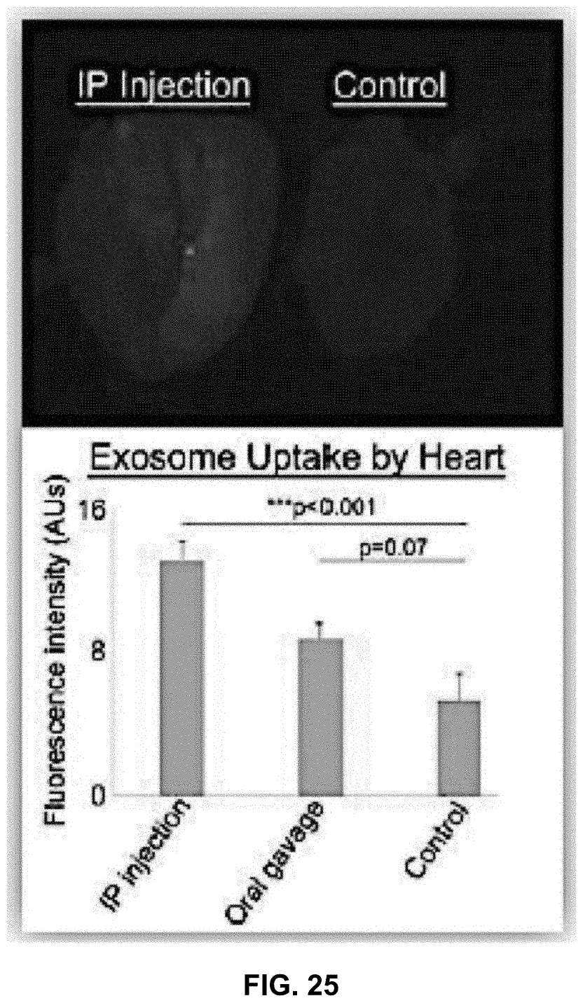

The engineered vesicles can be capable of passing across biological barriers. Such barriers might include from the gut into the blood circulation, from the exterior of the skin into the dermis and other tissues, through the skin into the circulation, across all types of epithelial and endothelial barriers, across the blood-brain barrier, blood eye barrier, and the barriers between body fluids (e.g., blood, cerebral spinal, lymph and so on) and all tissues and organs, including the brain, lungs, heart, kidney, spinal cord, muscle, liver, blood vessels, testes, ovaries, and so on. For example, milk exosomes can pass across the gut following oral gavage into a heart injured by myocardial infarction, as well as from the peritoneal cavity into a heart injured by myocardial infarction (see e.g. FIG. 25).

The engineered vesicle can also shield other cargo compounds from being broken down or otherwise destroyed by the subject's body prior to reaching a target. This can improve efficacy of these compounds and/or allow for smaller amounts to be delivered, which can improve toxicity profiles. For example, peptides can be broken down when they are just delivered straight to the subject by enzymes (e.g. peptidases). By being incorporated into the engineered vesicle as described in greater detail below, the peptides can reach their target cell without degradation. By allowing smaller doses to be effective, the engineered vesicles can allow for the use of less toxic doses (and result in less side effects) or allow for compounds that are toxic to be used to treat and/or prevent a disease, disorder, and/or condition, when delivered by an engineered vesicle described herein because a lower dose can be used and/or targeted delivery can be achieved.

Methods for the physical characterization and quantification of EVs and their cargos are known to those skilled in the art (PMID: 27495390; PMID: 24009896 PMID: 27035807; PMID: 27018079; PMID: 25536934, which are incorporated by reference). Approaches can include, but are not limited to, standard protein assays such as the Bradford assay, UV spectrophotometry, HPLC, TMS, Western blotting, Elisa as well as and/or in conjunction with the Nanosight instrument, and ExoELISA (System Biosciences). The methods cited, as well as other methods known to those skilled in the art, can be used to quantify the invention provided herein for purposes that include EV purification, determining EV yield, determining EV dosage, determining loading efficiency of the loaded therapeutic and other parameters that can provide the parameter desired from the EV invention described herein. For the purposes of the EV described herein, measurements of particle size, particle density, protein concentration, nucleic acid concentration, EV Cx43 levels, EV marker level (e.g., CD9, CD63, CD81, TSG101, MFGE8/lactadherin, HSP90B1, calnexin, GM130) and assays for the EV cargo compound, including expressed as a function of the aforementioned measurements (e.g., [aCT11]/particle density, [J M peptide]/[total protein] etc.).

Methods of Making the Engineered Vesicles

The EVs described herein can be produced by synthetic methods known in the art. Liposomes can be produced by a variety of methods (for a review; see, e.g.; Cullis et al. (1987)). Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation. During formation, engineered connexin 43 polypeptides and/or engineered connexons thereof can be included such that they are incorporated as connexons in the self-assembling lipid bilayer.

Extracellular vesicles of the present disclosure can be exosomes, nanovesicles or microvesicles. A variety of methods known in the art for the isolation of exosomes (see; for example, Lane et al., Scientific Reports, 5, 2015; incorporated herein by reference in its entirety) can be used in the present disclosure. Thus, in cells expressing the engineered connexin 43 polypeptides, endosomes and/or macrovesicles that contain the engineered connexin 43 polypeptides and engineered connexons thereof can be incorporated by the cells into the exosomes and/or macrovesicles. The exosomes and/or macrovesicles can be secreted by the cells into the surrounding medium and can be collected. In some aspects, exosomes can be isolated from cells after formation but prior to secretion. Methods of collecting, purifying, and/or isolating exosomes and/or macrovesicles are generally known in the art.

Various methodologies such as sonication, homogenization, French Press application and milling can be used to prepare engineered vesicles of a smaller size from larger vesicles already produced. Generally, extrusion (U.S. Pat. No. 5,008,050, incorporated herein by reference) can be used to size reduce vesicles, that is to produce vesicles having a predetermined mean size by forcing the vesicles, under pressure, through filter pores of a defined, selected size. Tangential flow filtration (WO89/008846, incorporated herein by reference) can also be used to regularize the size of engineered vesicles, that is, to produce a population of vesicles having less size heterogeneity, and a more homogeneous, defined size distribution.

The engineered vesicles produced by the methods disclosed herein can be populations of monodisperse engineered vesicles. In some aspects, the diameters of the vesicles can be within about 2% to about 20%, In some aspects, the diameters of the vesicles can be within about 20%, 15%, 10%, 5%, 4%, 3%, or 2% of each other.

After making the engineered vesicles, they can be stored for later use. The engineered vesicles can be stored frozen with or without cryoprotectants to prevent ice crystal formation. Examples of cryoprotectants that can be used include sugars (e.g., glucose, sucrose, trehalose) and glycols (e.g., ethylene glycol, propylene glycol and glycerol). Dimethyl sulfoxide (DMSO) can also be used as a cryoprotectant. In some aspects, the engineered vesicles can be stored following lyophilization or other non-disruptive technique that reduces the composition to a dried powder. This powder can be stored frozen or not and reconstituted in buffer for later use.

The engineered vesicles can be made by producing them in cells in vitro as previously described or can be made by harvesting exosomes, from a bodily fluid (blood, milk, urine, spinal fluid) of transgenic or non-transgenic animals. The harvested exosomes can be engineered exosomes already containing one or more engineered hemichannels described herein (e.g. those produced from transgenic animals). In some aspects, the harvested exosomes, (for example, from milk) are further modified after harvesting (e.g. introducing one or more engineered hemichannels, adding a targeting moiety, and/or loading a cargo molecule, etc.). Methods of making transgenic animals are generally known in the art and are discussed elsewhere herein.

Methods of Loading the Engineered Vesicles with a Cargo Compound

The engineered vesicles describe herein can include one or more cargo compounds. The cargo compound(s) can be contained in one or more of the internal compartments of the engineered vesicles and/or be integrated within the engineered vesicle membrane. It will be appreciated that where the cargo compound integrates (aqueous internal compartment vs. engineered vesicle membrane) can depend on the exact make of the engineered vesicle membrane and cargo compounds included. As described in greater detail below, any compound capable of passing through a pore that can be formed in the engineered vesicle when the engineered connexon is in an open configuration can be loaded into the engineered vesicle. In some embodiments, the molecular mass of the cargo compound is about 3,000 Daltons or less. In other embodiments, the molecular mass of the cargo compound is about 30,000 Daltons or less (e.g. miRNAs). In other embodiments, the molecular mass of the cargo compound is about 300,000 Daltons or less.

Cargo Compounds

The cargo compound can include any small molecule able to be transferred via the engineered connexons to the interior of the engineered vesicle, entrapped within the EV, transported by EVs to the site of therapy and transferred to target cells by gap junction channels at the site of therapy. Cargo compounds that can be loaded onto into an engineered vesicle can include, but are not limited to, DNA, RNA, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, anti-arrhythmic compounds, anti-epileptics, compounds that recover drug sensitivity in resistant patients and labels. Cargo compounds matching the parameters specified herein can be found in the Pharmacopoeia in the United States Pharmacopoeia (http://www.usp.org), The International Pharmacopoeia (https://web.archive.org/web/20060328053011/http://www.who.int/medicines/publications/pharmacopoeia/overview/en/) and other in other pharmacopoeias, which are incorporated by reference herein.

Suitable hormones include, but are not limited to, amino-acid derived hormones (e.g. melatonin and thyroxine), small peptide hormones and protein hormones (e.g. thyrotropin-releasing hormone, vasopressin, insulin, growth hormone, luteinizing hormone, follicle-stimulating hormone, and thyroid-stimulating hormone), eiconsanoids (e.g. arachidonic acid, lipoxins, and prostaglandins), and steroid hormones (e.g. estradiol, testosterone, tetrahydro testosteron cortisol).

Suitable immunomodulators include, but are not limited to, prednisone, azathioprine, 6-MP, cyclosporine, tacrolimus, methotrexate, interleukins (e.g. IL-2, IL-7, and IL-12), cytokines (e.g. interferons (e.g. IFN-α, IFN-β, IFN-ε, IFN-κ, IFN-ω, and IFN-γ), granulocyte colony-stimulating factor, and imiquimod), chemokines (e.g. CCL3, CCL26 and CXCL7), cytosine phosphate-guanosine, oligodeoxynucleotides, glucans, antibodies, and aptamers).

Suitable antipyretics include, but are not limited to, non-steroidal anti-inflammants (e.g. ibuprofen, naproxen, ketoprofen, and nimesulide), aspirin and related salicylates (e.g. choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g. alprazolam, bromazepam, chlordiazepoxide, clonazepam, clorazepate, diazepam, flurazepam, lorazepam, oxazepam, temazepam, triazolam, and tofisopam), serotonergic antidepressants (e.g. selective serotonin reuptake inhibitors, tricyclic antidepressants, and monoamine oxidase inhibitors), mebicar, afobazole, selank, bromantane, emoxypine, azapirones, barbituates, hydroxyzine, pregabalin, validol, and beta blockers.

Suitable antipsychotics include, but are not limited to, benperidol, bromperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, tiotixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzaprine, paliperidone, perospirone, quetiapine, remoxipride, risperidone, sertindole, trimipramine, ziprasidone, zotepine, alstonie, befeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, non-steroidal anti-inflammants (e.g. ibuprofen, naproxen, ketoprofen, and nimesulide), COX-2 inhibitors (e.g. rofecoxib, celecoxib, and etoricoxib), opioids and non-opioids (e.g. morphine, codeine, oxycodone, hydrocodone, heroine, levorphanol, meperidine, methadone, propoxyphene, fentanyl, naloxone, buprenorphine, butorphanol, nalbuphine, and pentazocine, dihydromorphine, pethidine, buprenorphine), tramadol, norepinephrine, flupiretine, nefopam, orphenadrine, pregabalin, gabapentin, cyclobenzaprine, scopolamine, methadone, ketobemidone, piritramide, and aspirin and related salicylates (e.g. choline salicylate, magnesium salicylate, and sodium salicylate).

Suitable antispasmodics include, but are not limited to, mebeverine, papverine, cyclobenzaprine, carisoprodol, orphenadrine, tizanidine, metaxalone, methodcarbamol, chlorzoxazone, baclofen, dantrolene, baclofen, tizanidine, and dantrolene.

Suitable anti-inflammatories include, but are not limited to, prednisone, non-steroidal anti-inflammants (e.g. ibuprofen, naproxen, ketoprofen, and nimesulide), COX-2 inhibitors (e.g. rofecoxib, celecoxib, and etoricoxib), and immune selective anti-inflammatory derivatives (e.g. submandibular gland peptide-T and its derivatives).

Suitable anti-histamines include, but are not limited to, H₁-receptor antagonists (e.g. acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbromapheniram ine, dexchlorpheniramine, dimenhydrinate, dimetindene, diphenhydramine, doxylamine, ebasine, embramine, fexofenadine, hydroxyzine, levocetirzine, loratadine, meclozine, mirtazapine, olopatadine, orphenadrine, phenindamine, pheniramine, phenyltoloxamine, promethazine, pyrilamine, quetiapine, rupatadine, tripelennamine, and triprolidine), H₂-receptor antagonists (e.g. cimetidine, famotidine, lafutidine, nizatidine, rafitidine, and roxatidine), tritoqualine, catechin, cromoglicate, nedocromil, and β2-adrenergic agonists.

Suitable anti-infectives include, but are not limited to, amebicides (e.g. nitazoxanide, paromomycin, metronidazole, tnidazole, chloroquine, and iodoquinol), aminoglycosides (e.g. paromomycin, tobramycin, gentamicin, amikacin, kanamycin, and neomycin), anthelmintics (e.g. pyrantel, mebendazole, ivermectin, praziquantel, abendazole, miltefosine, thiabendazole, oxamniquine), antifungals (e.g. azole antifungals (e.g. itraconazole, fluconazole, posaconazole, ketoconazole, clotrimazole, miconazole, and voriconazole), echinocandins (e.g. caspofungin, anidulafungin, and micafungin), griseofulvin, terbinafine, flucytosine, and polyenes (e.g. nystatin, and amphotericin b), antimalarial agents (e.g. pyrimethamine/sulfadoxine, artemether/lumefantrine, atovaquone/proquanil, quinine, hydroxychloroquine, mefloquine, chloroquine, doxycycline, pyrimethamine, and halofantrine), antituberculosis agents (e.g. aminosalicylates (e.g. aminosalicylic acid), isoniazid/rifampin, isoniazid/pyrazinamide/rifampin, bedaquiline, isoniazid, ethanmbutol, rifampin, rifabutin, rifapentine, capreomycin, and cycloserine), antivirals (e.g. amantadine, rimantadine, abacavir/lamivudine, emtricitabine/tenofovir, cobicistat/elvitegravir/emtricitabine/tenofovir, efavirenz/emtricitabine/tenofovir, avacavir/lamivudine/zidovudine, lam ivudine/zidovudine, emtricitabine/tenofovir, emtricitabine/opinavir/ritonavir/tenofovir, interferon alfa-2v/ribavirin, peg interferon alfa-2b, maraviroc, raltegravir, dolutegravir, enfuvirtide, foscarnet, fomivirsen, oseltamivir, zanamivir, nevirapine, efavirenz, etravirine, rilpiviirine, delaviridine, nevirapine, entecavir, lamivudine, adefovir, sofosbuvir, didanosine, tenofovir, avacivr, zidovudine, stavudine, emtricitabine, xalcitabine, telbivudine, simeprevir, boceprevir, telaprevir, lopinavir/ritonavir, fosamprenvir, dranuavir, ritonavir, tipranavir, atazanavir, nelfinavir, amprenavir, indinavir, sawuinavir, ribavirin, valcyclovir, acyclovir, famciclovir, ganciclovir, and valganciclovir), carbapenems (e.g. doripenem, meropenem, ertapenem, and cilastatin/imipenem), cephalosporins (e.g. cefadroxil, cephradine, cefazolin, cephalexin, cefepime, ceflaroline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, cefizoxime, and ceftazidime), glycopeptide antibiotics (e.g. vancomycin, dalbavancin, oritavancin, and telvancin), glycylcyclines (e.g. tigecycline), leprostatics (e.g. clofazimine and thalidomide), lincomycin and derivatives thereof (e.g. clindamycin and lincomycin), macrolides and derivatives thereof (e.g. telithromycin, fidaxomicin, erthromycin, azithromycin, clarithromycin, dirithromycin, and troleandomycin), linezolid, sulfamethoxazole/trimethoprim, rifaximin, chloramphenicol, fosfomycin, metronidazole, aztreonam, bacitracin, beta lactam antibiotics (benzathine penicillin (benzatihine and benzylpenicillin), phenoxymethylpenicillin, cloxacillin, flucoxacillin, methicillin, temocillin, mecillinam, azlocillin, mezlocillin, piperacillin, amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, nafcillin, cefazolin, cephalexin, cephalosporin C, cephalothin, cefaclor, cefamandole, cefuroxime, cefotetan, cefoxitin, cefiximine, cefotaxime, cefpodoxime, ceftazidime, ceftriaxone, cefepime, cefpirome, ceftaroline, biapenem, doripenem, ertapenem, faropenem, imipenem, meropenem, panipenem, razupenem, tebipenem, thienamycin, azrewonam, tigemonam, nocardicin A, taboxinine, and beta-lactam), quinolones (e.g. lomefloxacin, norfloxacin, ofloxacin, qatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g. sulfamethoxazole/trimethoprim, sulfasalazine, and sulfasoxazole), tetracyclines (e.g. doxycycline, demeclocycline, minocycline, doxycycline/salicyclic acid, doxycycline/omega-3 polyunsaturated fatty acids, and tetracycline), and urinary anti-infectives (e.g. nitrofurantoin, methenamine, fosfomycin, cinoxacin, nalidixic acid, trimethoprim, and methylene blue).

Suitable chemotherapeutics include but are not limited to Abiraterone Acetate, ABITREXATE (Methotrexate), ABRAXANE (Paclitaxel Albumin-stabilized Nanoparticle Formulation), ADCETRIS (Brentuximab Vedotin), Ado-Trastuzumab Emtansine, ADRIAMYCIN (Doxorubicin Hydrochloride), ADRUCIL (Fluorouracil), Afatinib Dimaleate, AFINITOR (Everolimus), ALDARA (Imiquimod), Aldesleukin, Alemtuzumab, ALIMTA (Pemetrexed Disodium), ALOXI (Palonosetron Hydrochloride), AMBOCHLORIN (Chlorambucil), AMBOCLORIN (Chlorambucil), Aminolevulinic Acid, Anastrozole, Aprepitant, AREDIA (Pamidronate Disodium), ARIMIDEX (Anastrozole), AROMASIN (Exemestane), ARRANON (Nelarabine), Arsenic Trioxide, ARZERRA (Ofatumumab), Asparaginase Erwinia chrysanthemi, AVASTIN (Bevacizumab), Axitinib, Azacitidine, Bendamustine Hydrochloride, Bevacizumab, Bexarotene, BEXXAR (Tositumomab and I 131 Iodine Tositumomab), Bleomycin, Bortezomib, BOSULIF (Bosutinib), Cabazitaxel, Cabozantinib-S-Malate, CAM PATH (Alemtuzumab), CAMPTOSAR (Irinotecan Hydrochloride), Capecitabine, Carboplatin, Carfilzomib, CEENU (Lomustine), CERUBIDINE (Daunorubicin Hydrochloride), Cetuximab, Chlorambucil, Cisplatin, CLAFEN (Cyclophosphamide), Clofarabine, COMETRIQ (Cabozantinib-S-Malate), COSMEGEN (Dactinomycin), Creatine, Crizotinib, Cyclophosphamide, CYFOS (Ifosfamide), Cytarabine, Dabrafenib, Dacarbazine, DACOGEN (Decitabine), Dactinomycin, Dasatinib, Daunorubicin Hydrochloride, Decitabine, Degarelix, Denileukin Diftitox, Denosumab, Dexrazoxane Hydrochloride, Docetaxel, Doxorubicin Hydrochloride, EFUDEX (Fluorouracil), ELITEK (Rasburicase), ELLENCE (Epirubicin Hydrochloride), ELOXATIN (Oxaliplatin), Eltrombopag Olamine, EMEND (Aprepitant), Enzalutamide, Epirubicin Hydrochloride, ERBITUX (Cetuximab), Eribulin Mesylate, ERIVEDGE (Vismodegib), Erlotinib Hydrochloride, ERWINAZE (Asparaginase Erwinia chrysanthemi), Etoposide, Everolimus, EVISTA (Raloxifene Hydrochloride), Exemestane, FARESTON (Toremifene), FASLODEX (Fulvestrant), FEMARA (Letrozole), Filgrastim, FLUDARA (Fludarabine Phosphate), Fludarabine Phosphate, FLUOROPLEX (Fluorouracil), Fluorouracil, Folinic acid, FOLOTYN (Pralatrexate), Fulvestrant, Gefitinib, Gemcitabine Hydrochloride, Gemtuzumab Ozogamicin, GEMZAR (Gemcitabine Hydrochloride), GILOTRIF (Afatinib Dimaleate), GLEEVEC (Imatinib Mesylate), HALAVEN (Eribulin Mesylate), HERCEPTIN (Trastuzumab), HYCAMTIN (Topotecan Hydrochloride), Ibritumomab Tiuxetan, ICLUSIG (Ponatinib Hydrochloride), Ifosfamide, Imatinib Mesylate, Imiquimod, INLYTA (Axitinib), INTRON A (Recombinant Interferon Alfa-2b), Iodine 131 Tositumomab and Tositumomab, Ipilimumab, IRESSA (Gefitinib), Irinotecan Hydrochloride, ISTODAX (Romidepsin), Ixabepilone, JAKAFI (Ruxolitinib Phosphate), JEVTANA (Cabazitaxel), Kadcyla (Ado-Trastuzumab Emtansine), KEOXIFENE (Raloxifene Hydrochloride), KEPIVANCE (Palifermin), KYPROLIS (Carfilzomib), Lapatinib Ditosylate, Lenalidomide, Letrozole, Leucovorin Calcium, Leuprolide Acetate, Lomustine, LUPRON (Leuprolide Acetate, MARQIBO (Vincristine Sulfate Liposome), MATULANE (Procarbazine Hydrochloride), Mechlorethamine Hydrochloride, MEGACE (Megestrol Acetate), Megestrol Acetate, MEKINIST (Trametinib), Mercaptopurine, Mesna, METHAZOLASTONE (Temozolomide), Methotrexate, Mitomycin, MOZOBIL (Plerixafor), MUSTARGEN (Mechlorethamine Hydrochloride), MUTAMYCIN (Mitomycin C), MYLOSAR (Azacitidine), MYLOTARG (Gemtuzumab Ozogamicin), Nanoparticle Paclitaxel (Paclitaxel Albumin-stabilized Nanoparticle Formulation), NAVELBINE (Vinorelbine Tartrate), Nelarabine, NEOSAR (Cyclophosphamide), NEUPOGEN (Filgrastim), NEXAVAR (Sorafenib Tosylate), Nilotinib, NOLVADEX (Tamoxifen Citrate), NPLATE (Romiplostim), Ofatumumab, Omacetaxine Mepesuccinate, ONCASPAR (Pegaspargase), ONTAK (Denileukin Diftitox), Oxaliplatin, Paclitaxel, Paclitaxel Albumin-stabilized Nanoparticle Formulation, Palifermin, Palonosetron Hydrochloride, Pamidronate Disodium, Panitumumab, Pazopanib Hydrochloride, Pegaspargase, Peg interferon Alfa-2b, PEG-INTRON (Peg interferon Alfa-2b), Pemetrexed Disodium, Pertuzumab, PLATINOL (Cisplatin), PLATINOL-AQ (Cisplatin), Plerixafor, Pomalidomide, POMALYST (Pomalidomide), Ponatinib Hydrochloride, Pralatrexate, Prednisone, Procarbazine Hydrochloride, PROLEUKIN (Aldesleukin), PROLIA (Denosumab), PROMACTA (Eltrombopag Olamine), PROVENGE (Sipuleucel-T), PURINETHOL (Mercaptopurine), Radium 223 Dichloride, Raloxifene Hydrochloride, Rasburicas, Recombinant Interferon Alfa-2b, Regorafenib, REVLIMID (Lenalidomide), RHEUMATREX (Methotrexate), Rituximab, Romidepsin, Romiplostim, RUBIDOMYCIN (Daunorubicin Hydrochloride), Ruxolitinib Phosphat, Sipuleucel-T, Sorafenib Tosylate, SPRYCEL (Dasatinib), STIVARGA (Regorafenib), Sunitinib Malate, SUTENT (Sunitinib Malate), SYLATRON (Peg interferon Alfa-2b), SYNOVIR (Thalidomide), SYNRIBO (Omacetaxine Mepesuccinate), TAFINLAR (Dabrafenib), Tamoxifen Citrate, TARABINE PFS (Cytarabine), TARCEVA (Erlotinib Hydrochloride), TARGRETIN (Bexarotene), TASIGNA (Nilotinib), TAXOL (Paclitaxel), TAXOTERE (Docetaxel), TEMODAR (Temozolomide), Temozolomide, Temsirolimus, Thalidomide, TOPOSAR (Etoposide), Topotecan Hydrochloride, Toremifene, TORISEL (Temsirolimus), Tositumomab and I 131 Iodine Tositumomab, TOTECT (Dexrazoxane Hydrochloride), Trametinib, Trastuzumab, TREANDA (Bendamustine Hydrochloride), TRISENOX (Arsenic Trioxide), TYKERB (Lapatinib Ditosylate), Vandetanib, VECTIBIX (Panitumumab), VeIP, VELBAN (Vinblastine Sulfate), VELCADE (Bortezomib), VELSAR (Vinblastine Sulfate), Vemurafenib, VEPESID (Etoposide), VIADUR (Leuprolide Acetate), VIDAZA (Azacitidine), Vinblastine Sulfate, Vincristine Sulfate, Vinorelbine Tartrate, Vismodegib, VORAXAZE (Glucarpidase), Vorinostat, VOTRIENT (Pazopanib Hydrochloride), WELLCOVORIN (Leucovorin Calcium), XALKORI (Crizotinib), XELODA (Capecitabine), XGEVA (Denosumab), XOFIGO (Radium 223 Dichloride), XTANDI (Enzalutamide), YERVOY (Ipilimumab), ZALTRAP (Ziv-Aflibercept), ZELBORAF (Vemurafenib), ZEVALIN (Ibritumomab Tiuxetan), ZINECARD (Dexrazoxane Hydrochloride), Ziv-Aflibercept, Zoledronic Acid, ZOLINZA (Vorinostat), ZOMETA (Zoledronic Acid), and ZYTIGA (Abiraterone Acetate), including any formulation (e.g. liposomal, pegylated) any salt or any brand name of any generic agent included herein.

Suitable peptides include, but are not limited to Peptide 5, Gap19, L2, Cx43 sic peptide, aCT peptides, aCT1, aCT11 aCT11-I, aCT1-1, JM peptides and other peptides that are able to permeate hemichannels. See e.g. WO2013163423 A1, WO2008157840 A3, U.S. Pat. No. 7,888,319 B2, US20160166637 A1, U.S. Pat. No. 9,345,744 B2, WO2009148552 A2, WO2013131040 A1, PubMed IDs: 28712848, 23734129, 19317641, 28694772, 23664811, 17576073, 28063303, 27856346, 25652199, 28931622, and 25591543. The peptide or portion thereof can have an amino acid sequence with at least 65%, 70%, 75%, 80%, 85%, 90%, 95%, 96%, 97%, 98%, 99% to/or 100% sequence identity to PRPDDLEI (SEQ ID NO: 33), RPDDLE (SEQ ID NO: 115), RPRPDDLEI (SEQ ID NO: 13), RPRPDDELI (SEQ ID NO: 116), or RPRPDDLE (SEQ ID NO: 14), SEQ ID NO: 111, or SEQ ID NO: 112.

Suitable nucleic acid molecules can include, but are not limited to, those set forth in e.g. WO2005059111, PubMed IDs: 21986484, 15033581, 16037090, 28655327, 28497038, 27612280, 26773301, 26514375, 28962871, RNAi such as siRNA, shRNA, and miRNA. Manipulating the cellular process of RNA interference (RNAi) is an effective method for suppressing the expression of a specific gene to study its function. RNAi pathways are activated by various forms of double-stranded (ds) RNAs that contain sequences which are homologous to the mRNA transcript of a target gene. RNAi includes small interfering RNA (siRNA), short hairpin RNA (shRNA) and micro RNA (miRNA). Short hairpin RNA (shRNA) transcripts adopt a stable stem-loop structure in solution; can be easily be expressed from a cloned oligonucleotide template; and are a convenient and reproducible means of activating RNAi in cells. Small interfering RNA (siRNA) is a class of double-stranded RNA molecules about 20-25 nucleotides in length. siRNA interferes with the expression of specific genes with complementary nucleotide sequences by causing mRNA to be broken down after transcription, resulting in no translation.

Suitable antiarrhythmic compounds include, but are not limited to, class Ia drugs, e.g., Quinidine, Procainamide, Disopyramide, class Ib drugs e.g., Lidocaine, Phenytoin, Mexiletine, class Ic drugs e.g., Flecainide, Propafenone, Moricizine, class II drugs e.g., Propranolol, Esmolol, Timolol, Metoprolol and Atenolol, class III drugs, e.g., Amiodarone, Sotalol, Ibutilide and Dofetilide, class IV drugs, e.g., Verapamil, Diltiazem and class V drugs e.g., Adenosine and Digoxin.

Suitable antiepileptics, include but are not limited to, carbamazepine, clorazepate (Tranxene) clonazepam (Klonopin), ethosuximide (Zarontin), felbamate (Felbatol), fosphenytoin (Cerebyx), gabapentin (Neurontin), lamotrigine (Lamictal), levetiracetam (Keppra), oxcarbazepine (Trileptal), phenobarbital (Luminal), phenytoin (Dilantin), pregabalin (Lyrica), primidone (Mysoline), tiagabine (Gabitril), topiramate (Topamax), valproate semisodium (Depakote), valproic acid (Depakene), zonisamide (Zonegran), clobazam (Frisium) and vigabatrin (Sabril), retigabine, brivaracetam, and seletracetam, diazepam (Valium, Diastat) and lorazepam (Ativan), Paral, midazolam (Versed), and pentobarbital (Nembutal), acetazolamide (Diamox), progesterone, adrenocorticotropic hormone (ACTH, Acthar), various corticotropic steroid hormones (prednisone), or bromide.

Suitable labels can include dyes (e.g. fluorescent dyes and compounds, infrared dyes, far infrared dyes), imaging agents (e.g. paramagnetic ions and materials), theranostic agents, and radio isotopes.

The cargo compound described herein can be loaded into the engineered extracellular vesicle at an amount that when delivered an effect amount is provided to the subject. The cargo compound can be provided as a pharmaceutically acceptable salt of a cargo compound described herein as appropriate. Suitable salts include, but are not limited to, sulfate, citrate, acetate, oxalate, chloride, creatine, hydrochloride, bromide, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

A microRNA (abbreviated miRNA) is a small non-coding RNA molecule (containing about 22 nucleotides) found in plants, animals and some viruses, which functions in RNA silencing and post-transcriptional regulation of gene expression. Over 1900 miRNAs are expressed in humans and these molecules can pass through connexons and are thus suitable cargoes for the disclosed invention. Suitable miRNAs include those listed in mirBase (http://www.mirbase.org/cgi-bin/mirna_summary.pl?org=has) such as human MIRLET7A1 MIRLET7A2, MIRLET7A3, MIRLET7B, MIRLET7C, MIRLET7D, MIRLET7E, MIRLET7F1, MIRLET7F2, MIRLET7G, MIRLET7I, MIR10A, MIR10B, MIR1-1, MIR1-2, MIR15Ak MIR15Bk MIR17k MIR18Ak MIR18Bk MIR19Ak MIR19B1, MIR19B2, MIR20A, MIR20B, MIR21, MIR22, MIR23A, MIR23B, MIR23C, MIR25, MIR26A1, MIR26A2, MIR26B, MIR27A, MIR27B, MIR28, MIR29A, MIR29B1, MIR29B2, MIR29C, MIR30A, MIR30B, MIR30C1, MIR30C2, MIR30D, MIR30E, MIR31, MIR32, MIR33A, MIR33B, MIR34A, MIR34B, MIR34C, MIR7-1, MIR7-2, MIR7-3, MIR9-1, MIR9-2, MIR92A1, MIR92A2, MIR92B, MIR9-3, MIR93, MIR95, MIR96, MIR98, MIR99A, MIR99B, MIR100, MIR103A1, MIR103A2, MIR103B1, MIR103B2, MIR106A, MIR106B, MIR107, MIR122, MIR125A, MIR125B1, MIR125B2, MIR126, MIR127, MIR130A, MIR130B, MIR132, MIR133A1, MIR133A2, MIR133B, MIR134, MIR135A1, MIR135A2, MIR135B, MIR136, MIR137, MIR139, MIR140, MIR141, MIR142, MIR143, MIR144, MIR145, MIR146A, MIR146B, MIR147A, MIR147B, MIR148A, MIR148B, MIR149, MIR150, MIR151A, MIR151B, MIR152, M1R154, M1R155, MIR16-1, MIR16-2, MIR181A1, MIR181A2, MIR181B1, MIR181B2, MIR181C, MIR181D, MIR182, MIR183, MIR184, MIR185, MIR186, MIR187, MIR188, MIR190A, MIR190B, MIR191, MIR192, MIR193A, MIR193B, MIR195, MIR196A1, MIR196A2, MIR196B, MIR197, MIR198, MIR199A1, MIR199A2, MIR199B, MIR200A, MIR200B, MIR200C, MIR202, MIR203A, MIR203B, MIR204, MIR205, MIR206, MIR208A, MIR208B, MIR210, MIR211, MIR212, MIR214, MIR215, MIR216A, MIR216B, MIR217, MIR219A1, MIR219A2, MIR219B, MIR221, MIR222, MIR223, MIR224, MIR24-1, MIR24-2, MIR296, MIR297, MIR298, MIR299, MIR300, MIR301A, MIR301B, MIR302A, MIR302B, MIR302C, MIR302D, MIR302E, MIR302F, MIR320A, MIR320B1, MIR320B2, MIR320C1, MIR320C2, MIR320D1, MIR320D2, MIR320E, MIR323A, MIR323B, MIR324, MIR325, MIR326, MIR328, MIR330, MIR331, MIR335, MIR337, MIR338, MIR339, MIR340, MIR342, MIR345, MIR346, MIR361, MIR362, MIR363, MIR365A, MIR365B, MIR367, MIR369, MIR370, MIR371A, MIR371B, MIR372, MIR373, MIR374A, MIR374B, MIR374C, MIR375, MIR376A1, MIR376A2, MIR376B, MIR376C, MIR377, MIR378A, MIR378B, MIR378C, MIR378D1, MIR378D2, MIR378E, MIR378F, MIR378G, MIR378H, MIR3781, MIR378J, MIR379, MIR380, MIR381, MIR382, MIR383, MIR384, MIR409, MIR410, MIR411, MIR412, MIR421, MIR422A, MIR423, MIR424, MIR425, MIR429, MIR431, MIR432, MIR433, MIR448, MIR449A, MIR449B, MIR449C, MIR450A1, MIR450A2, MIR450B, MIR451A, MIR451B, MIR452, MIR454, MIR455, MIR466, MIR483, MIR484, MIR485, MIR487A, MIR487B, MIR488, MIR489, MIR490, MIR491, MIR492, MIR493, MIR494, MIR495, MIR496, MIR497, MIR498, MIR499A, MIR499B, MIR500A, MIR500B, MIR501, MIR502, MIR503, MIR504, MIR505, MIR506, MIR507, MIR508, MIR510, MIR511, MIR513A1, MIR513A2, MIR513B, MIR513C, MIR514A1, MIR514A2, MIR514A3, MIR514B, MIR516A1, MIR516A2, MIR516B1, MIR516B2, MIR517A, MIR517B, MIR517C, MIR518A1, MIR518A2, MIR518B, MIR518C, MIR518D, MIR518E, MIR518F, MIR519A1, MIR519A2, MIR519B, MIR519C, MIR519D, MIR519E, MIR520A, MIR520B, MIR520C, MIR520D, MIR520E, MIR520F, MIR520G, MIR520H, MIR522, MIR523, MIR524, MIR525, MIR526A1, MIR526A2, MIR526B, MIR527, MIR532, MIR539, MIR541, MIR542, MIR543, MIR544A, MIR544B, MIR545, MIR548AA1, MIR548AA2, MIR548AB, MIR548AC, MIR548AD, MIR548AE1, MIR548AE2, MIR548AG 1, MIR548AG2, MIR548AH, MIR548A1, MIR548AJ1, MIR548AJ2, MIR548AK, MIR548AL, MIR548AM, MIR548AN, MIR548AY, MIR548AZ, MIR548A1, MIR548A2, MIR548A3, MIR548B, MIR548BA, MIR548BB, MIR548C, MIR548D1, MIR548D2, MIR548E, MIR548F1, MIR548F2, MIR548F3, MIR548F4, MIR548F5, MIR548G, MIR548H1, MIR548H2, MIR548H3, MIR548H4, MIR548H5, MIR54811, MIR54812, MIR54813, MIR54814, MIR548J, MIR548K, MIR548L, MIR548M, MIR548N, MIR5480, MIR54802, MIR548P, MIR548Q, MIR548S, MIR548T, MIR548U, MIR548V, MIR548W, MIR548X, MIR548X2, MIR548Y, MIR548Z, MIR549A, MIR550A1, MIR550A2, MIR550A3, MIR550B1, MIR550B2, MIR551A, MIR551B, MIR552, MIR553, MIR554, MIR555, MIR556, MIR557, MIR558, MIR559, MIR561, MIR562, MIR563, MIR564, MIR566, MIR567, MIR568, MIR569, MIR570, MIR571, MIR572, MIR573, MIR574, MIR575, MIR576, MIR577, MIR578, MIR579, MIR580, MIR581, MIR582, MIR583, MIR584, MIR585, MIR586, MIR587, MIR588, MIR589, MIR590, MIR591, MIR592, MIR593, MIR595, MIR596, MIR597, MIR598, MIR599, MIR600, MIR601, MIR602, MIR603, MIR604, MIR605, MIR606, MIR607, MIR608, MIR609, MIR610, MIR611, MIR612, MIR613, MIR614, MIR615, MIR616, MIR617, MIR618, MIR619, MIR620, MIR621, MIR622, MIR623, MIR624, MIR625, MIR626, MIR627, MIR628, MIR629, MIR630, MIR631, MIR632, MIR633, MIR634, MIR635, MIR636, MIR637, MIR638, MIR639, MIR640, MIR641, MIR642A, MIR642B, MIR643, MIR644A, MIR645, MIR646, MIR647, MIR648, MIR649, MIR650, MIR651, MIR652, MIR653, MIR654, MIR655, MIR656, MIR657, MIR658, MIR659, MIR660, MIR661, MIR662, MIR663A, MIR663B, MIR664A, MIR665, MIR668, MIR670, MIR671, MIR675, MIR676, MIR708, MIR711, MIR718, MIR744, MIR758, MIR759, MIR760, MIR761, MIR762, MIR764, MIR765, MIR766, MIR767, MIR769, MIR770, MIR802, MIR873, MIR874, MIR875, MIR876, MIR877, MIR885, MIR887, MIR888, MIR889, MIR890, MIR891A, MIR891B, MIR892A, MIR892B, MIR892C, MIR920, MIR921, MIR922, MIR924, MIR933, MIR934, MIR935, MIR936, MIR937, MIR938, MIR939, MIR940, MIR942, MIR943, MIR944, MIR101-1, MIR101-2, MIR105-1, MIR105-2, MIR1178, MIR1179, MIR1180, MIR1181, MIR1182, MIR1183, MIR1193, MIR1197, MIR1199, MIR1200, MIR1202, MIR1203, MIR1204, MIR1205, MIR1206, MIR1207, MIR1208, MIR1224, MIR1225, MIR1226, MIR1227, MIR1228, MIR1229, MIR1231, MIR1234, MIR1236, MIR1237, MIR1238, MIR124-1, MIR124-2, MIR124-3, MIR1243, MIR1245A, MIR1245B, MIR1246, MIR1247, MIR1248, MIR1249, MIR1250, MIR1251, MIR1252, MIR1253, MIR1255A, MIR1255B1, MIR1255B2, MIR1256, MIR1257, MIR1258, MIR1260A, MIR1260B, MIR1261, MIR1262, MIR1263, MIR1264, MIR1265, MIR1266, MIR1267, MIR1268A, MIR1268B, MIR1269A, MIR1269B, MIR1270, MIR1271, MIR1272, MIR1273A, MIR1273C, MIR1273D, MIR1273E, MIR1273F, MIR1273G, MIR1273H, MIR1275, MIR1276, MIR1277, MIR1278, MIR1279, MIR128-1, MIR1281, MIR128-2, MIR1282, MIR1284, MIR1286, MIR1287, MIR1288, MIR1290, MIR129-1, MIR1291, MIR129-2, MIR1292, MIR1293, MIR1294, MIR1295A, MIR1296, MIR1297, MIR1298, MIR1299, MIR1301, MIR1303, MIR1304, MIR1305, MIR1306, MIR1307, MIR1321, MIR1322, MIR1323, MIR1324, MIR1343, MIR138-1, MIR138-2, MIR1468, MIR1469, MIR1470, MIR1471, MIR153-1, MIR153-2, MIR1537, MIR1538, MIR1539, MIR1587, MIR1825, MIR1827, MIR1908, MIR1909, MIR1910, MIR1911, MIR1912, MIR1913, MIR1914, MIR1915, MIR194-1, MIR194-2, MIR1973, MIR1976, MIR2052, MIR2053, MIR2054, MIR2110, MIR2113, MIR2114, MIR2115, MIR2116, MIR2117, MIR218-1, MIR218-2, MIR2276, MIR2277, MIR2278, MIR2355, MIR2392, MIR2467, MIR2681, MIR2682, MIR2861, MIR2909, MIR3064, MIR3065, MIR3074, MIR3115, MIR3117, MIR3120, MIR3121, MIR3122, MIR3123, MIR3124, MIR3125, MIR3126, MIR3127, MIR3128, MIR3129, MIR3131, MIR3132, MIR3133, MIR3134, MIR3135A, MIR3135B, MIR3136, MIR3137, MIR3138, MIR3139, MIR3140, MIR3141, MIR3142, MIR3143, MIR3144, MIR3145, MIR3146, MIR3147, MIR3148, MIR3149, MIR3150A, MIR3150B, MIR3151, MIR3152, MIR3153, MIR3154, MIR3155A, MIR3155B, MIR3157, MIR3159, MIR3161, MIR3162, MIR3163, MIR3164, MIR3165, MIR3166, MIR3167, MIR3168, MIR3169, MIR3170, MIR3171, MIR3173, MIR3174, MIR3175, MIR3176, MIR3177, MIR3178, MIR3181, MIR3182, MIR3183, MIR3184, MIR3185, MIR3186, MIR3187, MIR3188, MIR3189, MIR3190, MIR3191, MIR3192, MIR3193, MIR3194, MIR3195, MIR3196, MIR3197, MIR3200, MIR3201, MIR329-1, MIR329-2, MIR3529, MIR3591, MIR3605, MIR3606, MIR3609, MIR3610, MIR3611, MIR3612, MIR3613, MIR3614, MIR3615, MIR3616, MIR3617, MIR3618, MIR3619, MIR3620, MIR3621, MIR3622A, MIR3622B, MIR3646, MIR3649, MIR3650, MIR3651, MIR3652, MIR3653, MIR3654, MIR3655, MIR3656, MIR3657, MIR3658, MIR3659, MIR3660, MIR3661, MIR3662, MIR3663, MIR3664, MIR3665, MIR3666, MIR3667, MIR3668, MIR3671, MIR3672, MIR3674, MIR3675, MIR3677, MIR3678, MIR3679, MIR3681, MIR3682, MIR3683, MIR3684, MIR3685, MIR3686, MIR3689A, MIR3689B, MIR3689C, MIR3689D1, MIR3689D2, MIR3689E, MIR3689F, MIR3690, MIR3691, MIR3692, MIR3713, MIR3714, MIR3907, MIR3908, MIR3909, MIR3911, MIR3912, MIR3915, MIR3916, MIR3917, MIR3918, MIR3919, MIR3920, MIR3921, MIR3922, MIR3923, MIR3924, MIR3925, MIR3927, MIR3928, MIR3929, MIR3934, MIR3935, MIR3936, MIR3937, MIR3938, MIR3939, MIR3940, MIR3941, MIR3942, MIR3943, MIR3944, MIR3945, MIR3960, MIR3972, MIR3973, MIR3974, MIR3975, MIR3976, MIR3977, MIR3978, MIR4251, MIR4252, MIR4253, MIR4254, MIR4255, MIR4256, MIR4257, MIR4258, MIR4259, MIR4260, MIR4261, MIR4262, MIR4263, MIR4264, MIR4265, MIR4266, MIR4267, MIR4268, MIR4269, MIR4270, MIR4271, MIR4272, MIR4273, MIR4274, MIR4275, MIR4276, MIR4277, MIR4278, MIR4279, MIR4280, MIR4281, MIR4282, MIR4284, MIR4285, MIR4286, MIR4287, MIR4288, MIR4289, MIR4290, MIR4291, MIR4292, MIR4293, MIR4294, MIR4295, MIR4296, MIR4297, MIR4298, MIR4299, MIR4300, MIR4301, MIR4302, MIR4303, MIR4304, MIR4305, MIR4306, MIR4307, MIR4308, MIR4309, MIR4310, MIR4311, MIR4312, MIR4313, MIR4314, MIR4316, MIR4317, MIR4318, MIR4319, MIR4320, MIR4321, MIR4322, MIR4323, MIR4324, MIR4325, MIR4326, MIR4327, MIR4328, MIR4329, MIR4330, MIR4417, MIR4418, MIR4419A, MIR4419B, MIR4420, MIR4421, MIR4422, MIR4423, MIR4424, MIR4425, MIR4426, MIR4427, MIR4428, MIR4429, MIR4430, MIR4431, MIR4432, MIR4433A, MIR4433B, MIR4434, MIR4436A, MIR4436B1, MIR4437, MIR4438, MIR4439, MIR4440, MIR4441, MIR4442, MIR4443, MIR4445, MIR4446, MIR4447, MIR4448, MIR4449, MIR4450, MIR4451, MIR4452, MIR4453, MIR4454, MIR4455, MIR4456, MIR4457, MIR4458, MIR4459, MIR4460, MIR4461, MIR4462, MIR4463, MIR4464, MIR4465, MIR4466, MIR4467, MIR4468, MIR4469, MIR4470, MIR4471, MIR4473, MIR4474, MIR4475, MIR4476, MIR4477A, MIR4477B, MIR4478, MIR4479, MIR4480, MIR4481, MIR4482, MIR4483, MIR4484, MIR4485, MIR4486, MIR4487, MIR4488, MIR4489, MIR4490, MIR4491, MIR4492, MIR4493, MIR4494, MIR4495, MIR4496, MIR4497, MIR4498, MIR4499, MIR4500, MIR4501, MIR4502, MIR4503, MIR4504, MIR4505, MIR4506, MIR4507, MIR4508, MIR4510, MIR4511, MIR4512, MIR4513, MIR4514, MIR4515, MIR4516, MIR4517, MIR4518, MIR4519, MIR4521, MIR4522, MIR4523, MIR4524A, MIR4525, MIR4526, MIR4527, MIR4528, MIR4529, MIR4530, MIR4531, MIR4532, MIR4533, MIR4534, MIR4535, MIR4537, MIR4538, MIR4539, MIR4540, MIR4632, MIR4633, MIR4634, MIR4635, MIR4636, MIR4637, MIR4638, MIR4639, MIR4640, MIR4641, MIR4642, MIR4643, MIR4644, MIR4645, MIR4646, MIR4647, MIR4648, MIR4649, MIR4651, MIR4652, MIR4653, MIR4654, MIR4655, MIR4656, MIR4657, MIR4658, MIR4659A, MIR4659B, MIR4660, MIR4661, MIR4662A, MIR4662B, MIR4663, MIR4664, MIR4665, MIR4666A, MIR4667, MIR4668, MIR4669, MIR4670, MIR4671, MIR4672, MIR4673, MIR4674, MIR4675, MIR4676, MIR4677, MIR4678, MIR4680, MIR4681, MIR4682, MIR4683, MIR4684, MIR4685, MIR4686, MIR4687, MIR4688, MIR4689, MIR4690, MIR4691, MIR4692, MIR4693, MIR4694, MIR4695, MIR4696, MIR4697, MIR4698, MIR4699, MIR4700, MIR4701, MIR4703, MIR4704, MIR4705, MIR4706, MIR4707, MIR4708, MIR4709, MIR4710, MIR4711, MIR4712, MIR4713, MIR4714, MIR4715, MIR4716, MIR4717, MIR4718, MIR4719, MIR4720, MIR4721, MIR4722, MIR4723, MIR4724, MIR4725, MIR4726, MIR4727, MIR4728, MIR4729, MIR4730, MIR4731, MIR4732, MIR4733, MIR4734, MIR4735, MIR4736, MIR4737, MIR4738, MIR4739, MIR4740, MIR4741, MIR4742, MIR4743, MIR4744, MIR4745, MIR4746, MIR4747, MIR4748, MIR4749, MIR4750, MIR4751, MIR4752, MIR4753, MIR4754, MIR4755, MIR4756, MIR4757, MIR4758, MIR4759, MIR4760, MIR4761, MIR4762, MIR4763, MIR4764, MIR4765, MIR4766, MIR4767, MIR4768, MIR4769, MIR4770, MIR4772, MIR4774, MIR4775, MIR4777, MIR4778, MIR4779, MIR4780, MIR4781, MIR4782, MIR4783, MIR4784, MIR4785, MIR4786, MIR4787, MIR4788, MIR4789, MIR4790, MIR4791, MIR4792, MIR4793, MIR4794, MIR4795, MIR4796, MIR4797, MIR4798, MIR4799, MIR4800, MIR4801, MIR4802, MIR4803, MIR4804, MIR486-1, MIR486-2, MIR5047, MIR509-1, MIR509-2, MIR509-3, MIR5095, MIR5096, MIR512-1, MIR512-2, MIR515-1, MIR515-2, MIR521-1, MIR521-2, MIR5739, MIR5787, MIR6068, MIR6069, MIR6070, MIR6071, MIR6072, MIR6073, MIR6074, MIR6075, MIR6076, MIR6077, MIR6078, MIR6079, MIR6080, MIR6081, MIR6082, MIR6083, MIR6084, MIR6085, MIR6086, MIR6087, MIR6088, MIR6089, MIR6090, MIR6124, MIR6125, MIR6126, MIR6127, MIR6128, MIR6129, MIR6130, MIR6131, MIR6132, MIR6133, MIR6134, MIR6165, MIR6499, MIR6500, MIR6501, MIR6502, MIR6503, MIR6504, MIR6505, MIR6506, MIR6507, MIR6508, MIR6509, MIR6510, MIR6511A1, MIR6511A2, MIR6511A3, MIR6511A4, MIR6511B1, MIR6511B2, MIR6512, MIR6513, MIR6514, MIR6515, MIR6516, MIR6715A, MIR6715B, MIR6716, MIR6717, MIR6718, MIR6719, MIR6720, MIR6721, MIR6722, MIR6723, MIR6726, MIR6727, MIR6728, MIR6729, MIR6730, MIR6731, MIR6732, MIR6733, MIR6734, MIR6735, MIR6736, MIR6737, MIR6738, MIR6739, MIR6740, MIR6741, MIR6742, MIR6743, MIR6744, MIR6745, MIR6746, MIR6747, MIR6748, MIR6749, MIR6750, MIR6751, MIR6752, MIR6753, MIR6754, MIR6755, MIR6756, MIR6757, MIR6758, MIR6759, MIR6760, MIR6761, MIR6762, MIR6763, MIR6764, MIR6765, MIR6766, MIR6767, MIR6768, MIR6769A, MIR6769B, MIR6771, MIR6772, MIR6773, MIR6774, MIR6775, MIR6776, MIR6777, MIR6778, MIR6779, MIR6780A, MIR6780B, MIR6781, MIR6782, MIR6783, MIR6784, MIR6785, MIR6786, MIR6787, MIR6788, MIR6789, MIR6790, MIR6791, MIR6792, MIR6793, MIR6794, MIR6795, MIR6796, MIR6797, MIR6798, MIR6799, MIR6800, MIR6801, MIR6802, MIR6803, MIR6804, MIR6805, MIR6806, MIR6807, MIR6808, MIR6809, MIR6810, MIR6811, MIR6812, MIR6813, MIR6814, MIR6815, MIR6816, MIR6817, MIR6818, MIR6819, MIR6820, MIR6821, MIR6822, MIR6823, MIR6824, MIR6825, MIR6826, MIR6827, MIR6828, MIR6829, MIR6830, MIR6831, MIR6832, MIR6833, MIR6834, MIR6835, MIR6836, MIR6837, MIR6838, MIR6839, MIR6840, MIR6841, MIR6842, MIR6843, MIR6844, MIR6845, MIR6846, MIR6847, MIR6848, MIR6849, MIR6850, MIR6851, MIR6852, MIR6853, MIR6854, MIR6855, MIR6856, MIR6857, MIR6858, MIR6860, MIR6861, MIR6863, MIR6864, MIR6865, MIR6866, MIR6867, MIR6868, MIR6869, MIR6870, MIR6871, MIR6872, MIR6873, MIR6874, MIR6875, MIR6876, MIR6877, MIR6878, MIR6879, MIR6880, MIR6881, MIR6882, MIR6883, MIR6884, MIR6885, MIR6886, MIR6887, MIR6888, MIR6889, MIR6890, MIR6891, MIR6892, MIR6893, MIR6894, MIR6895, MIR7106, MIR7107, MIR7108, MIR7109, MIR7110, MIR7111, MIR7112, MIR7113, MIR7114, MIR7150, MIR7151, MIR7152, MIR7153, MIR7154, MIR7155, MIR7156, MIR7157, MIR7158, MIR7159, MIR7160, MIR7161, MIR7162, MIR7515, MIR7702, MIR7703, MIR7704, MIR7705, MIR7706, MIR7843, MIR7844, MIR7845, MIR7846, MIR7847, MIR7848, MIR7849, MIR7850, MIR7851, MIR7852, MIR7853, MIR7854, MIR7855, MIR7856, MIR7974, MIR7975, MIR7976, MIR7977, MIR7978, MIR8052, MIR8053, MIR8054, MIR8055, MIR8056, MIR8057, MIR8058, MIR8059, MIR8060, MIR8061, MIR8062, MIR8063, MIR8064, MIR8065, MIR8066, MIR8067, MIR8068, MIR8070, MIR8072, MIR8073, MIR8074, MIR8075, MIR8076, MIR8077, MIR8078, MIR8079, MIR8080, MIR8081, MIR8082, MIR8083, MIR8084, MIR8085, MIR8086, MIR8087, MIR8088, MIR8089, MIR8485, MIR941-1, MIR941-2, MIR941-3, MIR941-4, MIR941-5, MIR9500, MIR1184-1, MIR1184-2, MIR1184-3, MIR1185-1, MIR1185-2, MIR1233-1, MIR1233-2, MIR1244-1, MIR1244-2, MIR1244-3, MIR1244-4, MIR1254-1, MIR1254-2, MIR1283-1, MIR1283-2, MIR1285-1, MIR1285-2, MIR1289-1, MIR1289-2, MIR1302-1, MIR1302-2, MIR1302-3, MIR1302-4, MIR1302-5, MIR1302-6, MIR1302-7, MIR1302-8, MIR1302-9, MIR1972-1, MIR1972-2, MIR3116-1, MIR3116-2, MIR3118-1, MIR3118-2, MIR3118-3, MIR3118-4, MIR3119-1, MIR3119-2, MIR3130-1, MIR3130-2, MIR3156-1, MIR3156-2, MIR3156-3, MIR3158-1, MIR3158-2, MIR3160-1, MIR3160-2, MIR3179-1, MIR3179-2, MIR3179-3, MIR3179-4, MIR3180-1, MIR3180-2, MIR3180-3, MIR3180-4, MIR3180-5, MIR3198-1, MIR3198-2, MIR3199-1, MIR3199-2, MIR3202-1, MIR3202-2, MIR3648-1, MIR3648-2, MIR3670-1, MIR3670-3, MIR3670-4, MIR3680-1, MIR3687-1, MIR3687-2, MIR3688-1, MIR3688-2, MIR3910-1, MIR3910-2, MIR3913-1, MIR3913-2, MIR3914-1, MIR3914-2, MIR3926-1, MIR3926-2, MIR4283-1, MIR4283-2, MIR4315-1, MIR4315-2, MIR4435-1, MIR4435-2, MIR4444-1, MIR4472-1, MIR4472-2, MIR4509-1, MIR4509-2, MIR4509-3, MIR4520-1, MIR4520-2, MIR4536-1, MIR4650-1, MIR4650-2, MIR4679-1, MIR4679-2, MIR4771-1, MIR4771-2, MIR4773-1, MIR4773-2, MIR4776-1, MIR4776-2, MIR5701-3, MIR6724-1, MIR6724-2, MIR6724-3, MIR6724-4, MIR6770-1, MIR6770-2, MIR6770-3, MIR6859-1, MIR6859-2, MIR6859-3, MIR6859-4, MIR6862-1, MIR6862-2, MIR7641-1, MIR7641-2, MIR7973-1, MIR7973-2, MIR8069-1, MIR8069-2, MIR8071-1, MIR8071-2, MIR1302-10, MIR1302-11, combinations thereof, or their cognates in other species.

In some aspects, the cargo compound is a gene editing molecule. Gene editing molecules include, but are not limited to Zinc Finger nucleases, TALENS, and CRISPR/Cas system molecules (e.g. CRISPR guide sequences and/or Cas proteins).

The EV cargo can include any small molecule able to be transferred via hemichannels to the EV interior, entrapped within the EV, transported by EVs to the site of therapy and transferred to target cells by gap junction channels at the site of therapy. Such therapeutic molecules can include drugs, amino acids, small peptides and peptidergic molecules, nucleotides and nucleotidic molecules, lipids and lipidic molecules, microRNAs, long non-coding RNAs and all other hemichannel-permeant molecules. The provided EV invention can take-up, carry as cargo and deliver any drug or small molecule capable of permeating a hemichannel. Usually, these molecules can be membrane non-permeant so that they are retained within the EV membrane once taken up via hemichannels. They can also be membrane-permeant, but become membrane non-permeant once inside the EV. For example, certain drugs can have chemical groups bonded by ester linkage to the molecule that promote movement across the exosomal membrane enabling loading of the EV composition. Once inside the EV these ester bonds can be cleaved by an esterase, or ester bonding breaking activity, which can disable its ability to permeabilize back through the EV membrane and also restore chemically modified molecules such as peptides to structures that they can assume in nature. Drug cargo molecules with ester bonded chemical groups as detailed here can also be used to load exosomal producing cells or tissues. EVs produced by the cells that have encapsulated the drug cargo can then be isolated from the cells or media conditioned by cells, and these employed in the methods and treatments specified herein. In some aspects, the esterase or ester bond breaking activity may be incorporated into exosomes not already having such activity by directly transducing exosomes with esterase enzymes or by genetically modifying cells, tissues or organisms that can produce exosomes. Drug matching the parameters specified herein can be found in the Pharmacopoeia in the United States Pharmacopoeia (http://www.usp.org), The International Pharmacopoeia (https://web.archive.org/web/20060328053011/http://www.who.int/medicines/publications/pharmacopoeia/overview/en/) and other in other pharmacopoeias and these citations are incorporated by reference.

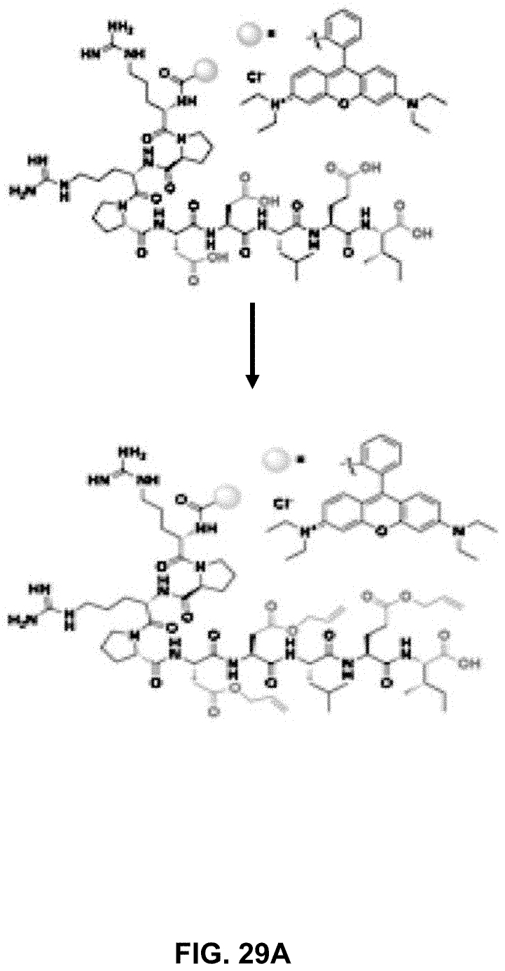

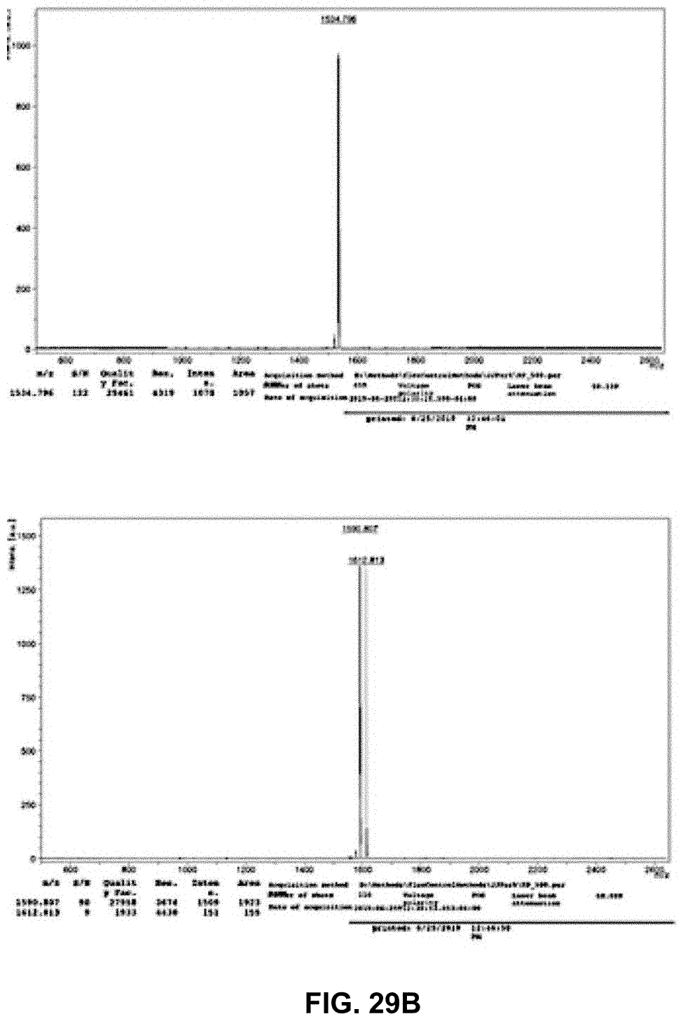

In some aspects, cargo peptides can have one or more ester bonded chemical groups (e.g., a methyl group) at one or more glutamate (E) and/or aspartate (D) residues, or at the carboxyl terminus of the polypeptide to aid translocation of the peptide into the exosome. The charge of the molecule can be modified by shielding chemical groups to aid this translocation in an ion gradient. In some aspects this gradient can be a pH gradient. In some aspects, the pH gradient is formed between the inside of the EV and the outside EV environment. In some aspects, the cargo molecule can include one or more charge shielding groups. In some aspects, charge shielding group is also an ester bonded chemical group. The charge shielding group can mask one or more charged groups on the cargo molecule to effectively change the overall charge of the cargo group. This can improve or allow for the use of a pH gradient to drive loading of the EV. The shielding groups can be a methyl group as exemplified in RhodB aCT11 with ester bonded methyls (see e.g. FIG. 29A). In other aspects, the estergroup can be an allyl group, an alcohol (ethanol, n-propanol, isopropanol, butanol, tera-butanol), aromatic alcohols (benzyl alcohol) as well as reactive alkynes (propargyl alcohol), glycerols, as well as alkenes (allyl alcohol), which can be used to install other chemical groups. In some aspects, more than one such ester group can be included, which can increase the loading efficiency. Depending on the cargo, shielding and/or addition of ester-bonding of cleavable groups at multiple locations on the molecule can be included to achieve the desired property. RhodB aCT11 with ester bonded methyl is a non-limiting example of this concept, wherein groups are placed at all 3 of its D and E residues, as well as its former carboxyl terminus. Charge on nucleic acid molecules (e.g., miRNAs) can enable preferential accumulation inside exosomes in response to an ion and/or pH gradient and these charges can also be modified by shielding groups to achieve a desired chemical property.

In some aspects the cargo compound can be functionalized to incorporate one or more COOH or OH groups available to from an ester linkage with a second molecule. Methods of functionlizing various peptides, polypeptides, polynucleotides, and other compounds to include such functionalizations will be appreciated by one of ordinary skill in the art in view of this disclosure. In some aspects, the cargo compound contains a reactive group that can form an ester linkage with another molecule.

In some aspects, the cargo compounds can be peptides that can include, without limitation, gap19, L2, Cx43 src peptide, aCT peptides (e.g. aCT1, aCT11, aCT1-I, aCT11-1), JM peptides and other peptides that are able to permeate hemichannels—examples of which can be found in the following citations—PMIDs: 28712848, 23734129, 19317641, 28694772, 23664811, 17576073, 28063303, 27856346, 25652199, 28931622, 25591543 doi.org/10.1016/j.drudis.2014.10.003, doi.org/10.1016/j.drudis.2013.05.011, and patents/patent applications WO2013163423 A1, WO2008157840 A3, U.S. Pat. No. 7,888,319 B2, US20160166637 A1, U.S. Pat. No. 9,345,744 B2, WO2009148552 A2, WO2013131040 A1, and listed as a compendium at http://www.usp.org/biologics/peptides—which together with the exemplary uses and aspects provided by these peptides are incorporated herein by reference. In some aspects, the peptide or fragment thereof can have a sequence that is about 90% to 100% identical to any one of SEQ ID NOs: 13-47, 49-116, 133 or a combination thereof. In some aspects, the cargo molecule is ACT1 (SEQ ID NO: 111). In some aspects, the cargo molecule is ACT1-I (SEQ ID NO: 112). In some aspects, the cargo molecule is a polypeptide comprising a sequence 90-100 percent identical to SEQ ID NO: 13 or 14 or a combination thereof.

Nucleic acid molecules (e.g., siRNA, miRNAs) can permeate hemichannels and thus can be loaded and delivered by the provided compositions (WO2005059111 A3—which herein incorporated by reference). Examples of such molecules can be found in doi: 10.1016/j.chembiol.2011.12.008, the references listed at the web page http://www.nature.com/focus/rna-based-therapies/index.html, PMIDs 21986484, 15033581, 16037090, 28655327, 28497038, 27612280, 26773301, 26514375, 28962871 doi: 10.1113/jphysiol.2005.090985 and the patents WO2008079412 B1 and WO2005059111 A3. The compositions and exemplary uses and aspects of the nucleic acids in the citations in this paragraph are incorporated herein by reference.

Methods for the physical characterization and quantification of EVs and their cargoes are known to those skilled in the art (PMID: 27495390; PMID: 24009896 PMID: 27035807; PMID: 27018079; PMID: 25536934—these citations are incorporated by reference). Approaches can include, but are not limited to, standard protein assays such as the Bradford assay, UV spectrophotometry, HPLC, TMS, Western blotting, Elisa as well as and/or in conjunction with the Nanosight instrument, and ExoELISA (System Biosciences). The methods cited, as well as other methods known to those skilled in the art, can be used to quantify the invention provided herein for purposes that include EV purification, determining EV yield, determining EV dosage, determining loading efficiency of the loaded therapeutic and other parameters that can provide the parameter desired from the EV invention described herein. For the purposes of the EV invention herein measurements of particle size, particle density, protein concentration, nucleic acid concentration, EV Cx43 levels, EV marker level (e.g., CD9, CD63, CD81, TSG101, MFGE8/lactadherin, HSP90B1, calnexin, GM130) and assays for the EV cargo including expressed as a function of the aforementioned measurements (e.g., [aCT11]/particle density, [JM peptide]/[total protein] and so on).

Loading the Engineered Vesicles with a Cargo Compound

Cells used to produce the extracellular vesicles can be loaded with one or more cargo compounds described herein, thus when they produce an extracellular vesicle, the cargo compound is incorporated by the cellular formation pathway (e.g. budding and endocytosis) into the extracellular vesicle.

The cargo compound can be loaded into formed engineered vesicle as well through the engineered connexon. Chemical gating of the engineered vesicles, such as manipulation of Ca²⁺ concentration or alkalinity can be used to load or release compounds from the engineered vesicles. As previously discussed, the engineered connexon can be responsive to calcium or alkalinity. An empty engineered vesicle can be placed in solution with a concentration of calcium that stimulates opening of the engineered connexon(s) (e.g. a low calcium concentration. For example, Ca²⁺ concentration in the solution may vary between 0 to 0.1 mM. Ca²⁺ concentration in the solution may also vary between 0 to 2 mM, depending on the presence of other chemicals in the solution that may affect the manner in which the connexon Ca²⁺ sensor senses the concentration, causing it to gate open. For example, a low calcium concentration can be achieved, by the addition of EDTA and/or EGTA to remove or bind calcium, in the presence or absence of calcium. The solution can also contain one or more cargo compounds. When the engineered connexons are open, the one or more cargo compounds present in the solution move via diffusion into the empty engineered vesicle through the open engineered connexon. After loading, the concentration of calcium in the solution can be adjusted to a high concentration stimulate closing of the engineered connexons and the loaded engineered vesicles can be removed. For example, Ca²⁺ concentration in the solution may be increased to 0.2 mM or more. Ca²⁺ concentration in the solution may also be below 0.2 mM to effect channel closure, depending on the presence of other chemicals in the solution that buffer and or release calcium in a manner that the connexon Ca²⁺ sensor senses the concentration, causing it to gate closed. For example, an increased calcium concentration can be achieved, by addition of the photolabile chelator, o-nitrophenyl EGTA which binds calcium, but then in response to an appropriate light wavelength releases calcium. Thus, by exposure to light the concentration of calcium can be manipulated thereby causing an opening or closing of the connexon. Other examples of inducible calcium release include light sensitive membrane channels designed to release calcium in response to light. In some aspects the molecular weight of the cargo compound to be loaded via this mechanism can be 2000 daltons or less. Connexons have shown facility for passing molecules of linear geometries such as peptides and miRNAs. Thus, in some cases the molecule transiting the pore may be greater than 2000 daltons and be up to 8000 daltons. The effective concentration of Ca²⁺ to open and close can vary depending on cell type and type of connexin expressed.

In some aspects, the cargo compound can be loaded directly into the engineered vesicle by manipulation by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques EVs may be stably or transiently loaded.

As previously discussed, in some aspects, the cargo compound can contain permeating chemical groups linked by ester bonds to the cargo compound. Once inside an exosome containing an esterase or other ester bonding breaking activity, the ester bonds can be cleaved thus making the cargo compound substantially impermeable to the EV membrane and effectively trapped in the EV. Thus, in some aspects, after the EVs are loaded with the said cargo compound, if not already active, esterases present in the EV can be activated and break the ester bonds linking the membrane permeating chemical groups to the cargo compound. For example, attachment of moieties such as methyl groups by ester bonds to negatively charged aspartic (D) and glutamic (E) amino acids and the carboxyl terminal group of aCT11 can cause the molecule to take on the characteristics of a weak base. Conversely, masking positive charges by attached chemical groups can enhance the acidic character of a molecule. A characteristic of acidic and basic molecules is that they respond to pH gradients by undergoing net translocation across membranes, followed by accumulation in proportion to the magnitude of the pH gradient. Thus, if pH in the external solution is more alkaline than within the exosome, the pH gradient can drive basic molecules into the interior of the exosome, providing for efficient loading of EVs with drug molecules. The same is true for acidic molecules, including nucleic acids (e.g., miRNAs), excepting that the direction of the gradient is reversed—i.e., exosomal exterior is alkaline relative to the exterior solution.

Esterases that can be present or included in the EVs can include, but are not limited to, CNP 280752 2′, 3′-cyclic nucleotide 3′ phosphodiesterase SMPD1 505097 sphingomyelin phosphodiesterase 1, acid lysosomal CES4A 529706 carboxylesterase 4A LCAT 510960 lecithin-cholesterol acyltransferase SMPDL3B 518699 sphingomyelin phosphodiesterase, acid-like 3B CES3 513112 carboxylesterase 3 ENPP7 505388 ectonucleotide pyrophosphatase/phosphodiesterase 7 L00100849541 100849541 glycerophosphodiester phosphodiesterase domain-containing protein 4-like LOC790012 790012 1-phosphatidylinositol 4,5-bisphosphate phosphodiesterase delta-1 PCED1B 540367 PC-esterase domain containing 1B PDE6C 281975 phosphodiesterase 6C, cGMP-specific, cone, alpha prime PDE4D 539556 phosphodiesterase 4D, cAMP-specific ACOT13 504870 acyl-CoA thioesterase 13 BREH1 497207 retinyl ester hydrolase type 1 CES5A 513992 carboxylesterase 5A IAH1 614320 isoamyl acetate-hydrolyzing esterase 1 homolog (S. cerevisiae) LOC101906659 101906659 GDSL esterase/lipase At1g29670-like LOC615277 615277 acyl-coenzyme A thioesterase THEM4 NOTUM 525682 notum pectinacetylesterase homolog (Drosophila) PCED1A 614835 PC-esterase domain containing 1A PDE10A 506061 phosphodiesterase 10A PDE6H 281978 phosphodiesterase 6H, cGMP-specific, cone, gamma SMPD3 514201 sphingomyelin phosphodiesterase 3, neutral membrane (neutral sphingomyelinase II) ACOT8 504360 acyl-CoA thioesterase 8 BCHE 534616 butyrylcholinesterase ENPP4 538583 ectonucleotide pyrophosphatase/phosphodiesterase 4 (putative) ENPP5 512304 ectonucleotide pyrophosphatase/phosphodiesterase 5 (putative) NXPE2 782358 neurexophilin and PC-esterase domain family, member 2 NXPE4 515648 neurexophilin and PC-esterase domain family, member 4 PDE1C 526211 phosphodiesterase 10, calmodulin-dependent 70 kDa PTER 782020 phosphotriesterase related CPPED1 104968445 calcineurin-like phosphoesterase domain containing 1 CPPED1 537938 calcineurin-like phosphoesterase domain containing 1 ENPP1 615535 ectonucleotide pyrophosphatase/phosphodiesterase 1 MPPED1 526018 metallophosphoesterase domain containing 1 PDE4B 100124505 phosphodiesterase 4B, cAMP-specific PDE8A 506787 phosphodiesterase 8A PPME1 535390 protein phosphatase methylesterase 1 UCHL3 520170 ubiquitin carboxyl-terminal esterase L3 (ubiquitin thiolesterase) ENPP3 529405 ectonucleotide pyrophosphatase/phosphodiesterase 3 ESD 535653 esterase D and combinations thereof.

The EVs can include other enzymes, including but not limited to Acyl-protein thioesterase 1 ACOT1 25 kDa 2′,3′-cyclic-nucleotide 3′-phosphodiesterase CN37 45 kDa Isoamyl acetate-hydrolyzing esterase 1 homolog IAH1 28 kDa, Apolipoprotein A-IV APOA4, and combinations thereof.

Gradients of pH can be achieved by adjusting the exosomal buffer solution to a pH of above or below neutral pH 7, for example to pH 6.6 or 8.5. To enhance the gradient, exosomes can be placed in a low Ca2+ solution (e.g., to 0.5 mM or below) that is buffered below pH 7.0 (e.g. to pH 6) to acidify the exosome interior. We have measured cow milk at a pH of ˜6.6. Exosomes can be subject to manipulations to cause temporary changes in permeability in the presence of buffered solutions such that the interior of the exosome assumes the pH, or other desired characteristics, of the exterior buffered solutions, including for cargo loading. Such temporary changes can include raising and lowering temperature between 4-55 degrees for brief periods once, or in cycles, such that exchange across the exosomal membrane occurs due to changes in membrane fluidity, subsequently leaving the membrane largely intact and activities such as the ester bond breaking activity inside the exosome (e.g. esterase enzymes) functional. Transient permeabilization can be achieved by electric fields/electroporation, freeze thawing, sonication, cavitation, high ion concentrations, detergents, saponin, hemichannel opening or by ionophores. The effect of such transient permeabilizing manipulations can applied singly, multiply or in combination to achieve the desired effect on loading the exosome interior with the desired species. Following incubation at the targeted pH, the pH of the exterior buffer can be adjusted to generate a pH gradient between the exosome exterior and interior that can provide efficient loading of EVs with drug molecules with basic or acidic molecules. In one example, ammonium sulfate can be used to generate a pH gradient and for the encapsulation of cargo molecules. In other examples, pH or ion gradient, sulphate-, phosphate-, citrate- or acetate-salt gradient, EDTA-ion gradient, ammonium-salt gradient, an alkylated ammonium-salt gradient, Mn2+−, Cu2+−, Na+−, K+− gradient, and/or ionophores can be used to generate the gradient between the EV interior and exterior that drives cargo loading into the EV.

The THPdb (http://crdd.osdd.net/raghava/thpdb/) repository contains a list of Food and Drug Administration (FDA) approved therapeutic peptides and proteins. These compounds and other molecules can be loaded as cargo molecules in EVs by the methods described herein, including variant molecules incorporating D and E residues and other modifications to enable linkage of membrane permeant chemical groups via ester bonds. Examples of such modifiable cargo molecules can include pexi-ganan, plecanatide, etel-calcetide, semaglutide, corticotropin, crea-tine, tafazzin, lypressin, vasopressin, angiotensins, oxytocin, eledoisin, somatostatin, fely-pressin, calcitonin, orni-pressin, desmopressin, terlipressin, amba-mustine, tetracosactide, elcatonin, saralasin, cargutocin, buserelin, leuprorelin, thymo-pentin, enalapril, triptorelin, calcitonin, goserelin, lisinopril, octreotide, romurtide, thymosin, elami-pre-tide, m tp1 3 1, elcatonin, eledoisin, enalapril, bivalirudin, cemadotin, exena-tide, ziconotide. chlorotoxin I-135 conjugate, elisi-depsin, dalaza-tide, and SOR—C13.

alphaCT11-1 Peptide and Variants Thereof

As previously discussed, the alphaCT11-1 (SEQ ID NO: 14) peptide can be provided as a cargo molecule contained in an EV described herein. In some aspects, the alphaCT11-1 peptide can comprise or be composed only of a peptide that is identical to SEQ ID NO: 14. In some aspects, the aCT11-1 peptide is coupled to an N-terminal antennapedia sequence and can form a sequence identical to SEQ ID NO: 112 and is also referenced herein as ACT1-I. In some aspects, the alphaCT11-1 peptide can be provided as a cargo molecule be composed only of a peptide that is identical to SEQ ID NO: 14. In some aspects, the peptide identical to SEQ ID NO:14 can be operatively coupled to an antennapedia internalization sequence to form ACT1-I (SEQ ID NO: 112), In some aspects, the alphaCT11-1 and/or aCT1-I peptides can be included in a pharmaceutical formulation. In some aspects, the aCT11-I and/or aCT1-I peptides are provided in a delivery vesicle, such as an EV described herein. In some aspects, the alphaCT11-1 and/or aCT1-I peptides are not provided in a delivery vesicle such as an EV described herein. In other words, in some aspects, the aCT1-I or aCT11-1 peptides are provided in a formulation that does not include them being encapsulated or otherwise included in an EV. Additional details of the pharmaceutical formulations that include ACT11-I or ACT1-I) peptides are described elsewhere herein.

Pharmaceutical Formulations

The engineered vesicles (with or without a cargo molecule), alphaCT11-I, and/or ACT1-I peptides described herein can be included as part of, such as an active ingredient, a pharmaceutical formulation. As such, also described herein are pharmaceutical formulations that can include an amount of an engineered vesicle and a pharmaceutically acceptable carrier. As such, also described are pharmaceutical formulations containing one or more of the engineered vesicles and salts thereof, or pharmaceutically acceptable salts thereof described herein.

The engineered vesicles, alphaCT11-I, and/or ACT1-I peptides, or pharmaceutical formulations thereof can be administered by any suitable route to a subject. As discussed in greater detail herein subject can have a disease or suspected of having a disease, condition, and/or disorder. As discussed in greater detail herein, the engineered vesicles, alphaCT11-I, and/or ACT1-I peptides, and/or pharmaceutical formulations thereof can be co-administered with another formulation or treatment modality. In some aspects, the engineered vesicles, alphaCT11-I, and/or ACT1-I peptides described herein are used in the manufacture of a medicament for the treatment or prevention of a disease, condition, and/or disorder in a subject.

Pharmaceutically Acceptable Carriers and Auxiliary Ingredients and Agents

The pharmaceutical formulations containing an amount of an engineered vesicle, alphaCT11-I, and/or ACT1-I peptides described herein can further include a pharmaceutically acceptable carrier. Suitable pharmaceutically acceptable carriers include, but are not limited to water, milk, milk products, milk components, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition. Isolated EVs can be added to milk or a milk product to afford the benefits that EVs can derive from suspension in this media. For example, EVs loaded with aCT11 peptide can be placed in a chocolate milkshake in order to orally administer the therapeutic EVs to a heart attack patient. In a further example, aCT11 peptide in an exosomal vector in a carrier may be given to patients with atrial arrhythmia on a daily, multi-day or weekly basis to control said arrhythmias.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In addition to the amount of an engineered vesicle alphaCT11-I, and/or ACT1-I peptides described herein, the pharmaceutical formulations can also include an effective amount of auxiliary active agents, including but not limited to, antisense or RNA interference molecules, chemotherapeutics, or antineoplasic agents, hormones, antibiotics, antivirals, immunomodulating agents, antinausea, analgesics, anti-inflammatory agents, antipyretics, antibiotics, and/or antibodies or fragments thereof.

Amounts of the Engineered Vesicles, alphaCT11-I, and/or ACT1-I Peptides, and Auxiliary Active Agents

The amount, including an effective amount, of the engineered vesicle, alphaCT11-I, and/or ACT1-I peptides, or auxiliary agent (when included the formulation in the pharmaceutical formulation) can range from about 0.001 micrograms to about 1000 grams. The amount, including an effective amount, can range from about 0.001 micrograms to about 0.01 micrograms. The amount, including an effective amount, can range from about 0.01 micrograms to about 0.1 micrograms. The amount, including an effective amount, can range from about 0.1 micrograms to about 1.0 grams. The amount, including an effective amount, can range from about 1.0 grams to about 10 grams. The amount, including an effective amount, can range from about 10 grams to about 100 grams. The amount, including an effective amount, can range from about 100 grams to about 1000 grams.

The amount, including an effective amount, can range from about 0.01 IU to about 1000 IU. The amount, including an effective amount, can range from 0.001 mL to about 1000 mL. The amount, including an effective amount, can range from about 1% w/w to about 99% w/w of the total pharmaceutical formulation. The amount, including an effective amount, can range from about 1% v/v to about 99% v/v of the total pharmaceutical formulation. The amount, including an effective amount, can range from about 1% w/v to about 90% w/v of the total pharmaceutical formulation.

The auxiliary active agent can be included in the pharmaceutical formulation or can exist as a stand-alone compound or pharmaceutical formulation that can be administered contemporaneously or sequentially with the compound, derivative thereof, or pharmaceutical formulation thereof. In aspects where the auxiliary active agent is a stand-alone compound or pharmaceutical formulation, the effective amount of the auxiliary active agent can vary depending on the auxiliary active agent used and can be as described above. The auxiliary active agent can be simultaneously or sequentially administered with the engineered vesicles, alphaCT11-I, and/or ACT1-I peptides, or pharmaceutical formulation thereof.

Dosage Forms

The pharmaceutical formulations described herein can be in a dosage form. The dosage form can be administered to a subject in need thereof via a suitable administration route. The subject in need thereof can have, be suspected of having, and/or be at risk of developing a disease, condition, and/or disorder.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, ocular, and intradermal. Other suitable routes for administration are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions, water-in-oil liquid emulsions, oil-in-water liquid microemulsions, or water-in-oil liquid microemulsions. In some aspects, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. The subject in need thereof can have, be suspected of having, and/or be at risk of developing a disease, condition, and/or disorder.

Where appropriate, the dosage forms described herein can be microencapsulated. The dosage form can also be prepared to prolong or sustain the release of any ingredient. In some aspects, the compound or derivative thereof is the ingredient whose release is delayed. In other aspects, the release of an auxiliary ingredient or auxiliary active agent is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water soluble polymer, water insoluble polymers, and/or pH dependent polymers, with or without water insoluble/water soluble non polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these aspects, compound, derivative thereof, auxiliary active ingredient, and/or pharmaceutically acceptable salt thereof are incorporated into a liposome. In some aspects, an engineered vesicle, alphaCT11-I, and/or ACT1-I peptides, auxiliary active ingredient, and/or pharmaceutically acceptable salts thereof is integrated into the lipid membrane of the liposome (separate from the engineered vesicle described herein). In other aspects, an engineered vesicle, alphaCT11-I, and/or ACT1-I peptides, auxiliary active ingredient, and/or pharmaceutically acceptable salt thereof are contained in the aqueous phase of the liposome (separate from the engineered vesicle described herein). Where the dosage form is a liposome, the pharmaceutical formulation is thus a liposomal formulation. The liposomal formulation can be administered to a subject in need thereof. The subject in need thereof can have, be suspected of having, and/or be at risk of developing a disease, condition, and/or disorder.

Dosage forms adapted for topical administration can be formulated as ointments, creams, suspensions, lotions, powders, solutions, pastes, gels (e.g. poloxamer gel), sprays, aerosols, or oils. In some aspects for treatments of the eye or other external tissues, for example the mouth or the skin, the pharmaceutical formulations are applied as a topical ointment or cream. When formulated in an ointment, the compound, derivative thereof, auxiliary active ingredient, and/or pharmaceutically acceptable salt thereof can be formulated with a paraffinic or water-miscible ointment base. In other aspects, the active ingredient can be formulated in a cream with an oil-in-water cream base or a water-in-oil base. Dosage forms adapted for topical administration in the mouth include lozenges, pastilles, and mouth washes.

In some aspects the provided pharmaceutically acceptable carrier is a poloxamer. Poloxamers, referred to by the trade name Pluronics®, are nonionic surfactants that form clear thermoreversible gels in water. Poloxamers are polyethylene oxide-polypropylene oxide-polyethylene oxide (PEO-PPO-PEO) tri-block copolymers. The two polyethylene oxide chains are hydrophilic but the polypropylene chain is hydrophobic. These hydrophobic and hydrophilic characteristics take charge when placed in aqueous solutions. The PEO-PPO-PEO chains take the form of small strands where the hydrophobic centers can come together to form micelles. The micelle, sequentially, tend to have gelling characteristics because they come together in groups to form solids (gels) where water is just slightly present near the hydrophilic ends. When it is chilled, it can liquefy, but it can harden when warmed. This characteristic makes it useful in pharmaceutical compounding because it can be drawn into a syringe for accurate dose measurement when it is cold. When it warms to body temperature (e.g., when applied to skin) it can thicken to a useful consistency (especially when combined with soy lecithin/isopropyl palmitate) to facilitate proper inunction and adhesion. Pluronic® F127 (F127) may be used in some aspects. F127 has a EO:PO:EO ratio of 100:65:100, which by weight has a PEO:PPO ratio of 2:1. Pluronic gel is an aqueous solution and typically contains 20-30% F127. Thus, the provided compositions can be administered in F127.

Dosage forms adapted for nasal or inhalation administration include aerosols, solutions, suspension drops, gels, or dry powders. The engineered vesicles, auxiliary active ingredient, and/or pharmaceutically acceptable salt thereof in a dosage form adapted for inhalation is in a particle-size-reduced form that is obtained or obtainable by micronization. In some aspects, the particle size of the size reduced (e.g. micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active ingredient, which may be generated by various types of metered dose pressurized aerosols, nebulizers, or insufflators. The nasal/inhalation formulations can be administered to a subject in need thereof. The subject in need thereof can have, be suspected of having, and/or be at risk of developing a disease, condition, and/or disorder.

In some aspects, the dosage forms are aerosol formulations suitable for administration by inhalation. In some of these aspects, the aerosol formulation contains a solution or fine suspension of a compound, derivative thereof, auxiliary active ingredient, and/or pharmaceutically acceptable salt thereof a pharmaceutically acceptable aqueous or non-aqueous solvent. Aerosol formulations can be presented in single or multi-dose quantities in sterile form in a sealed container. For some of these aspects, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other aspects are contained in a pump-atomizer. The pressurized aerosol formulation can also contain a solution or a suspension of an engineered vesicle as described herein, auxiliary active ingredient, and/or pharmaceutically acceptable salt thereof. In further aspects, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, 5, or more times daily, in which 1, 2, 4, or more doses are delivered each time. The aerosol formulations can be administered to a subject in need thereof. The subject in need thereof can have, be suspected of having, and/or be at risk of developing a disease, condition, and/or disorder.

For some dosage forms suitable and/or adapted for inhaled administration, the pharmaceutical formulation is a dry powder inhalable formulations. In addition to the engineered vesicles, alphaCT11-I, and/or ACT1-I peptides described herein, auxiliary active ingredient, and/or pharmaceutically acceptable salt thereof, such a dosage form can contain a powder base such as lactose, glucose, trehalose, mannitol, and/or starch. The engineered vesicles described herein, alphaCT11-I, and/or ACT1-I peptides described herein, auxiliary active ingredient, and/or pharmaceutically acceptable salt thereof can be included in a particle-size reduced form. A performance modifier, such as L-leucine or another amino acid, cellobiose octaacetate, and/or metals salts of stearic acid, such as magnesium or calcium stearate.

The aerosol formulations can be arranged so that each metered dose of aerosol contains a predetermined amount of an active ingredient, such as the one or more of the compounds described herein.

Dosage forms can be adapted for ocular administration and can be liquid, gel, and/or aerosol as described elsewhere herein.

Dosage forms can be adapted for vaginal administration can be presented as pessaries, tampons, creams, gels, pastes, foams, or spray formulations. Dosage forms adapted for rectal administration include suppositories or enemas. The vaginal and/or rectal formulations can be administered to a subject in need thereof. The subject in need thereof can have, be suspected of having, and/or be at risk of developing a disease, condition, and/or disorder.

Dosage forms adapted for parenteral administration and/or adapted for injection can include aqueous and/or non-aqueous sterile injection solutions, which can contain anti-oxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some aspects, from sterile powders, granules, and tablets. The parenteral formulations can be administered to a subject in need thereof. The subject in need thereof can have, be suspected of having, and/or be at risk of developing a disease, condition, and/or disorder.

For some aspects, the dosage form contains a predetermined amount of an engineered vesicle, alphaCT11-I, and/or ACT1-I peptides described herein per unit dose. The predetermined amount of the engineered vesicle, alphaCT11-I, and/or ACT1-I peptides can be an effective amount of the compound and/or derivative thereof to treat, prevent, or mitigate one or more symptoms of a disease, disorder, or condition. The predetermined amount of the engineered vesicle(s), alphaCT11-I, and/or ACT1-I peptides can be an appropriate fraction of the total amount to be administered in a total dose (which can be based on e.g. a time frame (e.g.) minute, hour, day, month, year) or a total amount to treat a disease condition or disorder). Such unit doses may therefore be administered once or more than once a day (e.g. 1, 2, 3, 4, 5, 6, or more times per day). Such unit doses may therefore be administered once or more than once a week (e.g. 1, 2, 3, 4, 5, 6, or more times per week). Such unit doses may therefore be administered once or more than once a week (e.g. 1, 2, 3, 4, 5, 6, or more times per month). Such unit doses may therefore be administered once or more than once a year (e.g. 1, 2, 3, 4, 5, 6, or more times per year). Such pharmaceutical formulations may be prepared by any of the methods well known in the art. Unit dosages can be adapted for bolus dosing or continuous dosing as desired.

Effective dosages and schedules for administering the compositions provided herein may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual doctor in the event of any counter-indications. Dosage can vary, and can be administered in one or more dose administrations daily, for one or several days. Guidance can be found in the literature for appropriate dosages for given classes of pharmaceutical products. The range of dosage largely depends on the application of the compositions herein, severity of condition, and its route of administration.

For example, in applications as a laboratory tool for research, the compositions can be used in doses as low as 0.01% w/v. The dosage can be as low as 0.02% w/v and possibly as high as 2% w/v in topical skin wound treatments. Significantly higher concentrations of the compositions by themselves or in combination with other compounds may be used in applications like cancer/tumor therapy or as an early concentrated bolus immediately following an acute tissue injury. Thus, upper limits of the provided polypeptides may be up to 5% w/v or v/v if given as an initial bolus delivered, for example, directly into a tumor mass. Recommended upper limits of dosage for parenteral routes of administration for example intramuscular, intracerebral, intracardiac and intraspinal could be up to 1% w/v or v/v depending on the severity of the injury. This upper dosage limit may vary by formulation, depending for example on how the composition is combined with other agents promoting its action or acting in concert with it.

For continuous delivery of the provided EVs, alphaCT11-I, and/or ACT1-I peptides for example, in combination with an intravenous drip, upper limits of 0.01 g/Kg body weight over time courses determined by the doctor based on improvement in the condition can be used. In another example, upper limits of concentration of the provided EVs, alphaCT11-I, and/or ACT1-I peptides delivered topically, for example, in skin wounds can be 0.1-10 μg/cm² of wound, depending, for example, on how the composition is combined with other agents promoting or acting in concert with its action. This can be repeated at a frequency determined by a medical practitioner or otherwise empirically derived method acceptable to medical practice on improvement. In another example, upper limits of concentration of the provided EVs, alphaCT11-I, and/or ACT1-I peptides delivered internally for example, intramuscular, intracerebral, intracardiac and intraspinal can be 50-100 μg/ml of solution. Again, the frequency can be determined by the Doctor or otherwise empirically derived method acceptable to medical practice on improvement.

Materials Incorporating the Engineered Vesicles, alphaCT11-1, and/or ACT1-I Peptides, and Pharmaceutical Formulations Thereof

Also described herein are materials that can include the engineered vesicles, alphaCT11-I, and/or ACT1-I peptides, and/or pharmaceutical formulations thereof described herein. These materials can be used to treat a disease, condition, and/or disorder in a subject. In some aspects the materials described herein can be used to treat wounds, wherein the materials are coated with the provided EVs alphaCT11-I, and/or ACT1-I peptides. Non-limiting examples of materials used to treat wounds include bandages, steri-strip, sutures, staples, or grafts (e.g., skin grafts).

For example, the material (e.g., bandage, steri-strip, suture, staple, graft) can be soaked in the provided composition. The material can then be dried and sealed in a sterile container. The material can also be immersed in liquid 10-30% pluronic gel at 4° C. containing provided composition. The material can then be brought to approximate room temperature so that the gel polymerizes, leaving a coat of EV, alphaCT11-I, and/or ACT1-I pepetide-impregnated gel surrounding the material, which can be sealed in a sterile container. The provided EVs, alphaCT11-I, and/or ACT1-I peptides can also be incorporated into a cross-linkable hydrogel system, such as the poly(lactic-co-glycolic acid) (PLGA) or polyurethane, which can then be fashioned into materials for treating wounds (e.g., bandage, steri-strip, suture, staple, graft). Thus, described herein are composite hydrogel-EV, alphaCT11-I, and/or ACT1-I peptide materials.

Also disclosed are medical implants that can be coated with the engineered vesicles, alphaCT11-I, and/or ACT1-I peptides, and/or pharmaceutical formulations thereof described herein before implantation in a subject. For example, a common problem in such implant surgeries is the formation of a contraction capsule around the implant from scar tissue formation that leads to undue hardening, contraction and ultimately misshaping of the tissue of interest. The use of the present composition in or on the implant can reduce or prevent this misshaping. Non-limiting examples of medical implants include: limb prostheses, breast implants, penile implants, testicular implants, artificial eyes, facial implants, artificial joints, heart valve prostheses, vascular prostheses, dental prostheses, facial prosthesis, tilted disc valve, caged ball valve, ear prosthesis, nose prosthesis, pacemakers, cochlear implants, and skin substitutes (e.g., porcine heterograft/pigskin, BIOBRANE, cultured keratinocytes).

Diseases, Disorders, and Conditions

The engineered vesicles, alphaCT11-I, and/or ACT1-I peptides and formulations thereof can be used to deliver a cargo compound to a subject. The subject can have, be suspected of having, or be at risk of developing a disease, disorder, and/or condition. Thus, the engineered vesicles and pharmaceutical formulations thereof can be used to treat and/or prevent a disease, disorder, and/or condition in a subject.

Such diseases, disorders, and conditions can include, but are not limited to, external and internal wounds and tissue injuries, cancer, ischemic and/or hypoxic injuries (e.g. myocardial infarction and/or stroke), multiple sclerosis, psoriasis, scleroderma, acne, eczema, or a disease of the skin and/or connective tissues, cardiac diseases or disorders, neurodegenerative diseases or disorders, neurological disorders, atherosclerosis, pathologies involving epithelial permeablization and/or neovascularization (e.g., angiogenesis or vasculogenesis), respiratory distress syndrome (RDS), reperfusion injuries, dermal vascular blemish or malformation, macular degeneration, neovascularization of choriocapillaries through Bruch's membrane, diabetic retinopathy, (inflammatory and inflammation-related diseases and disorders), and radiation dermatitis.

Wounds can be chronic wounds or wounds that appear to not completely heal. Wounds that have not healed within three months, for example, are said to be chronic. Chronic wounds include, diabetic foot ulcers, ischemic, venous ulcers, venous leg ulcers, venous stasis, arterial, pressure, vasculitic, infectious, decubitis, burn, trauma-induced, gangrenous and mixed ulcers. Chronic wounds include wounds that are characterized by and/or chronic inflammation, deficient and overprofuse granulation tissue differentiation and failure of re-epithelialization and wound closure and longer repair times. Chronic wounds can include ocular ulcers, including corneal ulcers. Use of the disclosed invention in wound healing and tissue regeneration can include in humans and agricultural, sports and pet animals.

Tissue injuries can result from, for example, a cut, scrape, compression wound, stretch injury, laceration wound, crush wound, bite wound, graze, bullet wound, explosion injury, body piercing, stab wound, surgical wound, surgical intervention, medical intervention, host rejection following cell, tissue or organ grafting, pharmaceutical effect, pharmaceutical side-effect, bed sore, radiation injury, radiation illness, cosmetic skin wound, internal organ injury, disease process (e.g., asthma, cancer), infection, infectious agent, developmental process, maturational process (e.g., acne), genetic abnormality, developmental abnormality, environmental toxin, allergen, scalp injury, facial injury, jaw injury, sex organ injury, joint injury, excretory organ injury, foot injury, finger injury, toe injury, bone injury, eye injury, corneal injury, muscle injury, adipose tissue injury, lung injury, airway injury, hernia, anus injury, piles, ear injury, skin injury, abdominal injury, retinal injury, eye injury, corneal injury, arm injury, leg injury, athletic injury, back injury, birth injury, premature birth injury, toxic bite, sting, injury to barrier function, injury to endothelial barrier function, injury to epithelial barrier function, tendon injury, ligament injury, heart injury, heart valve injury, vascular system injury, cartilage injury, lymphatic system injury, craniocerebral trauma, dislocation, esophageal perforation, fistula, nail injury, foreign body, fracture, frostbite, hand injury, heat stress disorder, laceration, neck injury, self-mutilation, shock, traumatic soft tissue injury, spinal cord injury, spinal injury, sprain, strain, tendon injury, ligament injury, cartilage injury, thoracic injury, tooth injury, trauma, nervous system injury, burn, burn wound, wind burn, sun burn, chemical burn, aging, aneurism, stroke, surgical radiation injury, digestive tract injury, infarct, or ischemic injury.

Cardiac diseases and disorders can include, but are not limited to, myocardial infarction, cardio myopathies (e.g. hypertrophic cardiomyopathy), arrhythmias, congestive heart failure. The regenerative effects of the provided composition may result in beneficial changes in membrane excitability and ion transients of the heart. There are many different types of arrhythmia that can lead to abnormal function in the human heart. Arrhythmias include, but are not limited to bradycardias, tachycardias, alternans, automaticity defects, reentrant arrhythmias, fibrillation, AV nodal arrhythmias, atrial arrhythmias and triggered beats, Long QT syndrome, Short QT syndrome, Brugada syndrome, premature atrial Contractions, wandering Atrial pacemaker, Multifocal atrial tachycardia, Atrial flutter, Atrial fibrillation, Supraventricular tachycardia, AV nodal reentrant tachycardia is the most common cause of Paroxysmal Supraventricular Tachycardia, Junctional rhythm, Junctional tachycardia, Premature junctional complex, Wolff-Parkinson-White syndrome, Lown-Ganong-Levine syndrome, Premature Ventricular Contractions (PVC) sometimes called Ventricular Extra Beats, alternans and discordant alternans, Accelerated idioventricular rhythm, Monomorphic Ventricular tachycardia, Polymorphic ventricular tachycardia, Ventricular fibrillation, First degree heart block, which manifests as PR prolongation, Second degree heart block, Type 1 Second degree heart block, Type 2 Second degree heart block, Third degree heart block, and several accessory pathway disorders (e.g., Wolff-Parkinson-White syndrome (WPW)).

Neurodegenerative and neurological disorders include, but are not limited to dementia, Alzheimer's disease, Parkinson's disease and related PD-diseases, amyotrophic lateral sclerosis (ALS), motor neuron disease, schizophrenia, spinocerebellar ataxia, prion disease, Spinal muscular atrophy (SMA), multiple sclerosis, epilepsy and other seizure disorders, and Huntington's disease.

Inflammatory diseases and inflammatory-related diseases and disorders can be asthma, eczema, sinusitis, atherosclerosis, arthritis (including but not limited to rheumatoid arthritis), inflammatory bowel disease, cutaneous and systemic mastocytosis, psoriasis, and multiple sclerosis. As used herein, the term “inflammatory disorder” can include diseases or disorders which are caused, at least in part, or exacerbated, by inflammation, which is generally characterized by increased blood flow, edema, activation of immune cells (e.g., proliferation, cytokine production, or enhanced phagocytosis), heat, redness, swelling, pain and/or loss of function in the affected tissue or organ. The cause of inflammation can be due to physical damage, chemical substances, micro-organisms, tissue necrosis, cancer, or other agents or conditions.

Inflammatory disorders include acute inflammatory disorders, chronic inflammatory disorders, and recurrent inflammatory disorders. Acute inflammatory disorders are generally of relatively short duration, and last for from about a few minutes to about one to two days, although they can last several weeks. Characteristics of acute inflammatory disorders include increased blood flow, exudation of fluid and plasma proteins (edema) and emigration of leukocytes, such as neutrophils. Chronic inflammatory disorders, generally, are of longer duration, e.g., weeks to months to years or longer, and are associated histologically with the presence of lymphocytes and macrophages and with proliferation of blood vessels and connective tissue. Recurrent inflammatory disorders include disorders which recur after a period of time or which have periodic episodes. Some inflammatory disorders fall within one or more categories. Exemplary inflammatory disorders include, but are not limited to atherosclerosis; arthritis; inflammation-promoted cancers; asthma; autoimmune uveitis; adoptive immune response; dermatitis; multiple sclerosis; diabetic complications; osteoporosis; Alzheimer's disease; cerebral malaria; hemorrhagic fever; autoimmune disorders; and inflammatory bowel disease. In some aspects, the inflammatory disorder is an autoimmune disorder that, in some aspects, is selected from lupus, rheumatoid arthritis, and autoimmune encephalomyelitis.

In some aspects, the inflammatory disorder is a brain-related inflammatory disorder. The term “brain-related inflammatory” disorder is used herein to refer to a subset of inflammatory disorders that are caused, at least in part, or originate or are exacerbated, by inflammation in the brain of a subject. It has been determined that the EVs, alphaCT11-I, and/or ACT1-I peptides and pharmaceutical formulations thereof can be particularly suitable for treating such disorders as those compositions are able to cross the blood-brain barrier and effectively be used to deliver the therapeutic agents (e.g., curcumin or JSI-124) to the brain of a subject.

Kits

The engineered vesicles, alphaCT11-I, and/or ACT1-I peptides described herein and/or pharmaceutical formulations thereof described herein can be presented as a combination kit. As used herein, the terms “combination kit” or “kit of parts” refers to the compounds, or pharmaceutical formulations and additional components that are used to package, sell, market, deliver, and/or administer the combination of elements or a single element, such as the active ingredient, contained therein. Such additional components include but are not limited to, packaging, syringes, blister packages, bottles, and the like. When one or more of the components (e.g. active agents) contained in the kit are administered simultaneously, the combination kit can contain the active agents in a single pharmaceutical formulation (e.g. a tablet) or in separate pharmaceutical formulations.

When the agents are not administered simultaneously, the combination kit can contain each agent in separate pharmaceutical formulations. The separate pharmaceutical formulations can be contained in a single package or in separate packages within the kit.

The combination kit can also include instructions printed on or otherwise contained in a tangible medium of expression. The instructions can provide information regarding the content of the compound or pharmaceutical formulations contained therein, safety information regarding the content of the compound(s) or pharmaceutical formulation(s) contained therein, information regarding the dosages, indications for use, and/or recommended treatment regimen(s) for the compound(s) and/or pharmaceutical formulations contained therein. The instructions can provide directions for administering the compounds, compositions, pharmaceutical formulations, or salts thereof to a subject having, suspected of having, or predisposed to a disease, disorder, or condition described elsewhere herein. The instructions can provide directions for administering the compounds, compositions, pharmaceutical formulations, or salts thereof to a subject having, suspected of having, or predisposed to developing diabetes or a symptom thereof. The instructions can provide directions for preparing, loading, and/or administering the engineered vesicles and/or co-treatments described herein that can be included in the kit.

Methods of Using the Engineered Vesicles, alphaCT11-I, and/or ACT1-I Peptides and Pharmaceutical Formulations Thereof

An amount of the engineered vesicles, alphaCT11-I, and/or ACT1-I peptides, or pharmaceutical formulation thereof described herein can be administered to a subject in need thereof one or more times per day, week, month, or year. In aspects, the amount administered is the effective amount of the engineered vesicles, alphaCT11-I, and/or ACT1-I peptides or pharmaceutical formulation thereof. For example, the engineered vesicles, alphaCT11-I, and/or ACT1-I peptides or pharmaceutical formulation thereof can be administered in a daily dose. This amount may be given in a single dose per day. In other aspects, the daily dose may be administered over multiple doses per day, in which each containing a fraction of the total daily dose to be administered (sub-doses). In some aspects, the amount of doses delivered per day is 2, 3, 4, 5, or 6. In aspects, the engineered vesicles, alphaCT11-I, and/or ACT1-I peptides or pharmaceutical formulation thereof can be administered one or more times per week, such as 1, 2, 3, 4, 5, or 6 times per week. In aspects, the engineered vesicles, alphaCT11-I, and/or ACT1-I peptides or pharmaceutical formulation thereof be administered one or more times per month, such as 1 to 5 times per month. In aspects, the engineered vesicles, alphaCT11-I, and/or ACT1-I peptides or pharmaceutical formulation thereof can be administered one or more times per year, such as 1 to 12 times per year.

The subject in need thereof is a subject can have, can be suspected to having, can be at risk of having, can be is predisposed to developing a disease, disorder, or condition as described elsewhere herein. In some aspects the subject in need thereof has a chronic wound. In some aspects, the subject suffers from diabetic foot ulcers, ischemic, venous ulcers, venous leg ulcers, varicose veins, radiation injury, venous stasis, arterial, pressure, vasculitic, infectious, decubitis, burn, trauma-induced, gangrenous, mixed ulcers, or a combination thereof.

In aspects where more than one of compounds, formulations, additional therapeutic agents, salts thereof, or pharmaceutically acceptable salts thereof are administered to a subject in need thereof sequentially; the sequential administration may be close in time or remote in time. For example, administration of the second engineered vesicle, alphaCT11-I, and/or ACT1-I peptides or pharmaceutical formulation thereof, compound, formulation, or other therapeutic agent can occur within seconds or minutes (up to about 1 hour) after administration of the first engineered vesicle, alphaCT11-I, and/or ACT1-I peptides, or pharmaceutical formulation thereof, compound, formulation, or other therapeutic agent (close in time). In other aspects, administration of the second engineered vesicle, alphaCT11-I, and/or ACT1-I peptides or pharmaceutical formulation thereof, compound, formulation, or other therapeutic agent occurs at some other time that is more than an hour after administration of the first engineered vesicle, alphaCT11-I, and/or ACT1-I peptides or pharmaceutical formulation thereof, compound, formulation, or other therapeutic agent.

The amount of compounds, formulations, salts thereof (including pharmaceutically acceptable formulations and salts thereof) described herein can be administered in an amount ranging from about 0.01 mg to about 1000 mg per day, as calculated as the free engineered vesicle loaded with a cargo compound.

The compounds and formulations described herein can be administered in combinations with or include one or more other auxiliary agents or be given as a co-therapy as described elsewhere herein. Suitable auxiliary agents include, any of the cargo compounds listed herein. The auxiliary agents as discussed here are not contained within the engineered vesicle and based on the description elsewhere herein, the additional auxiliary agents may already be present and loaded in the engineered vesicle. The engineered vesicles, and/or formulation(s), alphaCT11-I, and/or ACT1-I peptides and/or additional therapeutic agent(s) can be administered simultaneously or sequentially by any convenient route in separate or combined pharmaceutical formulations. The additional therapeutic agents can be provided in their optically pure form or a pharmaceutically acceptable salt thereof. Suitable administration routes are described elsewhere herein.

Accordingly, also describe herein are methods of treating or preventing a disease, condition, or disorder and/or a symptom thereof in a subject by administering an engineered vesicle as described herein. It will be appreciated that the disease, condition, and disorder treated by any specific engineered vesicle described herein can be due in part to the cargo compound(s) that can be loaded in the engineered vesicle.

In some aspects, two topical applications of the engineered vesicles, alphaCT11-I, and/or ACT1-I peptides at 0.02% w/v; one applied acutely and the second applied 24 hours later can reduce inflammation, promote healing, reduce scarring, increase tensile strength, and promote tissue regeneration. However, in a clinical setting an increased frequency of up to 3 applications per day topically at a concentration of up to 5% is recommended until significant improvement is achieved as determined by a medical practitioner. For internal administration, for example, intravenously, intramuscularly, intracerebral, intracardiac and intraspinally and increased frequency of up to 3 dosages of 1% w/v or v/v per day is recommended until significant improvement is determined by the medical practitioner.

Following administration of the engineered vesicle, alphaCT11-I, and/or ACT1-I peptides for promoting wound healing, the efficacy of the therapeutic composition can be assessed in various ways well known to the skilled practitioner. For instance, one of ordinary skill in the art will understand that a composition, such as the EVs, alphaCT11-I, and/or ACT1-I peptides, and/or pharmaceutical formulations thereof disclosed herein can be efficacious in promoting wound healing in a subject by observing that the composition can reduce scar tissue formation, reduce fibrotic tissue formation, improve tissue regeneration, or reduce inflammation in the subject following tissue injury. Methods for measuring these criteria are known in the art and discussed herein.

Also described herein are methods of promoting wound healing, decreasing scarring, or decreasing inflammation in a subject, comprising administering to a subject an amount of an engineered vesicle, alphaCT11-I, and/or ACT1-I peptides or pharmaceutical formulation thereof as described herein. The wound may be a slow healing wound, a diabetic foot ulcer, a pressure ulcer, a neural injury, a dental injury, a cardiac injury, an ischemic brain injury, a spinal cord injury, a periodontal injury, a tendon or ligament injury, a venous leg ulcer, an ischemic ulcer, a bed sore, radiation injury, or a corneal ulcer. The wound may result from a muscle atrophy disease, a neurodegenerative disease (e.g., Alzheimer's disease, Parkinson's disease, Huntington's disease, a motor neuron disease, dementia, an extrapyramidal or movement disorder), a heart disease, metabolic syndrome, an eye disease, or a disease of the skin or other organ systems of the body. The subject may have a wound or injury to or of the skin or cartilage. The provided EV, alphaCT11-I, and/or ACT1-I peptides and/or pharmaceutical formulations thereof can be administered to the subject topically or parenterally. The EVs, alphaCT11-I, and/or ACT1-I peptides can be included in a pharmaceutical formulation as previously discussed.

Also described herein are methods of treating an inflammatory eye disease in a subject, comprising administering to the subject an amount of engineered vesicles, alphaCT11-I, and/or ACT1-I peptides, or a pharmaceutical formulation thereof described herein of the present invention to a subject. The inflammatory eye disease can be age related macular degeneration, a diabetic eye disease, a retinopathy, or a retinopathy of prematurity. The pharmaceutical formulation can be eye drops or gels. The method may further comprise administering, injecting, or introducing the EVs, alphaCT11-I, and/or ACT1-I peptides or pharmaceutical formulations thereof into the eye of the subject. For example, the EVs, alphaCT11-I, and/or ACT1-I peptides can be administered, injected, or introduced into the vitreous of the eye.

Also described herein are methods to treat external wounds caused by, but not limited to scrapes, cuts, lacerated wounds, bite wounds, bullet wounds, stab wounds, burn wounds, sun burns, chemical burns, surgical wounds, bed sores, radiation injuries, all kinds of acute and chronic wounds, wounds or lesions created by cosmetic skin procedures by administering an engineered vesicle as described herein or a pharmaceutical formulation thereof that is loaded with a peptide or alphaCT11-I, and/or ACT1-I peptides or pharmaceutical formulations thereof described herein to a subject in need thereof.

Also described herein are methods to treat, mitigate, or ameliorate the effects of skin aging by administering an engineered vesicle as described herein or a pharmaceutical formulation thereof that is loaded with a peptide or alphaCT11-I, and/or ACT1-I peptides s or pharmaceutical formulations thereof described herein to a subject in need thereof.

Also described herein are methods to accelerate wound healing in an external wounds and/or improve the cosmetic appearance of wounded areas, or skin subject to aging and disease by administering an engineered vesicle as described herein or a pharmaceutical formulation thereof that is loaded with a peptide or alphaCT11-I, and/or ACT1-I peptides or pharmaceutical formulations thereof described herein to a subject in need thereof.

Also described herein are methods of treating an internal injury caused by, but not limited to, disease, surgery, gunshots, stabbing, accidents, infarcts, ischemic injuries, to organs and tissues including but not limited to heart, bone, brain, spinal cord, retina, peripheral nerves and other tissues and organs commonly subject to acute and chronic injury, disease, congenital and developmental malformation and aging processes by administering an engineered vesicle as described herein or a pharmaceutical formulation thereof that is loaded with a peptide or alphaCT11-I, and/or ACT1-I peptides or pharmaceutical formulations thereof described herein to a subject in need thereof.

Co-Treatments

The engineered vesicles, alphaCT11-I, and/or ACT1-I peptides can be part of a treatment or preventive regimen that includes as a co-therapy or co-treatment with one or more other therapies or treatment or preventive modalities.

Co-treatments can include stem cells. Stem cells can include bone-marrow derived stem cells (BMSCs) and BMSCs can be substituted by other stem cell types including totipotent, omnipotent, pluripotent, multipotent, oligopotent and unipotent stem cell types, including embryonic, fetal, and adults stem cells, amniotic stem cells and other stem cells derived from the various stem cell niches and fluids found within or emanating from the bodies, mesenchymal stem cells, tissue and lineage specific stem cells and induced progenitor stem cells. Other differentiated cell types may also provide benefit with co-administration of an engineered vesicle described herein. For example, a treatment of skin wounds with a toroid of bone marrow stem cells BMSCs (prepared as described in Gourdie and Potts, Compositions and Methods for Tissue Engineering, Tissue Regeneration and Wound Healing. US Patent application US201 10086068) and the engineered vesicles described herein can significantly enhance regenerative healing and inhibit scarring over that occurring for treatments with a BMSC toroid alone or the peptide alone. In another example, treatment of skin wounds with a toroid of BMSCs and TGF-beta3 and the engineered vesicles described herein can significantly enhance regenerative healing and/or inhibit scarring over that occurring for treatments with a BMSC toroid alone or the peptide alone. In some aspects, the engineered vesicles, alphaCT11-I, and/or ACT1-I peptides and formulations thereof disclosed herein can be used to promote processes similar to embryonal scarless healing in the neonate, postnate or adult.

The engineered vesicles, alphaCT11-I, and/or ACT1-I peptides, and formulations thereof described herein can be included in co-treatments known to improve healing and/or reduce scarring. The treatment can include, e.g., aCT1, GAP26, GAP27, GAP19, GAP134, ZP123, danepeptide, rotigaptide, AAP10, connexin domain peptides and mimetics, connexin extracellular loop domain peptides and mimetics, connexin cytoplasmic loop domain peptides and mimetics, osteopontin, platelet-derived growth factor (PDGF), transforming growth factor and beta, TGF-B 1-3, TGFb or Cx43 antisense or peptides can be of significant benefit. Other molecules, and derivative peptides therefrom, that are contemplated for use with the present disclosure include bone morphogenetic proteins (BMP), epidermal growth factors (EGF), erythropoietins (EPO), fibroblast growth factors (FGF), platelet derived growth factors (PDGFs), ligands for the seven transmembrane helix family, granulocyte-colony stimulating factor (GCSF), granulocyte-macrophage colony-stimulating factor (GMCSF), growth differentiation factor-9 (GDF9), hepatocyte growth factor (HGF), hepatoma derived growth factor (HDGF), human growth hormones (HGH), interleukins (IL), insulin growth factors (IGF), insulin growth factor binding proteins (IGFBP), myostatins (GDF-8), nerve growth factors (NGF) and other neurotrophies, thrombopoietins (TPO), vascular endothelial growth factors (VEGF), caveolins, matricellular proteins (e.g., periostin, CCNs, thrombospondins), osteopontin, canonical (e.g., WntI, Wnt3a) and non-canonical W Ts (e.g., Wnt5a, WntI I), interleukins, tumor necrosis factors (TNFs), Notch-Delta, hyaluronin and related molecules, Hyaluronic synthetic enzymes (e.g., HAS2, HAS3), relaxins, acetylcholine, chitosan, DMSO, N-acetyl-glucosamine, catecholamines, lipids, poly unsaturated fats, estrogens and related/derivative molecules, androgens and related molecules, inhibitors of collagen processing (e.g., prolyl 4-hydroylase, C-proteinase and lysyl hydoxylase, HRT peptidases) and NADPH oxidases, factors effecting connective tissue growth factors (CTGFs), endothelins, and angiotensins, complement proteins, bioactive fragments or polymers of these molecules, genetic or cellular vectors producing these molecules, binding proteins, molecules targeting the receptors or downstream signal transduction mediators and combinations thereof. As these molecules and their different family members can have opposing effects in different circumstances ligands, agonists (activating factors) and antagonists (or inhibiting factors) of these molecules will be used in the disclosed invention.

Regenerative processes that can be aided by the present engineered vesicles, alphaCT11-I, and/or ACT1-I peptides, and pharmaceutical compositions thereof described herein, but are not limited to internal and external injury, regeneration of tissues, organs, or other body parts, healing and restoration of function following vascular occlusion and ischemia, brain stroke, myocardial infarction, spinal cord damage, brain damage, peripheral nerve damage, ocular damage (e.g., to corneal tissue), bone damage and other insults to tissues causing destruction, damage or otherwise resulting from, but not limited to, injury, surgery, cancer, congenital and developmental malformation, and diseases causing progressive loss of tissue structure and function, including but not limited to diabetes, bacterial, viral and prion-associated diseases, Alzheimer's disease, Parkinson's disease, HIV infection or AIDS, and other genetically determined, environmentally determined or idiopathic disease processes causing loss of tissue/organ/body part structure and function. In addition, the composition can be administered with drugs or other compounds promoting tissue and cellular regeneration including, but not limited to, trophic factors in processes including, but not limited to, brain, retina, spinal cord and peripheral nervous system regeneration (e.g., NGFs, FGFs, Neurotrophins, Neuregulins, Endothelins, GDNFs, BDNF, BMPs, TGFs, Wnts).

The engineered vesicles, alphaCT11-I, and/or ACT1-I peptides, or pharmaceutical formulations thereof can be used for repair after cosmetic and/or clinical procedures involving, but not limited to, controlled damage—e.g., corneal laser surgery, laser and dermabrasion/dermaplaning, skin resurfacing, and punch excision. Application of the present treatment immediately after surgery or any cosmetic procedure can be used to reduce or substantially eliminate scarring. Keloid scars are common in darker skinned people, e.g., of Asian, African, or Middle Eastern descent. Keloid scar is a thick, hypertrophic puckered, itchy cluster of scar tissue that grows beyond the edges of a wound or incision. Keloid scars are sometimes very nodular in nature, and they are often darker in color than surrounding skin. They occur when the body continues to produce tough, fibrous protein (known as collagen) after a wound has healed. Application of the present treatment can reduce or ameliorate formation of Keloid or hypertrophic scars.

The engineered vesicles, alphaCT11-I, and/or ACT1-I peptides, and formulations thereof can be a co-treatment with radiation therapy, alternatively or in addition to cancer chemotherapy. Radiation therapy treatment for glioma at a total dose of 50-65 Gy in fraction sizes of 1.8-2.0 Gy has been recommended (see Laperriere N et al., Radiother Oncol. 2002 September; 64(3):259-73).

The engineered vesicles, alphaCT11-I, and/or ACT1-I peptides, and formulations thereof can be a co-treatment with conventional arrhythmia treatments including anti-arrhythmic compounds, anticoagulant therapies, electrical treatments, electrical cautery, cryo-ablation, radio frequency ablation, implantable cardioverter-defibrillator, implantable pacemakers and combinations thereof.

The engineered vesicles, alphaCT11-I, and/or ACT1-I peptides, and formulations thereof can be a co-treatment with conventional congestive heart treatments, including but not limited to, commonly used vasodilators (nitroglycerin, diuretics such as furosemide) and in longer-term management of the disease including therapies such as angiotensin-converting enzyme (ACE) inhibitors (i.e., enalapril, captopril, lisinopril, ramipril), or in patients with severe cardiomyopathy, in conjunction with a implanted automatic defibrillator. In peripheral vascular diseases (PVD) arterial and/or venous flow is lowered, causing an imbalance between the supply of blood and proper levels of oxygenation of tissue. PVD includes acute arterial thrombosis, chronic peripheral arterial occlusive disease (PAOD), acute arterial thrombosis and embolism, Raynaud's phenomenon, inflammatory vascular disorders and venous and arterial disorders. It is contemplated that said composition can be used as a treatment of PVD.

The engineered vesicles, alphaCT11-I, and/or ACT1-I peptides, and formulations thereof can be a co-treatment with conventional drugs or therapy in the treatment of epilepsy, including but not limited to, a ketogenic diet, electrical stimulation, vagus nerve stimulation, responsive neurostimulator system (rns), deep brain stimulation, invasive or noninvasive surgery, avoidance therapy, warning systems, alternative or complementary medicine.

The engineered vesicles, alphaCT11-I, and/or ACT1-I peptides, and formulations thereof can be a co-treatment with conventional drugs or therapy in the treatment of retinopathy (including diabetic retinopathy and retinopathy of prematurity) and/or macular degeneration, including but not limited to, laser surgery, injection of triamcinolone into the eye, peripheral retinal ablation, cryotherapy, and vitrectomy.

SEQUENCES

SEQ ID NO: 1 Wild-Type Human connexin 43. NCBI Reference Sequence: NP_000156.1 (Gap
Junction alpha-1 protein [Homo sapiens]) The first AA and 225^{th} amino acid residue are noted.
The c-terminal region is underlined and extends from amino acid 225 to 382. Underlining and
Bold indicates the extracellular loops.

| | |
|---|---|
| 1 | M₁GDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNI₂₂₅IELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAGSTISNS HAQPFDFPDD NQNSKKLAAG HELQPLAIVD |
| 361 | QRPSSRASSR ASSRPRPDDL EI₃₈₂ |

SEQ ID NO: 2 gap junction beta-2 protein [Homo sapiens] GenBank ID: AHB08964.1
Extracellular loops indicated in bold and underlined.

| | |
|---|---|
| 1 | MDWGTLQTIL GGVNKHSTSI GKIWLTVLFI FRIMILVVAA KEVWGDEQAD FVCNTLQPGC |
| 61 | KNVCYDHYFP ISHIRLWALQ LIFVSTPALL VAMHVAYRRH EKKRKFIKGE IKSEFKDIEE |
| 121 | IKTQKVRIEG SLWWTYTSSI FFRVIFEAAF MYVFYVMYDG FSMQRLVKCN AWPCPNTVDC |
| 181 | FVSRPTEKTV FTVFMIAVSG ICILLNVTEL CYLLIRYCSG KSKKPV |

SEQ ID NO: 3 gap junction alpha-1 protein [Homo sapiens] S368A Mutant (Modified amino
acid is underlined and bold).

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAGSTISNS HAQPFDFPDD NQNSKKLAAG HELQPLAIVD |
| 361 | QRPSSRAA₃₆₈SR ASSRPRPDDL EI |

SEQ ID NO: 4 gap junction alpha-1 protein [Homo sapiens] S325A-S328A-S330A Mutant
Mutated amino acids are bold and underlined.

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAGA₃₂₅TIA₃₂₈NA₃₃₀ HAQPFDFPDD NQNSKKLAAG HELQPLAIVD |
| 361 | QRPSSRASSR ASSRPRPDDL EI |

SEQ ID NO: 5 gap junction alpha-1 protein [Homo sapiens] 258stop. Truncated gap-junction
alpha 1 protein based on SEQ ID NO: 1. Truncation is at AA 258 of SEQ ID NO: 1.

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAK |

SEQ ID NO: 6 gap junction alpha-1 protein [Homo sapiens] 357stop Truncated gap junction
alpha-1 protein based on SEQ ID NO: 1. Truncation is at AA 257 of SEQ ID NO: 1.

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAGSTISNS HAQPFDFPDD NQNSKKLAAG HELQPLA |

SEQ ID NO: 7 gap junction alpha-1 protein [Homo sapiens] 356stop Truncated gap junction
alpha-1 protein based on SEQ ID NO: 1. Truncation is at AA 356 of SEQ ID NO: 1.

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAGSTISNS HAQPFDFPDD NQNSKKLAAG HELQPL |

SEQ ID NO: 8 gap junction alpha-1 protein [Homo sapiens] 379stop Truncated gap junction
alpha-1 protein based on SEQ ID NO: 1. Truncation is at AA 379 of SEQ ID NO: 1.

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAGSTISNS HAQPFDFPDD NQNSKKLAAG HELQPLAIVD |
| 361 | QRPSSRASSR ASSRPRPDD |

SEQ ID NO: 9 gap junction alpha-1 protein [Homo sapiens] 324stop Truncated gap junction
alpha-1 protein based on SEQ ID NO: 1. Truncation is at AA 324 of SEQ ID NO: 1.

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAG |

SEQ ID NO: 10 gap junction alpha-1 protein [Homo sapiens] 325stop Truncated gap junction
alpha-1 protein based on SEQ ID NO: 1. Truncation is at AA 325 of SEQ ID NO: 1.

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAGS |

SEQ ID NO: 11 gap junction alpha-1 protein [Homo sapiens] 378stop Truncated gap junction
alpha-1 protein based on SEQ ID NO: 1. Truncation is at AA 378 of SEQ ID NO: 1.

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAGSTISNS HAQPFDFPDD NQNSKKLAAG HELQPLAIVD |
| 361 | QRPSSRASSR ASSRPRP |

SEQ ID NO: 12 gap junction alpha-1 protein [Homo sapiens] 363stop Truncated gap junction
alpha-1 protein based on SEQ ID NO: 1. Truncation is at AA 363 of SEQ ID NO: 1.

| | |
|---|---|
| 1 | MGDWSALGKL LDKVQAYSTA GGKVWLSVLF IFRILLLGTA VESAWGDEQS AFRCNTQQPG |
| 61 | CENVCYDKSF PISHVRFWVL QIIFVSVPTL LYLAHVFYVM RKEEKLNKKE EELKVAQTDG |
| 121 | VNVDMHLKQI EIKKFKYGIE EHGKVKMRGG LLRTYIISIL FKSIFEVAFL LIQWYIYGFS |
| 181 | LSAVYTCKRD PCPHQVDCFL SRPTEKTIFI IFMLVVSLVS LALNIIELFY VFFKGVKDRV |
| 241 | KGKSDPYHAT SGALSPAKDC GSQKYAYFNG CSSPTAPLSP MSPPGYKLVT GDRNNSSCRN |
| 301 | YNKQASEQNW ANYSAEQNRM GQAGSTISNS HAQPFDFPDD NQNSKKLAAG HELQPLAIVD |
| 361 | QRP |

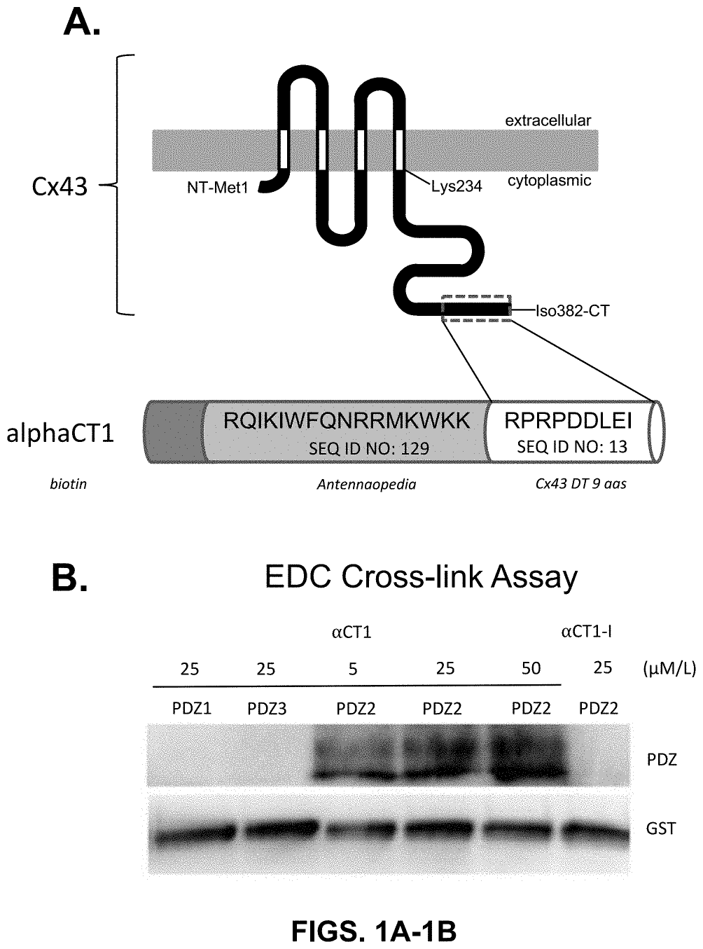

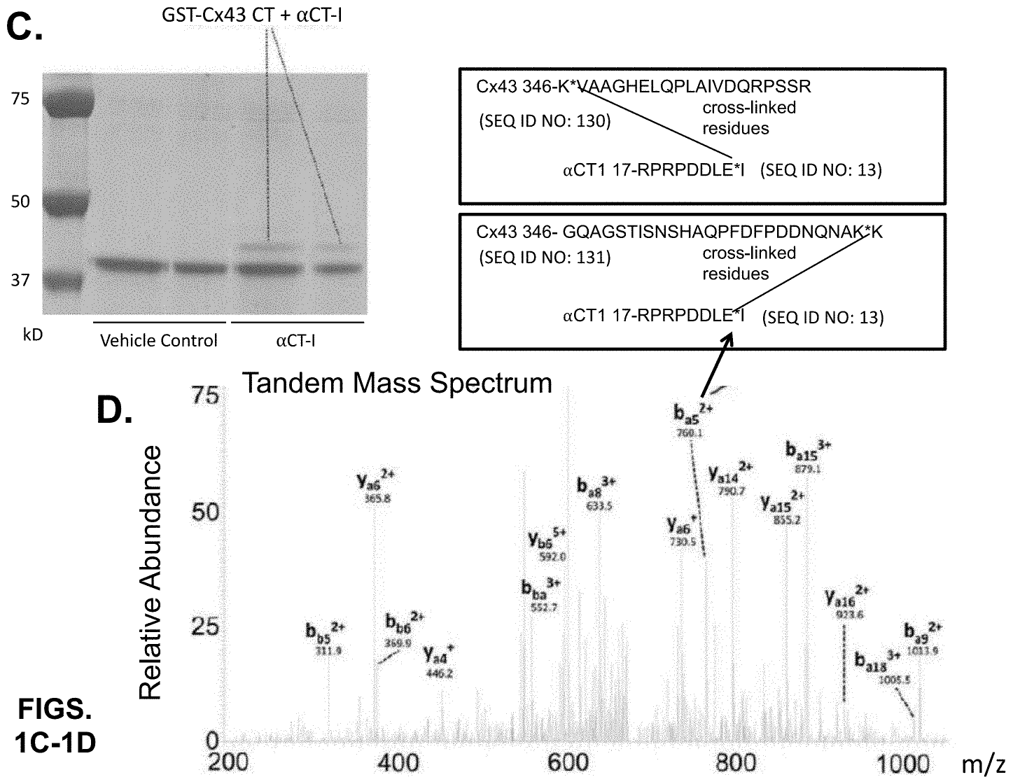

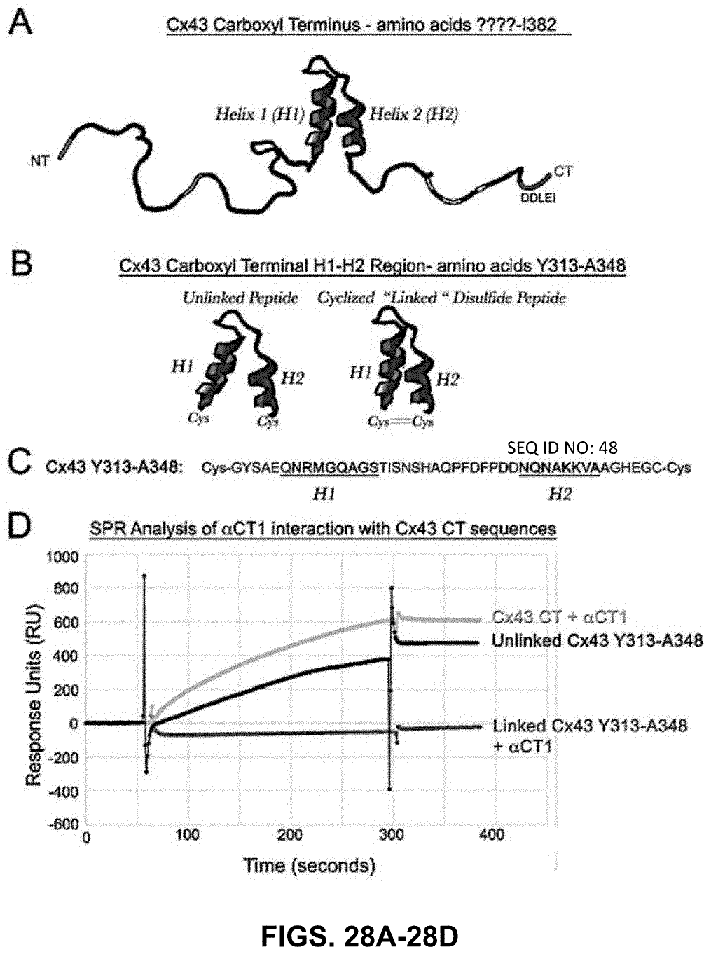

Synthetic Connexin Fragments Cargo Molecules
αCT and αCT-like
SEQ ID NO: 13 RPRPDDLEI (also referred to herein as aCT11, alpha CT11, or ACT11)
SEQ ID NO: 14 RPRPDDLE (also referred to herein as aCT11-I, alpha CT11-I, or ACT11-I)
SEQ ID NO: 15 RPRPDD
SEQ ID NO: 16 SRPRPDDLEI
SEQ ID NO: 17 SRPRPDDLE
SEQ ID NO: 18 SRPRPDD
SEQ ID NO: 19 IVDQRPSSRASSRASSRPRPDD
SEQ ID NO: 20 PSSRASSRASSRPRPDDLEI
SEQ ID NO: 21 RARPDDLDV
SEQ ID NO: 22 GDGKNSWWI
SEQ ID NO: 23 GRARPEDLAI
SEQ ID NO: 24 RD G K TVWI
SEQ ID NO: 25 GRTQSSDSAYW
SEQ ID NO: 26 KASS KARSD DSW
SEQ ID NO: 27 CSGK SKKPW
SEQ ID NO: 28 IVDQRPSSRASSR ASSRPRPDD
SEQ ID NO: 29 PSSRASSRASSRPRPDDLEI
SEQ ID NO: 30 DDLEI
SEQ ID NO: 31 DLEI
SEQ ID NO: 32 LEI
SEQ ID NO: 33 PRPDDLEI
SEQ ID NO: 133 RPDDLEI
SEQ ID NO: 34 PDDLEI
SEQ ID NO: 115 RPDDLE
SEQ ID NO: 116 RPRPDDELI
aCT Conservative Variant
SEQ ID NO: 35 KPRPDDLEI
SEQ ID NO: 36 RPRPDDLEV
SEQ ID NO: 37 RPRPDDVPV
SEQ ID NO: 38 RPKPDDLEI
SEQ ID NO: 39 SSRASSRASSRPKPDDLEI
SEQ ID NO: 40 RPKPDD
SEQ ID NO: 41 SSRASSRASSRPRPDDLDI
SEQ ID NO: 42 SSRASTRASSRPRPDDLEI
SEQ ID NO: 43 RPRPEDLEI
SEQ ID NO: 44 SSRASSRASSRPRPEDLEI
SEQ ID NO: 45 GDGKNSVWV
SEQ ID NO: 46 SKAGSNKSTASSKSGDGKNSVWV
SEQ ID NO: 47 GQKPPSRPSSSASKKLYV
SEQ ID NO: 50 DRPRPDDLEI
SEQ ID NO: 51 EERPRPDDLEI
SEQ ID NO: 52 ERPRPDDEL
SEQ ID NO: 53 DDRPRPDDELI
Cx43 JM peptides and variants
SEQ ID NO: 54 VFFKGVKDRVKGKSD
SEQ ID NO: 55 VFFKGVKDRV
SEQ ID NO: 56 VFFKGVKDRVKGRSDPYHAT
SEQ ID NO: 57 FFKGVKDRV
SEQ ID NO: 58 FKGVKDRV
SEQ ID NO: 59 VFFKGVKDR
SEQ ID NO: 60 VFFKGVKD
SEQ ID NO: 61 DRVKGRSDPYHAT
SEQ ID NO: 62 VKGRSDPYHAT
SEQ ID NO: 63 VFFKGVKDRVKGQSD
SEQ ID NO: 64 VFFKGIKDRVKGRND
SEQ ID NO: 65 VFFKGVKDRVKGRID
SEQ ID NO: 66 VFFKGIKDRVKGKSD
SEQ ID NO: 67 FFKGVKDRVKGKSD
SEQ ID NO: 68 FKSVKDRIKGRSD
SEQ ID NO: 69 VFFRSVKDHVKGKSD
SEQ ID NO: 70 VFFKRIKDRVKG
SEQ ID NO: 71 VLFKQIKDRVKGR
SEQ ID NO: 72 VLFKRIKDRVKGR
SEQ ID NO: 73 VFF KGV KDRV KGKSD
SEQ ID NO: 74 VFF KGV KDRV
SEQ ID NO: 75 IFF KGV KDRV KGKSD
SEQ ID NO: 76 IFF KGV KDRV
SEQ ID NO: 77 VIF KRM KDQI RESEK
SEQ ID NO: 78 VFF KGV KDRV KGKTD
SEQ ID NO: 79 VFF KGV KDRV KGRSD
SEQ ID NO: 80 VFF KGV KDRV RGKSD
SEQ ID NO: 81 VFF KGV KDKV KGKSD
SEQ ID NO: 82 IIF RGV RDRV RG RSD
SEQ ID NO: 83 VIF KRM KDQI RESEK
SEQ ID NO: 84 VIF KRM KDQI REREK
SEQ ID NO: 85 VIF KRM KDKI REREK
SEQ ID NO: 86 VFF KRV KDRI RERSK
SEQ ID NO: 87 VFFKGVKDRVKGRSD
Cx43 JM/SRC peptides
SEQ ID NO: 88 DPYHATSGALSPAKDCGSQKYAYFNGCSSPTAPLSPMSP
SEQ ID NO: 89 AYFNGCSSPTAPLSPMSP
SEQ ID NO: 90 PTAPLSPMSP
SEQ ID NO: 91 PTAPLSPM
SEQ ID NO: 92 APLSPMSP
Cx43 H2 peptides
SEQ ID NO: 93 HAQPFDFPDDNQNSKKLAAGHELQPLAIVD
SEQ ID NO: 94 NQNSKKLAAG
SEQ ID NO: 95 NSKKLAAG
SEQ ID NO: 96 HELQPLAIVD
Cx43 C-Loop peptides
SEQ ID NO: 97 KQIEIKKFK
SEQ ID NO: 98 DGANVDMHLKQIEIKKFKYGIEEHGK
SEQ ID NO: 99 KQIEIKKFKYG
Cx43 E-Loop peptides
SEQ ID NO: 100 VDCFLSRPTEKT
SEQ ID NO: 101 SRPTEKTIFII
SEQ ID NO: 102 SRPTEKTIFLL
SEQ ID NO: 103 SRPTEKT
SEQ ID NO: 104 ESRPTEKT
SEQ ID NO: 105 ADCFLSRPTEKT
SEQ ID NO: 106 VACFLSRPTEKT
SEQ ID NO: 107 VDCFLSRPTAKT
SEQ ID NO: 108 VDCFLSRPTEAT
SEQ ID NO: 109 CFLSRPTEKT
SEQ ID NO: 110 LSRPTEKT
αCT1 (SEQ ID NO: 13 with N-terminal antennapedia sequence) Underlined is antennapedia
sequence. Also refered to herein as alphaCT1, aCT1, αCT1, ACT1.
SEQ ID NO: 111 RQPKIWFPNRRKPWKKRPRPDDLEI
αCT1-I (SEQ ID NO: 14 with N-terminal antennapedia sequence) Underlined is antennapedia
sequence. Also refered to herein as alphaCT1-I, aCT1-I, αCT1-I, ACT1-I.
SEQ ID NO: 112 RQPKIWFPNRRKPWKKRPRPDDLE
M3
M3 (SEQ ID NO: 114 with N-terminal antennapedia intake seuqence. Underlined is
antennapedia sequence)
SEQ ID NO: 113 RQPKIWFPNRRKPWKKRPRPDDLAI
SEQ ID NO: 114 RPRPDDLAI
Other Polypeptides
Control Peptide
SEQ ID NO: 117 QPKIWFPNRRKPWKKIELDDPRPR
M1 peptide with antennapedia intake sequence (underlined is antennapedia)
SEQ ID NO: 118 RQPKIWFPNRRKPWKK RPRPAALAI
M2 peptide with antennapedia modification (underlined is antennapedia)
SEQ ID NO: 119 RQPKIWFPNRRKPWKK RPRPAALEI
M4 Scrambled control polypeptide (underlined is antennapedia)
SEQ ID NO: 120 RQPKIWFPNRRKPWKK LPAARIAPR
M1 peptide
SEQ ID NO: 121 RPRPAALAI
M2 peptide
SEQ ID NO: 122 RPRPAALEI
Scrambled control alpha CT11 peptide
SEQ ID NO: 123 DRDPEIPLR
Biotin labeled SEQ ID NO: 13
SEQ ID NO: 124 biotin-RPRPDDLEI
Biotin labeled SEQ ID NO: 123
SEQ ID NO: 125 biotin-DRDPEIPLR
Biotin labeled SEQ ID NO: 114
SEQ ID NO: 126 biotin- RPRPDDLAI
FAM (5,6) labeled SEQ ID NO: 13
SEQ ID NO: 127 (FAM 5,6)-RPRPDDLEI
FAM (5,6) labeled SEQ ID NO: 123
SEQ ID NO: 128 (FAM 5,6)- DRDPEIPLR
Antennapedia Sequence
SEQ ID NO: 129 RQIKIWFQNRRMKWKK
Additional Sequences from Figures
CX43 Segment (FIG. 1C)
SEQ ID NO: 130 KVAAGHELQPLAIVDQRPSSR
CX43 Segment (FIG. 1D)
SEQ ID NO: 131 GQAGSTISNSHAQPFDFPDDNQNAKK
CX43 Segment (FIG. 1D)
SEQ ID NO: 132 HAQPFDFPDDNQNSKKLAAGHELQPLAIVDQRPSSRASSRASSRPRPDDLEI
CX43 Y313-A348 Seqment (FIG. 28C)
SEQ ID NO: 48 GYSAEQNRMGQAGSTISNSHAQPFDFPDDNQNAKKVAAGHEGC

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

Use of JM peptides in causing a potent decrease of collagen synthesis by scar forming fibroblasts. The present disclosure describes compositions referred to as JM1 (JM=juxtamembrane) and JM2 that were found to have a strong inhibitory effect on collagen synthesis, processing and secretion from scar forming cells or fibroblasts. The synthetic JM peptides used in these experiments were of the amino acid sequence: VFFKGVKDRVKGRSD (JM2) (SEQ ID NO: 87) and VFFKGVKDRV (JM1) (SEQ ID NO: 45). The peptides can be loaded into the provided EVs and can elicit results similar to those observed for naked peptide as follows.

The amino acids (aas) sequences given are based on the juxtamembrane sequence of the gap junction protein Cx43 (connexin 43, e.g. SEQ ID NO: 1). JM1 is based on aas 231 to 241 of Cx43. JM2 is based on aas 231 to 246 of Cx43.

Isolation and treatment of Neonatal Cardiac Fibroblasts with Cx43 based peptides (peptides used included ACT1, JM1, JM2, Antennapedia [ANT], reverse ACT1 [Rev], poly Arginine [poly r]). Said peptides with and without internalization vectors can be loaded into the provided EVs and can elicit results similar to those observed for naked peptide as follows. Neonatal cardiac fibroblasts (NHFs) were isolated from 3-4 day old rat hearts by collagenase digestion (100 U/mL) and differential attachment as previously described (Borg et ah, 1984). All cells were maintained in Dulbecco's Modified Eagle Medium (DMEM) supplemented with 10% Fetal Bovine Serum and 100 U/mL penicillin G and 100 μg/mL streptomycin and used prior to passage four. For experiments, 40,000 NHFs were plated into the wells of a 24-well tissue culture plate and grown for 24-48 hours. On the day of treatment, media was removed from each well and replaced with fresh media containing 50 μg/mL L-ascorbic acid-2-phosphate; Sigma Chemical Co., St. Louis, Mo.). The appropriate volume of each peptide (resuspended in sterile, deionized 18 MO resistivity water) was added to achieve the desired final concentration (30, 90, 180 μM peptide concentrations were tested). Culture plates were incubated overnight in a 37° C. incubator with 5% C02.

Protein Isolation and Examination of Collagen Synthesis by Western Blotting. Conditioned culture media was collected from each well and stored at −20° C. for analysis of soluble collagen. Cellular protein, including insoluble collagen and collagen still within the NHFs, were isolated by adding 100-200 μL, of cell lysis buffer (0.01 M Tris, pH 7.4, 0.001 M Sodium Orthovanadate, 1% sodium dodecylsulfate [SDS]) to each well and incubating 10 minutes at room temperature. Prior to addition, cell lysis buffer was warmed to facilitate solubilization of SDS and 100 μL of Halt protease inhibitor (Pierce Biotechnology, Rockford, Ill.) was added per 10 mL buffer to be used. After incubation, the well bottoms were scraped and liquid transferred to a microcentrifuge tube for storage at −20° C. Protein concentrations of cell lysates were determined using a Micro BCA assay (Pierce). SDS-PAGE samples were prepared by combining either 10 μg of cell lysate or 30 μL of conditioned media with XT loading buffer (BioRad, Hercules, Calif.), dithiothreitol and boiled for five minutes. Samples were loaded onto 3-8% Tris-Acetate Criterion XT gels (BioRad, Hercules, Calif.) and proteins separated at 140V. After electrophoresis, proteins were transferred onto 0.45 μM nitrocellulose membranes (BioRad) overnight at room temperature (Transfer buffer: 25 mM Tris, 192 mM Glycine, 20% Methanol, 0.01% SDS). The presence of collagen was determined by probing the membranes with a rabbit anti-mouse collagen type I antibody (MD Biosciences) at 1:20,000 dilution in blocking buffer (5% milk in Tris-buffered saline) followed by a goat anti-rabbit IgG horseradish peroxidase conjugated antibody at 1:100,000 (Southern Biotech Associates) and detection with Pierce SuperSignal Femto West detection reagent (Pierce). To assess the activity of JM1 and JM2 peptides with respect to collagen production and their potential in mediating wound healing, cardiac fibroblasts were treated with these two peptides and their effectiveness compared to that for the previous described Cx43 peptide ACT1. NHFs were treated with various concentrations of JM1, JM2, ACT1, and ANT (Antennapedia) peptides, vehicle (water) or left untreated and the production of collagen both in the culture media and cell-associated collagen assessed by western blotting. Treatment of NHFs with ACT1 resulted in a dose-dependent reduction in the secretion of mature, fully processed collagen whereas treatment with ANT, vehicle (lane labeled HC180) or untreated (UT) samples showed high levels of mature collagen type I. Treatment with JM1 and JM2 also yielded a dose-dependent decrease in the production of mature, type I collagen; however, at the highest dose of JM1 and JM2 tested (180 μM), no mature type I collagen was detected in conditioned media. Even at the middle dose of 90 μM, JM 1 and JM2 demonstrate more than a than 50% reduction in mature type I collagen produced compared to ACT 1. Data from NHF cell lysate samples, revealed a similar trend in that treatment with JM 1 and JM2 had a more profound reduction in the amount of mature type I collagen than treatment with the ACT1 peptide. To evaluate the impact of the poly-Arginine (poly-r)N-terminal sequence on JM1 and Jm2 activity, NHF cells were treated with a poly-r peptide. At equivalent concentrations (about 90 μM) the amount of collagen produced by NHFs treated with JM1 and JM2 was less than half of that produced by cells treated with the poly-r peptide indicating that the effects of JM 1 and JM2 on collagen production were largely due to the Cx43 sequence and not the presence of the poly-r sequence. These results indicate that JM1 and JM2 can have a more potent wound healing effects than those demonstrated by the ACT1 peptide.

The potency of JM peptides can be gauged by comparison to ACT1 (RQPKIWFPNRRKPWKKRPRPDDLEI (SEQ ID NO: 111)) a Cx43 sequence developed by the Gourdie laboratory. ACT1 has been also shown to promote wound healing, regeneration and tissue repair (Gourdie et al, U.S. Pat. No. 7,786,074). ACT1 incorporates aas 373 to 382 of Cx43 (RPRPDDLEI (SEQ ID NO: 13)) and is distinct from JM1 and JM2. In the same assay on cultured fibroblasts ACT1 also reduced collagen processing and secretion, but this reduction was less than that caused by JM 1 and JM2.

### Example 2

Use of JM Peptides in Experiments on Cx43 Expression in Cultured Cells

The first tests of JM 1 and JM2 were performed and the experiments centered on the basic cell biology of the peptides. To this end, a HeLa cell line stably expressing Cx43 (Cx43-HeLa was used. Initially, cells were treated with 1, 2, 5, or 10 μM of either JM1 or JM2 and observed over a 24-hour period. Cell viability was assessed by acridine orange/ethidium bromide staining. No differences in cell death were observed in any of the treatment groups indicating that JM peptides showed no obvious toxicity. At 24 hours JM2 treated cells were more confluent than control cells indicating increased proliferation and survival in the JM2 treated cells.

Given that the 10 μM concentration of peptide was not toxic to cells, the inventors treated Cx43-HeLa cells with 10 μM JM1 or JM2 for 2, 4, 24, or 48 hours followed by fixation and immunofluorescent labeling of Cx43 and ZO-1. Said peptides can be loaded into the provided EVs and can elicit results similar to those observed for naked peptide as follows. For both JM1 and JM2, greater cytoplasmic Cx43 was observed, particularly in perinuclear regions. However, the most striking effects were on ZO-1 organization. In control cells ZO-1 localized to cell borders, often at sites of small, finger-like projections between the cells. Cytoplasmic ZO-1 was also notable. In JM-treated cells a strong contrast in the ratio of cell border to cytoplasmic ZO-1 was found, with relative levels at cell borders being increased over controls. Thus, in JM1 treated cells, ZO-1 cell border labeling was enhanced. In JM2 treated cells ZO-1 levels had well defined cell-cell interfaces and the monolayer appeared to be more epithelia-like. There was also a noticeable increase in the number of cells per area of field, supporting the earlier observation that JM2 treated cells appeared to proliferate and survive at an increased rate.

### Example 3

In Vitro Scratch Injury

The potency of the provided composition carrying an ACT peptide (RPRPDDLEI (SEQ ID NO: 13)) can be gauged by comparison to ACT1 a Cx43 sequence developed by the Gourdie laboratory that has been also shown to promote wound healing, regeneration and tissue repair (Gourdie et ah, U.S. Pat. No. 7,786,074, which is incorporated herein by reference). In Example 3, the effect of ACT1 treatment is thus described to provide an example of the use and results for JM peptides. As described in Hunter et al. (2005), myocytes from neonatal rats were grown until forming a near-confluent monolayer on a tissue culture dish according to standard protocols. The cultures were subsequently allowed to culture for a further 5 days culture medium comprising 30 μM ACT1 peptide, 30 μM non-active control peptide (RQPKIWFPNRRKPWKKIELDDPRPR (SEQ ID NO: 117)) or phosphate buffered saline (PBS) containing no peptide or control peptide. The non-active control peptide comprises a polypeptide with a carboxyl terminus in which the peptide sequence has been reversed. The amino terminus of active and control peptides are both biotinylated, enabling detection (e.g., assay) of the peptides in the cell cytoplasm using standard microscopic or biochemical methods based on high affinity streptavidin binding to biotin.

Culture media with added peptides or vehicle control was changed every 24 hours during the experiment. The peptide greatly increased the extent of Cx43 gap junction formation between myocytes relative to the control conditions (Hunter et al. (2005).

The transformed fibroblast line NIH-3T3 cells were grown over 2-3 days until forming a near-confluent monolayer on a tissue culture dish according to standard protocols and the monolayer was then pre-treated with peptide for 24 hrs, and “scratch-injured” with a p200 pipette tip. The “scratch injury” was subsequently allowed to repopulate for 24 hours in the presence of 30 μM active peptide dissolved in the culture media or in presence of two control conditions. In the first control condition, the “scratch-injured” cells were allowed to repopulate for 24 hours in the presence of a non-active control peptide dissolved in the culture media at a concentration of 30 μM. In the second control condition, phosphate buffered saline (PBS) was added to the culture media and the “scratch-injured” cells were allowed to repopulate in the presence of this vehicle control solution containing no active peptide or control inactive peptide. The “scratch injury” of active peptide-treated cells remained relatively repopulated after 24 hours, with few cells repopulating the area within the initial “scratch injury” edges. The peptide treated cells also can show reduced proliferation of the cells in the experimental cellular model.

### Example 4

In Vivo Skin Wound Healing

In Example 4 the effect of ACT 1 treatment is described to provide an example of use and results for the provided compositions when containing an ACT peptide. The results described in Example 4 were published in Ghatnekar et al. (2009) and in Gourdie et al, U.S. Pat. No. 7,786,074, which are incorporated herein by reference. The results of clinical trials with ACT1 for diabetic foot ulcers, venous leg ulcers and normal skin wound healing have also been published and these citations are also incorporated by reference (PMID 27856288, 25703647, 25072595).

Neonatal mouse pups were desensitized using hypothermia. A 4 mm long incisional skin injury was made using a scalpel through the entire thickness of the skin (down to the level of the underlying muscle) in the dorsal mid line between the shoulder blades. 30 μL of a solution of 20% pluronic (F-127) gel containing either no (control) or dissolved ACT1 peptide at a concentration of 60 μM was then applied to the incisional injuries. Pluronic gel has mild surfactant properties that may aid in the uniform dispersion of the peptide in micelles. More importantly, 20% pluronic gel stays liquid at temperatures below 15° C., but polymerizes at body temperature (37° C.). This property of pluronic gel probably aided in the controlled release of peptide into the tissue at the site of incisional injury, protecting the peptide from break-down in the protease-rich environment of the wound and also enabling active concentrations of the peptide to be maintained over prolonged periods. Inactive control or active peptide containing gel was applied subsequently 24 hours after the initial application. No further application of peptide containing gel was made after the second application. By 48 hours it can be noted that the treated injury was significantly more closed, less inflamed, less swollen (note ridges at the wound edge), and generally more healed in appearance than the control injury. These differences in inflammation, swelling and healing between the control and treatment and control persisted at the 72 and 96 hour time points. At 7 days, the active peptide treated wound, had a smoother and less scarred appearance than the control peptide-treated injury.

Anesthetized adult mice had 8 mm wide circular excisional skin injuries made by scalpel down to the underlying muscle in the dorsal mid line between the shoulder blades. The boundary of the injury was demarcated by an 8 mm wide circular template cut in a plastic sheet. 100 μL of a solution of 30% pluronic gel containing either no (control) or dissolved ACT1 peptide at a concentration of 100 μM was then applied to the excisional injuries. Peptide containing gel was applied subsequently 24 hours after the initial application. No further applications were made after the second application. The treated excisional injuries closed faster, were less inflamed in appearance, healed faster and scarred less than the control injuries over a 10-14 day time course. Histochemical analyses confirmed that active peptide treated wounds healed with less redness/inflammation and area of scar tissue, as well demonstrating partial regeneration of epidermal and vascular organization. The compositions and engineered vesicles including such compositions can be used as treatment for dermal injuries.

### Example 5

In Vivo Healing of Chronic Skin Wounds

Poor healing or chronic wounds such as venous ulcers of the leg, diabetic foot ulcers, or pressure ulcers are a common cause of morbidity, can be recurrent for a given patient and are difficult and expensive treat. There are few if any approved or effective pharmacological treatments of such poor healing wounds. In one example, patients clinically diagnosed by their Doctor as having ulceration of venous origin can be treated with JM peptide. Diagnosis can include measurement of the ratio of ankle to brachial systolic pressure and a determination that this pressure was abnormal (e.g., >0.8). Other aids to diagnosis can include arterial and venous Doppler, venous outflow strain-gauge plethysmography, and photoplethysmography. Treatment of the wound can occur every 1, 2, 3, 4 or 5 days for periods of 12 weeks, or longer if required and as indicated by a qualified wound care specialist. Prior to treatment the ulcer can be irrigated with a saline solution, ACT Peptide at 100 μM dissolved in a 2-10% ethylcellulose gel or other suitable vehicle (such as contained in an engineered vesicle described in the present application) can then be applied to the wound such that it evenly covered it. The volume of gel applied can depend on ulcer size and within the skill of the medical practitioner to determine. The wound can then be covered with a dry gauze dressing and the dressing can be held in place by a toe-to-knee elastic compression bandage. The progression of healing can be monitored by a medical practitioner and the initial healing process can be considered complete when full re-epithelialisation had occurred. The patient can return to the clinic at subsequent intervals after healing to ensure that recurrence had not occurred. In the case of recurrence, treatment can be repeated until complete healing was observed.

In another example hydroxyethylcellulose (HEC) is a suitable gelling agent and acceptable carrier of the drug product when treating skin wounds. In one aspect, the gelling agent is Hydroxyethylcellulose (HEC), 250 HHX. In one as, the percent (w/w) of HEC is in the range of 1-5%. In a further aspect, the percent (w/w) of HEC is 1.25%. In the manufacture of HEC, a purified cellulose is reacted with sodium hydroxide to produce a swollen alkali cellulose. The alkali-treated cellulose is more chemically reactive than cellulose. By reacting the alkali cellulose with ethylene oxide, a series of hydroxyethylcellulose ethers is produced. In this reaction, the hydrogen atoms in the hydroxyl groups of cellulose are replaced by hydroxyethyl groups, which confer water solubility to the gel. It is contemplated in this invention that a single HEC ether may be used, or a mixture of HEC ethers of difference molecular weight and structure may be used. Suitable grades of HEC for pharmaceutical purposes are well known and full described in the pharmaceutical literature. Suitable commercially available brands of HEC include but are not limited to Fuji HEC-HP; Fuji HEC-AG 15; NATRO-SOL 250HR; NATROSOL 250 MH; NATROSOL 250G; CELLOSIZE QP 30000; TYLOSE H SERIES; NATROSOL 180L; NATROSOL 300H; TYLOSE P-X; NATROSOL 250M; CELLOSIZE WP 4400; CELLOSIZE UT 40; NATROSOL 250H4R; Tylose H 20P; NATROSOL LR; TYLOSE MHB; NATROSOL 250HHP; HERCULES N 100; CELLOSIZE WP 300; TYLOSE P-Z SERIES; NATROSOL 250H; TYLOSE PS-X; Cellobond HEC 400; CELLOSIZE QP; CELLOSIZE QP 1500; NATRO-SOL 250; HYDROXYETHYL CELLULOSE ETHER; HESPAN; TYLOSE MHB-Y; NATROSOL 240JR; HYDROXYETHYL STARCH; CELLOSIZE WP; CELLOSIZE WP 300H; 2-HYDROXYETHYL CELLULOSE ETHER; BL 15; CELLOSIZE QP 4400; CELLOSIZE QP3; TYLOSE MB; CELLULOSE HYDROXY-ETHYLATE; CELLOSIZE WPO 9H17; CELLOSIZE 4400H16; CELLULOSE HYDROXYETHYL ETHER; Hydroxyethyl Cellulose; Hydroxyl Ethyl Cellulose (HEC); Hydroxyethyl Cellulose 100H (celocell 100h); TYLOSE MH-XP; NATROSOL 250HX; Natrosol; Daicel EP 500; HEC-Unicel; HEC (Hydroxyethyl cellulose); Cellosize; HEC-AI 5000; Fuji HEC-AL 15; HEC-Unicel QP 09L; Cellulose, ethers, 2-hydroxyethyl ether; Unicel QP 52000H; HEC-QP 4400; SP 250 (cellulose); Hetastarch; Cellulose, ethers, 2-hydroxyethyl ether; Glutofix 600; FL 52; Fuji HEC-AX 15F; Tylose H 300P; HEC-Unicel QP 300H; Tylose H 300; Daicel SP 550; Daicel SE 600; Unicel QP 15000; HEC-QP 100 MH; HEC-QP 9H; OETs; Daicel EP 850; H. E. Cellulose; Cellobond 25T; Unicel QP 100 MH; Tylose H 4000; SE 850K; Tylomer H 20; Daicel SE 850K; Tylose H 30000YP; Unicel QP 4400; SP 407; Tylose H 100000; Daicel SP 200; Culminal HEC 5000PR; Tylopur H 300; Daicel SP 750; Sanhec; BL 15 (cellulose derivative); Unicel QP 300H; Tylomer H 200; J 164; Tylose H 10; Tylose H 20; AH 15; Daicel SP 600; Daicel SE 900; HEC-Unicel QP 4400H; AX 15; Daicel SP 800; Fuji HEC-AW 15F; HEC-SE 850; HEC-A 5-25CF; Metolose 90SEW; AW 15 (polysaccharide); Cellobond HEC 5000; HEC-QP 100M; Cellobond HEC 15A; Tylose H 15000YP2; Walocel HT 6.000 PFV; 2-Hydroxyethyl cellulose (Natrosol Type 250HRCS); Fuji HEC-BL 20; Fuji HEC-SY 25F; Telhec; HEC-SP 200; HEC-AH 15; HEC-Unicel QP 30000H; see; HEC 10A; Daicel SP 400; Admiral 3089FS; Fuji HEC-A 5000F; HEC-SP 400; Hydroxyethyl Methyl Cellulose (HEMC); HYDROXYETHYL CELLULOSE (HEC); Hydroxyethyl Starch (CAS No: 9004-62-0); Hydroxy Ethyl Cellulose; “Natrosol” [Aqualon]; HEC; 2-HYDROXYETHYL CELLULOSE; NATROSOL 150L; TYLOSE MHB-YP; HYDROXYETHYL ETHER CELLULOSE; NATROSOL 250L; CELLOSIZE WP 400H; TYLOSE P; CELLULOSE, 2-HYDROXYETHYL ETHER; TYLOSE MH-K; NATROSOL 250HHR.

In some aspects, the present invention includes a method of wound treatment comprising administering to a subject in need thereof a topical formulation comprising at least one alpha connexin polypeptide and hydroxyethylcellulose gel, wherein the hydroxyethylcellulose gel stabilizes the alpha connexin polypeptide. The wound treated may be an acute surgical wound or a chronic, non-infected, full-thickness lower extremity ulcer.

In a certain aspect, the drug product of the present invention may be used to mitigate excessive scar formation associated with acute surgical wounds. In this aspect, the drug product of the present invention may be applied at the time of surgical incision closure, 1 hour after surgical incision closure, 2 hours after surgical incision closure, 3 hours after surgical incision closure, 4 hours after surgical incision closure, 5 hours after surgical incision closure, 6 hours after surgical incision closure, 7 hours after surgical incision closure, 8 hours after surgical incision closure, 9 hours after surgical incision closure, 10 hours after surgical incision closure, 11 hours after surgical incision closure, 12 hours after surgical incision closure, 13 hours after surgical incision closure, 14 hours after surgical incision closure, 15 hours after surgical incision closure, 16 hours after surgical incision closure, 17 hours after surgical incision closure, 18 hours after surgical incision closure, 19 hours after surgical incision closure, 20 hours after surgical incision closure, 21 hours after surgical incision closure, 22 hours after surgical incision closure, 23 hours after surgical incision closure, 24 hours after surgical incision closure, 48 hours after surgical incision closure, 72 hours after surgical incision closure, or thereafter.

In another aspect, the drug product of the present invention may be used to treat chronic ulcers. For example, ulcers may include diabetic foot ulcers, venous leg ulcers, and pressure ulcers. These ulcers may be chronic, non-infected, full-thickness lower extremity ulcers. In one aspect, the drug product of the present invention may be applied to a chronic ulcer in a daily regimen, a regimen of every other day, a regimen of once a week, or in various other regimens until healing of the chronic ulcer is apparent. In another aspect, the drug product of the present invention may be applied to a chronic ulcer in a regimen at day 0, 3, 7, 14, 21, and 28. In another aspect, the drug product of the present invention may be applied to a chronic ulcer in a regimen at day 0, day 3, week 1, week 2, week 3, week 4, week 5, week 6, week 7, week 8, week 9, week 10, week 11, and week 12. In another aspect of the present invention, the drug product is manufactured with the following steps:

Step 1: In a suitable size of beaker, add propylene glycol, glycerin, methylparaben and propylparaben. Mix with a propeller until the parabens are completely dissolved.

Step 2: In a manufacturing vessel, add purified water (part I), EDTA, monobasic sodium phosphate, dibasic sodium phosphate and D-mannitol. Mix with a propeller until a clear solution is obtained.

Step 3: Add the solution from step 1 to the manufacturing vessel. Rinse the beaker with purified water (part II, divided into approximately 3 equal portions) and add the rinse back to the vessel. Continue with propeller mixing until the solution is visually homogeneous.

Step 4: With homogenization mixing, add hydroxyethyl cellulose into the manufacturing vessel from Step 3. Mix until the polymer is fully dispersed.

Step 5: In a separate beaker, add purified water (part III) and an EV containing alpha connexin polypeptide (e.g., RPRDDLEI). Mix with a stir bar or propeller mixer until the peptide is completely dissolved and a gel is formed.

Step 6: With continuous propeller mixing, add the drug solution from step 5 to the manufacturing vessel. Rinse the beaker with purified water (part IV, divided into approximately 3 equal portions) and add the rinse back to the vessel. Mix until the gel is homogeneous.

### Example 6

In vivo wound healing in association with a stem cell treatment and aCTI treatment is described in Example 6 and can demonstrate use and the EV composition carrying a ACT or JM peptide cargo compound. The results described in Example 6 for aCTI peptide were described in Gourdie and Potts, US Patent application US20110086068, which is incorporated herein by reference.

Stem cells were primed using the method described herein prior to engraftment into a wound. Adult bone marrow stromal cells (BMSC—mesenchymal stem cells) were isolated from adult rat femurs and passaged and cultured to produce a pure population of BMSC. A small biopsy punch (8 mm) was used to create a small, 8 mm diameter round wound on the back of the animal. The punch site was inlayed with the preformed collagen cell containing the BMSC cells (configured in a toroid as per Gourdie and Potts, US201 10086068) and/or peptide and two 4-0 prolene stitches were placed in the skin at the biopsy sight to hold the gel in place. The collagen gel (1 mg/ml) was polymerized in a sterile hood and BMSC cells were treated with the aCTI peptide (150 μM) and then added either on top of the 1.5 mm gel (toroid) or mixed into the polymerizing gel. Wounds were also treated with the gel only, gel plus peptide alone, gel plus cells alone and toroids with an inactive control peptide. Animals were allowed to heal for 30 days and then sacrificed and the pelts were removed and the wounds excised and surrounding skin was processed for standard embedding in paraffin epidermal surface-up.

From wound edge to wound edge every 30th section was mounted on a glass slide and stained with H&E histochemistry. Images of the granulation in each section were then recorded as single images or montages of 2-3 images. Generally, 15-30 serial 300 um-spaced sections were recorded per wound. The granulation tissue area, length of epidermal surface and number of follicles intersecting the epidermis were then counted or measured using Image J software from each wound montage. Estimates of wound granulation tissue volume and the granulation tissue area measurements were recorded for each section. Similarly, scar surface area was estimated as was follicle density in the scar epidermis. T-tests for paired samples were carried using MS Excel (p<0.05). Measurements on treatments wounds within individual rats were normalized to the gel only control wound as a baseline.

The peptide-alone treated wound had a scar area and scar tissue volume that were significantly (p<0.05) smaller than the controls and most other treatments. However, the wound that received both the BMSC toroid and the peptide had a scar that was even smaller in surface area than the peptide-alone treated wound. This finding of improved healing for the combinatorial treatment over all other treatments/controls was a consistent result. It was also noted that these same 2 wounds, Gel+aCT1 and Gel+BMSC Toroid+active peptide, showed consistent significantly faster closure rates than the other 4 wounds. Qualitative and quantitative appraisals of the wounds indicated the following pattern of variance in scar size: Gel+BMSC toroid+active peptide<(smaller than) Gel+active peptide<Gel+BMSC Toroid<Gel alone=Gel+BMSCs (non-toroidal)+active peptide wound=Gel+BMSC Toroid+Rev control wound. Importantly, the combinatorial treatment of gels containing the toroid of BMSCs and active peptide consistently had the smallest scars at the end of the 30-day experiment. The provided composition is thus contemplated to provide a treatment of dermal injuries in association with stem cells.

Thus, it is expected that healing will occur in a similar way when the JM peptide is loaded into and delivered via an engineered vesicle as described in this application.

### Example 7

In Vivo Cardiac Wound Healing and Arrhythmia Reduction

In Example 7 the effect of ACT1 treatment is described and can demonstrate use of the engineered vesicles described in the present application that are loaded with ACT1. The results described in Example 7 were published in O'Quinn et al. (2011); Gourdie et al, US patent application US20100286762; and Norris et al. (2008), which are incorporated herein by reference. One of the commonest injuries to the heart is a myocardial infarction (MI) that occurs as a sequalae to coronary heart disease (CHD). CHD is the biggest killer of people in developed countries. During an MI or “heart attack” there is a sudden failure of coronary circulation. If the patient survives, the MI scar may cause sickness or death from loss of cardiac function (heart failure) or prompt the development of life-threatening arrhythmias. The compositions described herein be deployed to reduce scarring following MI and thus ameliorating morbidity and mortality associated with CHD.

A new method for injuring the heart in an animal model was developed that was specifically designed to increase the ability to determine whether our therapeutic approach causes regeneration rather than the normal process of formation of scar tissue following an injury such as MI. This method involved delivering a freezing injury to the heart that always generated a non-transmural wound of consistent size and depth in the left ventricular wall muscle. Because wound size was consistent between mice, the inventors can be certain of the exact amount of scar tissue that can be deposited in the heart in each animal injured. More importantly, the consistency of the lesion enabled us to determine with certainty that has not been previously achievable by others as whether newly regenerated muscle was present in the healed injury.

To undertake the injury, 12-24 week female CD1 mice (Charles River) were used. Mice were anesthetized (isoflurane), intubated and a left thoracotomy was performed at the 4th intercostal space. The LV wall was cryo-injured by exposure for 5 sec to a liquid-\hat{ }chilled 3 mm circular flat-tip probe (Brymill: CRY-AC-3) such that the LV surface was slightly depressed. In the case of treatment of the animal model with the composition cryo-injury, the mouse receives EMT-primed BMSCs in gel together with 3 ng/ml of TGF-beta3 over the cryo-injury, and the gel is then held by 2 small dissolving sutures on the surface of the epicardium. Cel-Tak™ adhesive (BD Biosciences) or other surgical adhesive can also be used to secure the gel to the wound. Surgical wounds are then closed using 6-0 silk sutures (Ethicon) and sealed with Nexabond™.

Using the said cardiac-injury model, we have showed (i.e., p<0.05), that release of ACTI from a methyl-cellulose patch on the injury results in significant improvement in LV diastolic and systolic function over a 8 week time course. This improvement in mechanical function was associated with significantly increased scar uniformity. Treated hearts also showed higher and more uniform, intercalated-disk-localized and pS368 phosphorylated Cx43 in myocytes bordering the scar. Consistent with evidence that downregulated and disordered Cx43 at the infarct border zone is a key factor in cardiac conduction disturbance, we determined that there was a dramatically reduced (p<0.05) frequency and severity of arrhythmias in peptide-treated animals as assessed by electrophysiological studies (pacing and S1-S2 protocols).

In another example of the injury method, analysis of heart pump function by echocardiography showed that one week following injury in a second group of treatment mice (mice in which bone marrow containing stem cells were infected in vivo with a periostin shRNA lentivirus) and control mice (i.e., mice similarly receiving a control virus) showed a similar (−20%) decline in the efficiency of heart pumping function—as measured by % ejection fraction from the left ventricle (PMID: 27339799). Periostin shRNA can be cargoed in the present EV compositions. Ejection fraction is a standard clinical measure of cardiac pumping efficiency. This decline indicated that just after freeze wounding both treatment and control hearts had received a similar initial degree of injury as reflected by their similar reduction in function over the first week. However, at the end of the following 4 weeks, a stage that t the healing of the injury to the heart and scar formation can be expected to be nearing completion, cardiac pump function of the treatment had improved to be <98% better than that of controls. Remarkably, by 4 weeks heart pump function in the treatment had recovered to levels identical to those of a normal uninjured heart. Meanwhile in controls, pump function had declined at the 4 week period by 50% compared to uninjured hearts. The improvement in % fractional shortening of the left ventricle is another clinically used measurement of cardiac function and contractility. Percent fractional shortening improved by more than 120% in the treatment relative to control at 4 weeks following injury. As was the case with ejection fraction, treatment caused a recovery of % fractional shortening levels to those of a normal, uninjured heart at 4 weeks, whereas controls continued to show significant declines in this index of cardiac contractile function.

The systolic and diastolic volume of the left ventricle during the cardiac contraction cycle are two other commonly used indices of cardiac function. Increases in these indices are recognized as indicative of a loss of cardiac function and are viewed by clinicians as disease markers for the development of eventual heart dilation, heart failure and death. The diastolic volume of the left ventricle of treatment was significantly improved, being 40% less dilated than that of control. More remarkably, left ventricular systolic dimension was improved to be >75% lower than controls. Putting this another way, at 46.5, the left ventricular volume of control at systole was 5-times more dilated at systole than that of the 10.61 value measured from the echocardiograms of treatment. Treatment also caused both left ventricular volume indices to recover to levels found in the normal, uninjured heart. No such recovery to normality has ever been noted to occur in controls. The data at 4 weeks post-injury led us to conclude that the mice that had received our standardized cardiac injury and treatment unexpectedly recovered to normal cardiac pump function and contractility. In further contrast to controls, there was no sign of pathological cardiac dilation indicating that treated hearts were progressing to heart failure and eventual death. Echocardiographic measurements of % ejection fraction, % fractional shortening, and left ventricular volume at diastole and systole were repeated at 6 weeks. These measurements indicated that the improvement in these parameters found at 4 weeks were sustained 6 weeks following treatment and injury. By contrast, none of these cardiac function parameters showed any improvement in the control at 6 weeks and were for most part were similar to the depressed measurements taken in controls at 4 weeks. Indeed, left ventricular volume at diastole showed further significant deterioration in the control indicating a continuing progression toward heart failure in the untreated control.

Second, the unexpectedly large beneficial effects on regeneration of cardiac muscle and reduction of scar in the injured heart were noted. Following echocardiography at 6 weeks, hearts were removed for morphological and histological analyses. A large pale scar was evident on control hearts with no sign of regeneration. This large scar extended to fully incorporate the boundaries of the initial injury. By contrast, the area of initial injury in a treated heart showed only a minimal amount of visible scar at the 6-week time point. In quantitative terms, less than 10% of the initially injured area on the control heart is cardiac muscle. By contrast, the treated heart showed a 70-90% regeneration of normal cardiac muscle. Thus, in summary our unexpected ability to prompt a full recovery of function in treated hearts is correlated with an equally impressive and unexpectedly extensive regeneration of normal cardiac muscle at the injury.

That regenerated muscle was present was further confirmed by histology of the hearts. Myocytes in treated hearts were found throughout the scar with a particular concentration of these cells near the epicardial border of the scar. This sub-epicardial population was notable for a number reasons. First, it is direct evidence for myocardial regeneration. The freeze injury is via a liquid nitrogen-cooled probe applied to the outer surface of the heart generating a hemi-spherical injury volume. During the freeze injury, the broadest sector of lethally frozen tissue is at the epicardium just under the freezing probe, i.e., the site where we see the “new myocytes” after 4 weeks of healing. Thus, this zone of sub-epicardial “new myocytes” must have regenerated over old necrotic tissue frozen near the epicardium—the previous cells at this location could not have survived the freeze injury. Indeed, in more than 20 control hearts subject to our standardized freeze injury evidence of regeneration at the sub-epicardium was never seen. The myocytes in this sub-epicardial zone were compact and highly aligned. This means that our treatment method had not only induced “new myocytes”, it had also the regenerated the precise tissue organization that existed at this locale in the heart prior to injury. Thus, our treatment had unexpectedly regenerated structure at both cellular and tissue scales—i.e., in addition to restoring function at the organ level. Thirdly, we note that these “new myocytes” are contiguous with adjacent myocardium. Cx43 immunolabeling indicates that these new myocytes also express the gap junction protein. Such tissue organization is consistent with electromechanical integration with surrounding myocardial tissues and the lowering the likelihood of arrhythmia. As noted previously, we contemplate that our novel composition will prevent arrhythmias. It can also be noted that the collagen staining appears significantly paler in the treated hearts indicating that collagen organization is different from that of controls. Whereas much cardiac research is focused on attempting to promote adult myocyte cell cycle re-entry to regenerate cardiac muscle, our novel approach leads to modification of scar organization in vivo. We posit that the scar in the treated animals is a “better scar”, permitting a new type of remodeling of this region with new myocytes. Finally, the section reveals that the extent of scar tissue as indicated by comparing the area of scar tissue is significantly less (>60-70% less) in the treatment compared to controls. This means that our treatment has an unexpectedly profound effect of tipping the balance between scar formation, organization, and inducing a multiscalar regeneration of functional myocardium in the injured heart.

In a further example in heart, the provided composition can be introduced via keyhole surgery in a human subject who has suffered an MI (i.e., preferably within 1 week of the MI) under full anesthetic by a surgeon into the minimally disrupted pericardial sac of the subject via a catheter. The composition can also be delivered by intravenous, intraarterial, intracardiac, or intraperitoneal injection. In another example, the composition can be sutured or secured by sterile surgical adhesive into place over an acutely healing MI while the subject's heart is exposed during coronary artery bypass graft surgery (CABG) and the like. Following CABG surgery the healing of the myocardium of the subject can be monitored for improvement in cardiac function by routine EKG, ambulatory EKG, echocardiography, blood assays and other tests of cardiac well-being and healing that a qualified clinician deemed necessary for the recovery of the subject. The provided composition can thus provide a treatment for injury to the heart and cardiovascular system. Thus, it is expected that healing will occur in a similar way when the ACT1 peptide is loaded into and delivered via an engineered vesicle as described in this application.

### Example 8

In Vivo Brain and Spinal (CNS) Wound Heating

In Example 8 the effect of ACT1 treatment is described to provide an example of contemplated use and results of the provided compositions when loaded with ACT peptide. See e.g. Gourdie et al, U.S. Pat. No. 7,786,074, which is incorporated herein by reference. In one example, anesthetized adult rats were positioned in a stereotaxic apparatus. A midline incision was made on the scalp to expose the skull. A stereotaxic drill was sighted 2 mm posterior to the bregma and 2 holes were drilled with a 1 mm spherical bit, each at 2.5 mm to the right and left of the bregma, and 3.5 mm below the dura. A cerebral lesion was made by inserting an 18-gauge needle. The coordinates were determined from the atlas by Paxinos and Watson (1986). The hollow fiber membrane (HFM) was inserted in the hole and external skin sutures were placed to cover the stab. The ACT peptide was dissolved at 100 μM concentration in a 2% collagen vehicle solution contained within the HFM. Studies of isolated HFMs indicated that these bioengineered constructs were capable of slow release of detectable levels of peptide (as assayed by biotin-streptavidin reaction) in aqueous solutions for periods of at least 7 days. Reactive astrocytosis associated with inflammation and subsequently with glial scar formation follows a well-characterized time course after brain injury in rodent models (Fawcett and Asher, 1999). Typically, the astrocytic response in rat brain peaks after a week, together with loss of neurons and other aspects of brain tissue complexity. Subsequently with the emergence of glial scar tissue, the density of GFAP-positive astrocytes decreases. In the control tissue, a high density of immunolabeled GFAP-positive astrocytes was observed near the site of injury caused by the HFM. The density of these cells appeared to diminish slightly distal from the injury. By contrast, a much lower density of GFAP-positive astrocytes was observed adjacent the HFM filled with peptide. Indeed, the levels of GFAP positive cells were not dissimilar to those seen in normal uninjured brain tissue. In the brain injury treated by active peptide, it was seen that GFAP-positive astrocytes were not only less numerous, but are also smaller than those seen in the control injury.

In the control tissue, a high density of immunolabeled GFAP-positive astrocytes and low density of NeuN immunolabeled neurons were observed near the site of injury caused by the HFM. The density of these cells appeared to diminish and increase distal from the HFM, respectively. By contrast, a much lower density of GFAP-positive astrocytes and higher numbers NeuN immunolabeled neurons was observed proximal (as well as distal) to the HFM filled with peptide. These results indicate that the high density of neurons associated with treatment can be from generation of new neurons. The peptide can also increase neuronal density in part by sparing neurons from cell death following brain injury. Subjects with acute spinal cord injuries to the central nervous system (CNS) represent a seriously problematic group for whom even a small neurological recovery of function can have a major influence on their subsequent independence. The provided composition can thus be useful in patients with a complete cord injury who normally have a very low chance of recovery. For optimal recovery of function the composition can be applied acutely or sub-acutely within 1 week of the initial injury. The prognosis of incomplete cord syndromes can also be improved by the composition. In a related example, spinal cord experiments were carried out on adult SD rats as previously described by Banik and co-workers (Sribnick et al, 2006). Rats are anesthetized and laminectomies are performed at T-12. Trauma is administered by dropping a weight of 5 g from a height of 8 cm onto an impounder (0.3 cm in diameter; 40 g·cm force) gently placed on the spinal cord. 30 μM peptide and control treatments (as per eye and heart injury) were immediately applied and wounds sutured closed. Spinal cord edema is assessed at 48 hrs post-injury, as described above. Cell death caused by compression injury was also assessed acutely on 5μιη sections of spinal cord from the lesion, which are co-labeled with NeuN and TU EL staining as a marker for neurons and cell death respectively. Assessment of inflammatory cell infiltration (e.g., microglia and macrophages) was done using 0X42 and ED2 antibodies. To determine the long-term benefits of treatment of treatment the functional and behavioral recovery of rats were tracked over time courses up 6 months following injury and NeuN and GFAP immunohistology will be used to assess glial scar and neurogenesis across the scar as described above for the brain injury. The provided composition can thus provide a treatment for injury to the brain.

In another non-limiting example, a subject with an acute anterior cord injury due to a flexion injury of the cervical spine can have surgery performed to expose the dorsal aspect of spinal cord at the level of the injury. A gel containing the composition described herein can then be placed directly on the injury. This gel can also contain neurogenic stem cells co-delivered with the provided composition to promote regenerative healing of the spinal cord. Single or multiple compositions are applied depending on severity of the injury. The surgical wound exposing the spinal cord injury is then sutured shut, enclosing the composition in situ. Improvement in function is assessed by a doctor at intervals (e.g., 6, 12, 26 and 52 weeks) following treatment by neurological outcome tests including assessments designed to measure motor activity, pinprick skin sensitivity and recovery of sensation. CT/MRI of the spine at the level of injury is also undertaken to monitor the healing progression of the subject. Medium- and long-term management can then be directed towards rehabilitation, including physiotherapy and occupational therapy to enable as full recovery of function as is possible following the treatment. The provided composition can thus provide a treatment for injury to the spinal cord.

In one aspect the recovery of spinal function will occur because of regeneration of new spinal cord neural connections from stem cells. This reparative aspect will occur in other CNS and PNS (peripheral nervous system) tissues. In another aspect, the recovery of spinal cord function will be contributed to by reduction in inflammation, swelling, edema and tissue loss associated with placement of the composition. Assay of this can be tested in animal models. For example, following injury to rat spinal cord in vivo, rats are treated with the composition. Soluble fluorescein-isothionate-tagged BSA (bovine serum albumin) or Evans blue dye is then injected into the tail vein. Control animals show leakage of the dye from the vascular system into tissues within and surrounding the spinal cord. However, animals treated with the composition demonstrated only limited dye leakage, with it majorly being confined with intact vascular structures. In the case of the CNS tissues such as the brain and spinal cord, this is due to the composition promoting the maintenance of the blood-brain barrier. However, the maintenance of barrier function should in some aspect be seen in all tissues of the body. The results indicate that leakage of the capillary-vascular system is not restricted to the CNS (e.g., spinal cord, brain, retina) and that a broader range of medical applications, such as for treatment of conditions of blood vessels, can benefit from treatment with the provided composition.

### Example 9

In Vivo Treatments of the Eye

In Example 9 the effect of ACT 1 treatment is described to provide an example of use and results of the provided composition carrying an ACT peptide. The results described in Example 9 were published in Rohrer and Gourdie, alpha-Connexin c-terminal (act) peptides for treating age-related macular degeneration, PCT/US2008/067944, Jun. 23, 2008 and Gourdie and Potts, US20110086068, and PMID: 28132078, which are incorporated herein by reference.

Normal eyesight is dependent on the transparency and regular curvature of the cornea. The histoarchitecture of the cornea is similar to that of skin-consisting of a stratified epithelium overlying a collagen-rich stromal matrix embedded with fibroblastic cells (e.g., keratocytes), although is largely avascular except at the periphery. Severe injury, surgery (Corneal refractive surgeries (CRS) such as photorefractive keratectomy (PRK)) and certain disease processes can lead to the loss of corneal transparency via activation of fibrotic/scarring processes in the corneal stroma. The resultant severe fibrosis of the cornea is difficult to treat and typically requires corneal transplant, which may lead to further complications. A safe and effective approach to reducing corneal scarring complication such as provided by the compositions described herein thus be welcomed by ophthalmologists and eye surgeons alike.

Minor scratches on the cornea are common and the composition is not envisaged to be used for normally healing minor injuries. However, the composition described herein can be of use in the treatment of more serious injuries to the cornea that may occur from small flying particles when drilling, sawing, chiseling, grinding, lawn mowing, and so on without eye protection and also from chemical burns such as that resulting from caustic solutions, acids, wet concrete and the like. Also the composition(s) described herein can be used in patients receiving CRS/PRK surgeries that may present high risk profiles such as those displaying wide pupils or evidence of poor wound healing such as might occur in a diabetic patient.

Following standard sub-acute stabilization and cleansing by a clinician, a subject suffering a severe chemical burn can have a collagen gel containing 180 μM JM peptide prepared, placed directly on the injury. The treatment can be undertaken within 1 week of the initial injury. Single or multiple compositions can be applied depending on severity of the injury. Antibiotic eye drops can then be placed in the eye to prevent infection. The composition can also be placed in association membrane to further aid healing. The eyelid can then be temporarily sutured closed, to retain the composition and a bandage can then be placed over the closed eye. Painkillers such as paracetamol or ibuprofen can be used to ease pain over the subsequent healing process. 7-14 days later the lids can be released and repair of the cornea assessed by an ophthalmologist for inflammation, scarring and other clinical indications of corneal healing. Improvement in function is assessed by a doctor at intervals (e.g., 6, 12, 26 and 52 weeks) following treatment by vision tests. An eye patch to cover the eye can not normally be advised after 10-14 days following injury as this may impair the healing process. An animal model of corneal injury (Chen et ah, 2009). In this model, adult (12 week) SD rats were anesthetized and the central cornea treated with 20% ethanol for 30 seconds using a 3-mm marker placed on the corneal surface. The cornea is then thoroughly rinsed with saline and the loosened epithelial layer removed using a detaching spatula. A treatment (i.e., PBS containing ACT1 peptide) or control gel was then placed in the alcohol burn injury and the eye-lid sutured shut for 48 hours to hold the gel in place. Corneal wound closure was determined by administering 0.25% fluorescein sodium eye drops and digitally capturing the cornea under a fluorescent stereomicroscope at 0, 48, 72, 96, and 120 (closure is usually complete by 120 hours in rat) hours post-injury. Levels of scar tissue deposition and transparency were assessed on whole mounts of isolated corneas 30 days post injury. Corneal tissue was subject to standard histological and immunohistochemical studies on tissues sections to assess corneal epithelial and endothelial integrity and collagen organization and myofibroblast (alpha-SMA) density in the stroma. Corneas treated with active peptide showed faster closure and more complete corneal regeneration than control corneas. The provided composition is thus contemplated to provide a treatment for injury to the cornea of the eye.

Trans-epithelial resistance (TER) measurements, using ARPE19 cell (immortalized human RPE cells) mono layers has revealed that VEGF leads to rapid deterioration, which was blocked by pre-treating the cells with the ACTI peptide US2008067944. Thus, this peptide can prevent damage to RPE/Bruch's membrane. The peptide contains a NT cell internalization sequence (CIS). Together with a mild detergent that is used in ocular applications, Brij-78, the CIS assists in permeation of peptide into interior fluids and tissues of the eye. In some aspects, thus JM peptides can enter the internal fluids and tissues of eye and this is a mode of action of CIS containing peptides in treating diseases of the eye such as macular degeneration. The provided composition can thus provide a treatment for promoting stabilization of RPE cells and tissues to permeation in response to VEGF increase.

Application of peptide in a solution containing 0.05% Brij-78 to the cornea of mouse eyes resulted in a detectable level of ACTI in the internal fluids of the anterior chamber (i.e., the aqueous humor) 20 and 40 minutes post application. Lower levels of peptide could also be detected by Western blotting in fluid from the posterior chamber of eye 20 and 40 minutes, i.e., the vitreous humor. Following application of peptide in a solution containing 0.05% Brij-78 to the cornea of mouse eyes, peptide was detectable in the retinal pigment epithelial layer of eye minutes post-application. Moreover, peptide was immunohistochemically detected in the retinal pigment epithelial layer of eyes exposed to the peptide, but not to the vehicle control solution via corneal application. Three CD1 mice were anesthetized by IP injection of ketamine per standard protocol.

ACTI peptide (final cone 100 μM) was dissolved in a solution containing normal saline and 0.05% Brij-78 was gently dripped onto the corneal surface of both eyes and allowed to permeate for 20 or 40 min. 0.05% Brij-78 in saline was used on a control mouse. The mice were sacrificed in a C02 chamber and cervically dislocated at 20, 40 min (the control mouse sacrificed at 20 min). The eyes were removed and rinsed in PBS. A small incision was made in the anterior chamber and the aqueous humor (−10 fIL) was transferred to tube and flash frozen in a dry ice ethanol bath. The total sample was dissolved in 2× samples loading buffer and loaded on a 10-20% Tris-Tricine gel. Gel was transferred to a PDVF membrane and stained using RBT Sigma anti-CX43 C-terminal antibody (1:10000) and a goat anti-RBT AP secondary (1:15000) to reveal the ACTI band at <10 kDa. Application of peptide to the cornea in Brij-78 was the same as described above. After sacrifice the mouse eyes were removed, washed in PBS briefly, and transferred to 5% paraformaldehyde overnight. The eyes were embedded in paraffin, sectioned, and stained with Sigma Rbt anti-Cx43, streptavidin and Hoeschst stain and placed at 4 degrees overnight. Peptide is detectable in the interior fluids and tissues of the eye following a simple corneal exposure.

Electroretinography (ERG) to assess level of CNV damage can be recorded using similar protocols to those published by Gresh et al. (2003). Mice are dark-adapted overnight, anesthetized and pupils dilated. Body temperature is stabilized at 37° C. (DC-powered heating pad). A ground-electrode is placed in the tail, a reference-electrode in the forehead. ERG responses are measured using contact lenses with a gold-ring electrode held in place by methylcellulose. ERGs are recorded (EPIC-2000, LKC Technologies), using a Grass strobe-flash stimulus (gain of 2k, notch filter set at 60 Hz). Responses are band-pass filtered (0.1-1500 Hz) and digitized (1 kHz, 12 bit accuracy). Stimuli to isolate rods consist of 10 μs single-flashes at a fixed intensity (2.48 photopic cd-s/m²) under scotopic conditions. Single-flash responses are averaged 2-4× with an inter-stimulus interval of 120 sec. Cone responses can then be recorded under light-adapted conditions, using stroboscopic illumination (1-30 Hz) for stimulation. A-wave amplitude is measured from baseline to the a-wave trough; b-wave amplitude from the a-wave trough or baseline to the peak of the b-wave, and implicit time from onset of stimulus to a-wave trough or b-wave peak.

In studies in vivo it has been shown that: 1) ACTI can be formulated to permeate into the chambers of the eye following corneal application (e.g., intravitreal injection not required); and 2) in a laser-induced choroidal neovascularization (CNV) mouse model of retinal macular degeneration, peptide treatment reduced CNV injury spread and improved retinal function (as measured by electro-retinal gram (ERG), relative to controls. These results parallel our published data that the peptide reduces inflammation, time to heal and scar tissue formation following dermal wounding. The provided composition is thus contemplated to provide a treatment for macular degeneration.

Thus, it is expected that treatment for macular degeneration will be effective when the composition(s) discussed above is/are loaded into and delivered via an engineered vesicle as described in this application.

### Example 10

Uses in Tissue Engineering

Results described in Example 10 were published in part in Gourdie and Potts, US201 10086068 and Soder et al. (2009), which are incorporated herein by reference.

Loss of skeletal muscle mass is an important problem for surgeons. Skeletal muscle has some ability to regenerate from endogenous stem cells called satellite cells. However, if the injury is large, this natural reparative ability can be overwhelmed. In such cases, muscle is not regenerated and scar tissue replaces lost muscle—if the patient is fortunate. One clinically important example of injuries involving muscle that can be difficult to repair are ventral hernias (also known as incisional hernias). Annually, over 2 million abdominal operations are performed in the United States. (Millikan, 2003). Given a failure rate for abdominal closures of 11 to 20 percent, it is not surprising that over 100,000 ventral hernia repairs are attempted each year in the United States alone. The incidence of ventral hernias has remained relatively stable over the last 75 years despite many medical advances.

The repair of ventral hernias typically involves the closing the hernia with a synthetic mesh or more recently decellularized human dermis (Alloderm, LifeCell). Although these methods effectively “patch the hole” they lack the ability to reconstitute the lost abdominal muscle. The mesh imparts no contractile function and with large hernias it is ineffective at producing counter pressure from the contracture of remaining abdominal musculature. These repair techniques do little to reestablish the dynamic role of the abdominal wall in support of the torso and lumbar spine. With dynamic repairs, force vector summation of abdominal wall contraction is focused on the repair itself. Mesh repairs are also associated with bowel obstruction (5%), enterocutaneous fistulae (2-5%), and infection (1-2%). The aggregate incidence of long term complications associated with mesh repair approaches 27% (Mudge and Hughes, 1985). In the following example we outline how our invention can be used to repair an experimental ventral hernia in a rat-by extension in a human subject.

To create the ventral hernia model, 250 gram male Sprague Dawley rats are used. This size male rat has sufficient tissue for isolation of satellite cells, creation of the abdominal defect and has matured sufficiently to be considered adult in phenotype. After general anesthesia is achieved, the animal is prepped in standard surgical manner. A 1 cm×1 cm excisional wound is then generated in the abdominal muscle through to the cavity of the abdomen. To isolate autologous satellite cells from skeletal muscle of the same rat, a muscle biopsy (0.5 mm×0.5 mm×0.2 mm=0.05 cm³) is extracted from the vastus lateralis and placed in mosconas on ice. This provides the 10 to 1 expansion of cells required to repair the defect. The biopsy wound is approximated and closed by suture. The sampled muscle tissue is rinsed vigorously with PBS at least three times to remove blood. The tissue is then minced thoroughly with scissors to dislodge adherent fat and washed several times with cold PBS. Warmed and gassed protease is added (sigma #P-5147; 1.25 mg/ml in Krebs Ringer Bicarb. Buffer (Cat #K4002)) to the tube with the tissue at a concentration of 1:5 (enzyme: tissue), followed by 1.25 hours shaking incubation at 37° C. The tube is centrifuged and the pellet is resuspended in 25-30 ml of high serum media (DMEM+25% Fetal Bovine Serum+1% Pen/Strep antibiotic+0.1% Gentamycin). DNAse is added and the tube is shaken vigorously and centrifuged to collect the sample. Spun supernatants are then panned onto 150 mm dishes with 25-30 ml media for 1.5 hours at 37° C. in the incubator. The cells are dislodged with 0.25% trypsin-EDTA when cells are at least 90% confluent, counted and seed onto CtCs. A sister culture of satellite cells is then created in collagen coated culture dishes. The cells are then characterized by immunolabeling for Pax 7, Myf5, MyoD, and sarcomeric myosin (MF20). In previous studies, the satellite cell cultures are 80+% positive for Pax7 and MyoD. For generation of skeletal muscle stem cells, 30-50 collagen gels are prepared in 2 cm diameter circular wells as described above. Dispersed satellite cells (12×10⁶ per well) are then applied to the well. The cells are allowed to attach and culture of the collagen substrate for 24 hours and then the gel is released as per standard practice for the disclosed invention. Alternately, the gels can be released after cell attachment is achieved, static or dynamic strain is then applied to generate preferred alignment and differentiation potential of the adherent cells. The gels (containing cells or no cells) can also be soaked for example in 100 μM JM peptide, assisting muscle regeneration by the stem cells. Following a 24 hour period in culture, circular gels containing peptide and stem cells can then be stacked within a single well, each layer being adhered to the next by small dab of Cell-Tak™ at the gel edge. The cylindrical 3D assembly of gel layers of skeletal stem muscle cells then has a suture threaded through the middle of its long axis, removed from the culture well and then placed in the open excisional wound in the abdominal muscle of the rat. The suture thread through the cylinder of stem cells stabilizes the assembly and also is used to secure it in place. To increase the robustness of the repair multiple 3D tissue engineered constructs of satellite cells can be applied to the ventral hernia. The repair site is then covered with an appropriate surgical membrane and wound dressing to protect the wound and implanted tissue engineered device. Animals are then sampled at time points between initial wounding and 16 weeks.

In the rat model, inflammatory response, scarring and skeletal muscle regeneration can be assessed using histochemistry and immunohistochemistry (e.g., Pax7, MyoD, MF20 expression) of the repaired abdominal tissues using standard approaches. Functional assessment of live tissue from the repair can be done by taking regenerated muscle from the repair placing in a muscle bath, oxygenated (95% O₂ and 5% CO₂) Krebs solution maintained at 37° C. at pH 7.4, and undertaking physiological tests of muscle function: isometric contraction, length/tension relationship determination, and breaking stress and strain. In human subjects, closure of the hernia, assessments of scarring and restoration of abdominal muscle function as assessed by a qualified clinician can be undertaken. Small biopsies of the repair can also be taken for direct assessment of muscle regeneration by histology by a qualified histotechnologist under the supervision of a clinician. However, it can be advisable to keep such invasive diagnoses to a minimum. The provided composition can modulate the wound-healing response to a cellularized tissue engineered implant, promoting its integration and maintenance in the human body. An engineered vesicle as described herein can be used as a co-therapy or be integrated with the compositions demonstrated in this Example.

### Example 11

In another example that illustrates the untility of the present invention when ACT peptide is contained in the provided EVs, silicone disks coated with either vehicle control or ACT 1 peptide were implanted submuscularly into male Sprague-Dawley rats. Capsulectomies were performed on days 1, 2, 3, 14, and 28 of that method described in Soder et al 2009 (PMID: 19407614). The implant capsules and surrounding tissue were analyzed histologically and biochemically. The peptide modulated the wound-healing and foreign body responses to silicone implants by attenuating neutrophil infiltration, increasing vascularity of the capsule tissue, reducing type I collagen deposition around the implant, and reducing the continued presence of contractile myofibroblasts. Thus, ACT1 can thus provide a technology for modulating the wound-healing response to silicone breast implants, as well as all other types of devices implanted in the body, promoting integration of implanted materials and tissue-engineered devices in the human body. Incorporation of the ACT1 peptide into an engineered vesicle as described in this patent application is expected to modulate the wound-healing response to implants, promoting integration of implanted materials and tissue-engineered devices in the human body in a similar fashion as delivery alone.

### Example 12

Uses in Cancer Treatment.

Results described in Example 12 were published in part in abstract form as Zhu et al and given to provide an example of the results of the provided compositions carrying ACT peptide. 2007 at the Pediatric Academic Societies 2007 Annual Meeting, May 5-8, 2007, Toronto Canada, which is incorporated herein by reference.

The infiltration of glioma cells within the central nervous system (CNS) accounts for high rates of mortality and morbidity. This infiltration requires cellular attachment, cytoskeletal-dependent motility, and protease-dependent invasiveness. Recent research has revealed that a hallmark of many glioma cell lines is the aberrant expression of gap junctions, intercellular membrane channels that allow direct cell-to-cell communication. Gap junction channels are composed of protein subunits called connexins, which are maintained and organized by many scaffolding proteins and cytoskeletal components. One such scaffolding protein is zonula occludens-1 (ZO-1), which binds to the carboxyl terminus of connexin43 (Cx43), a major gap junction protein subtype.

In many malignant glioma cell lines, Cx43 gap junction organization may play important roles in tumorigenicity, and more specifically, in invasiveness. A peptide, called ACT-1 and based on the of Cx43, was designed to be a competitive inhibitor of Cx43 and ZO-1 interaction and has been previously shown to alter gap junction dynamics in fibroblasts. In this study, U87 MG glioblastoma cells (which express Cx43) treated with the peptide displayed a higher degree of aggregation, a significant aspect of tumor cell migration. In contrast, the adhesive properties of the Cx43-deficient C6 glioma cell line did not change in response to peptide treatment. Interestingly, the C6 cells did display altered morphology after treatment with the peptide, suggesting that the peptide also influences cytoskeletal organization, another important factor in glioma migration. These results provide insight into the role of the Cx43 in malignancy. The provided composition can thus provide a new approach for cancer treatment.

These and other modifications and variations to the present disclosure can be practiced by those of ordinary skill in the art, without departing from the spirit and scope of the present disclosure, which is more particularly set forth in the appended claims. In addition, it should be understood that aspects of the various aspects can be interchanged both in whole or in part. Furthermore, those of ordinary skill in the art will appreciate that the foregoing description is by way of example only, and is not intended to limit the disclosure. Further examples of the use of ACT and JM peptides in cancer that are contemplated to provide benefit when administered in the provided compositions herein can be found in the citations PMIDs 27863440, 26542214, and WO2015017034 A1, which are incorporated by reference. For example, in Murphy et al 2015 (PMID: 26542214), it was reported that resistance of glioblastoma (GBM) to the front-line chemotherapeutic agent temozolomide (TMZ) continues to challenge GBM treatment efforts. The repair of TMZ-induced DNA damage by O-6-methylguanine-DNA methyltransferase (MGMT) confers one mechanism of TMZ resistance. Paradoxically, MGMT-deficient GBM patients survive longer despite still developing resistance to TMZ. Recent studies indicate that the gap junction protein connexin 43 (Cx43) renders GBM cells resistant to TMZ through its carboxyl terminus (CT). In this study, we reported insights into how Cx43 promotes TMZ resistance. Cx43 levels were inversely correlated with TMZ sensitivity of GBM cells, including GBM stem cells. Moreover, Cx43 levels inversely correlated with patient survival, including as observed in MGMT-deficient GBM patients. Addition of the C-terminal peptide mimetic aCT1, a selective inhibitor of Cx43 channels, sensitized human MGMT-deficient and TMZ-resistant GBM cells to TMZ treatment. Moreover, combining aCT1 with TMZ-blocked AKT/mTOR signaling, induced autophagy and apoptosis in TMZ-resistant GBM cells. Our findings indicate that combining ACT peptides in the present composition with TMZ can enhance therapeutic responses in GBM, and perhaps other TMZ-resistant cancers.

In another example, JM peptides can selectively target cancer stem cells CSCs. JM2 specific interaction with microtubules concomitantly with a loss of Cx43/microtubule complexing and a decrease in cell-cell communication in CSCs derived from patient tumors was confirmed. JM2 decreases CSC survival in vitro and in vivo. Current research includes the development of JM2-loaded biodegradable nanoparticles for JM2 sustained delivery in preparation for future clinical trials. In sum, the Cx43 mimetic peptide JM2 represents a novel and potent therapeutic opportunity to target chemoresistant CSCs in cancer treatment. This was described in (Lamouille et al., targeting glioblastoma cancer stem cells with a novel Connexin43 mimetic peptide [abstract]. In: Proceedings of the American Association for Cancer Research Annual Meeting 2017; 2017 Apr. 1-5; Washington, D.C. Philadelphia (Pa.): AACR; Cancer Res 2017; 77 (13 Suppl):Abstract nr 4765. doi:10.1158/1538-7445.AM2017-4765), which is incorporated by reference. Further results can indicate that cancer stem cells derived from other cancers respond similarly to JM peptides. For example, JM2 effectively reduced the viability and self-renewal of cancer stem cells from patients with colon cancer. Thus, JM peptides cargoed in the engineered vesicles described herein can be in selectively targeting and killing cancer stem cells in all cancers characterized by these unique cells including in glioma and multiple myeloma and brain, colon, breast, lung, pancreatic, ovarian, prostate, melanoma, and non-melanoma skin cancers.

### Example 13

In Example 13 the effect of JM peptide treatment is described to provide an example of use and results of the provided composition carrying a JM peptide. JM2 Peptide can Decrease Inflammation and Scarring and Promotes Regenerative Healing associated with Silicon Implants

Animals

Harlan Sprague-Dawley (Indianapolis, Ind.) male rats weighing approximately 200-300 g were used throughout this work. Animals were managed in the institutional animal care facility in compliance with the Guide for the Care and Use of Laboratory Animals published by the National Academy of Sciences and all animal protocols were approved by the University of South Carolina Institutional Animal Care and Use Committee (IACUC).

JM2 Preparation

25% pluronic F127 gel (Sigma-Aldrich, St. Louis, Mo. 63103), which is liquid at 4° C., but gels at 37° C., was used as a delivery vehicle for JM2 peptide. Pluronic gel has mild surfactant properties that aid in peptide dispersion. The JM2 peptide was reconstituted in IX PBS containing the 25% pluronic gel to a final concentration of 180 micomolar.

Implantation Procedure

Animals were anesthetized with 2.75% isoflurane balance oxygen gas. After achievement of general anesthesia, the surgical site consisting of the animal's upper back was prepped by clipping fur down to skin and applying betadine scrub solution in triplicate. Sterile towels were draped to define the surgical field. PWAS Silicone sensors (5 mm diameter) were autoclave sterilized and warmed to 37° C. prior to implantation. For the treatment group, implants were dipped twice in JM2 pluronic solution prior to implantation. For the vehicle control group, the implants were dipped in saline only. This coating procedure produced an even coating of the implant. A muscle pocket was created under the latissimus dorsi muscle and implants were inserted. 50 μL of the corresponding solution was also injected into the muscle pocket prior to closure. The muscles were reapproximated with 4-0 Prolene (Ethicon Inc, Somerville, N.J. 08876) and the skin closed with 4-0 Prolene and skin staples. Upon recovery from anesthesia animals were given a bolus of 3 ml normal saline subcutaneously and 0.1 mg/kg Buprenorphine HCl (Reckitt Benckiser Healthcare Ltd., Hull, England HU8 7DS) intra-muscularly to alleviate pain.

Capsule morphometric analysis Nine animals were organized into three groups, 24 hours post implantation, 72 hours post implantation, and 4 weeks post implantation. In each group, three animals JM2 treatment and one control. PWAS Silicone disks (5 mm diameter) were implanted as previously described. At each time point post implantation, four animals from each group underwent capsulectomy to remove the implant and surrounding tissue capsule. The tissue was vibrotome sectioned and stained for H&E and Masson's trichrome. Three tissue sections from each animal were examined with light microscopy. Presence of the JM2 peptide decreased inflammation and reduced skeletal muscle necrosis associated with a silicone implant in vivo. Treatment with JM2 peptide improved healing and decreased capsule formation, scarring and fibrosis associated with the silicon implant, as well as promoting the long-term maintenance, and/or growth and regeneration of skeletal muscle and other tissues surrounding the silicone implant.

### Example 14

JM Peptides can Inhibit Cx43 Hemichannel Activity

In Example 14 the effect of JM peptide treatment is described to provide an example of use and results of the provided composition carrying a JM peptide and was reported in Rhett et al., 2017 (PMID: 28701358). The hypothesis that ACT1, JM1, and/or JM2 can inhibit Cx43 hemichannei activity, thereby preventing release of the inflammatory activator ATP, was tested in the following experiments. Data was generated regarding the mechanism of JM peptides on Cx43 GJ channels and hemichannels. The effect of JM2 on GJ channels was tested as follows. Cx43-HeLa cells were treated with 10 μM JM2 or vehicle for 2, 6, and 24 hrs in standard cell culture conditions. Vehicle treated controls were generated. The cells were labeled for Cx43, N-Cadherin, and the nucleus. Cells were fixed with 2% paraformaldehyde and labeled with Cx43 antibody, N-cadherin antibody (to indicate cell-cell apposing membranes), and Hoechst nuclear stain. A typical punctate Cx43 GJs in control cells was observed. In contrast, cells treated for 2 hrs with JM2 displayed little GJ labeling. Whether this lack of labeling is a result of changes in expression level or GJ formation will have to be address by Western analysis, as proposed below. Interestingly, GJ labeling began to return at 6 hrs, and appeared normal after 24 hrs. These data indicate that JM2 temporarily reduces cell-surface Cx43 in cultured cells. Similar results were observed with JM1 peptide. As JM1 and JM2 are based on part of a putative juxtamembrane microtubule binding motif of Cx43, cells were also labeled for microtubules using an anti-a-tubulin antibody. The 2 hr time point was focused on as it seemed to have the greatest effect on Cx43 labeling. A decrease cell-surface Cx43 labeling in JM2 treated Cx43-HeLa cells, and what appeared to be an increase in intracellular Cx43 labeling, was again observed. Importantly, the inventors also observed disruption of microtubule organization. Since the JM region also shows homology to protein phosphatase interaction domains and thus additional complexity for the molecular mechanism may exist.

These labeling studies provided direct evidence for an effect of JM2 on GJ channels, not hemichannels. However, the observed increase in intracellular Cx43 labeling suggested the possibility that targeting microtubules with JM2 affects Cx43 trafficking to, or stability in, the membrane. Therefore, studies were carried out on hem ichannel activity as described for ACT1 in Rhett et al, 2011 (PMID: 21411628). It was found that JM2 was a highly effective hemichannel blocker. Specifically, treatment of Cx43-HeLa cells with 50 μM JM2 for 2 hrs (as compared to 180 μM ACT1 for 2 hrs in Rhett et al, 2011), significantly reduced hemichannel activity to the level of wild-type HeLa cells (i.e., not expressing Cx43).

The possibility that Cx43 acts as a mediator of inflammation through hemichannel mediated release of ATP was further examined. Studies on ATP release in the HeLa cell models, as well as primary human microvascular endothelial cells (HMVECs), were performed. Endothelial cells were chosen as a model for ATP release because of their direct access to the blood stream, through which neutrophils have been demonstrated to migrate in response to injury-generated purinergic signaling (McDonald et al, 2010; Baroja-Mazo et al, 2013). It was observed increased ATP release in response to cellular stress in the form of low Ca2+, a widely used trigger for connexin hemichannel opening, and the inflammatory cytokine IL-6. However, the inventors also observed that, in preliminary experiments, ATP released in response to low Ca²⁺ was not inhibited by treatment with mefloquine, a commonly used connexin channel blocker. Endothelial cells may also release ATP through vesicular exocytotic mechanisms in a Ca²⁺ dependent manner (Bodin and Burnstock, 2001; McDonald et al., 2010). Pannexin channels, which can also mediate ATP release, are similarly sensitive to mefloquine (Lohman et al, 2012; Bodin et al, 2001). Strategies for Promoting Survival of Satellite Cells Following Implantation

Cell transplantation therapies for muscle regeneration are currently challenged by low survival of implanted cells (typically 5-10%). aCTI, one of the compounds that was used in this project, inhibits Cx43 hemichannel activity in the perinexus (Rhett et at, 201 1; Rhett and Gourdie, 2012; Lohman et ah, 2012). JM peptides can be used in a Cx43-based targeting approach. Similar to aCTI, JM peptides also potently reduce Cx43 hemichannel activity. The molecular mechanism of aCTI and JM peptides can be distinct, raising the prospect for further increase in efficacy based on therapeutic approach combining the two novel compounds. In addition to Cx43 hemichannel targeting to improve survival of engrafted cells, pre-aggregation of satellite cells prior to implantation into injured skeletal muscle may be performed. The effect of bone marrow stem cells, an ‘immune-privileged’ cell type, on the survival of implanted aggregates is also being examined. Satellite cells and bone marrow stem cells (BMSCs) were from adult rats and aggregates have been generated from satellite cells using Morgan molds. Satellite cell survival following engraftment of these aggregates in a rat model in vivo can be performed.

The addition of JM2 peptide can block the function of Cx43 hemichannels. In the control images, profound inflammatory infiltrate were seen. The border zone between the tissue reaction area and the intact skeletal muscle was ill defined with what appears to be continued necrosis of the native muscle. In contrast, exposure to a JM1 or JM2 peptide resulted in a substantially narrower tissue reaction zone. The border zone between the intact muscle and implant reaction area is much better defined with little continued muscle necrosis at 24 hours.

15 male Sprague Dawley rats underwent unilateral implantation of silicon wafer implants. Three animals received implants only, three received implants plus exogenous ATP, three received implants plus exogenous apyrase, an enzyme that scavenges ATP, three underwent surgery alone without an implant, and three received a percutaneous injection of exogenous ATP into the latissimus dorsi muscle. The implants were harvested after 24 hours to evaluate the extent of inflammatory infiltrate and preservation of muscle. The implant alone causes significant inflammatory infiltrate as well as ill-defined boarder areas and coagulative necrosis of muscle fibers. The addition of exogenous ATP causes a profound increase in inflammatory infiltrate in the implant region, top right panel. Interestingly, treatment with apyrase at the implant site significantly reduced the inflammatory infiltrate. There are still some inflammatory cells present but the numbers are greatly reduced and the muscle is preserved, lower left panel. Finally, a simple percutaneous injection of ATP caused more inflammatory infiltrate than the surgery alone, further confirming our hypothesis that extracellular ATP plays a profound role in neutrophil targeting to damaged skeletal muscle tissue.

### Example 15

Effect of Loss of Cx43 Function on STEMI Repair of Mechanically Active Skeletal Muscle. Analysis of STEMI implants in the active skeletal muscle of the abdominal wall was performed. New muscle in the repair and reductions in the amount of scar tissue formation were observed. New skeletal muscle was generated that has fibrous scar tissue in-between most of the muscle fibers. It was hypothesized that there may be tissues that develop early on in development that are generic for the creation of vascular beds and for creating motor neuron connections. It was further hypothesized that these tissues may be affected by differentiating cells to proliferate and form blood vessels or motor neuron connections. For vascular bed formation, these cells can include endothelial cells and fibroblasts derived from the splanchnic mesoderm. To mimic this in an autologous transplant, stromal vascular fraction cells were isolated from adipose tissues and attempt to form endothelial cell tubule networks. For motor innervation, these cells were taken from the neural crest. The following data on STEMI repairs of active skeletal muscle was generated.

In attempting to quantify the neutrophil infiltrate using a myeloperoxidase stain, the inventors observed that some of the cells stained darker than others. Upon further investigation, these cells were not neutrophils as but rather were macrophages. It was further determined that at the 24 hour time point, untreated implants showed predominately neutrophils; however, when treated with ACT1 the primary inflammatory infiltrate was macrophages. This data supports the idea that the JM peptides can close Cx43 hemichannels and reduce or mute the ATP signal for inflammatory cell migration.

### Example 16

A further example of the invention is its use in preserving cells, tissues and organs for transplantation. For example, an ACT peptide contained within the provided EVs. In U.S. patent application Ser. No. 14/932,630 (which is incorporated by reference), ACT1 peptide stabilizes gap junctions (Cx43) and minimizes mitochondrial oxidant production (nitrotyrosine) and apoptosis (TUNEL and caspase) in porcine kidney models of cold ischemia. Punctate Cx43 staining in the membrane (gap junctions) were preserved in ACT1 peptide-treated kidneys and early control biopsies, while at 24 h Cx43 staining became more diffuse and appeared to localize to the cytoplasm of cells in the control kidneys. The 12 and 24 h sections demonstrated intense, localized nitrotyrosine staining in the apical and basolateral areas of control kidney cells in comparison to the ACT1 peptide-treated samples. There were no changes in nitrotyrosine staining in the presence of ACT1 peptide or in time zero control biopsies that were not subjected to cold ischemia. Apoptotic cells were also observed in the 24 h control.

These studies were conducted using kidneys procured from 2 standard criteria donor pigs. The organs were flushed via the aorta with preservation solution after 10 minutes of warm ischemia post-mortem. Biopsies were taken prior to treatment. The kidneys were then flushed with either cold Belzer's solution (control) or the same solution containing 100 μm ACT1 peptide, and stored in the respective solutions on ice for 24 h. Biopsies were taken at regular intervals and sections were stained for Cx43 and nitrotyrosine.

U.S. patent application Ser. No. 14/932,630 can show ACT1 peptide can protect endothelial cells. Storage of both epithelial and endothelial cell with Belzer's University of Wisconsin (UW) solution containing 100 μM ACT1 peptide significantly reduced cellular injury as compared to untreated controls. Further, supernatants and cell lysates were collected to measure IL-8 secretion and MHC II expression. Treatment of either cell type with UW solution supplemented with ACT1 peptide was associated with significant reduction in IL-8 secretion and MHC Class II expression.

These studies were conducted using a modification of the in vitro donor cold storage and reperfusion injury model (Casiraghi et al., 2009). Human umbilical vein endothelial cells (HUVECs) and mouse microvascular endothelial cells were grown to confluence on transwells and transendothelial resistance (TEER) was recorded. To model cold ischemia and reperfusion injury growth, media was removed from the cells and replaced with either ice cold UW solution or UW solution containing ACT1 peptide, and cultures were exposed to 6 h of hypoxia in a hypoxic chamber (Billups-Rothenberg, Del Mar, CA) at 4° C. Following hypoxic exposure, UW solution was removed, and to stimulate reperfusion, UW solution was replaced with fresh pre-warmed (37° C.) culture media, and cells were monitored for 24 h. TEER was measured at three time points post reperfusion as a marker of endothelial/epithelial cell death and dysfunction. A loss of electrical conductivity, as measured by TEER, across the cellular monolayer is associated with a loss of cell-cell communication (thus, gap junction and tight junction injury), cell injury, and a leaky endothelial cell lining. In vivo, this can translate as dysfunction of the cell layer and facilitate uncontrolled fluid trafficking, loss of vascular tone, reduced barrier function that can facilitate immune cell infiltration.

In a further example, ACT1 can prevent VEGF-induced deterioration of TER in ARPE-19 cells. Trans-epithelial resistance (TER) measurements, using ARPE 19 cell (immortalized human RPE cells) monolayers revealed that VEGF leads to rapid deterioration, which was blocked by pre-treating the cells with the ACT peptide. Thus, while not wishing to be bound by theory, stabilizing the tight junction proteins with the ACT peptide can prevent loss of tight-junction disintegration and thus damage to RPE/Bruch's membrane. ACT1 Peptide contains an amino terminal cell internalization sequence. Together with a mild detergent that is used in ocular applications, Brij-78 the antennapedia sequence assists in permeation of ACT1 into interior fluids and tissues of the eye. In some aspects, the ability of ACT1 to enter the internal fluids and tissues of eye is a mode of action of ACT1 in treating diseases of the eye such as macular degeneration. Application of ACT1 peptide in a solution containing 0.05% Brij-78 to the cornea of mouse eyes resulted in a detectable level of ACT1 in the internal fluids of the anterior chamber (i.e., the aqueous humor) 20 and 40 minutes post-application. Lower levels of ACT1 could also be detected by Western blotting in fluid from the posterior chamber of eye 20 and 40 minutes, i.e., the vitreous humor. Following application of ACT1 in a solution containing 0.05% Brij-78 to the cornea of mouse eyes, ACT1 was detectable in the retinal pigment epithelial layer of eye minutes post-application. Moreover, ACT1 was immunohistochemically detected in the retinal pigment epithelial layer of eyes exposed to the peptide, but not to the vehicle control solution via corneal application.

Three CD1 mice were anesthetized by IP injection of 0.2 mL Salazine/ketamine. 10 μL of 1 mM ACT1 peptide dissolved in a solution containing normal saline and 0.05% Brij-78 was gently dripped onto the corneal surface of both eyes and allowed to permeate for 20 or 40 min. 0.05% Brij-78 in normal saline was used on a control mouse. The mice were sacrificed in a CO₂ chamber and cervically dislocated at 20, 40 min (the control mouse sacrificed at 20 min). The eyes were removed and rinsed in PBS. A small incision was made in the anterior chamber and the aqueous humor (about 10 μL) was transferred to tube and flash frozen in a dry ice ethanol bath. The total sample was dissolved in 2× samples loading buffer and loaded on a 10-20% Tris-Tricine gel. Gel was transferred to a PDVF membrane and stained using RBT Sigma anti-CX43 antibody (1:10000) and a goat anti-RBT AP secondary (1:15000) to reveal the ACT1 band at <10 kDa.

Application of ACT1 to the cornea in Brij-78 was the same as described above. After sacrifice the mouse eyes were removed, washed in PBS briefly, and transferred to 5% Paraformaldehyde overnight. The eyes were embedded in paraffin, sectioned, and stained with Sigma Rbt anti-Cx43, streptavidin and Hoeschst stain and placed at 4 degrees overnight. As disclosed herein, ACT1 is detectable in the interior fluids and tissues of the eye following a simple corneal exposure.

The impact of ACT1 peptide supplementation of UW solution in a small animal transplant model was examined. ACT1 peptide therapy significantly reduces Evan's Blue sequestration into the transplanted heart as compared to controls, indicating that ACT1 peptide promotes gap junction and tight junction stability, and improved endothelial cell integrity. Heart allograft transplants were performed between Balb/c donors to B6 recipients. Balb/c donor hearts were removed, perfused with UW solution and then static cold stored in either UW solution alone or UW solution supplemented with 100 μM ACT1 peptide for 6 h at 4° C. Following storage, hearts were implanted into B6 recipients using an abdominal heart transplant procedure. To assess the impact of ACT1 peptide augmented cold storage on heart vascular permeability/damage, recipients were injected with Evan's Blue Dye immediately following reperfusion. Hearts were then harvested for 30 mins post reperfusion and assayed for Evan's Blue uptake. ACT1 peptide treatment improved endothelial barrier function was associated with reduced ischemia reperfusion injury (IRI). ACT1 peptide supplementation of UW solution improves cell-cell communication, thus minimizing cell injury, cell dysfunction, inflammation, and improves overall donor organ quality. Specifically: 1) ACT1 peptide prevents UW cold storage induced endothelial and epithelial injury; 2) reduces endothelial pro-inflammatory cytokine release; 3) reduces endothelial permeability post transplantation; 4) reduces heart graft injury post transplantation; and 5) reduces post transplantation inflammation.

Heart allograft transplants were performed between Balb/c donors to B6 recipients. Balb/c donor hearts were removed, perfused with UW solution and then static cold stored in either UW solution alone or UW solution supplemented with 100 μM ACT1 peptide for 6 hrs at 4° C. Following storage, hearts were implanted into B6 recipients using an abdominal heart transplant procedure. Storage in UW solution supplemented with ACT1 peptide significantly reduced cardiac injury, reduced serum cardiac troponin I and significantly reduced neutrophils.

ACT1 peptide protects endothelial cells from cold preservation induced damage, hypoxia, inflammation and reperfusion injury. The endothelium is the first point of contact between donor and recipient. Upon reperfusion, the endothelium becomes quickly activated and initiates pro-inflammatory, pro-coagulant, and co-stimulatory roles that lead to graft injury and activation of adaptive immune responses. In addition, the endothelium acts as a barrier between the transplanted organ and recipient, and modulates the trafficking of immune cells into the graft. Strategies to protect the endothelium from cold storage and reperfusion induced injury may reduce graft injury and acute rejection. Endothelial cells are anchored together by gap junctions (GJ) and tight junctions (TJ), the integrity of which is important for endothelial cell and barrier health. Breakdown of GJ and TJ is associated with endothelial death, injury and activation, and this breakdown occurs as consequence of cold storage, and reperfusion injury. Strategies to protect GJ and TJ integrity may protect the endothelium from injury early post transplantation and, further, may reduce IRI. Here, we explore the use of ACT1 peptide, which has been shown to stabilize and strengthen GJ and TJ in wound healing models (Ghatnekar et al., 2009). It was observed that stabilization of GJ and TJ with ACT1 peptide significantly inhibits post transplantation IRI.

In vitro studies can demonstrate that UW+ACT1 solution significantly reduced endothelial cell injury and inflammation post reperfusion as evidenced by improved TEER and reduced IL-8 secretion. ACT1 peptide treatment significantly protects endothelial cells from H₂O₂ to induce oxidative stress, and cold preservation, hypoxic reperfusion injury as measured by TEER, a marker of cell-cell interactions and cell injury. Further analysis of IL-8 secretion by endothelial cells exposed to cold preservation, hypoxia and reperfusion shows that ACT1 peptide treated cells are rendered less pro-inflammatory as compared to untreated cells. Further, the addition of ACT1 peptide to UW preservation solution significantly reduces ischemic reperfusion induced graft injury and inflammation in a cardiac heterotopic allograft model. Taken together, these novel findings propose a role for GJ and TJ in the pathogenesis of IRI and further demonstrate that stabilization of GJ and TJ with ACT1 peptide significantly inhibits post transplantation IRI.

HUVECs were exposed to either 18 h of cold storage in UW solution or UW/ACT1 followed by 48 h of reperfusion to model IRI in vitro, as previously described (Atkinson et al., 2013), or H₂O₂±ACT1 peptide to model oxidative stress. Efficacy was determined by TEER and IL-8 release.

Heterotopic abdominal heart transplants were performed between Balb/c and C57B1/6 mice, as previously described (Gao et al., 2014). Donor hearts were cold preserved in UW or UW/ACT1 (1000 μM) for 6 hrs at 4° C. Following storage, hearts were then implanted and harvested at 48 h post transplantation to access the impact of ACT1 peptide post-treatment on IRI. Post-transplant injury was assessed by analyses of serum Cardiac Troponin I and histological scoring of cardiac graft injury and inflammation; neutrophil and macrophage infiltration and pro-inflammatory cytokine.

### Example 17

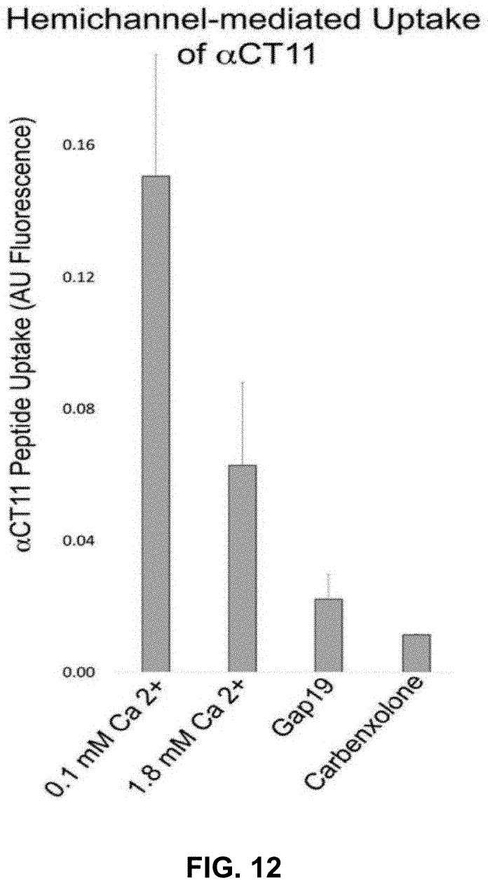

A further example of the several aspects described herein is its use in treatment of a patient suffering from an acute coronary syndrome. Parental Hela cells do not express gap junction-forming hemichannels, but can be made to do so by engineering them to heterologously express a connexin mutant (Cx43delCT) with a cytoplasmic truncation from amino acid (aa) 258 of the human Cx43 primary sequence. HeLa cells expressing Cx43 and/or Cx43delCT generate large numbers of exosomal EVs containing Cx43-formed hemichannels and these exosomes can be isolated and assayed using methods known to those skilled in the art (See attached “exosome data powerpoint.pptx”). EVs isolated from these HeLa cells can be placed in a solution containing a low Ca2+ concentration (e.g., <0.1 mM), causing the opening of their Cx43delCT hemichannels. The “hemichannel opening” solution also contains a concentration of a small therapeutic molecule that is able to pass through open hemichannels to the inside of EVs where its concentration equilibrates with the external solution. For example, the solution can contain a 50 μM concentration of aCT11 (RPRPDDLEI (SEQ ID NO: 13), —MW˜1100), a bioactive peptide that readily passes through hemichannels (FIG. 12) and provides cardioprotection following IR injury (Circulation. 2016; 134:A16380). The EV can then be transferred to a solution that causes hemichannels to close (e.g., by increasing [Ca2+]>1 mM), entrapping the concentrated cargo of therapeutic molecules. A composition comprising a Cx43delCT hemichannel-expressing EV containing a therapeutic concentration of aCT11 generated as described in steps 1 through 6 is one exemplary aspect.

The EVs in mg concentrations (as determined by a qualified medical professional from consideration of factors such as safe and efficacious dosage, patient body mass, patient health, co-morbidities, co-treatments and so on) can then be introduced by intravenous injection into the blood stream of the MI patient who has recently suffered an ischemic reperfusion (IR) injury to their heart. The EVs can also be provided continuously in an intravenous drip over periods of 30 minutes or more. The EV Cx43delCT-formed hemichannels are competent to dock with Cx43 hemichannels in the membrane of cells of the IR injured heart, forming gap junction channels that couple the EV with the cell. The conditions on the inside of the EV and the heart cell will be conducive to gap junction channel opening, enabling the transfer of the EV cargo, including the therapeutic small molecules to the cytoplasm of heart cells, where the molecules will mediate therapeutic effects. This can include effects attributable to aCT11 including protection of heart cells from the IR insult, reducing overall myocardial infarct size, preserving cardiac muscle and function, lessening the likelihood of progressive loss of heart function following MI, heart failure and death.

Conversely, heart tissue subject to IR injury can show altered pH or increased levels of ROS, affording conditions that trigger the opening of hemichannels formed by full length non-truncated Cx43. Conveniently, undocked hemichannels containing the Cx43delCT mutant remain closed in such conditions. This means that contents of the provided EVs do not prematurely release their therapeutic cargo of aCT11 molecules before docking with and transferring them to myocardial cells in the injured heart. The specific targeting of the provided invention to cells expressing Cx43 is a further aspect of the invention. Heart muscle cells express abundant Cx43. This membrane-associated Cx43 undergoes lateralized spreading from cardiomyocyte intercalated disks at cells ends following IR injury, redistributing to the sides of cardiomyocytes. Conveniently, the extracellular docking receptors of lateralized hemichannels are more accessible for a targeted interaction with EV Cx43delCT-formed hemichannels as a result of the lateralization/redistribution process. Thus, the provided EVs are selectively targeted to IR injured myocardial tissues. Repeated administration of the therapeutic EVs can be provided to the patient to enhance therapeutic benefit.

### Example 18

A further example of an aspect of the disclosure is its use in treatment of glioblastoma (GBM) in a subject. It has been reported that the Cx43 mimetic peptide JM2 represents a novel and potent therapeutic opportunity to target chemoresistant cancer stem cells (CSCs). This was described in the publication (http://cancerres.aacrjournals.org/content/77/13_Supplement/4765). CSCs are found in a variety of cancers, including glioblastoma, breast, lung liver, colon, pancreatic, ovarian and prostate cancers and are thought to provide “seeds” by which established and new tumors grow and metastasize. The therapeutic composition and use of the provided invention in this instance is as follows: Exosomal EVs (30-200 nm in diameter) can be isolated from Cx43delCT containing HeLa cells using methods known to those skilled in the art and as described in this specification. The EV can then be placed in a solution containing a low Ca2+ concentration (e.g., <0.1 mM), causing the opening of their Cx43delCT hemichannels. The “hemichannel opening” solution also contains a 100 μM concentration of a JM peptide (VFFKGVKDRVKGRSD (SEQ ID NO: 87))—a peptide therapeutic that has efficacy targeting cancer stem cells, including those found in GBM and colon cancer tumors (Lamouille et al. Targeting glioblastoma cancer stem cells with a novel Connexin43 mimetic peptide [abstract]. In: Proceedings of the American Association for Cancer Research Annual Meeting 2017; 2017 Apr. 1-5; Washington, D.C. Philadelphia (Pa.): AACR; Cancer Res 2017; 77 (13 Suppl):Abstract nr 4765. doi:10.1158/1538-7445.AM2017-4765). The EVs can then be transferred to a solution that causes hemichannels to close (e.g., by increasing [Ca2+]>1 mM), entrapping the concentrated cargo of JM molecules within the EVs. A composition comprising of Cx43delCT hemichannel-expressing EVs containing a therapeutic concentration of a JM peptide is one aspect.

The provided EVs in mg concentrations (as determined by a qualified medical professional from consideration of factors such as safe and efficacious dosage, delivery regimen, patient body mass, patient health, co-morbidities, co-treatments and so on) can be introduced by intravenous injection into the blood stream of a GBM patient. The EVs can also be provided by intravenous drip over periods of 30 minutes or more. The EVs can also be given continuously long-term using a small pump over days or for a week or more under a protracted venous infusion regime.

Conveniently, exosomal EVs can permeate the blood brain barrier, as well as being sufficiently small to diffuse through the narrow extracellular space of the brain parenchyma, enabling their access to the GBM tumor. Furthermore, GBM CSCs express high levels of Cx43, facilitating targeting of the EV Cx43delCT-formed hemichannels via docking with Cx43 hemichannels in the membrane of the CSCs. The conditions on the inside of the EV and the CSC will be conducive to gap junction channel opening, enabling the transfer of the EV cargo, including the movement of JM peptide into the cytoplasm of the CSCs, where the transferred molecules will mediate the desired therapeutic effects. These effects include the loss of CSCs and GBM cancer cells differentiating from those CSCs, reductions in tumor volume, decreases in tumor associated toxicity (e.g., glutamate release and epileptogenesis), decreases in GBM metastasis and other improvements in patient health, well-being, and quality of life and increased life span. Repeated administration of the therapeutic EVs can be given to the patient for therapeutic benefit. The GBM patient will be under the care of a doctor who will use the therapy described in 1 through 11 in combination with standard of care treatments that include surgical resection, radiation and temozolomide, as well as other approved therapies used for the treatment and/or amelioration of symptoms in GBM patients.

### Example 19

The compositions described herein can be used for treating or radiation dermatitis or other disease or condition in a subject undergoing radiotherapy. A carbopol gel (1% w/w), can be prepared by dispersing carbomer 940 NF resin (PCCA, Houston, Tex., USA) in distilled water (44 g), in which glycerol (5 g) has been previously added. The mixture can be stirred until thickening occurs and then neutralized by the drop wise addition of 50% (w/w) triethanolamine to achieve a transparent gel of pH 5.5. 100 mg of EVs containing aCT11 (RPRPDDLEI (SEQ ID NO: 13) at 100 μM) in 2 ml PBS are prepared and spun an ultrafiltration centrifuge tube (Thermo Fisher Scientific, Scoresby, Australia) at 2500 rpm for one hour to achieve a final volume of 760 μl. The EV solution can then be mixed into 2.25 mL 1% (w/w) with the carbopol gel by manual stirring for 5 minutes to ensure homogenous dispersion. The preparation of the EV formulation can be scaled accordingly using methods known to those skilled in the art. The EV containing gel can be used on female patients who are undergoing treatment breast cancer, including radiotherapy. Immediately following radiotherapy 2-5 mls of the gel can be applied to cover the area of the chest wall that has been exposed to radiation by a qualified physician. Thereafter the EV containing gel can be applied twice daily by the patient to this area of their chest wall from the first day of radiotherapy to two weeks after its completion. At the end of the first, second, third, fourth and fifth week of radiotherapy and two weeks following completion of treatment, all patients should be monitored by a radiation-oncologist to ensure that radiation dermatitis is reduced and that adverse reactions are not evident. Fields of skin on patients that have been exposed to radiation including from a “nuclear dirty bomb”, nuclear explosion or nuclear accident can also be treated using this regimen to reduce radiation injury and other manifestations of injury to the skin resulting from radiation exposure.

### Example 20

Targeting the CX43 Carboxyl Terminal H2 Domain Preserves Ventricular Function Following Ischemia-Reperfusion Injury.

Heart muscle cells are connected together by large numbers of gap junction (GJ) channels^{1, 2}. The main subunit protein of GJs in the mammalian ventricle muscle is Connexin 43 (Cx43 encoded by GJA1), which is preferentially localized in intercalated disks—zones of specialized electromechanical interaction between cardiomyocytes^{3, 4}. Following myocardial infarction in patients with ischemic heart disease, Cx43 remodels from its normal distribution in muscle tissue bordering the necrotic injury, redistributing from intercalated disks at cardiomyocyte ends to lateral domains of sarcolemma⁵. This process of Cx43 lateralized remodeling within the cell membrane is a hallmark of ischemic heart disease in humans and is thought to contribute to the arrhythmia-promoting characteristics of the infarct border zone.

Cx43 phospho-status has emerged as a factor of interest in pathogenic assignments of the protein in the wound healing response of cardiac muscle, and other tissues, including skin⁶. Pertinent to GJ remodeling in heart disease, ischemic conditioning results in retention of Cx43 at intercalated disks⁷. This occurs in association with increases in phosphorylation at serine 368 (S368)—a consensus Protein Kinase C (PKC) site in the cytoplasmic Carboxyl Terminal (CT) domain of Cx43. Cx43 S368 phosphorylation has also been linked to reduced activity of Cx43-formed channels,⁷⁻⁹, including undocked hemichannels¹⁰.

Previously, it was demonstrated that a peptide mimetic of the Cx43 Carboxyl Terminus (CT), incorporating its postsynaptic density-95/disks-large/ZO-1 (PDZ)-binding domain reduced Cx43 GJ remodeling in injury border zone tissues following cryo-infarction of the left ventricle in mice¹¹. The decreases in Cx43 remodeling prompted by treatment with this peptide (termed alpha CT1) were associated with a decreased propensity of the injured hearts to develop inducible arrhythmias¹¹, and sustained improvements in ventricular contractile performance over an 8-week study period¹². We further reported that the decreases in Cx43 lateralization observed in hearts treated with alpha CT1 were correlated with increased phosphorylation of S368¹¹, in line with results from other workers linking this post-translational modification to reduced GJ remodeling and cardioprotection⁷.

It was initially interpreted the induction of increased phosphorylation by alpha CT1 as a down-stream consequence of the well-characterized property of the peptide to disrupt interactions between Cx43 and its scaffolding protein ZO-1^{13, 14} However, in simple biochemical assays involving purified PKC enzyme, and a Cx43 CT substrate, we went on to show that alpha CT1 promoted S368 phosphorylation in vitro in a dose-dependent manner, without recourse to interaction with ZO-1¹¹. This result raised the prospect that alpha CT1 mode-of-action could have at least two independent aspects—one involving inhibition of interaction between Cx43 and ZO-1 and the other associated with PKC-mediated changes in Cx43 phospho-status.

The details of alpha CT1 molecular mechanism is of key translational significance as this therapeutic peptide is presently the subject of intensive testing in the clinic¹⁵. In Phase II clinical trials, alpha CT1 showed high level of efficacy in promoting the healing of two types of chronic, slow healing skin wounds¹⁶⁻¹⁸. Alpha CT1 is currently in pivotal Phase III testing on more than 500 patients, as a treatment for diabetic foot ulcers (GAIT1 trial)¹⁹. In this Example, details of the molecular mechanism of alpha CT1 are demonstrated, showing that the protective effects of alpha CT1 in ischemic injury to the ventricle is not related to ZO-1 interaction, but is likely associated with binding of the peptide to the Cx43 H2 alpha-helical region, a short stretch of the Cx43 CT adjacent to a serine-rich domain that includes S368.

Materials and Methods

Animals: Male C57BL/6 Mice 3-Month Old were Used.

Reagents: Peptides, cDNA Expression Constructs, and Antibodies

Sequences and a brief description of each Cx43-CT-based peptides used are shown in Table 1. Peptides were synthesized and quality checked for fidelity and purity using High Performance Liquid Chromatography and mass spectrometry (LifeTein, Hillsborough, N.J.). Biotinylated peptides were designed for surface plasmon resonance experiments. Glutathione-S-Transferase (GST) fusion protein constructs composed of the Cx43-CT (pGEX-6-P2 Cx43 CT amino acids 255-382), ZO-1 PDZ1, PDZ2 and PDZ3 were isolated and purified from isopropyl-b-D-thiogalactoside (IPTG)-induced BL21 bacteria using standard procedures, described in references^{13, 14, 20}. The pGEX6p2-Cx43 CT plasmid was obtained from Prof. Paul L. Sorgen (University of Nebraska Medical Center, USA). Cx43 CT mutant (Cx43 CT-KK/QQ; amino acids Lys345 Lys346 to Gln 345 Gln 346) was developed by site-directed mutagenesis of the pGEX6p2-Cx43 CT plasmid (Agilent technologies, QuikChange II Site-Directed Mutagenesis Kit). The mutation was verified by sequencing. For surface plasmon resonance experiments, the GST was removed using PreScission protease, yielding Cx43 CT protein (wild-type or mutant).

TABLE 1

| | | | Interaction | |
|---|---|---|---|---|
| | | | with: | |
| | Antennapedia | | Cx43 CT | |
| Peptide | Cx43CT | Modification | ZO-1 | PDZ2 |
| alpha | RQPKIWFPNRRK | Unmodified | +++ | +++ |
| CT1 | PWKKRPRPDD | alpha | | |
| | LEI | CT1 | | |
| | (SEQ ID | | | |
| | NO: 111) | | | |
| M1 | RQPKIWFPNRRK | CT DD & E | − | − |
| AALAI | PWKKRPRPAA | substituted | | |
| | LAI | with As | | |
| | (SEQ ID | | | |
| | NO: 118) | | | |
| M2 | RQPKIWFPNRRK | CT DD | −/+ | ++ |
| AALEI | PWKKRPRPAA | substituted | | |
| | LEI | with As | | |
| | (SEQ ID | | | |
| | NO: 119) | | | |
| M3 | RQPKIWFPNRRK | CT E | + | +++ |
| DDLAI | PWKKRPRPDD | substituted | | |
| | LAI | with A | | |
| | (SEQ ID | | | |
| | NO: 113) | | | |
| M4 | RQPKIWFPNRRK | Scrambled | − | − |
| scram. | PWKKLPAARI | control | | |
| | APR | | | |
| | (SEQ ID | | | |
| | NO: 120) | | | |
| alpha | RQPKIWFPNRRK | CT | +++ | − |
| CT1-I | PWKKRPRPD | isoleucine | | |
| | DLE | deleted | | |
| | (SEQ ID | | | |
| | NO: 112) | | | |
| alpha | RPRPDDLEI | No NT | +++ | +++ |
| CT11 | (SEQ ID | antennapedia | | |
| | NO: 13) | seq. | | |

Antibodies: Phospho-Connexin43 (Ser368) (Cell Signaling, 3511S, Danvers, Mass.), anti-Cx43 produced in rabbit (Sigma: C6219, St. Louis, Mo.), anti-GST produced in goat (GE, 27457701, Little Chalfont, UK). NeutrAvidin-HRP (Thermo, 31030, MA).

Western Blotting

Protein samples from all related experiments (PKC and EDC cross-linking assays and Westerns on heart lysates) were processed in lithium dodecyl sulfate sample loading buffer (Bio-Rad, 1610737 CA), heated at 95° C. for 5 minutes. Samples from PKC and cross-linking assays were loaded on 18% Tris-Glycine Stain-Free gels (Bio-Rad, 5678073 CA); samples from heart lysates were loaded on 10% Tris-Glycine Stain-Free el (Bio-Rad: 5678033 CA), resolved by SDS-PAGE, transferred to PVDF FL membrane on a Turbo Transfer System (Bio-Rad, 1704155 CA). alpha CT1 eluted from cross-linking reactions was detected on blots against biotin with HRP-NeutrAvidin (ThermoFisher, 31001, MA). Signals were detected by HR-based chemiluminescence (ThermoFisher, 34095, MA) and exposed to ECL Chemidoc (Bio-Rad, 1708280 CA) and digitized using Image Lab software (Bio-Rad, 1709692 CA). Detailed methods have also been previously described^{11, 13, 14.}

Surface Plasmon Resonance

Efficacy of the interaction of each alpha CT1 variant with Cx43 CT or Cx43 CT-KK/QQ was tested using surface plasmon resonance (SPR) as described previously 20. In brief, SPR experiments were performed using a Biacore T200 (GE Healthcare). Equal amounts (response units/RU) of biotin-alpha CT1 variants were immobilized on each flow cell of a streptavidin-coated sensor chip (Biacore Inc) using immobilization buffer (in mM: 10 HEPES, 1 EDTA, 100 NaCl, 0.005% Tween-20) at pH 7.4. Measurements with wild-type (wt) Cx43 CT and mutant Cx43 CT-KK/QQ analytes were done in running buffer (in mM: 10 HEPES, 100 NaCl, pH 7.4) at a flow rate of 30 μl/min. Binding of analytes were verified at different concentrations, in random order (injection volume 120 μl). Interacting proteins were then unbound by injection of 10 v regeneration buffer (50 mM NaOH and 1 M NaCl) at a flow rate of 10 μl/min. Background levels were obtained from a reference cell containing a biotin-control peptide in which the reversed sequence of the last 9 amino acids of Cx43 was fused to biotin-antennapedia. The RU values obtained with biotin-control peptide were subtracted from the RU values obtained with the different biotin-alphaCT1 variants (wild-type or mutant) to generate the different response curves.

PKC-ε Cx43 CT S368 phosphorylation Assay

PKC assay conditions were used to evaluate the PKC-ε phosphorylation of Cx43-CT substrate at Ser368 as we have described previously, with modifications¹¹. 400 ng/ml PKC-v (Life, 37717L, Carlsbad, Calif.) was pre-diluted in enzyme dilution buffer (10 mM HEPES pH7.4, 0.01% CHAPS and 5 mM DTT) and assayed in 20 mM HEPES pH7.4, 10 mM/L MgCl2, 0.1 mM EGTA, 1× lipid mix (200 μg/ml phosphatidylserine (Avanti Polar Lipids 840032C),²⁰ μg/ml Diacylglycerol (Avanti Polar Lipids), 1 mM HEPES pH7.4, 0.03% CHAPS), 500 μM ATP (Sigma, A6419) and 14 μg/ml Cx43-CT substrate. Kinase assay buffer was supplemented with peptides to produce final concentrations of the reaction constituents, as indicated in figure legends. The mixture was incubated at 37° C. for 12 minutes and quenched by addition of LDS sample loading buffer (Bio Rad, 1610791). “XT sample buffer” is what was shown on the product label, whereas the component is similar as regular LDS buffer, containing about 5-10% lithium dodecyl sulphate. The reaction was Western blotted for pS368 Cx43 using the Phospho-Connexin43 (Ser368) antibody from Cell Signaling. Proteins were eluted off by stripping buffer (Millipore 2504) and re-probed for total Cx43 using the Sigma anti-rabbit antibody. Percent phosphorylation (% P) was quantified using Equation 1 and normalized with control group (PKC+, no peptide added).

$$\% P = ( \frac{p 368 c x 43}{total Cx 43} ) \times 100 \qquad ( Eq . 1 )$$

EDC Cross-Linking Assay

To characterize the interaction between the Cx43 CT substrate and peptides, the in vitro kinase assay was performed as above with modification and the constituents then subjected to a cross-linking reaction. The assay buffer used was 20 mM 3-(N-morpholino) propanesulfonic acid (MOPS), pH 7.2. The Cx43 CT substrate concentration was 30 μg/ml and peptide concentrations varied as indicated in figure legends. All other reagents present in the kinase reaction were maintained as described above. The reaction was allowed to proceed at 37° C. for 15 minutes. Afterwards, the carbodiimide crosslinker 1-Ethyl-3-(3-dimethylaminopropyl)carbodiimide HCl (EDC) (Thermo, 22980) was added to each solution for a final concentration of 20 mM. The solution was allowed to cross-link for one hour at room temperature. The reaction was stopped by the addition of 4×LDS loading buffer, boiled for 5 minutes and subsequently separated by PAGE. The resulting gel was stained in Coomassie brilliant blue (Sigma, B0770) for two hours and destained in a solution of 4% methanol 7% acetic acid overnight. Gel bands were subsequently excised for mass spectrometric analysis. For the direct interaction between protein and peptides, PBS, pH 7.5 was used as the coupling buffer. The protein (50 μg/ml) and peptide (25 μM) were allowed to react at room temperature for one hour before EDAC was added to the reaction mixture. The reaction was Western blotted for Cx43, GST or NeutroAvidin.

Tandem Mass Spectrometry

Gel bands corresponding to crosslinked Cx43 and alpha CT1 were excised and cut into 1 mm square pieces, destained with three consecutive washes with a 50:50 mixture of 50 mM ammonium bicarbonate and acetonitrile for 10 mins. 50 μL of 10 mM DTT was then added to the gel pieces and the gel pieces were incubated at 56° C. for one hour. 50 μL of 55 mM iodoacetamide was then added to the sample to alkylate cysteines. The sample was incubated at 25° C. in the dark for 45 mins. The gel was then dehydrated with three consecutive washes with a 50:50 mixture of 50 mM ammonium bicarbonate and acetonitrile for 10 min and completely dehydrated with 100% acetonitrile and dried in a speedvac. Gel pieces were rehydrated in 10-15 μL of solution containing 20 ng/μL trypsin (Promega, Madison, Wis.) in 50 mM ammonium bicarbonate for 15 min. 30 μL of 50 mM ammonium bicarbonate buffer was added to each sample and the samples were incubated at 37° C. for 18 hours. Peptides were extracted using 20% ACN/0.1% TFA once, 60% ACN/0.1% TFA twice, and 80% ACN/0.1% TFA once. The extracted samples were pooled and dried in a speedvac and reconstituted in 0.1% formic acid for subsequent LC-MS/MS analysis.

For LC-MS/MS analysis, tryptic peptides were directly separated on a one-dimensional fused silica capillary column (150 mm×100 μm) packed with Phenomenex Jupiter resin (3 μm mean particle size, 300 Å pore size). One-dimensional liquid chromatography was performed using the following gradient at a flow rate of 0.5 μL/min: 0-10 min: 2% ACN (0.1% formic acid), 10-50 min: 2-35% ACN (0.1% formic acid), 50-60 min: 35-90% ACN (0.1% formic acid) balanced with 0.1% formic acid. The eluate was directly infused into an LTQ Velos mass spectrometer (ThermoFisher, San Jose, Calif.) equipped with a nanoelectrospray source. The instruments were operated in a data dependent mode with the top five most abundant ions in each MS scan selected for fragmentation in the LTQ. Dynamic exclusion (exclude after 2 spectra, release after 30 sec, and exclusion list size of 150) was enabled²¹.

Molecular Modeling

Structural information for the Cx43 CT domain truncated at G251 was obtained from the Worldwide Protein Data Bank (DOI:10.2210/pdb1r5s/pdb). The protonated structure of the alpha CT1 peptide was obtained by truncating the 9 carboxyl terminal amino acids of the Cx43 CT. In order to model the interaction of the Cx43 CT with alpha CT1, the publically available protein-protein docking sever, Zdock (http://zdock.umassmed.edu/help.html) and SWISS-Model were used to model docking of alpha CT1 with the Cx43 CT in silico in low-energy conformations. Zdock is a Fast Fourier Transform-based protein docking program. Both alpha CT1 and the Cx43 CT were submitted to the ZDOCK server for possible binding modes in the translational and rotational space. Each pose was evaluated using an energy-based scoring function²².

Protein Thermal Shift (PTS) Assay

Thermal stability of recombinant GST-PDZ2 or Cx43 CT in the presence or absence of peptides was determined in a 96-well format. Each assay well was composed of 500 μg/mL protein, 25-100 μM of each peptide in PBS buffer, pH7.4. All assays were performed independently six times. Samples were generally prepared in 96-well plates at final volumes of 20 μL. The fluorescent dye SYPRO Orange (5000× concentrate in DMSO, ThermoFisher, S6650) was added to a final concentration of 8X. Reactions were run on QuantStudio 6 Flex Real-Time PCR system (Applied Biosystems, part of Life Technologies Corporation, CA) according to the manufacturer's recommendations using a melt protocol in 0.05-degree/sec increments from 25° C. to 95° C. The Reporter Dye was “ROX” and quencher Dye and passive reference were selected as “None” for the melt curve according to manufacturer's instructions. The data were analyzed using Protein Thermal Shift™ Software v1.3 package (Applied Biosystems, CA).

Ischemia-Reperfusion (I/R) Injury Model and LV Contractility

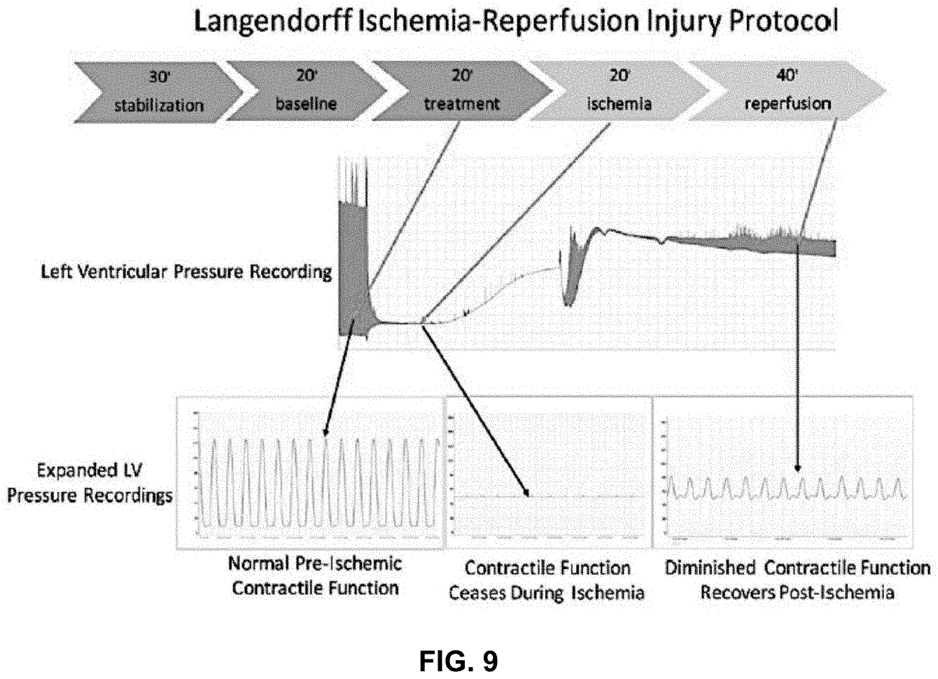

Male, 3-month-old, body weight 25±5 g C57BL/6 mice were used for this study and obtained from Charles River. Animals were randomly assigned to experimental groups and Left ventricular (LV) function was measured and myocardial ischemia-reperfusion injury (I/R) was induced as previously described²³. Briefly, 15 minutes after the injection of heparin at a dose of 200 U/10 g body weight, the mouse was anesthetized by inhalation of isoflurane vapor and subjected to cervical dislocation upon the cessation of respiration. Thoracotomy was immediately performed and the heart excised. The heart was arrested in ice-cold Krebs-Henseleit (KH) buffer (in mM: 25 NaHCO₃, 0.5 EDTA, 5.3 KCl, 1.2 MgSO4, 0.5 pyruvate, 118 NaCl, 10 glucose, 2.5 CaCl₂. The aorta was isolated and cannulated in a Langendorff perfusion system. The heart was then perfused at a constant pressure of 75 mmHg with KH buffer, which was continually bubbled with 5% CO₂/95% O₂ at 37° C. Effluent from the Thebesian veins was drained by a thin polyethylene tube (PE-10) pierced through the apex of the LV. A water-filled balloon made of polyvinylchloride film was inserted in the LV and connected to a blood pressure transducer (Harvard Apparatus, 733866, MA). After a 30-minute stabilization period, a balloon volume (BV) generating an LV end-diastolic-pressure (EDP) of 0 mmHg, was determined for the heart. The BV was then increased stepwise up through 1, 2, 5, 8, 10, 12, 15, 18, 20, 25, 30 μl increments of 1-5 μl and contractile performance were recorded for 10 seconds at each step. The indexes of cardiac function were amplified by a Transducer Amplifier Module (Harvard Apparatus, 730065, MA). Data was recorded and analyzed using PowerLab 4/35 (ADInstruments, PL3504, CO) and LabChart V7 (ADInstruments, CO). The BV was then adjusted to set EDP at about 8-10 mmHg and held constant during the ensuing steps of the protocol. Baseline function (determined by EDP at about 8-10 mmHg) was recorded for 5 minutes. The perfused beating heart were then treated with freshly prepared peptide stocks (0.2 mM), which were infused using syringe pump (Kent Sci, CT) into the perfusion buffer in a mixing chamber above the heart at 5% of coronary flow rate, to deliver final concentrations of 10-50 μM or equivalent vehicle for 20 minutes. At the end of the peptide infusion period hearts were subjected to global, no-flow normothermic ischemia by turning off the perfusion flow for 20 min, followed by a reperfusion phase for 40 min. BV was retaken through the stepwise sequence of 1-5 μl increments between 1 and 30 μl, with contractile performance again being recorded for 10 seconds at each step. Cardiac LV function was recorded throughout the procedure. In the case of post-ischemic treatment with alpha CT11 peptide, peptide infusion was begun at the initiation of the reperfusion phase, continued for 20 minutes and then contractile function by BV increments was taken as per the other hearts. A set of hearts were freeze-clamped immediately after peptide infusion for Western blotting. The protocol is illustrated in FIG. 9.

Laser Scanning Confocal Microscopy and Fluorescence Quantification of Peptide Perfused Hearts.

LV samples were Langendorff perfused with vehicle control, alpha CT1 and alpha CT11 solutions as described above and as summarized in FIG. 9. Immunofluorescent labeling and detection and quantification of biotinylated peptide were performed as previously described 11, 14, 24 on 10 μm cryosections of tissue. Samples were co-labeled with a rabbit antibody against either connexin43 (Sigma, C6219, 1:250), Dapi and streptavidin conjugated to AlexaFluor 647 (1:4000; ThermoFisher Scientific). Cx43 primary antibodies were detected by goat anti-rabbit AlexaFluor 488 (1:4000; ThermoFisher) secondary antibodies. Confocal imaging was performed using a TCS SP8 confocal microscope. Quantification of fluorescence intensity levels relative to background were performed using NIH ImageJ software.

Statistical Analysis.

Data were expressed as a mean±SE unless otherwise noted. Differences among treatments were compared by one-way, two-way or repeated measures ANOVA, followed by post hoc or Mann-Whitney tests, as appropriate. Probability values p<0.05 were considered significantly different. No strong evidence of divergence (p>0.05) from normality was found. Data analysis was performed using GraphPad7 (GraphPad Software, LaJolla, Calif.).

Results

Alpha CT1 Interacts with the Cx43 Carboxyl Terminus H2 Domain

The 25mer Cx43 mimetic peptide alpha CT1 incorporates a 16-amino acid N-terminal (NT) antennapedia (Antp) sequence followed by the carboxyl terminal (CT)-most 9 amino acids of Cx43: Arg-Pro-Arg-Pro-Asp-Asp-Leu-Glu-Iso or RPRPDDLEI (SEQ ID NO: 1) (FIG. 1A and Table 1). The last four amino acids of this sequence (DLEI) comprise a class II PDZ-binding motif, which has been shown to mediate a specific interaction with the second of the three PDZ (PDZ2) domains of ZO-1 14,^{25, 26}. It has been previously reported on binding of alpha CT1 with ZO-1, and the selectivity of this interaction for the ZO-1 PDZ2 domain over that of ZO-1 PDZ1 and PDZ3¹⁴. This selectivity of alpha CT1 for ZO-1 PDZ2 is illustrated in FIG. 1B. Consistent with reports by others²⁷, deletion of the CT isoleucine of the DLEI binding motif (e.g., as in the alpha CT1-I peptide, Table 1) abrogates interaction with ZO-1 PDZ2 (FIG. 1B). It has been previously shown that alpha CT1 upregulates a PKCε-mediated phosphorylation of Cx43 at serine 368 (S368) along its primary sequence¹¹. This induction of S368 phosphorylation (pS368) by alpha CT1 was observed both in vivo in a left ventricular (LV) injury model and in a biochemical assay of PKCε activity in vitro¹¹.

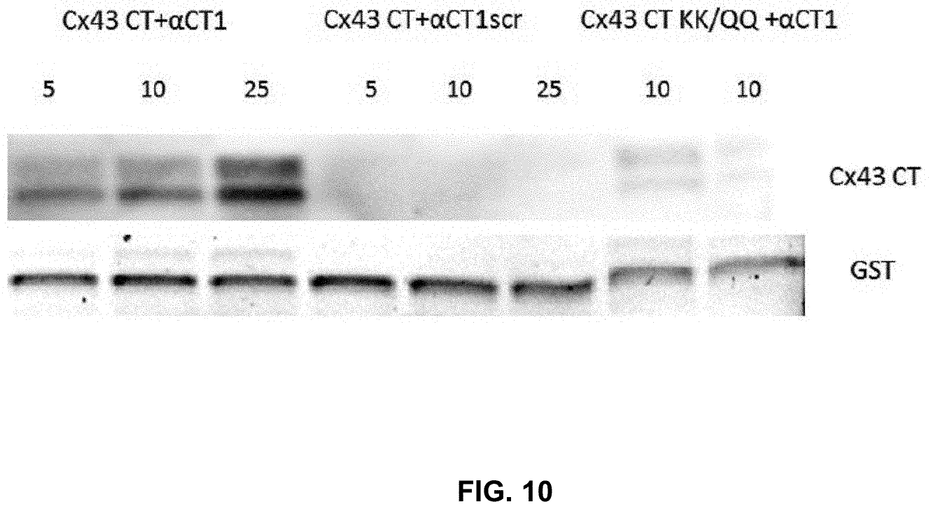

To identify the molecular determinants of alpha CT1-induced upregulation of S368 phosphorylation, the zero-length cross-linker 1-ethyl-3-(-3-dimethylaminopropyl) carbodiimide hydrochloride (EDC) was introduced into the in vitro PKCε phosphorylation assay. Zero-length cross-linking covalently bonds directly interacting proteins, enabling identification of partnering proteins in the reaction mixture. The components of the reaction mixture were then separated by SDS-PAGE and tandem mass spectrometry (MS/MS) was performed on the isolated polypeptides (FIGS. 1C and 1D). While no evidence for interaction between PKCε and alpha CT1 was observed, the analysis revealed that a band running just above GST-Cx43 CT corresponded to a covalently linked complex between the Cx43 CT substrate and alpha CT1 (FIG. 1C). Moreover, it was determined that a negatively charged glutamic acid (E381) within the PDZ-binding domain of alpha CT1, and a pair of aspartic acids (D378, D379), were involved in bonding with a pair positively charged lysines (K) at positions K345 and K346 of the Cx43 CT (FIGS. 1C and 1D). The site specificity of this interaction was further confirmed by streptavidin labeling of cross-linked products from kinase reaction mixtures containing Cx43 CT with alpha CT1 (FIG. 10 right hand blots) or a scrambled peptide (M4) unable to bind Cx43 CT (FIG. 10, right hand blots), as well as in reaction mixtures containing a mutated Cx43 CT (GST-Cx43 CT QQ/KK) substrate (FIG. 10, middle blots), in which the pair of positively charged lysine residues at K345 and K346 were substituted with neutral glutamines (Q). While no evidence of cross-linking between the scrambled peptide and Cx43 CT, or between alpha CT1 and the Cx43 CT QQ/KK mutant substrate was found, alpha CT1 was covalently linked by EDC to Cx43 CT in a concentration-dependent manner.

Alpha CT1 Interacts with the Cx43 CT H2 Domain

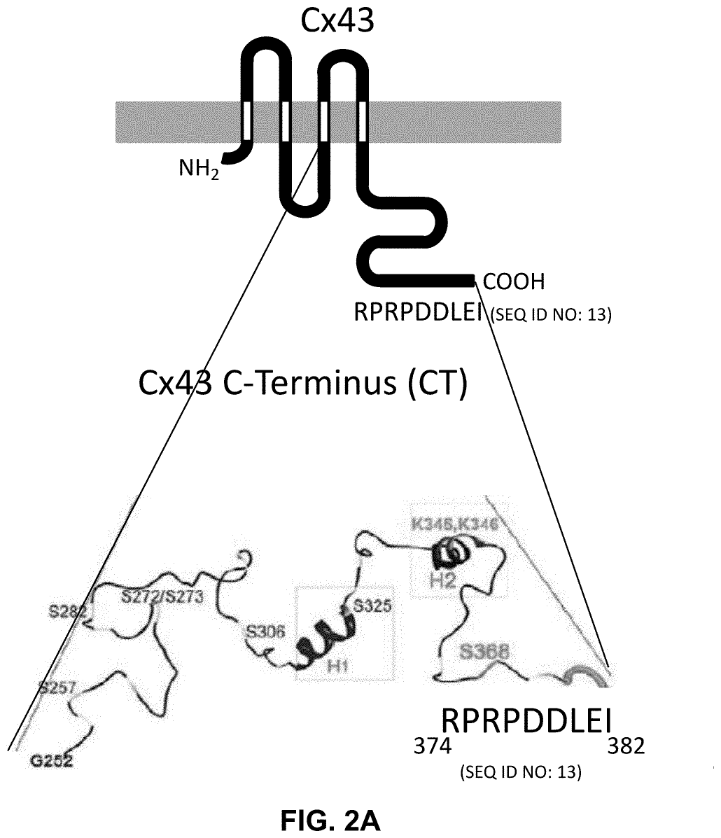

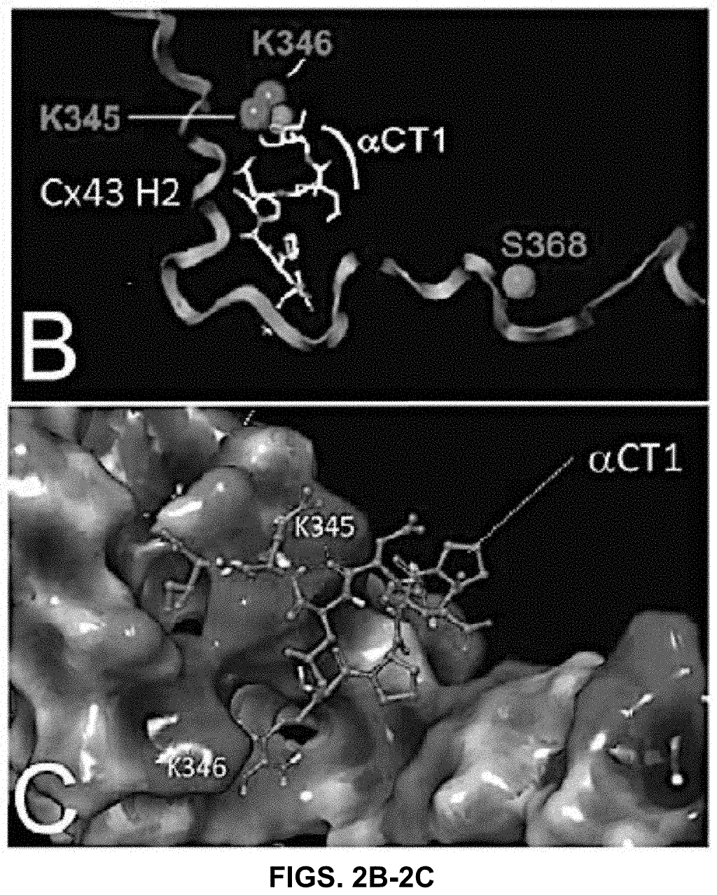

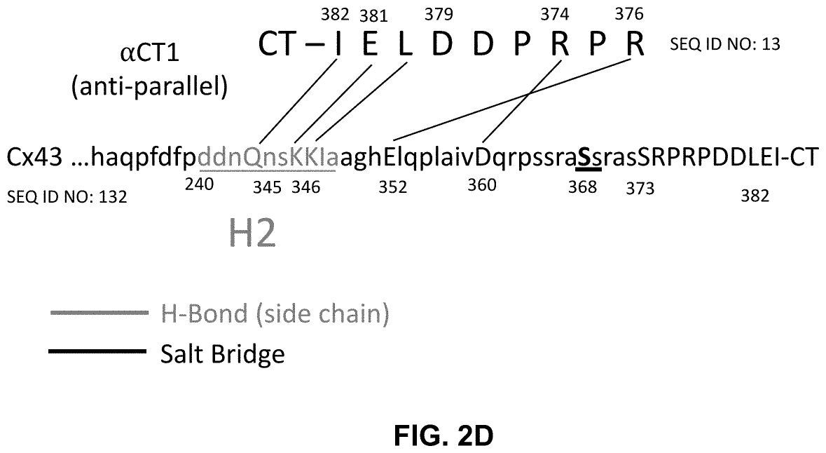

Structural studies by Sorgen and co-workers have shown that K345 and K346 fall within a short alpha-helical sequence along the Cx43 CT called H2 (for Helix 2)^{28, 29.} FIG. 2A provides a schematic of the secondary structure of the Cx43 CT showing the location of H2 (from³⁰), together with a second nearby stretch of the alpha-helical sequence (H1). To model alpha CT1:Cx43 CT H2 binding in silico, we submitted the interacting complex to the zDOCK protein modeling server²², initially fixing the interaction between the glutamic acid (E) at position −1 of alpha CT1 (i.e., E381 in full length Cx43) and the K346 residue of Cx43—as predicted by the MS/MS data (i.e., FIG. 1C). The interaction pose shown in FIG. 2B represents that based on the lowest energy minimization score from over 1800 possible variants of the complex. Using Schrodinger molecular modeling software, and without specifying the initial H2 K346 constraint, we confirmed that alpha CT1 could be optimally configured in an anti-parallel orientation with its available side-chains arrayed along the H2 sequence (FIGS. 2C-2D). As indicated by MS/MS, a salt bridge was predicted by this in silico analysis to form between the alpha CT1 glutamic acid (E) residue and Cx43 K346. The modeled interaction further anticipates hydrogen bonding between the side chains of four amino acids arrayed along alpha CT1 specifically at RPRPDDLEI (SEQ ID NO: 13) of aCT1 (SEQ ID NO: 111)—amino acids involved in hydrogen-bonds are bolded) and four amino acids between Q340 and E360 of the H2 sequence (FIG. 2D).

Substitution of Negatively Charged Amino Acids in Alpha CT1 Results in Loss of Cx43 CT Binding

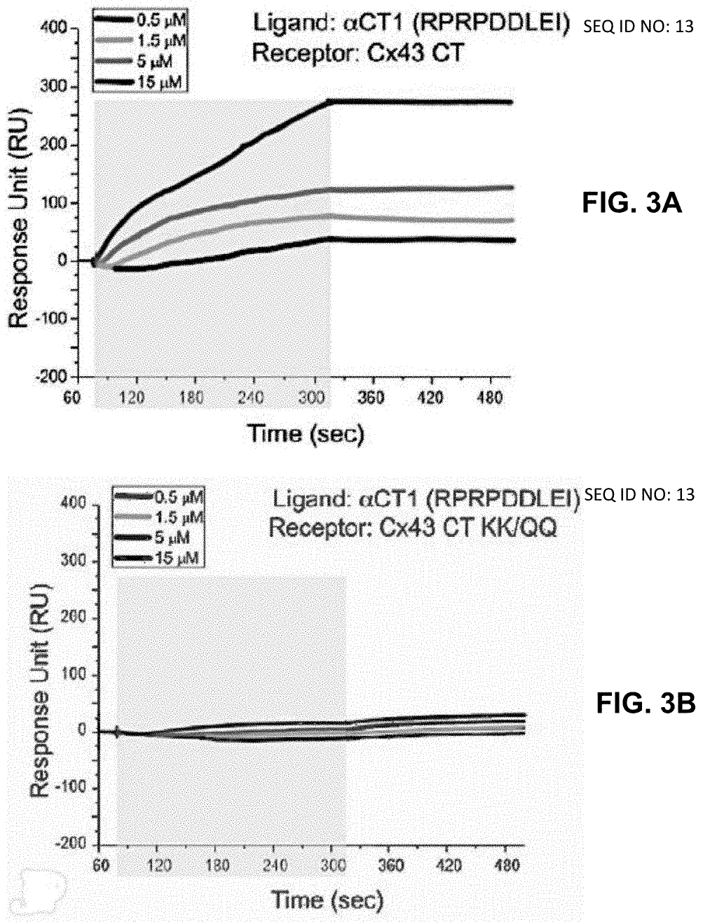

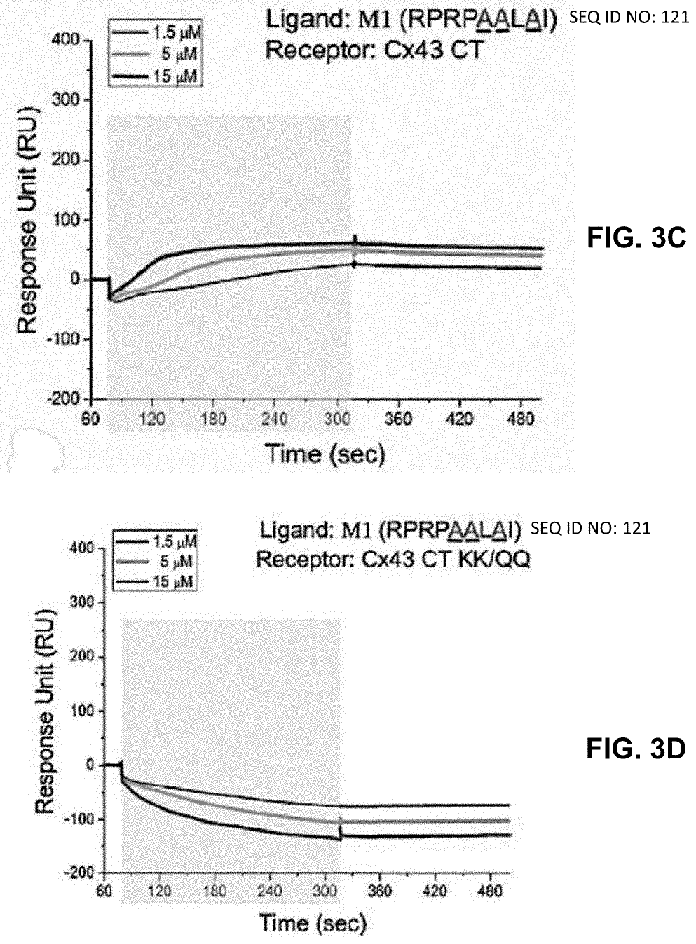

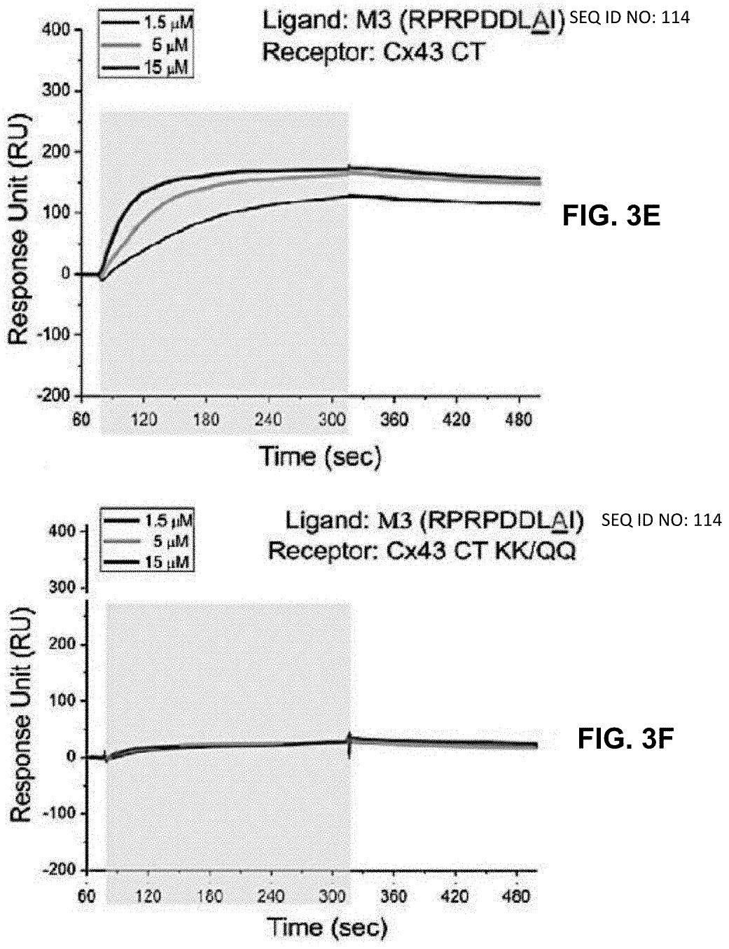

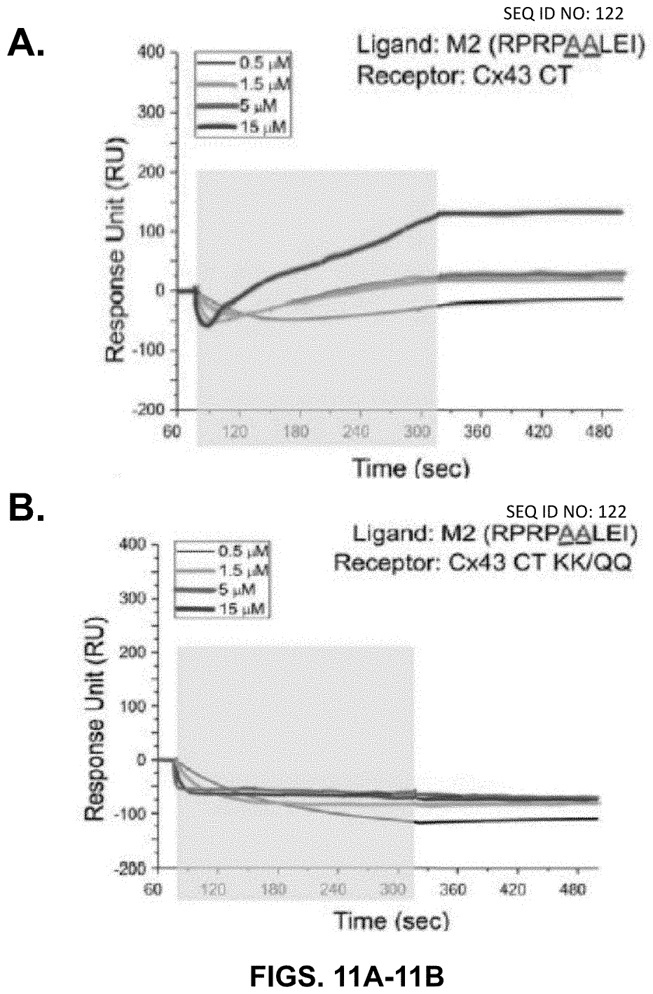

To further probe the alpha CT1 complex with Cx43 CT H2 region, and its consequence for phosphorylation of S368, three variant peptides based on alpha CT1 were prepared. In these peptides, negatively charged E and D amino acids in the RPRPDDLEI (SEQ ID NO: 13) sequence of alphaCT1 (i.e., those indicated by MS/MS to be likely involved in Cx43 CT interaction) were substituted by neutral alanines. These alpha CT1 variant peptides had the sequences RPRPAALAI (SEQ ID NO: 121), RPRPAALEI (SEQ ID NO: 122), and RPRPDDLAI (SEQ ID NO: 114) and are referred to as M1 AALAI, M2 AALEI and M3 DDLAI respectively. First, surface plasmon resonance (SPR) was used to analyze interactions of biotinylated versions of alpha CT1 and the alpha CT1 variants peptides, immobilized to streptavidin-coated sensor chips, with the Cx43 CT and Cx43 CT-KK/QQ proteins as analytes (FIGS. 3A-3F and FIGS. 11A-11B). The concentration of the analyte was varied between 0.5 and 15 μM. A concentration-dependent increase in Response Units was observed for Cx43 CT binding to biotin-alpha CT1 (FIG. 3A). M1 AALAI showed loss of Cx43 CT binding competence, consistent with having all negatively charged amino acids substituted with alanine (FIG. 3C). Substitution of D378/D379 (M2 AALEI) or E381 (M3 DDLAI) residues by alanines also abrogated peptide interaction with Cx43 CT (FIGS. 3E-3F and FIGS. 11A-11B). In complementary observations, SPR confirmed that the Cx43 CT KK/QQ mutant polypeptide was unable mediate interactions with alpha CT1, M1 AALAI, M2 AALEI or M3 DDLAI (FIGS. 3B, 3D, and 3F), consistent with the pair of lysines at K345 and K346 in H2 being necessary for interaction between Cx43 and alpha CT1.

Substitution of Negatively Charged Amino Acids in Alpha CT1 Fully and Partially Abrogate Interaction with Cx43 CT and ZO-1 PDZ2 Respectively.

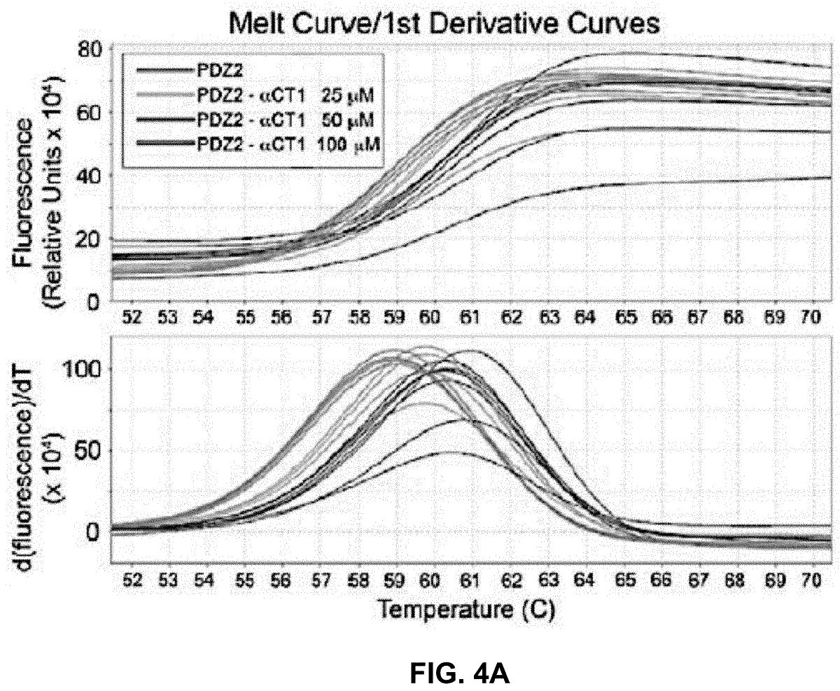

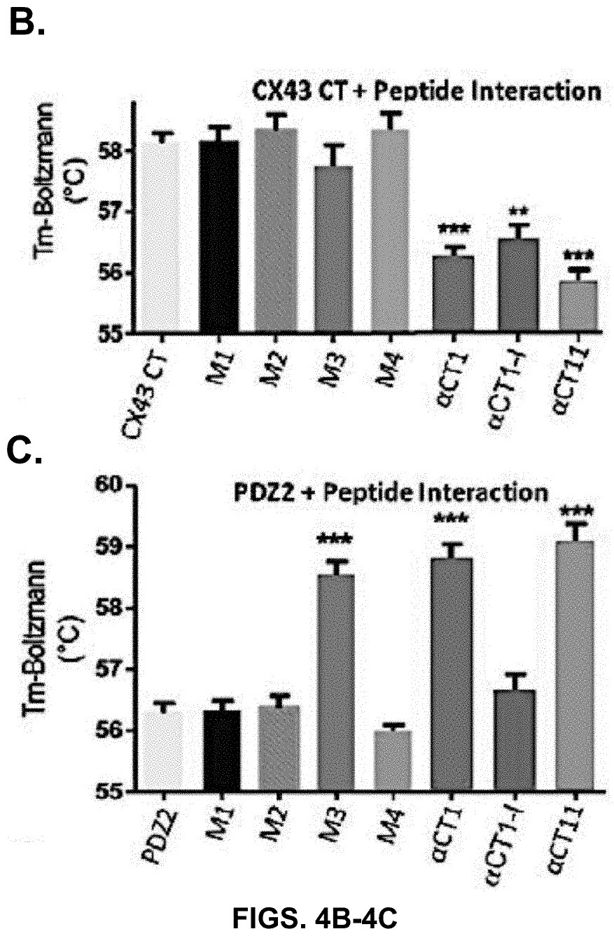

To further characterize the Cx43-binding characteristics of alpha CT1 and the alpha CT1 variants, thermal shift assays of peptide:protein interactions were performed (FIGS. 4A-4C). This assay provides quantitative data on the effect of interaction on protein secondary structure—with significantly increased or decreased thermal stability (as opposed to no change) being diagnostic of potential interaction. For example, in line with the known stabilizing effect of the last 10 amino acids of Cx43 CT on ZO-1 PDZ2 31, alpha CT1 concentrations of 25, 50 and 100 μM increased the melt temperature of PDZ2 in a dose-dependent manner (FIG. 4A). Thermal shift assays indicated that the peptides from Table 1 fell into two classes with respect to Cx43 CT interaction—those that provided evidence of interaction with Cx43 CT and those that were Cx43 CT interaction incompetent (FIG. 4B). Consistent with the SPR results, M1 AALAI, M2 AALEI and M3 DDLAI showed no propensity to alter Cx43 CT thermal stability, demonstrating no significant variance from Cx43 CT alone or Cx43 CT in the presence of the scrambled control peptide M4. By contrast, alpha CT1, alpha CT1-I and short variant of alpha CT1 comprising the Cx43 CT 9mer sequence RPRPDDLEI (SEQ ID NO: 13) (alpha CT11). All caused highly significant decreases in melt temperature, in line with interaction of these peptides disrupting secondary structure via binding to the Cx43 CT.

The effects of the alpha CT1 variants on thermal stability of ZO-1 PD2 were examined. Unlike in presence of the parent peptide alpha CT1 (FIGS. 4A-4C), M1 AALAI and M2 AALEI did not alter the melt temperature of PDZ2 (FIG. 4C), not differing significantly from PDZ2 alone, or PDZ2 in the presence of either scrambled peptide (M4) or alpha CT1-I—the two PDZ2 interaction incompetent peptides (FIGS. 1A-1D). The results were consistent with M1 AALAI or M2 AALEI having no, or limited, propensity to interact with the ZO-1 domain. However, M3 DDLAI, the most conservative substitution variant, showed evidence of significant interaction with its ZO-1-binding domain, with its effects on the thermal stability of PDZ2 being similar in this assay to those of alpha CT1 (FIG. 4C). Thus, although M3 DDLAI had no or limited competence to interact with Cx43 CT, this peptide did show evidence of ZO-1 PDZ2-binding activity not significantly different from unmodified alpha CT1.

Substitution of Negatively Charged Amino Acids in Alpha CT1 Abrogates Induction of S368 Phosphorylation.

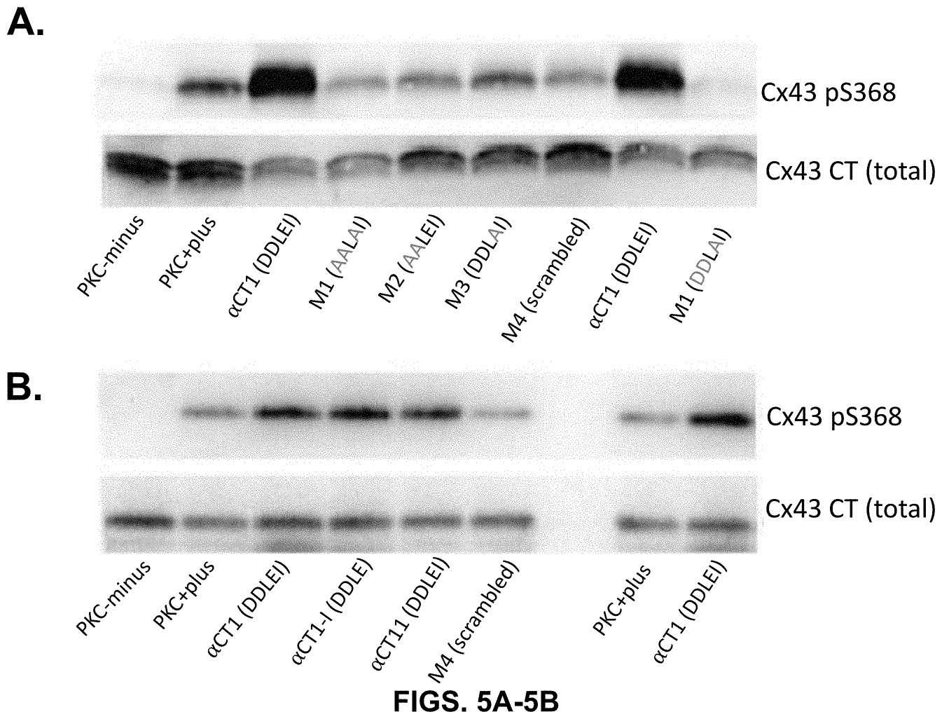

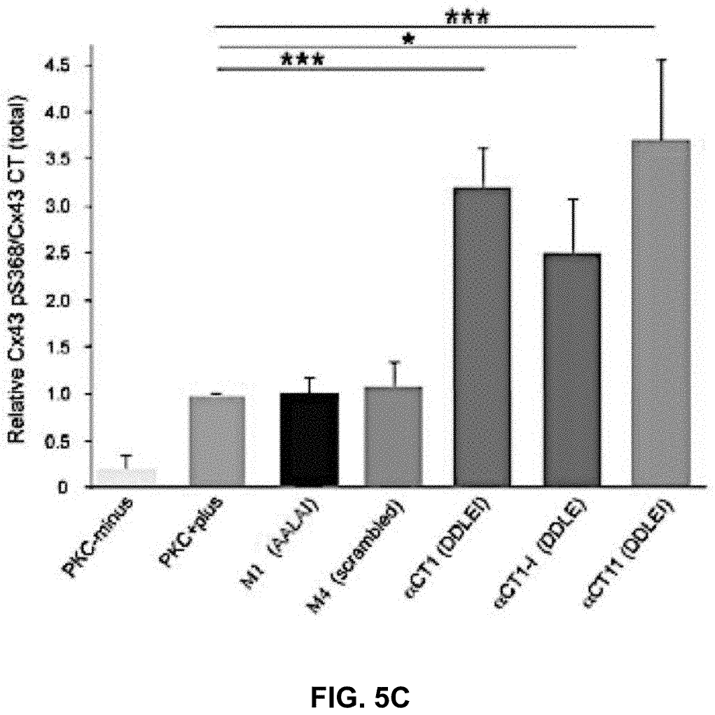

Next, it was examined how the mutant peptides performed in the PKC-ε kinase assay. Unlike alpha CT1, neither M1 AALAI, M2 AALEI nor M3 DDLAI increased Cx43 S368 phosphorylation above levels detected in the absence of peptide (PKC ε+plus lanes of FIG. 5A), or in the presence of scrambled control peptide (M4, FIGS. 5A-5B). Quantification of blots indicated that the ability of unmodified alpha CT1 to induce S368 phosphorylation was about 3-fold greater than that of either the PKC ε+plus control reaction (p<0.001) or reactions including M1 AALAI or M4 peptides (FIG. 5C). It was further determined that a 9 amino acid peptide comprising only RPRPDDLEI (SEQ ID NO: 13) (i.e., alpha CT11, which is alpha CT1 with its 16 amino acid NT antennapedia sequence truncated) robustly upregulated pS368 levels over control (vs PKC ε+plus control <0.001) (FIG. 5C). alpha CT1-I, the ZO-1-binding-deficient peptide with CT isoleucine truncated, also prompted a significant increase in PKC ε-mediated phosphorylation of Cx43 CT (p<0.05 vs. PKC ε+plus control). In sum, the results indicated that only those alpha CT1-based peptides competent to interact Cx43 CT (i.e., alpha CT1, alpha CT11 and alpha CT1-I), but not those unable to (i.e., M1 AALAI, M2 AALEI, M3 DDLAI and M4), increased pS368 above control levels. Also, given that M3 DDLAI is unable to induce pS368 increase, but does retain PDZ2 interaction ability, the data suggested that ZO-1-binding activity is dispensable for this phosphorylation.

Only Peptides Interacting with Cx43 CT Protect Hearts from Ischemic Injury

The biochemical characterizations indicated that alpha CT1 is capable of two distinct protein-protein interactions—one with ZO-1 PDZ2 and the other with the Cx43 H2 region. This raised the question as to whether or not the previously characterized effects of alpha CT1 in cardiac injury models^{11, 12} or indeed its wound healing effects at large^{16-18, 24,} could be accounted for by one or another of these protein-protein interactions. The series of alpha CT1-based variant peptides generated for the present study provided an opportunity to address this question. While alpha CT1-I is not competent to interact with ZO-1 PDZ2, this alpha CT1 variant does bind the Cx43 CT and upregulate S368 phosphorylation. Conversely, while M3 DDLAI showed no ability to bind Cx43 CT, or increase pS368, this peptide retained affinity for the ZO-1 PDZ2 domain. Finally, M1 AALAI showed no evidence of interaction with either PDZ2 or Cx43 CT, and demonstrated no ability to increase pS368 in the in vitro assay. We thus used the variant peptides, together with unmodified alpha CT1 in mouse hearts subjected to an ischemia-reperfusion (I/R) protocol to systematically assess which aspect of mode-of-action (i.e., peptide interaction with ZO-1 vs. Cx43) accounted for modulation of the I/R injury response by Cx43 CT mimetic peptides.

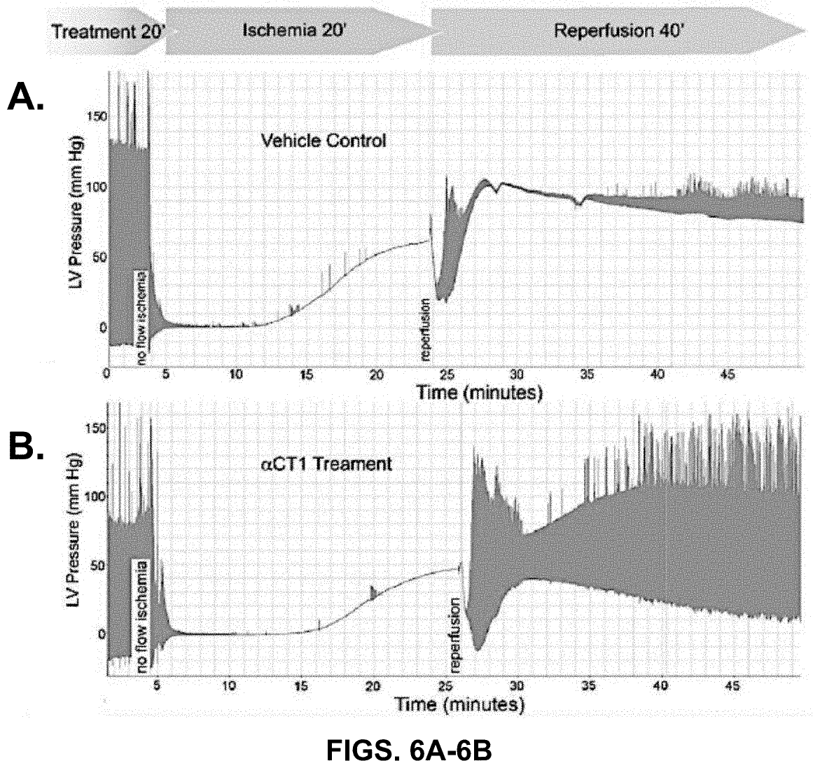

The protocol and experimental design for the cardiac I/R injury model is illustrated in FIG. 9. In summary, the protocol involved a 20-minute period of no flow ischemia period followed by 40 minutes of reperfusion. For treatment, peptides were infused into hearts over a 20-minute period just prior to the ischemic episode. Representative pressure traces from a vehicle control and alpha CT1-treated hearts are shown in FIGS. 6A and 6B, from which it can be qualitatively appreciated that pre-ischemic infusion of alpha CT1 results in preservation of LV contractile function upon reperfusion relative to vehicle control.

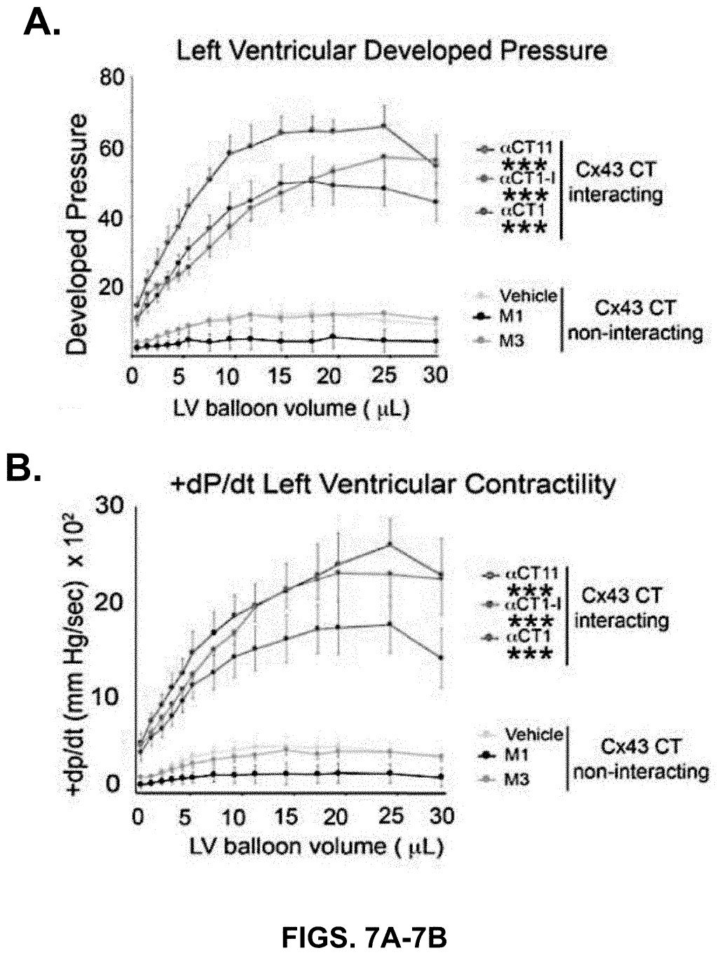

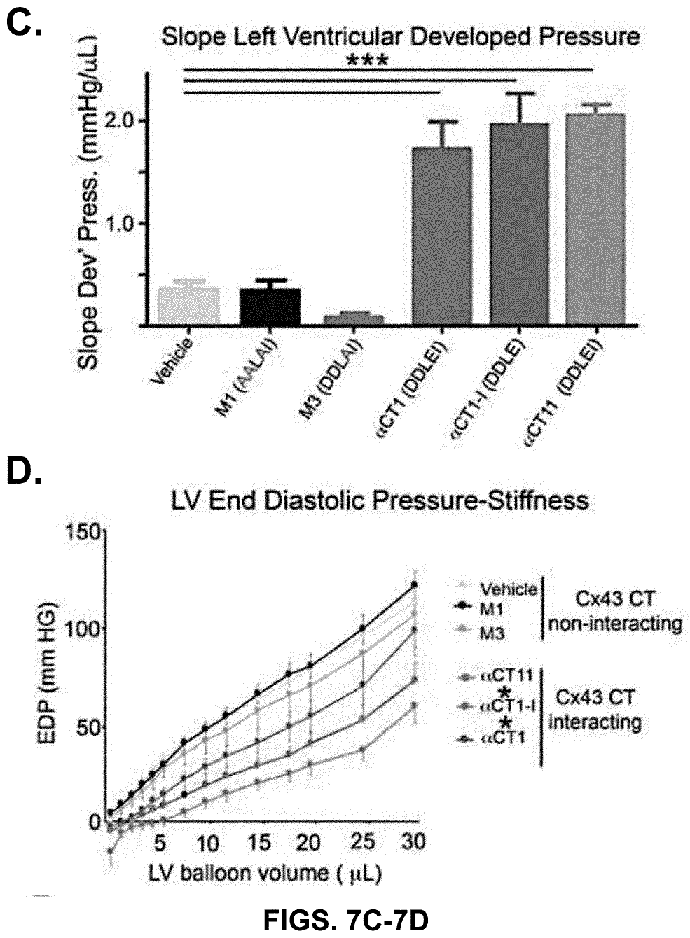

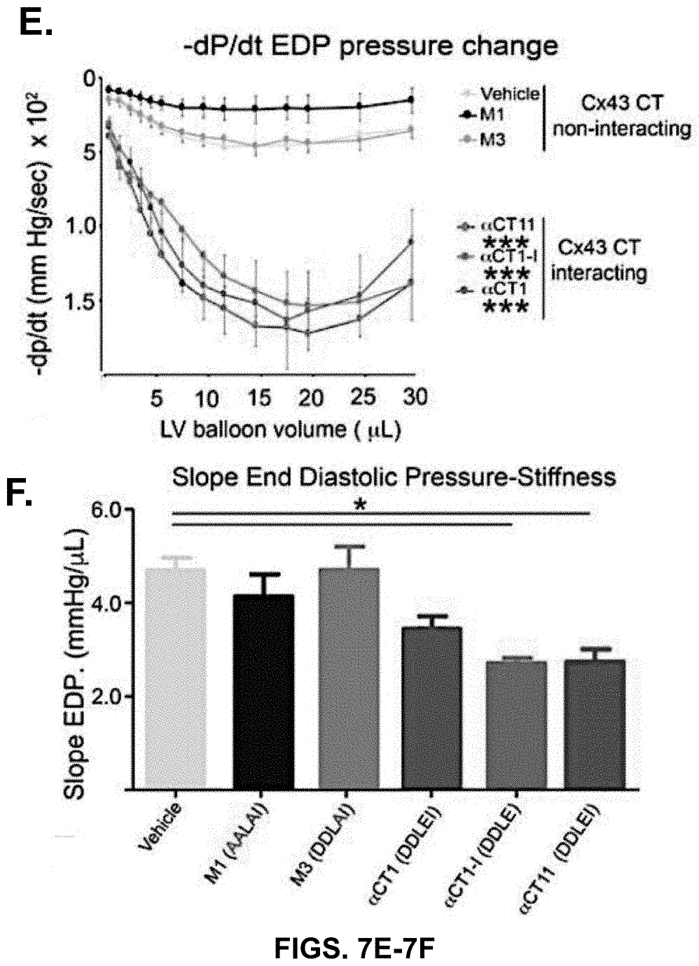

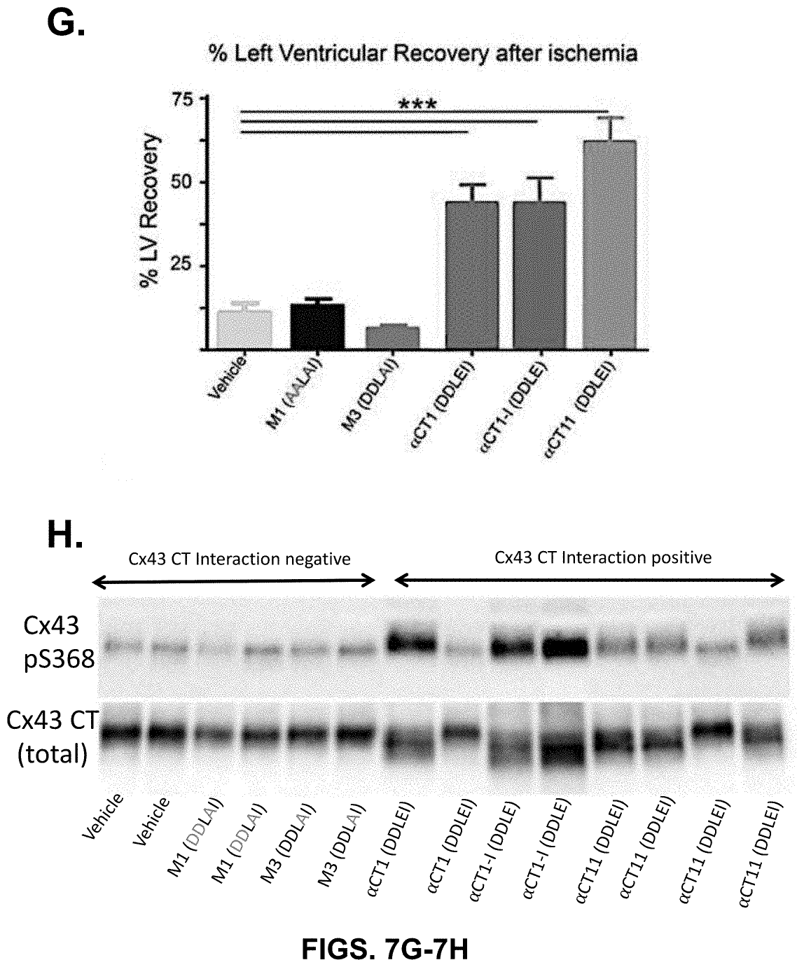

The effects of the alpha CT1 and the alpha CT1-variants on left ventricular (LV) systolic and diastolic contractile function showed a striking correlation with the Cx43 CT ability of peptides (FIGS. 7A-7H). Whereas the non-Cx43 CT interacting peptides M1 AALAI and M3 DDLAI showed no ability to improve recovery of either systolic (FIGS. 7A-7C) or diastolic (FIGS. 7D-7F) LV contractile performance during reperfusion, hearts pre-treated with the Cx43 CT-interacting peptides alpha CT1, alpha CT11 and alpha CT1-I demonstrated significant functional recovery after I/R injury, compared to vehicle control mice (FIGS. 7A-7G). Further, as alpha CT1-I is able to interact with Cx43 CT, but not PDZ2, the results suggested that ZO-1 binding was dispensable for induction of functional cardioprotection. Importantly, all Cx43 CT-binding peptides resulted in highly significant 3 to 5-fold improvements in functional recovery of LV contractile function during reperfusion following ischemic injury relative to vehicle control and the non-Cx43 CT interacting peptides (FIG. 7G). In line with the observations of the in vitro kinase assays (FIGS. 3A-3F), LV samples taken for Western blotting following pre-ischemic treatment of Langendorff-perfused mouse hearts with alpha CT1, alpha CT11, alpha CT1-I showed significant increases in phosphorylation at the Cx43 PKCμ-consensus locus S368 relative to vehicle control perfused hearts (FIG. 7H). By contrast, hearts exposed to peptides not competent to interact with Cx43 CT (i.e., M1 AALAI and M3 DDLAI), uniformly showed no propensity to upregulate S368 phosphorylation (FIG. 7H).

Post-Ischemic Treatment with the 9mer peptide alpha CT11 preserves LV Function alpha CT1 is in Phase III clinical testing in humans for pathologic skin wounds¹⁹. The results demonstrated in FIGS. 7A-7H indicated that pre-treatment with Cx43 CT binding peptides provided protection from injury in the ex vivo model studied. However, to be clinically useful to patients, such as those suffering a myocardial infarction, a drug would typically need to be given after an ischemic insult to the heart, i.e., after a myocardial infarction has been diagnosed. We thus treated hearts during the reperfusion phase following ischemic injury with alpha CT1, but determined that this did not result in significant recovery of LV function (data not shown). As alpha CT1 showed no evidence of post-infarction efficacy, we decided to explore an alternative approach. It was notable that the most striking recovery of post-ischemic LV function resulted from pre-ischemic treatment with the 9mer Cx43 CT-binding peptide alpha CT11 (Table 1). This is illustrated in FIG. 7A, where the curve for LV developed pressure for alpha CT11 conspicuously overarches that of the other two Cx43-interacting peptides, alpha CT1 and alpha CT1-I. This can also be observed in FIG. 7F, where the % of LV function recovery associated with alpha CT11 pre-infusion significantly exceeds that of alpha CT1 or alpha CT1-I (p<0.05). Based on these results suggestive of increased potency, it was examined whether alpha CT11 has a post-ischemic cardioprotective effect.

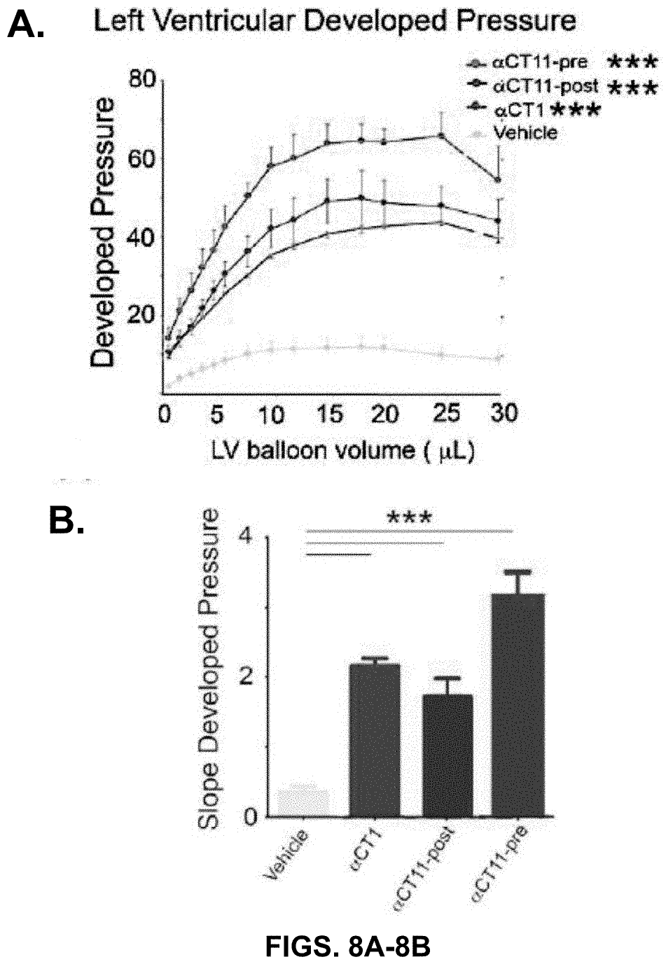

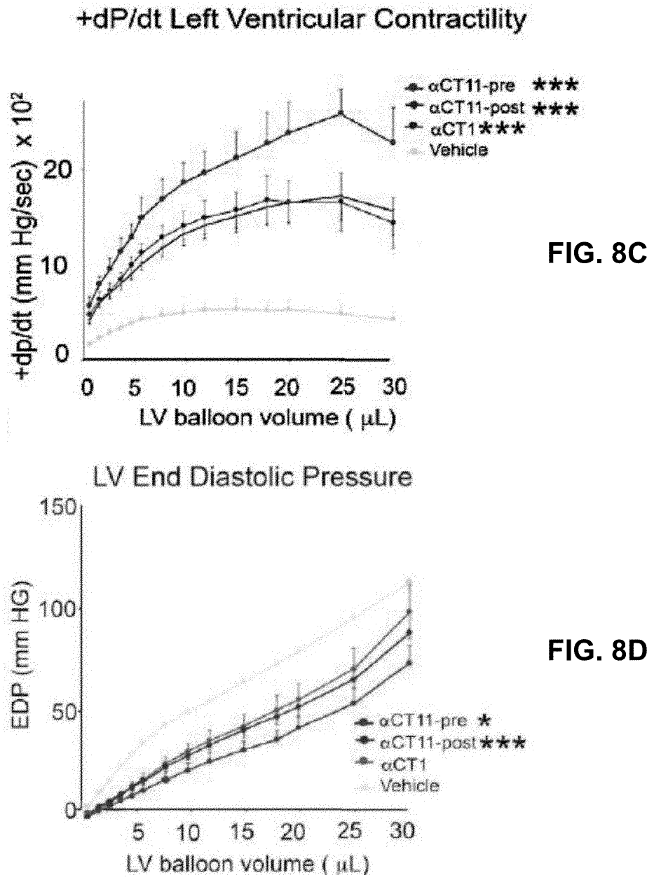

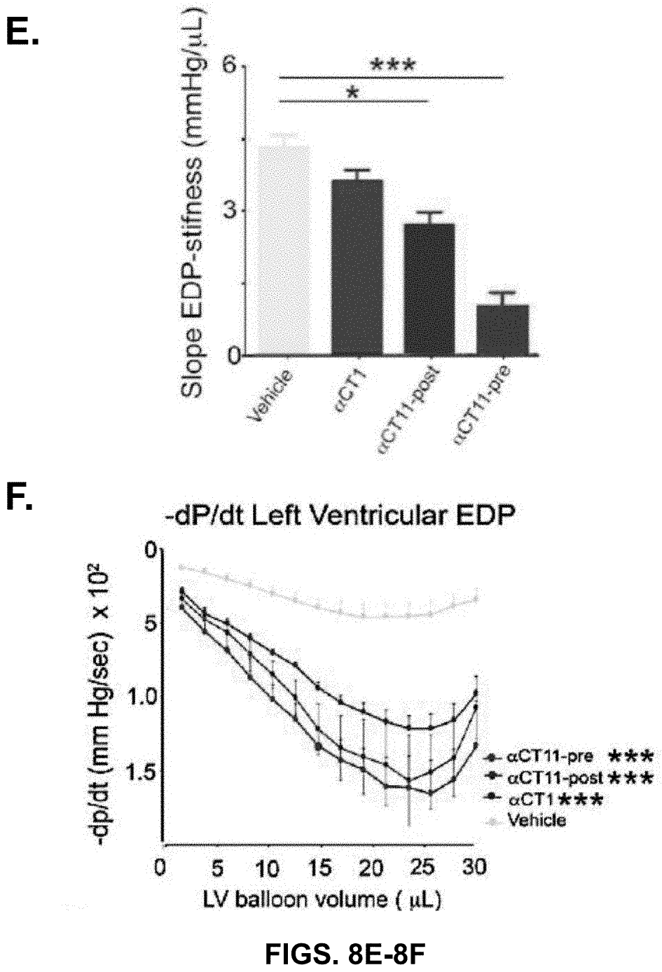

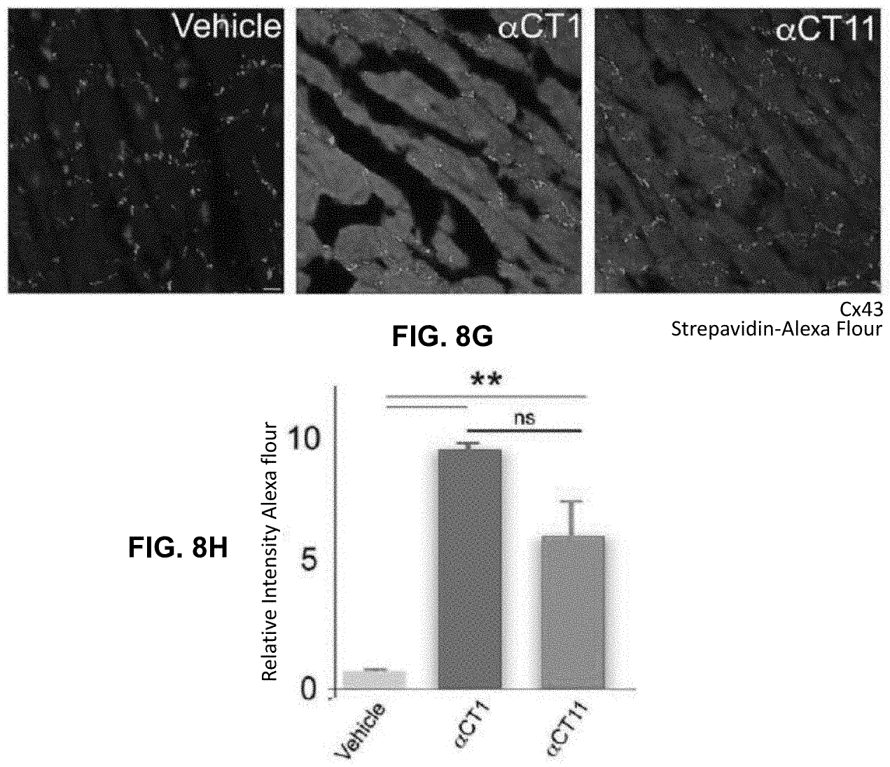

Alpha CT11 demonstrated an ability to significantly improve recovery of both systolic (FIGS. 8A-8C) and diastolic (FIGS. 8D-8F) LV contractile performance when infused in hearts during the reperfusion following ischemic injury. The level of cardioprotection achieved by this post-ischemic treatment was not as high as when alpha CT11 was provided prior to insult, but it was similar to that achieved for pre-ischemic treatment with alpha CT1. Given that alpha CT11 is missing a cell penetration sequence we were curious to determine whether the 9mer peptide (MW=1110 daltons) was being taken up into cardiomyocytes. Uptake of alpha CT11 in ventricular muscle was examined in mouse hearts that had been perfused with a biotynlylated alpha CT11 under the protocol summarized in supplementary FIGS. 1A-1D. Cardiomyocytes showed robust uptake of the 9mer alpha CT11 sequence, as detected by fluor conjugated streptavidin (FIG. 8G), and as compared to vehicle control perfused hearts. Relative to vehicle control, quantified levels of uptake of alpha CT11 in cells were comparable to those of alpha CT1, as indicated by measurement of relative fluorescence intensity levels in ventricular myocardial tissues (FIG. 8H).

Discussion

This Example can at least demonstrate that mimetic sequences incorporating the CT-most nine amino acids of Cx43 (amino acids R374 to 1382) complex with the Cx43 H2 sequence located between amino acids D340 and D360 of its carboxyl terminus (CT). This interaction causes disruption of polypeptide secondary structure, which in turn is associated with increases in a PKC-mediated phosphorylation in a serine residue at position 368 of Cx43-S368. Moreover, evidence is provided that the cardioprotective properties of Cx43 CT mimetic peptides, such as alpha CT1, may be explained to a significant degree by their propensity to interact with the Cx43 CT-binding competent peptides alpha CT1, alpha CT11, and alpha CT1-I preserve LV function following ischemic injury, whereas Cx43 interaction deficient variants of alpha CT1, M1, AALAI, and 3 DDLAI, do not. Alpha CT1 and H2 represent two spatially distinct sequences on the CT of native Cx43 molecules. Thus, the data suggest that interactions between or within Cx43 molecules in vivo can be involved in regulating Cx43 phosphorylation, which may be by controlling accessibility of PKCε to its Cx43 CT substrate.

These observations on the relationship between PKCε-mediated phosphorylation of S368 and cardioprotection are consistent with previous reports^{6, 7, 11, 32-39}. Phosphorylation Cx 43 at S368 and is correlated with reduced activity of Cx43-formed hemichannels^{6, 9, 10, 40}. Pro-inflammatory and injury spread signals resulting from unregulated opening of hemichannels in the myocyte sarcolemma are thought to be determinants of the severity of ischemia reperfusion damage to the heart⁴¹⁻⁵² Cx43 activity and pS368 phosphorylation events associated with mitochondrial membranes have also been linked to I/R injury severity^{42, 53, 54}. It has been reported that Cx43 CT sequences incorporating the Cx43 H2-binding sequence of interest herein result from alternative translation of the GJA-1 gene (Smyth et al 2013, PMID: 24210816). These include a 20 kDA isoform, termed GJA1-20k, which has been found to be enriched at the interface between mitochondria and microtubules⁵⁵. Similar to the results achieved with synthetic Cx43 CT mimetic sequences here, exogenous provision of GJA1-20k reduces infarct size in mouse hearts subjected to I/R injury⁵⁶.

The pH-dependent gating of Cx43-formed channels has been thought to involve the Cx43 CT in a “ball-and chain” mechanism^{57, 58}. The demonstration that the CT-most 10 amino acids of Cx43 (S373-1382 aka CT10) interacts with a region of the cytoplasmic loop domain of Cx43 referred to as L2, resulting in channel closure under acidic conditions, provides evidence supporting this hypothesis⁵⁹. It was demonstrated that a near-identical sequence to CT10 contained in alpha CT1 (i.e., R374-1382), also interacts with the H2 sequence of Cx43, doing so via precisely the same negatively charged amino acids required for L2 interaction²⁰. In addition to the shared affinity of the CT-most 9 amino acids of Cx43 for L2 and H2, comparison of L2 and H2 indicate other notable parallels. The L2 and H2 sequences of Cx43 have related secondary structures, both being marked by short stretches of alpha-helix. Further, L2 and H2 incorporate a pair of lysine (KK) residues. As demonstrated in this Example, these lysines are essential for alpha CT1 interaction, as substitution of K345 and K346 with neutral glutamines, as in the Cx43 CT QQ/KK construct, results in a loss of alpha CT1 binding to H2.

Taken together, the evidence suggests that the nine amino acid CT sequence of Cx43 mimicked by alpha CT1 is a multivalent ligand that participates in a number of protein-protein interactions. In addition to affinity for L2 and H2, this short segment of Cx43 includes the PDZ-binding-ligand necessary for linkage to ZO-1^{14, 60, 61}, as well as amino acids required for interaction with 14-3-3 theta⁶². Immediately proximal are consensus recognition sites for AKT (S373) 63, PKCε (S368)^{64, 65} and T-cell protein tyrosine phosphatase⁶⁶.

The results observed in this Example can indicate that the Cx43 CT-binding activity of alpha CT1, and not ZO-1 PDZ2 interaction, explains the cardioprotective effects of alpha CT1, at least in the model studied here. Whilst Cx43-ZO-1 interaction does not appear to have been a direct factor in the ex vivo model studied, potential roles for ZO-1 in regulating Cx43 phospho-status and hemichannel availability in vivo, including during ischemic injury, should not be discounted. ZO-1 is located at the edge of Cx43 GJs in a specialized zone of cell membrane known as the perinexus 63, 67, 68. In earlier studies, we have shown that high densities of hemichannels are found in this peri-junctional region 69, 70 and that PDZ-based interactions between ZO-1 and Cx43 govern the rate at which undocked connexons dock with connexons from apposed cells to form gap junctional channels, thereby regulating GJ size, as well as hemichannel availability within the cell membrane^{13, 14}. Recent work by two other groups have provided data supporting this hypothesis, and have also shown that phosphorylations at Cx43 S368 and S373 are central to how ZO-1 controls the accrual of perinexal hemichannels to the GJ^{63, 71}. The potential for regulatory interplay between PKC-ε and ZO-1 at the Cx43-CT is further suggested by earlier studies indicating that the presence of ZO-1 PDZ2 domain in the test tube-based PKC assay efficiently acts as a competitive inhibitor of alpha CT1 enhancement of S368 phosphorylation¹¹.

A key question raised by our study is whether the alpha CT1 Cx43-targeting mechanism determined as necessary for preservation of LV function also explains the primary mode-of-action of this therapeutic peptide in other tissues. In skin wounding experiments in mice and pigs, alpha CT1 has been shown to reduce inflammation, increase wound healing rates and decrease granulation tissue formation^{12, 24}. In related observations in Phase II clinical testing of humans, alpha CT1 treatment increased the healing rate of slow-healing skin wounds, including diabetic foot ulcers and venous leg ulcers^{16, 18}. Given the current results in heart, it will be of interest to determine whether the mode-of-action of alpha CT1 in wounded skin also involves Cx43 CT interaction and/or increased pS368. As the GAIT1 Phase III clinical trial on alpha CT1 moves forward on more than 500 patients with diabetic foot ulcers¹⁹, such insight on molecular mode-of-action will be useful in understanding the basis of any clinical efficacy identified in humans, as well as a step in building a safety profile for this therapeutic peptide.

Of further clinical translational note are these findings on the cardioprotective effect of post-ischemic treatment by the short alpha CT1 variant alpha CT11—a result that may have clinical implications. Interestingly, alpha CT11 does not have a cell penetration sequence, but it nonetheless is efficiently internalized by LV cardiomyocytes after intravascular perfusion in the ex vivo model used herein. The mechanism of this cellular uptake is presently under study by our group, but it may be explained by the small size (MW=1110 daltons) and linear, random coiled-coil 3D structure of alpha CT11—see FIG. 2A. Neijssen and co-workers reported that linear peptides with molecular masses below 1800 daltons readily diffuse through Cx43-formed channels⁷². Given alpha CT11 is a linear peptide with a molecular mass well below 1800 daltons, and that hemichannel opening is induced by ischemic insult⁴¹, the interesting prospect is raised that alpha CT11 reaches its cytoplasmic target (i.e., the CT domain of Cx43), via a short (<20 nm) transit through an open Cx43 hem ichannel pore. Future work would usefully test this hypothesis, as well as undertake testing of alpha CT11 in preclinical models of cardiac I/R injury in vivo as a prelude to Phase I testing of this therapeutic peptide in human patients with acute myocardial infarction.

### REFERENCES FOR EXAMPLE 20

1. Laird D W, Lampe P D and Johnson R G. Cellular Small Talk. Sci Am. 2015; 312:70-7.
2. Desplantez T, Dupont E, Severs N J and Weingart R. Gap junction channels and cardiac impulse propagation. J Membr Biol. 2007; 218:13-28.
3. Jansen J A, van Veen T A, de Bakker J M and van Rijen H V. Cardiac connexins and impulse propagation. J Mol Cell Cardiol. 2010; 48:76-82.
4. Martin P E and Evans W H. Incorporation of connexins into plasma membranes and gap junctions. Cardiovasc Res. 2004; 62:378-87.
5. Severs N J, Bruce A F, Dupont E and Rothery S. Remodelling of gap junctions and connexin expression in diseased myocardium. Cardiovasc Res. 2008; 80:9-19.
6. Solan J L and Lampe P D. Spatio-temporal regulation of connexin43 phosphorylation and gap junction dynamics. Biochim Biophys Acta. 2018; 1860:83-90.
7. Ek-Vitorin J F, King T J, Heyman N S, Lampe P D and Burt J M. Selectivity of connexin 43 channels is regulated through protein kinase C-dependent phosphorylation. Circ Res. 2006; 98:1498-505.
8. Richards T S, Dunn C A, Carter W G, Usui M L, Olerud J E and Lampe P D. Protein kinase C spatially and temporally regulates gap junctional communication during human wound repair via phosphorylation of connexin43 on serine368. J Cell Biol. 2004; 167:555-62.
9. Lampe P D, TenBroek E M, Burt J M, Kurata W E, Johnson R G and Lau A F. Phosphorylation of connexin43 on serine368 by protein kinase C regulates gap junctional communication. J Cell Biol. 2000; 149:1503-12.
10. Fiori M C, Figueroa V, Zoghbi M E, Saez J C, Reuss L and Altenberg G A. Permeation of calcium through purified connexin 26 hemichannels. J Biol Chem. 2012; 287:40826-34.
11. O'Quinn M P, Palatinus J A, Harris B S, Hewett K W and Gourdie R G. A Peptide Mimetic of the Connexin43 Carboxyl Terminus Reduces Gap Junction Remodeling and Induced Arrhythmia Following Ventricular Injury. Circ Res. 2011.
12. Ongstad E L, O'Quinn M P, Ghatnekar G S, Yost M J and Gourdie R G. A Connexin43 Mimetic Peptide Promotes Regenerative Healing and Improves Mechanical Properties in Skin and Heart. Adv Wound Care (New Rochelle). 2013; 2:55-62.
13. Rhett J M, Jourdan J and Gourdie R G. Connexin43 Connexon to Gap Junction Transition Is Regulated by Zonula Occludens-1. Mol Biol Cell. 2011.
14. Hunter A W, Barker R J, Zhu C and Gourdie R G. Zonula occludens-1 alters connexin43 gap junction size and organization by influencing channel accretion. Mol Biol Cell. 2005; 16:5686-98.
15. Laird D W and Lampe P D. Therapeutic strategies targeting connexins. Nat Rev Drug Discov. 2018.
16. Grek C L, Prasad G M, Viswanathan V, Armstrong D G, Gourdie R G and Ghatnekar G S. Topical administration of a connexin43-based peptide augments healing of chronic neuropathic diabetic foot ulcers: A multicenter, randomized trial. Wound Repair Regen. 2015; 23:203-12.
17. Grek C L, Montgomery J, Sharma M, Ravi A, Rajkumar J S, Moyer K E, Gourdie R G and Ghatnekar G S. A Multicenter Randomized Controlled Trial Evaluating a Cx43-Mimetic Peptide in Cutaneous Scarring. J Invest Dermatol. 2017; 137:620-630.
18. Ghatnekar G S, Grek C L, Armstrong D G, Desai S C and Gourdie R G. The effect of a connexin43-based Peptide on the healing of chronic venous leg ulcers: a multicenter, randomized trial. J Invest Dermatol. 2015; 135:289-98.
19. ClinicalTrials.gov. A Study of Granexin (□CT1) Gel in the Treatment of Diabetic Foot Ulcer. https://clinicaltrialsgov/ct2/show/NCT02667327. 2016; Phase III Clinical Trial.
20. D'Hondt C, lyyathurai J, Wang N, Gourdie R G, Himpens B, Leybaert L and Bultynck G. Negatively charged residues (Asp378 and Asp379) in the last ten amino acids of the C-terminal tail of Cx43 hemichannels are essential for loop/tail interactions. Biochem Biophys Res Commun. 2013; 432:707-12.
21. Wu S Y, Zou P, Fuller A W, Mishra S, Wang Z, Schey K L and McHaourab H S. Expression of Cataract-linked gamma-Crystallin Variants in Zebrafish Reveals a Proteostasis Network That Senses Protein Stability. J Biol Chem. 2016; 291:25387-25397.
22. Pierce B G, Wiehe K, Hwang H, Kim B H, Vreven T and Weng Z. ZDOCK server: interactive docking prediction of protein-protein complexes and symmetric multimers. Bioinformatics. 2014; 30:1771-3.
23. He H, Javadpour M M, Latif F, Tardiff J C and Ingwall J S. R-92L and R-92W mutations in cardiac troponin T lead to distinct energetic phenotypes in intact mouse hearts. Biophys J. 2007; 93:1834-44.
24. Ghatnekar G S, O'Quinn M P, Jourdan L J, Gurjarpadhye A A, Draughn R L and Gourdie R G. Connexin43 carboxyl-terminal peptides reduce scar progenitor and promote regenerative healing following skin wounding. Regen Med. 2009; 4:205-23.
25. Giepmans B N and Moolenaar W H. The gap junction protein connexin43 interacts with the second PDZ domain of the zona occludens-1 protein. Current Biology. 1998; 8:931-4.
26. Ambrosi C, Ren C, Spagnol G, Cavin G, Cone A, Grintsevich E E, Sosinsky G E and Sorgen P L. Connexin43 Forms Supramolecular Complexes through Non-Overlapping Binding Sites for Drebrin, Tubulin, and ZO-1. PLoS One. 2016; 11:e0157073.
27. Jin C, Martyn K D, Kurata W E, Warn-Cramer B J and Lau A F. Connexin43 PDZ2 binding domain mutants create functional gap junctions and exhibit altered phosphorylation. Cell Commun Adhes. 2004; 11:67-87.
28. Bouvier D, Spagnol G, Chenavas S, Kieken F, Vitrac H, Brownell S, Kellezi A, Forge V and Sorgen P L. Characterization of the structure and intermolecular interactions between the connexin40 and connexin43 carboxyl-terminal and cytoplasmic loop domains. J Biol Chem. 2009; 284:34257-71.
29. Spagnol G, A1-Mugotir M, Kopanic J L, Zach S, Li H, Trease A J, Stauch K L, Grosely R, Cervantes M and Sorgen P L. Secondary structural analysis of the carboxyl-terminal domain from different connexin isoforms. Biopolymers. 2016; 105:143-62.
30. Sosinsky G E, Solan J L, Gaietta G M, Ngan L, Lee G J, Mackey M R and Lampe P D. The C-terminus of connexin43 adopts different conformations in the Golgi and gap junction as detected with structure-specific antibodies. Biochem J. 2007; 408:375-85.
31. Chen J, Pan L, Wei Z, Zhao Y and Zhang M. Domain-swapped dimerization of Z O-1 PDZ2 generates specific and regulatory connexin43-binding sites. EMBO J. 2008; 27:2113-23.
32. Hawat G and Baroudi G. Differential modulation of unapposed connexin 43 hemichannel electrical conductance by protein kinase C isoforms. Pflugers Arch. 2008; 456:519-27.
33. Hatanaka T, Shimizu R and Hildebrand D. Expression of a Stokesia laevis epoxygenase gene. Phytochemistry. 2004; 65:2189-96.
34. Hund T J, Lerner D L, Yamada K A, Schuessler R B and Saffitz J E. Protein kinase Cepsilon mediates salutary effects on electrical coupling induced by ischemic preconditioning. Heart Rhythm. 2007; 4:1183-93.
35. Miura T, Miki T and Yano T. Role of the gap junction in ischemic preconditioning in the heart. Am J Physiol Heart Circ Physiol. 2010; 298:H1115-25.
36. Naitoh K, Yano T, Miura T, Itoh T, Miki T, Tanno M, Sato T, Hotta H, Terashima Y and Shimamoto K. Roles of Cx43-associated protein kinases in suppression of gap junction-mediated chemical coupling by ischemic preconditioning. Am J Physiol Heart Circ Physiol. 2009; 296:H396-403.
37. Jeyaraman M M, Srisakuldee W, Nickel B E and Kardami E. Connexin43 phosphorylation and cytoprotection in the heart. Biochim Biophys Acta. 2012; 1818:2009-13.
38. Jozwiak J and Dhein S. Local effects and mechanisms of antiarrhythmic peptide AAP10 in acute regional myocardial ischemia: electrophysiological and molecular findings. Naunyn Schmiedebergs Arch Pharmacol. 2008; 378:459-70.
39. Morel S, Christoffersen C, Axelsen L N, Montecucco F, Rochemont V, Frias M A, Mach F, James R W, Naus C C, Chanson M, Lampe P D, Nielsen M S, Nielsen L B and Kwak B R. Sphingosine-1-phosphate reduces ischaemia-reperfusion injury by phosphorylating the gap junction protein Connexin43. Cardiovasc Res. 2016; 109:385-96.
40. Bao X, Altenberg G A and Reuss L. Mechanism of regulation of the gap junction protein connexin 43 by protein kinase C-mediated phosphorylation. Am J Physiol Cell Physiol. 2004; 286:C647-54.
41. Shintani-Ishida K, Uemura K and Yoshida K. Hemichannels in cardiomyocytes open transiently during ischemia and contribute to reperfusion injury following brief ischemia. Am J Physiol Heart Circ Physiol. 2007; 293:H1714-20.
42. Boengler K, Stahlhofen S, van de Sand A, Gres P, Ruiz-Meana M, Garcia-Dorado D, Heusch G and Schulz R. Presence of connexin 43 in subsarcolemmal, but not in interfibrillar cardiomyocyte mitochondria. Basic Res Cardiol. 2009; 104:141-7.
43. Morel S and Kwak B R. Roles of connexins in atherosclerosis and ischemia-reperfusion injury. Curr Pharm Biotechnol. 2012; 13:17-26.
44. Gill R, Kuriakose R, Gertz Z M, Salloum F N, Xi L and Kukreja R C. Remote ischemic preconditioning for myocardial protection: update on mechanisms and clinical relevance. Mol Cell Biochem. 2015; 402:41-9.
45. Retamal M A, Schalper K A, Shoji K F, Orellana J A, Bennett M V and Saez J C. Possible involvement of different connexin43 domains in plasma membrane permeabilization induced by ischemia-reperfusion. J Membr Biol. 2007; 218:49-63.
46. Gourdie R G, Dimmeler S and Kohl P. Novel therapeutic strategies targeting fibroblasts and fibrosis in heart disease. Nat Rev Drug Discov. 2016; 15:620-38.
47. Saez J C, Schalper K A, Retamal M A, Orellana J A, Shoji K F and Bennett M V. Cell membrane permeabilization via connexin hemichannels in living and dying cells. Exp Cell Res. 2010; 316:2377-89.
48. Wang N, De Bock M, Decrock E, Bol M, Gadicherla A, Vinken M, Rogiers V, Bukauskas F F, Bultynck G and Leybaert L. Paracrine signaling through plasma membrane hemichannels. Biochim Biophys Acta. 2013; 1828:35-50.
49. Li F, Sugishita K, Su Z, Ueda I and Barry W H. Activation of connexin-43 hemichannels can elevate [Ca(2+)]i and [Na(+)]i in rabbit ventricular myocytes during metabolic inhibition. J Mol Cell Cardiol. 2001; 33:2145-55.
50. Kondo R P, Wang S Y, John S A, Weiss J N and Goldhaber J I. Metabolic inhibition activates a non-selective current through connexin hemichannels in isolated ventricular myocytes. J Mol Cell Cardiol. 2000; 32:1859-72.
51. Stout C E, Costantin J L, Naus C C and Charles A C. Intercellular calcium signaling in astrocytes via ATP release through connexin hemichannels. J Biol Chem. 2002; 277:10482-8.
52. Clarke T C, Williams O J, Martin P E and Evans W H. ATP release by cardiac myocytes in a simulated ischaemia model: inhibition by a connexin mimetic and enhancement by an antiarrhythmic peptide. Eur J Pharmacol. 2009; 605:9-14.
53. Boengler K, Dodoni G, Rodriguez-Sinovas A, Cabestrero A, Ruiz-Meana M, Gres P, Konietzka I, Lopez-Iglesias C, Garcia-Dorado D, Di Lisa F, Heusch G and Schulz R. Connexin 43 in cardiomyocyte mitochondria and its increase by ischemic preconditioning. Cardiovasc Res. 2005; 67:234-44.
54. Rodriguez-Sinovas A, Boengler K, Cabestrero A, Gres P, Morente M, Ruiz-Meana M, Konietzka I, Miro E, Totzeck A, Heusch G, Schulz R and Garcia-Dorado D. Translocation of connexin 43 to the inner mitochondrial membrane of cardiomyocytes through the heat shock protein 90-dependent TOM pathway and its importance for cardioprotection. Circ Res. 2006; 99:93-101.
55. Fu Y, Zhang S S, Xiao S, Basheer W A, Baum R, Epifantseva I, Hong T and Shaw R M. Cx43 Isoform GJA1-20k Promotes Microtubule Dependent Mitochondrial Transport. Front Physiol. 2017; 8:905.
56. Basheer W A, Xiao S, Epifantseva I, Fu Y, Kleber A G, Hong T and Shaw R M. GJA1-20k Arranges Actin to Guide Cx43 Delivery to Cardiac Intercalated Discs. Circ Res. 2017; 121:1069-1080.
57. Morley G E, Taffet S M and Delmar M. Intramolecular interactions mediate pH regulation of connexin43 channels. Biophysical Journal. 1996; 70:1294-302.
58. Moreno A P, Chanson M, Elenes S, Anumonwo J, Scerri I, Gu H, Taffet S M and Delmar M. Role of the carboxyl terminal of connexin43 in transjunctional fast voltage gating. Circ Res. 2002; 90:450-7.
59. Ponsaerts R, De Vuyst E, Retamal M, D'Hondt C, Vermeire D, Wang N, De Smedt H, Zimmermann P, Himpens B, Vereecke J, Leybaert L and Bultynck G. Intramolecular loop/tail interactions are essential for connexin 43-hemichannel activity. Faseb J. 2010.
60. Toyofuku T, Yabuki M, Otsu K, Kuzuya T, Hori M and Tada M. Direct association of the gap junction protein connexin-43 with ZO-1 in cardiac myocytes. J Biol Chem. 1998; 273:12725-31.
61. Giepmans Bn and Moolenaar Wh. The gap junction protein connexin43 interacts with the second PDZ domain of the zona occludens-1 protein. Current Biology. 1998; 8:931-4.
62. Smyth J W, Zhang S S, Sanchez J M, Lamouille S, Vogan J M, Hesketh G G, Hong T, Tomaselli G F and Shaw R M. A 14-3-3 mode-1 binding motif initiates gap junction internalization during acute cardiac ischemia. Traffic. 2014; 15:684-99.
63. Dunn C A and Lampe P D. Injury-triggered Akt phosphorylation of Cx43: a ZO-1-driven molecular switch that regulates gap junction size. J Cell Sci. 2014; 127:455-64.
64. Doble B W, Ping P and Kardami E. The epsilon subtype of protein kinase C is required for cardiomyocyte connexin-43 phosphorylation. Circ Res. 2000; 86:293-301.
65. Srisakuldee W, Jeyaraman M M, Nickel B E, Tanguy S, Jiang Z S and Kardami E. Phosphorylation of connexin-43 at serine 262 promotes a cardiac injury-resistant state. Cardiovasc Res. 2009; 83:672-81.
66. Li H, Spagnol G, Naslaysky N, Caplan S and Sorgen P L. T C-PTP directly interacts with connexin43 to regulate gap junction intercellular communication. J Cell Sci. 2014; 127:3269-79.
67. Barker R J, Price R L and Gourdie R G. Increased association of ZO-1 with connexin43 during remodeling of cardiac gap junctions. Circ Res. 2002; 90:317-24.
68. Baker S M, Kim N, Gumpert A M, Segretain D and Falk M M. Acute internalization of gap junctions in vascular endothelial cells in response to inflammatory mediator-induced G-protein coupled receptor activation. FEBS Lett. 2008; 582:4039-46.
69. Rhett J M, Ongstad E L, Jourdan J and Gourdie R G. Cx43 associates with Na(v)1.5 in the cardiomyocyte perinexus. J Membr Biol. 2012; 245:411-22.
70. Veeraraghavan R, Lin J, Hoeker G S, Keener J P, Gourdie R G and Poelzing S. Sodium channels in the Cx43 gap junction perinexus may constitute a cardiac ephapse: an experimental and modeling study. Pflugers Arch. 2015.
71. Thevenin A F, Margraf R A, Fisher C G, Kells-Andrews R M and Falk M M. Phosphorylation regulates connexin43/ZO-1 binding and release, an important step in gap junction turnover. Mol Biol Cell. 2017; 28:3595-3608.
72. Neijssen J, Herberts C, Drijfhout J W, Reits E, Janssen L and Neefjes J. Cross-presentation by intercellular peptide transfer through gap junctions. Nature. 2005; 434:83-8.

### Example 21

Heart disease is a primary cause of death in the United States, and can particularly affect minority and rural populations. A leading manifestation of heart disease is myocardial infarction. Whilst death rates from myocardial infarction have declined in the last 20 years due to improvide emergency care, such as percutaneous intervention to open blocked coronary arteries, it still remains a significant cause of chronic sickness and death. There is currently no approved clinical therapy for preserving cardiac muscle lost during the acute phase of a heart attached or for treating the chronic progression to heart failure. It is often referred to by the community as the “epidemic” of heart failure. The silent epidemic places huge burdens on health care systems. Moreover, with increasing rates of obesity, co-morbidities such as diabetes and an aging population, the burdens and costs associated with post-cardiac arrest are most-certainly going to increase in the coming years.

The therapeutic approach described in at least this Example, presents a multi-fold approach that can be applied not only to treatment of heart attack, but also a platform for delivery of any suitable cargo compound for treatment of any disease to which can be treated by said cargo compound.

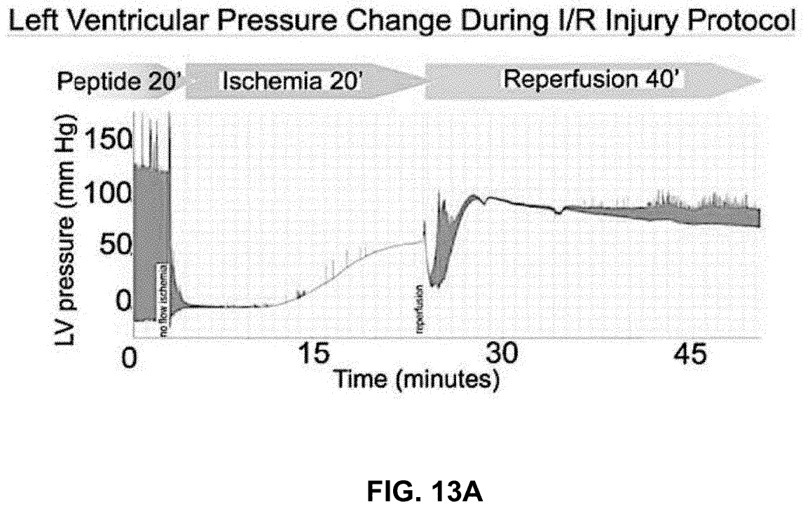

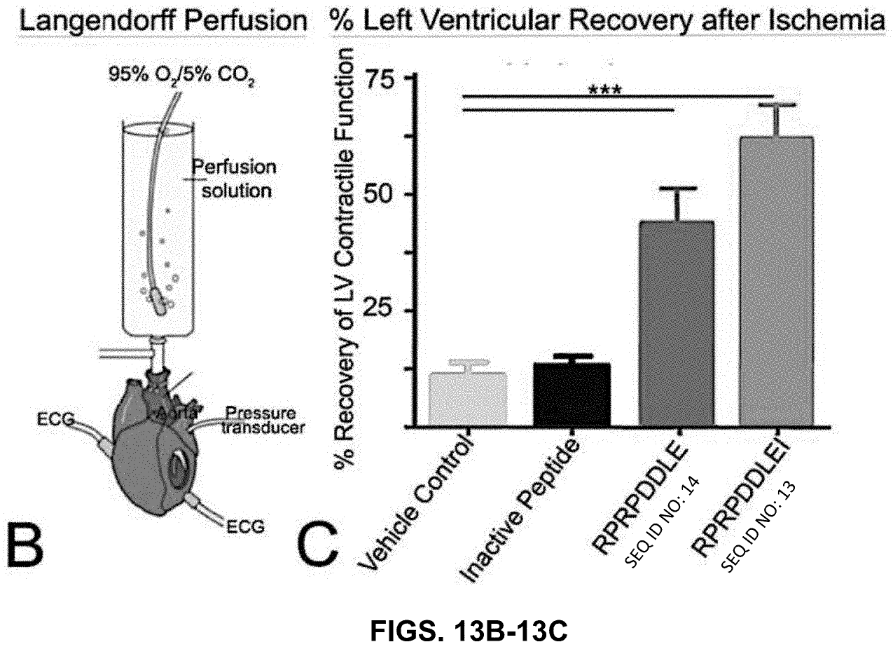

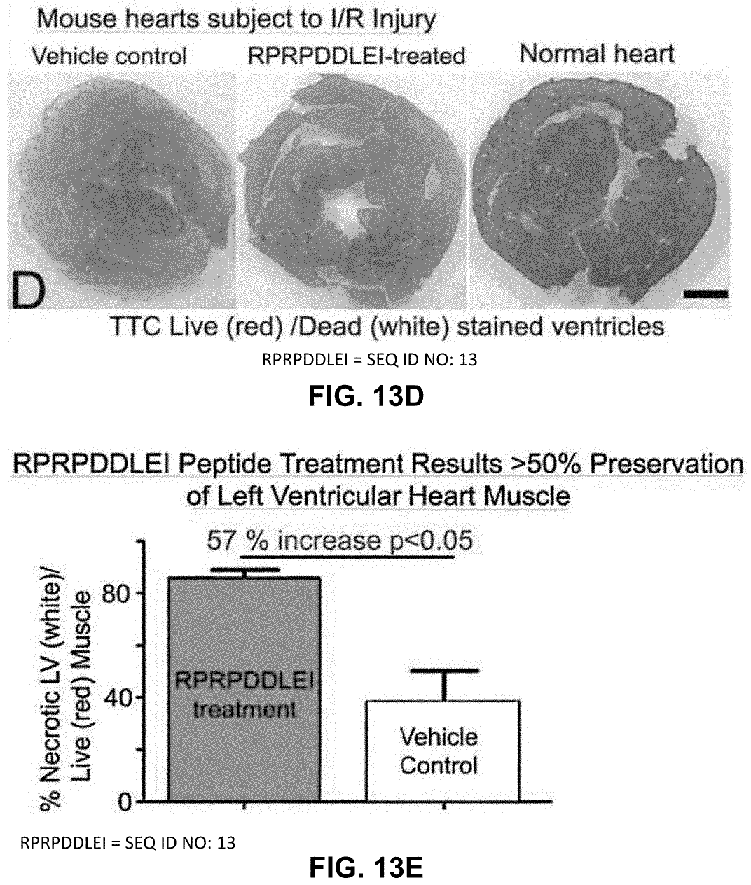

Ex vivo model data from perfused hearts isolated from mice can demonstrate that short peptides (e.g. alpha CT1, alpha CT11), which are based on the CT of connexin43/Gja1 can reduce cardiac muscle loss by more than half following an ischemia-reperfusion (I/R) injury, which simulates myocardical infarction (see e.g. FIGS. 13A-13E, 18A-18E, 20A-20B, 21).

FIGS. 13A-13E. Short peptides based on the Carboxyl-Terminus (CT) of the gap junction protein connexin 43 (Cx43) provide high levels of protection against ischemia reperfusion injury to the heart. Contractile function of the left ventricle (LV) of isolated beating mouse hearts was continuously recorded (FIG. 13A) during ex vivo perfusion (FIG. 13B) in a model simulating ischemia-reperfusion (I/R) injury to the heart. To induce an ischemic injury, hearts were subjected to a no flow ischemic injury for 20 minutes (indicated by loss of pressure recording on (FIG. 13A) and subsequently reperfused with oxygenated buffer solution for about 40 minutes. This was observed to result in about a 80-90% loss of LV contractile function in control hearts (FIG. 13C) By contrast, hearts treated for 20 minutes with either the Cx43 CT-based peptide RPRPDDLE (8 amino acids) (SEQ ID NO: 14) or RPRPDDLEI (9 amino acids) (SEQ ID NO: 13) both showed striking levels (p<0.001) of cardioprotection, with recovery of LV contractile function 5-6 times higher than that of hearts subject to vehicle control or inactive peptide control perfusions (FIG. 13C). To confirm cardioprotection, staining of hearts after measurement of contractile function was performed using 2,3,4-triphenyltetrazolium chloride (TTC) to indicate sectors of dead (white staining) and live (red staining) heart muscle. Treatment with therapeutic peptide resulted in dramatic improvements in preservation of live heart muscle (FIG. 13D), with treated hearts having about 57% (p<0.05) more muscle than control hearts subject to the I/R injury protocol (FIG. 13E).

FIGS. 18A-18E can demonstrate post-ischemic alpha CT11 results in dramatic preservation of LV contractile function in isolated, perfused hearts in association with alpha CT11 permenance into myocytes. Alpha CT1 was observed to spare left ventricular (LV) muscle and contractile function in an exvivo I/R injury model and determined that is mode-of action via binding the cytoplasmic H2 domain of Cx43, prompting the cardioprotectrive pS368 phosphoryalation. Further, it was determined that alphaCT11, which contains only the RPRPDDLEI (SEQ ID NO: 13) (no antennapedia sequence), is taken up by myocytes and can provide effective preservation of LV contractile function as shown in e.g. FIGS. 18A-18B.

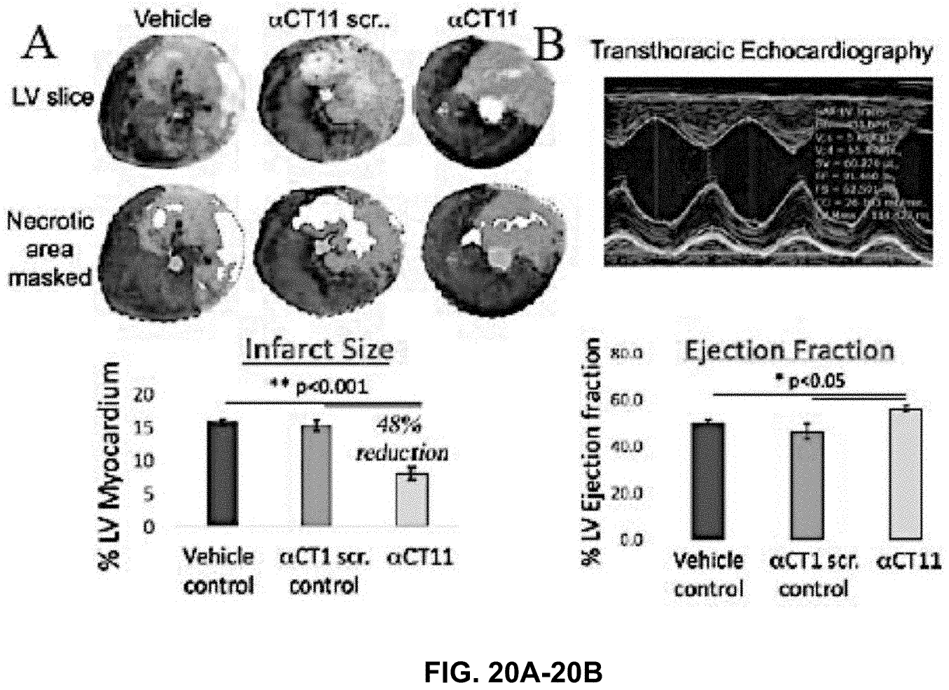

FIGS. 20A-20B can demonstrate that post-MI treatment with alpha CT11 can reduce infarct size by about 48% in a mouse in vivo myocardial infarction model. Briefly, within 10 minutes of confirmation of a successful reperfusion, mice were given an intraperitoneal (IP) injection (about 400 micrograms in 0.1 mL 0.9% NaCl) of alphaCT11, scrambled alphaCT11 control peptide or a matching vehicle solution (N=6 mice/group). In a blinded analysis performed on TTC/Phtalo blue-stained vibrotome sections 24 hours post-MI, alpha CT11 was found to reduce infarct size by about 48% (infract expressed as a percentage of left ventricle, p<0.0001 v. vehicle), as assessed by echocardiography. Based on this it can be demonstrated threat post-MI alpha CT11 showed evidence of significantly decreasing infarct size preserved LV function in this model.

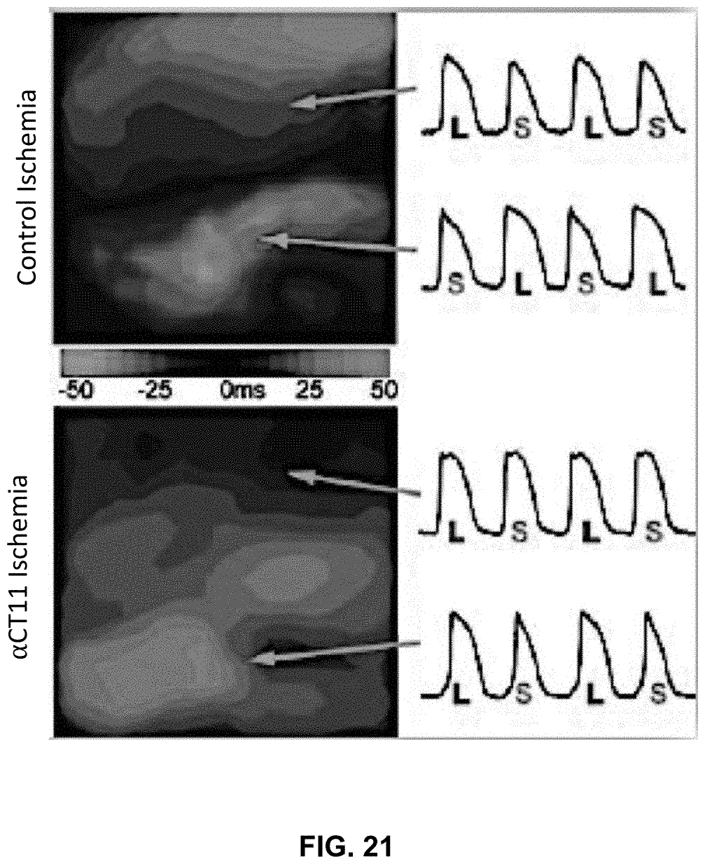

FIG. 21 can demonstrate that alpha CT11 can suppress discordant alterans in wedge preparations of ventricular tissue during ischemia. FIG. 21 can show transmural maps of wedge preparations during low flow ischemia. Upper panel: AP alternans magnitude (contour intensity) and phase (contour color, green is +phase, red is −phase) in a control shows distinct regions alternating with opposite phase depicted by green and red contours. On right are representative APs where duration alternates discordantly (L: long, S: short) between regions. FIG. 21, Lower Panel. At the same HR with alpha CT11, only concordant alternans (i.e. one color) is observed, with APs all alternating in same phase. 5 out of the 5 controls displayed disconcordant alternans, while none of the alpha CT11-treated wedges did. Thus, alpha CT11 can exhibit anti-arrhythimic activity in the setting of acute ischemia in this ex vivo model.

### Example 22

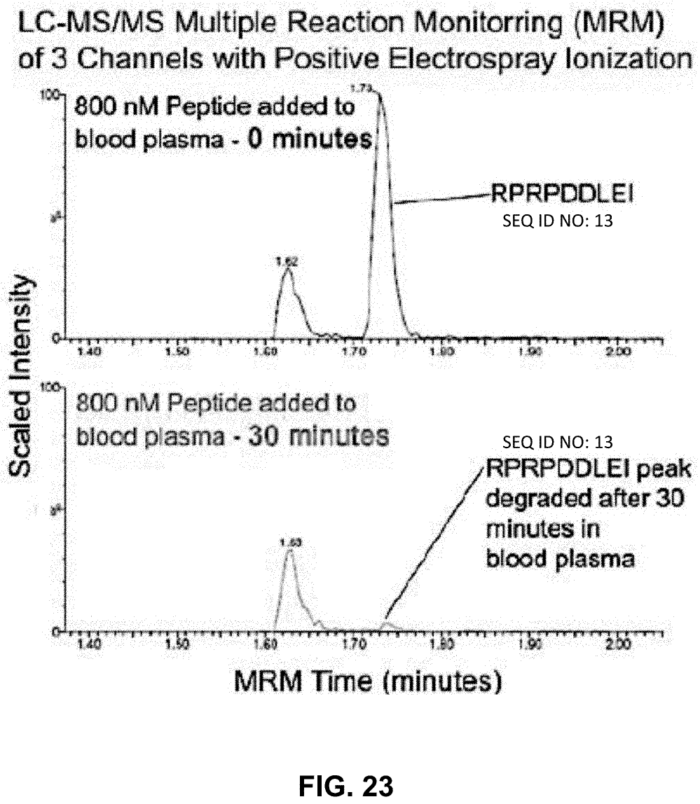

Many of the Examples provided herein present data that can demonstrate the efficacy of short peptides based on the CT of connexin43 for various diseases including myocardial infarction, diabetic foot ulcer, and wound healing. Despite efficacy of these peptides being delivered as unprotected peptide formulations, particularly when delivered topically, delivery via many routes can be impeded by degradation inside the body. Many other biologic and small molecule therapeutics also suffer from similar degradation issues. Indeed, most unprotected short peptides and polynucleotides (e.g. miRNAs) are rapidly degraded in body fluids in vivo, thereby limiting the interest of the pharmaceutical industry in such molecules. This consideration is particularly relevant in pathologic situations, such as the heart post-MI, where hypoxia, oxygen free radicals, and elevated pH prompt upregulation of degradation pathways. Although IP injection of peptides (e.g. alphaCT11) 30 minutes after induction of ischemia provided cardioprotection in vivo, in large animal models and human patients, a more stable formulation may be needed, at least for some delivery routes. As shown in e.g. FIG. 23, which shows mass spectrometry results that can demonstrate that alpha CT11 can be degraded after about 30 minutes in blood serum. As is demonstrated in at least this Example and as described elsewhere herein, engineered vesicles that incorporate engineered connexin43 hemichannels can be loaded with a cargo compound, e.g. alpha CT1 and/or alpha CT11 peptides. Protection by being loaded inside of an engineered vesicle can reduce degradation and/or can facilitate delivery of the cargo by forming channels with connexons present in cell membranes. See e.g. FIG. 17.

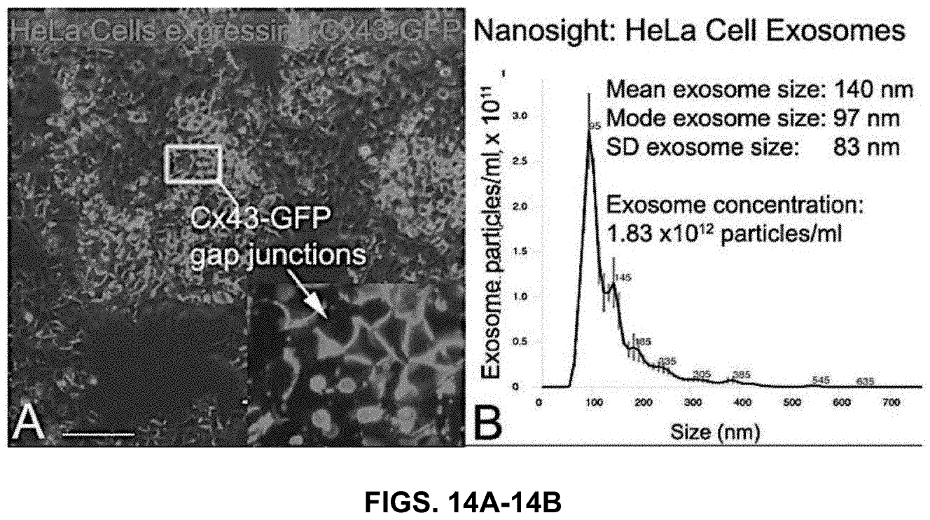

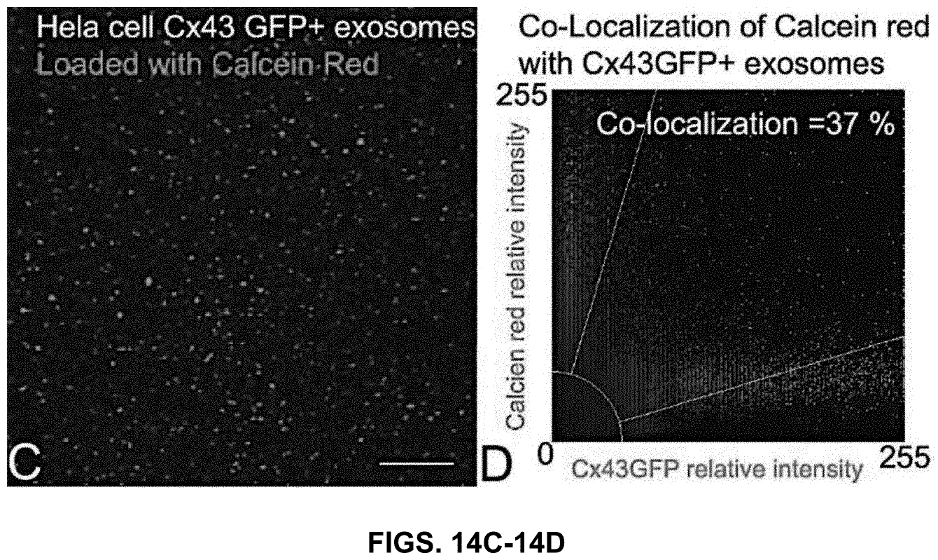

HeLa cells that heterogeneously express a recombinant Cx43 that is fused to a GFP (Cx43GFP) were generated. These cells were used to generate exosomes containing the recombinant Cx43GFP, which were subsequently isolated using standard ultracentrifugation-based methods as noted in Serrano-Pertierra et. al., Characterization of Plasma-Derived Extracellular Vesicels Isolated by Different Methods: A comparison Study. Bioengineering (Basel) 2016. FIGS. 14A-14E HeLa cell exosomes retain Calcein dye. Briefly, exosomes were isolated from HeLa cells expressing Cx43GFP using standard ultracentrifugation methods and assayed by Nanosight to ensure that isolated particles conformed to dimensions consistent with exosomes (50-200 nm) (FIGS. 14A-E). These exosomes were GFP+ and blotted for Cx43. As discussed elsewhere herein, Cx43 HCs can be opened by lowering external Ca2+ or by raising external pH above 7.4, e.g., to pH 8.5. Both these HC-opening prompts were tested by placing exosomes from HeLa Cx43GFP cells in a buffer solution containing an HC-permeant dye (Atto-565, 5 microM), in the presence of either 0.1 mM Ca2+ or pH 8.5 for 60 minutes (37 degrees C.) (FIG. 10C). Following re-isolation, exosomes were switched into buffer containing Ca2+ concentrations of 1.8 mM or at pH 7.2 to close HCs. Both hemichannel opening “switches” loaded exosomes with high efficiency (FIG. 10C).

Exosomes from HeLa Cx43-GFP cells have the advantage that they are readily visualized. However, the goal of viable clinical approach to exosomal delivery of alpha CT11 may require another source of EVs, due to yields attainable (we routinely obtain about 100 microg/ml from HeLa Cx43GFP cells) and expense of isolating exosomes from cultured cells. It was recently reported that milk is enriched in exosomes. This was confirmed to be the case, finding that the ultracentrifugation-based isolation methods provided EV yields from unpasteurized milk that were two orders of magnitude (10-12 mg/ml, EV median size=187 nm+/−67) greater than we were able to from cultured cells. We confirmed that these exosomes contained Cx43 and tested pH 8.5 loading with Atto-565 and FAM labeled alphaCT11. To assess cellular uptake, we labeled milk exosomes and found that EVs (0.35 microg/ml in 200 microL culture fluid) were efficiently taken up into the cytoplasm of HMEC-1 cells (express Cx43) over a 3-hour time course (FIG. 10D).

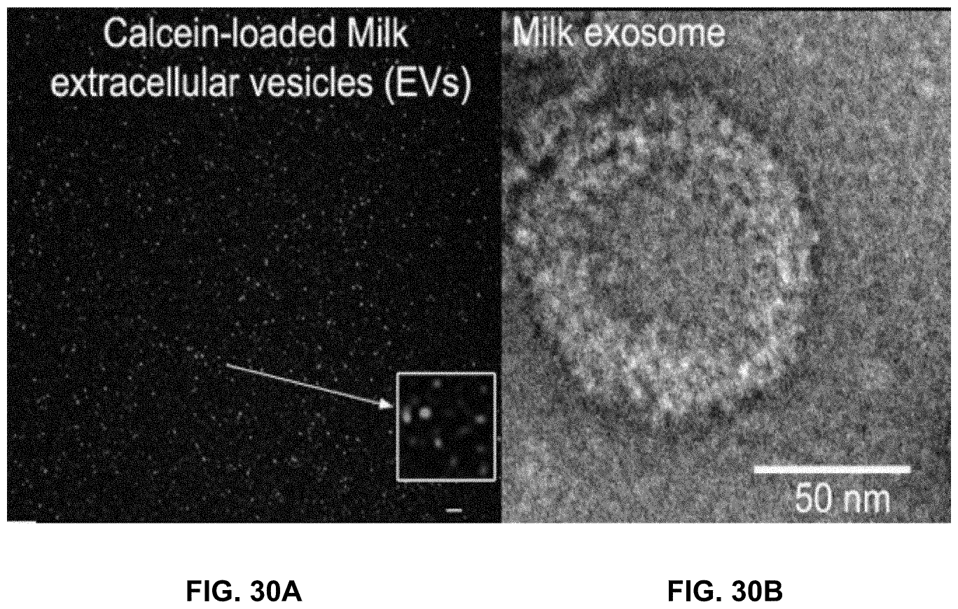

(FIG. 14A) HeLa cells engineered to express Cx43-GFP-inset shows Cx43GFP gap junctions (GJs). (FIG. 14B) Nanosight size distribution of Cx43GFP+ exosomes from HeLa cells. (FIG. 14C) Laser scanning confocal microscopy (LSCM) image of Cx43GFP+ exosomes loaded with Calcein red dye. (FIG. 14D) Significant co-localization of exosomal Cx43GFP+ with Calcein red measured at time points >60 minutes. This co-localization confirms exosomal retention of Calcein, indicating that its ester bonds have been cleaved and the dye was now trapped in the exosome. Retained Calcein within EVs provides a method for isolating and purifying EVs on the basis of fluorescence (e.g., by a FACS sorter or a like machine) or density (e.g., by centrifugation in a density gradient or by differential flow sorting)—as indeed can uptake of other molecules (e.g. sugars) into EVs by HCs or other methods described herein. Scale bars: A=100 μm, C=5 μm. Moreover, we determined that the efficiency of this uptake is increased by adjusting pH to generate a pH gradient between the inside and outside of the EV, see e.g. FIGS. 30-32.

FIG. 17 shows a schematic demonstrating suggested mechanisms of action for alpha CT11 activity and interaction with connexin43 and Connexin43 hemichannels and loading of an engineered exosome as described herein with an exemplary cargo (e.g. alpha CT11) compound, and delivery of a cargo compound. FIG. 17 shows on mechanism of cargo compound delivery that involves gap junction channel formation between connexins on the exosome and the cell to which the cargo can be delivered. In FIG. 17, this is connexon43 on both the exosome and cell. It will be appreciated other delivery methods are possible and described herein.

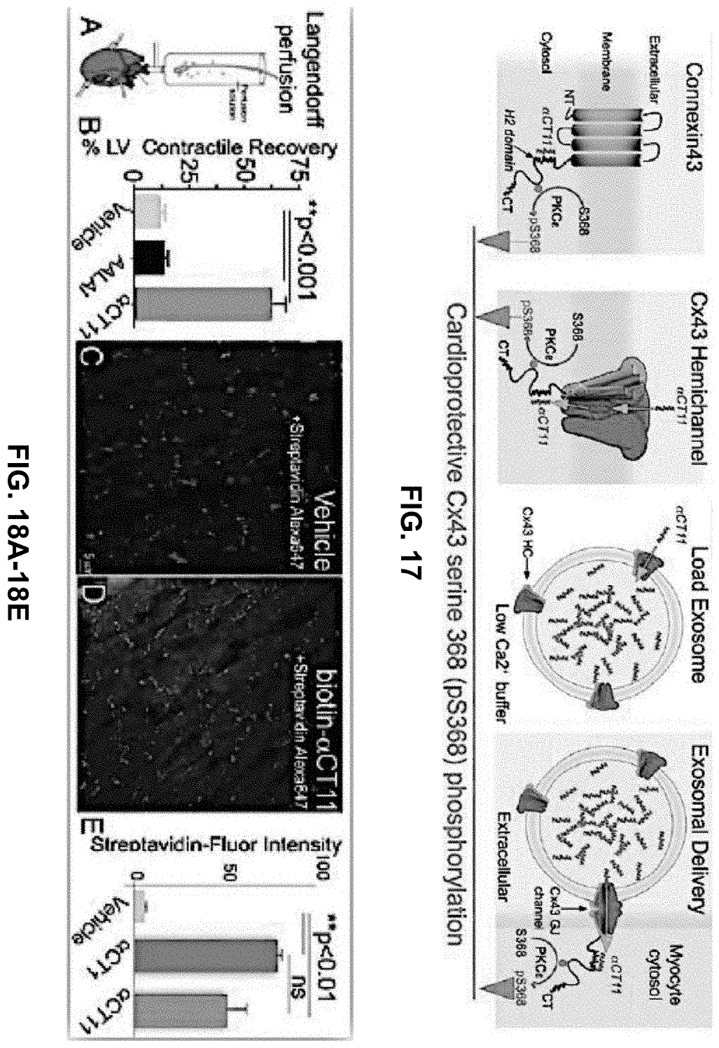

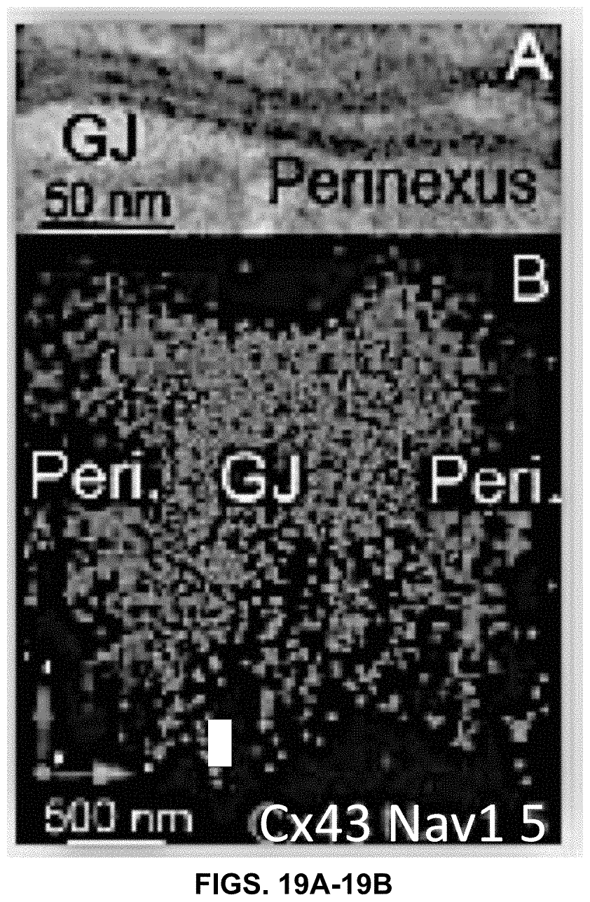

A perinexus is a specialized domain of intercellular interaction at the edge of gap junctions (GJs). Voltage-gated sodium channel (VGSC) subunit NAv1.5 complexed with Cx43 HCs in the perinexus structure. It has been previously determined that the perinexus could undergo dehiscence, with intermembrane distances widening to the point (>30 nm) where ephaptic coupling would no longer operate. Induction of perinexal widening prompted by induction of transient edema was accompanied by conduction slowing and arrhythmia, which was in line with computational models. It was believed that the beta1 subunit (Scn1b) of VGSCs facilitated perinexal adhesion, which provides an intercellular scaffold for trans-activating Nav1.5 channels within the narrow (<30 nm) perinexal cleft. Super resolution and electron microscopy, together with smart patch clamp (SPC) were used to c characterize the structure and function of this nanodomain (FIGS. 19A-19B). It was determined that beta1 knockout (KO) mouse ventricles shows profound perinexal dehiscence, in line with beta1 being important to maintaining adhesion. A synthetic peptide beta adp1, was designed to target the extracellular adhesion domain of beta1 and subsequently generated. Beta adp1 caused perinexal de-adhesion and also selectively reduced sodium currents at the edge of Cx43GFP labeled GJs in neonatal rat myocyte monolayers. Optical mapping studies of intact hearts and myocyte monolayers derived from human iPSCs revealed that beta adp1 caused arrhythmogenic conduction slowing. It was concluded that beta1-mediated adhesion at the perinexus can facilitate a non-canonical pathway for AP propagation between cardiomyocytes. FIGS. 19A-19B can demonstrate the Cx43 Gap Junction perinexus, which is a specialized zone of myocyte interaction at the edge of GJs. FIG. 19A shows an electron micrograph of GJ and adjacent perinexal cleft. FIG. 19B shows STORM super resolution image of a Cx43 GJ, with adjacent clusters of Nav1.5 VGSCs in the adjacent perinexus (Peri).

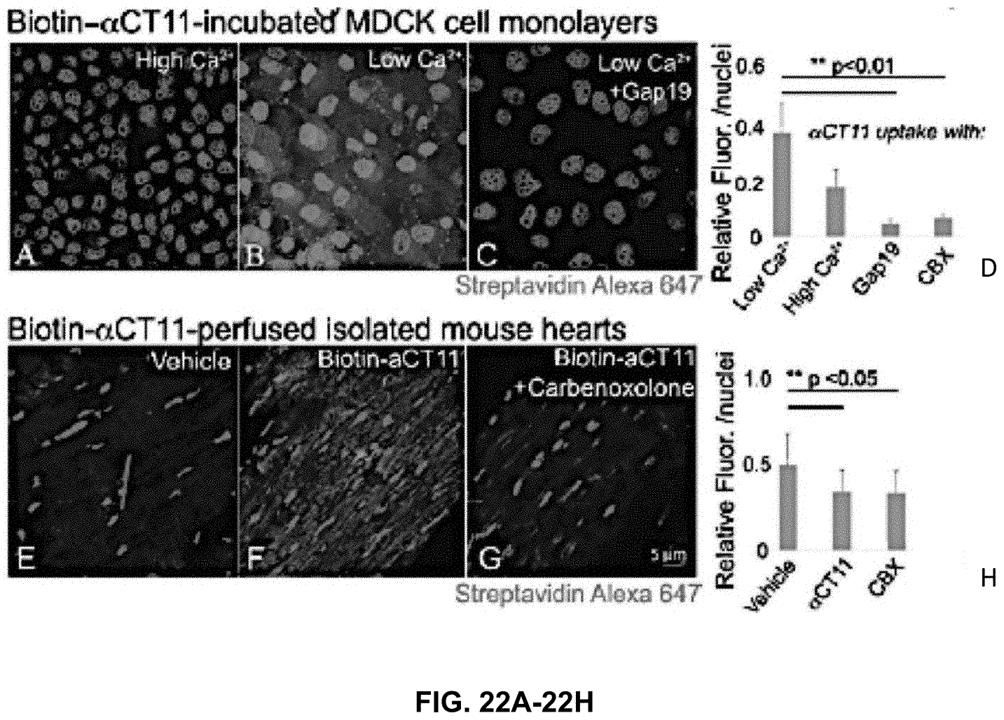

FIGS. 22A-22H can demonstrate that HC-mediated alpha CT11 uptake into the cytoplasm of MDCK Cx43 cells and LV myocytes in perfused mouse hearts. Further, HC-mediated uptake was observed to be dependent on calcium concentration. This feature was engineered into engineered Cx43 connexons and can be used as demonstrated in Example 23 to load and unload an engineered vesicle with a cargo compound. Briefly, isolated mouse hearts were perfused with 2 micromolar carbenoxolone for 20 minutes to block HC (hemichannel) activity prior to a 20 minute infusion with biotinylated alpha CT11 (biotin-alphaCT11) according to the method described in Example 20. As demonstrated in FIGS. 22A-22H, HCs was observed to mediate cellular uptake of alphaCT11, relative to hearts receiving alphaCT11 alone (FIG. 22F). Pre-treatment with carbenoxolone resulted in observed significant reductions (p<0.05) in cytoplastic levels of alpha CT11 in myocytes (FIG. 22H). Biotinylated alpha CT11 was detected and measured on LV cryosections by streptavidin Alexa647 as noted in association with FIGS. 18A-18E.

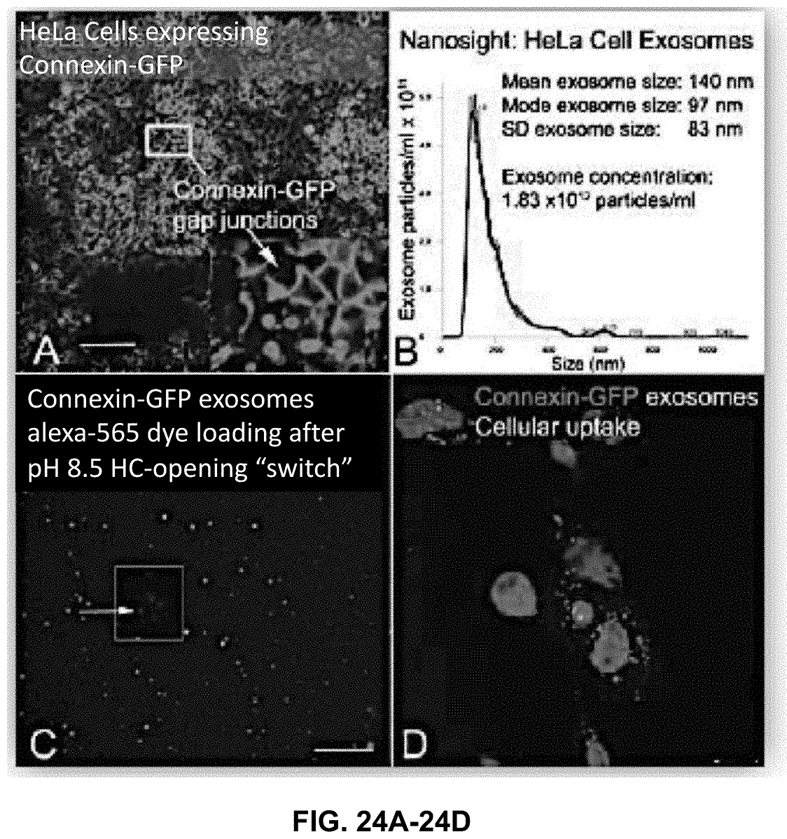

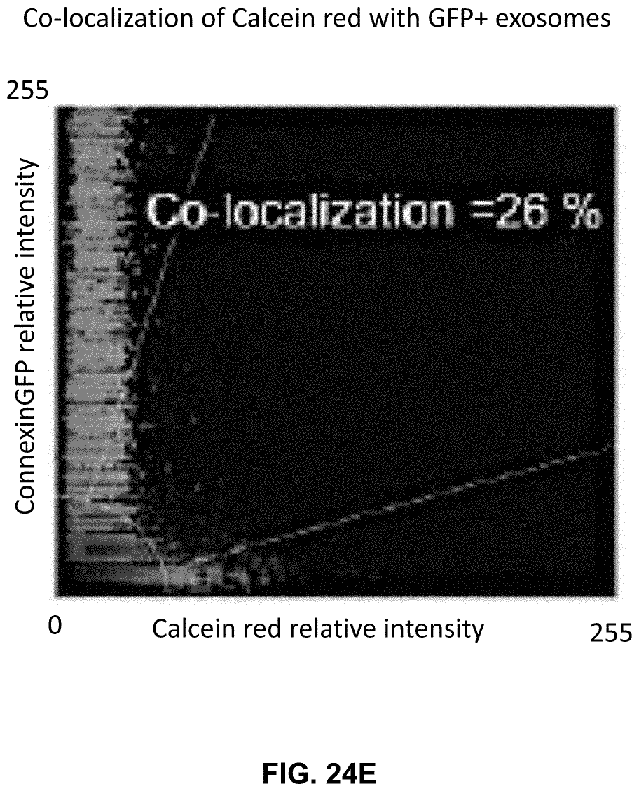

FIGS. 24A-24E can demonstrate isolation, cargo loading, and uptake of exosomes expressing Cx43GFP. (FIG. 24A) HeLa cells engineered to express Cx43GFP-show GFP+ GJs between cells. (FIG. 24B) Nanosight size and concentration of Cx43GFP exosomes. (FIG. 24C) Cx43GFP exosomes loaded with hemichannel permeant dye Atto-565 by increasing alkalinity of buffer. (FIG. 24D) Cellular uptake of exosomes. (FIG. 24E) Co-localization analysis can confirm hemichannel switch can allow for cargo compound loading (as demonstrated via dye loading) Scale A=100 μm, C, D=10 μm.

### Example 23

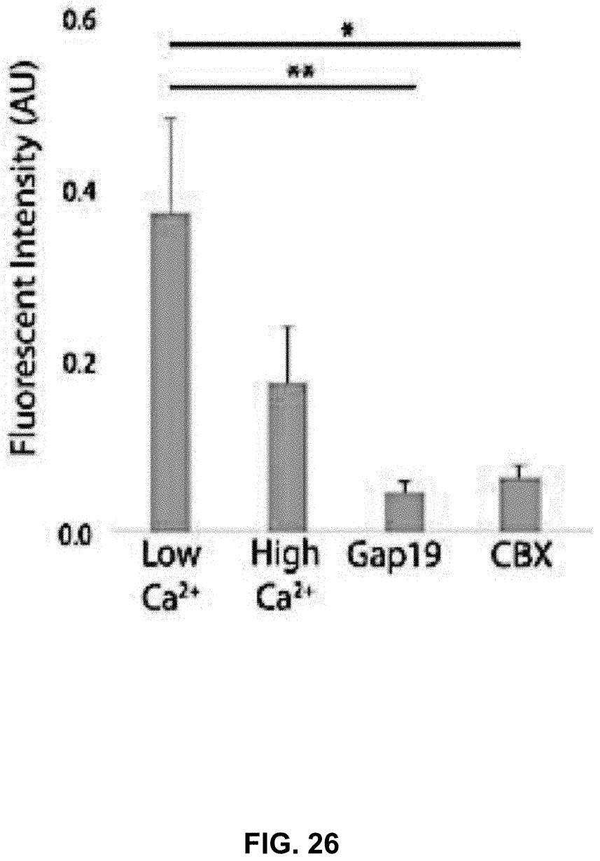

As described elsewhere herein, the engineered vesicles can include a calcium responsive connexin, e.g. connexin43 and/or engineered connexin43. This structural feature can be used to load and unload exosomes with cargo molecule(s) by altering calcium concentration in the environment, which can stimulate opening and closing of the hemichannel(s) in a concentration dependent fashion. FIG. 26 shows a graph that can demonstrate that a calcium switch (e.g. calcium concentration) can be used to allow RPRPDDLEI (SEQ ID NO: 13) to permeate *p<0.05, **p<0.001. Loading can be accomplished by exposing engineered vesicles containing a calcium responsive connexons (HCs) to a low calcium concentration (e.g. less than 0.2 mM), which can open the HCs and allowing diffusion to move cargo molecules through the open channels into the vesicles. The HCs can be closed to retain the cargo compound inside the engineered vesicles by raising the Calcium concentration.

### Example 24

Production of engineered vesicles, such as engineered exosomes, as described elsewhere herein can be produced using cells, such as tissue specific cells (e.g. cardiac cells) or from stem cells (e.g. iPSCs). However, these techniques may be unsuitable for some purposes. For example, production of exosomes via in vitro methods or culture-based methods can be expensive and yield-prohibitive for some large scale production.

This Example can demonstrate the production and use of milk exosomes as at least one way to address the problems associated with scaling production of exosomes. Use of these exosomes may be advantages for delivery to cardiac and other tissues as milk exosomes have previously demonstrated tissue bias and can preferentially accumulate in the brain, kidney, heart, liver and other organs after oral ingestion (see e.g. Manca et al. Sci Rep. 2018; 8:11321). Milk exosomes have been reported to be efficiently taken up by the heart in vivo. See e.g. 154.

Aqil F, Munagala R, Jeyabalan J, Agrawal A K, Kyakulaga A H, Wilcher S A and Gupta R C. Milk exosomes-Natural nanoparticles for siRNA delivery. Cancer Lett. 2019; Manca et al., Milk exosomes are bioavailable and distinct microRNA cargos have unique tissue distribution patterns. Sci Rep. 2018; 8:11321; 159. Li B, Hock A, Wu R Y, Minich A, Botts S R, Lee C, Antounians L, Miyake H, Koike Y, Chen Y, Zani A, Sherman P M and Pierro A. Bovine milk-derived exosomes enhance goblet cell activity and prevent the development of experimental necrotizing enterocolitis. PLoS One. 2019; 14:e0211431; and Betker J L, Angle B M, Graner M W and Anchordoquy T J. The Potential of Exosomes From Cow Milk for Oral Delivery. J Pharm Sci. 2018.

Milk exosomes were obtained from unpasteurized milk obtained from a creamery. The exosomal yields were about 15 mg/mL, which was over 2 orders of magnitude greater than the yield obtained using cell culture. Briefly, unpasteurized milk was centrifuged twice at low speed (about 1,200×g, at 4 degrees C., for about 10 minutes) to remove fat, cells, and large debris. The defatted supernatant was then centrifuged at a greater speed (about 21,500×g at 4 degrees C. for 30 min, 1h) to remove residual fat and casein. The clear supernatant (whey) was then ultracentrifuged (about 100,000×g for 4 degrees C. for about 90 minutes) and pelleted exosomes were resuspended in a phosphate buffered saline (PBS) solution. The latter ultracentrifugation step was repeated two more times to wash the exosome pellet. The final pellet was resuspended in an aliquot (about 1 mL) of PBS containing 25 mM trehalose as a cryoprotectant, and 200 microliter aliquots containing about 15 mg/mL of exosomal membrane was stored at about −70 degrees Celsius for later use

To test whether milk exosomes can target injured myocardial tissues, mice were subjected to MI (e.g., FIG. 25) followed by IP injection or oral gavage of 0.5 ml (185 micrograms/ml) Dil-labeled milk exosomes. Hearts isolated from mice 6 hours after receiving exosomes IP showed significant levels (p<0.001) of Dil fluorescence relative to mice that had not received exosomes (FIG. 25). Hearts from mice receiving exosomes by oral gavage, showed elevated fluorescence. This can demonstrate that milk-based exosomes may be appropriate delivery for various cargo molecules described herein. FIG. 16 discussed further in Example 25, can demonstrate loading of a milk exosome with a model cargo compound (Calcein dye).

### Example 25

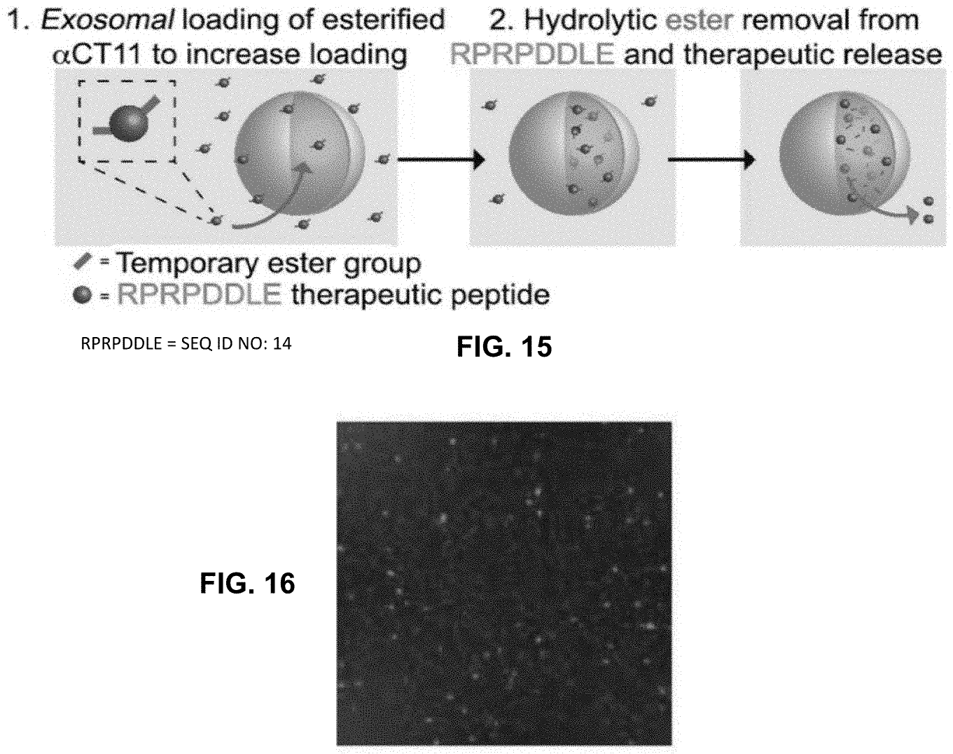

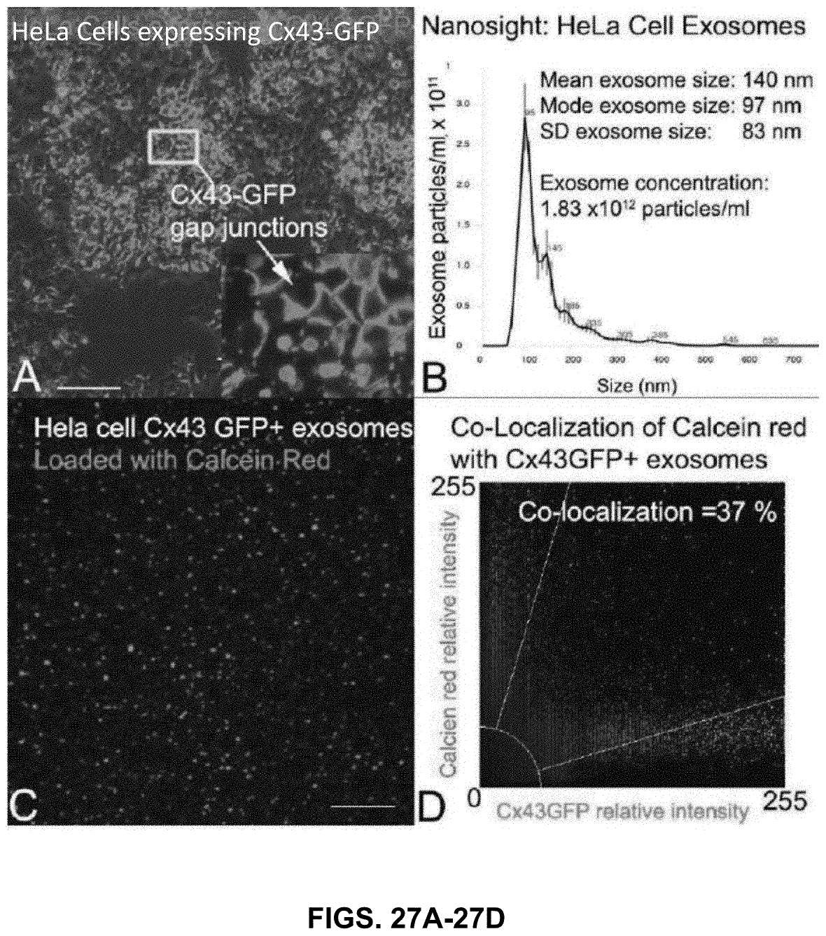

Although cargo compounds can enter in bulk via the HCs of the engineered vesicles described herein, some may escape being encapsulated by passing through the vesicle membrane. To improve loading efficiency and provide additional release control within an engineered vesicle described herein, the cargo compound can have chemical groups linked to it by ester bonds using an appropriate reaction. Exemplary reactions are described elsewhere herein. FIG. 15 shows a schematic that can demonstrate exosomal loading of cargo compound to increase loading efficiency of the exosome with the cargo molecule. Reversible linkage of chemical groups by ester linkages to the cargo compound can promote uptake of the cargo compound and can result in retention of the compound within the engineered vesicle (e.g. an exosome) until the ester bonds are removed via hydrolytic cleavage by an esterase or ester other ester bond breaking activity. Exosomes generated from the HeLa cells discussed in Example 22 were able to take up Calcein red dye. See FIGS. 27A-27D. Calcein has ester bonded chemical groups that can allow a molecule to pass through membranes. However, when ester bonds are cleaved by an ester bond breaking activity inside an exosome, the molecule loses its membrane permeability and thus becomes trapped within the membrane compartment unless some other release option is available. Exosomes were determined that they are capable of taking up and retaining Calcein for periods of at least 60 minutes or longer, which confirmed that the exosomes from the Hela cells contained esterase activity. FIG. 16 shows a fluorescent microscopic image that can demonstrate that milk exosomes retain Calcein dye, which indicates that the contain esterase activity similar to that demonstrated in connection with the HeLa cells previously. Thus, this Example can demonstrate that bonding of membrane permeable chemical groups by ester linkages to a cargo compound, coupled with the presence of one or more esterase in the exosome can provide a system for improved loading and retention efficiency in the exosome or other vesicle.

### Example 28

A 43 amino acid peptide mimetic encompassing amino acids Y313 through A348 of the Cx43 CT was synthesized, containing the H1 and H2 a-helical regions (FIGS. 28A-28D). In SPR assays, Cx43 Y313-A348 showed levels of interaction with aCT1 comparable to the full Cx43 CT sequence (about 150 amino acids, FIG. 28D) NMR solutions have indicated that ordered arrangements of the H1 and H2 alpha helices may include the formation of a loop-like domain near the middle of the CT 20 sequence, 28 (e.g., FIG. 28A). Cx43 Y313-A348 was designed to have cysteines at its NT and CT, which were used to disulfide cross-link the peptide into a cyclized conformation (FIG. 28B). SPR indicated that disulfide linkage of Cx43 Y313-A348 resulted in a complete loss of aCT1 binding, suggesting that interaction required a degree conformational flexibility. The Cx43 Y313-A348 peptide provides a means for screening for and identifying molecules like aCT1 (SEQ ID NO: 111), aCT1-I (SEQ ID NO: 112), aCT11 (SEQ ID NO: 13) and aCT11-I (SEQ ID NO: 14). that can interact with the Cx43 CT providing modified injury response benefit seen in examples herein (e.g., FIGS. 6, 8, 13, 20 and 21). The Cx43 Y313-A348 peptide can provide an assay for screening for novel Cx43 interacting drugs that provide these desirable clinical benefits.

### Example 29

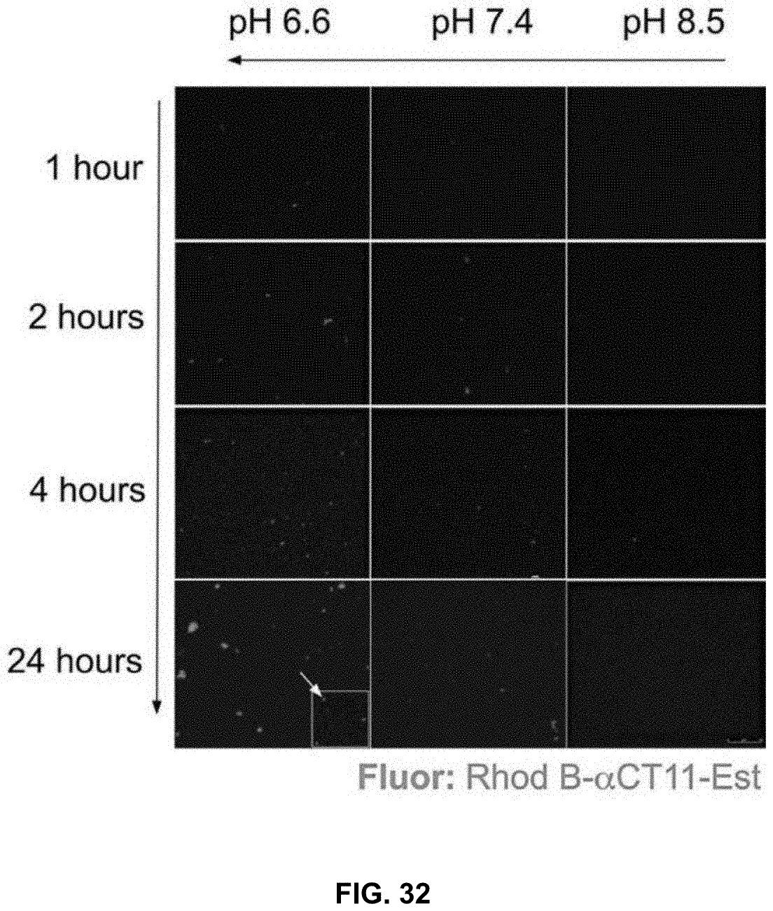

FIG. 29A provides exemplary EV drug cargo molecules. RhodamineB aCT11 peptide (top left). The bottom left shows acid-stable allyl protecting groups linked by ester bonds to peptide at aspartic (D) and glutamic (E) acid residues of aCT11. FIG. 29B Mass spectra (MALDI) of RhodamineB aCT11 peptide (top right) and RhodamineB aCT11 peptide with each of it D and E residues, as well as it terminal carboxylic acid group converted with ester bond linked protecting groups (bottom right). The peaks show molecular masses that correspond to the expected structure (non-methylated ‘VT’—TOP) and all 4 groups methylated (VT Me—Bottom) for the methylated version. The 2 peaks in each of the spectra shown correspond to the mass+hydrogen and mass+sodium. Examples of the useful properties of chemically modified peptides as described and demonstrated herein in uptake into EVs and cells are provided in FIGS. 32-34B.

### Example 30

EVs were isolated from cow milk and loaded with neutral non-fluorescent Calcein AM (10 μM) for 48 hours at 37 C in PBS buffer at pH 8.5 (FIG. 30A). This protocol resulted in efficient loading and retention of dye in the EVs (green spots in FIG. 30B)—owing to esterase activity that cleaved ester bonded shielding groups from Calcein AM converting it to negatively charged fluorescent Calcein. Calcein uptake into and retention within milk EVs was respectively inhibited and blocked by 0.1 and 1 μM PMSF, an inhibitor of carboxylesterases. Purity and integrity of exosomes isolated from cow milk was confirmed by negative stain electron microscopy (EM). FIG. 30B) illustrates an exosome isolated from cow milk. Scale bar=50 nm. The methods described herein of isolation from milk were adapted to obtain high yields of EV, taking particular care not to cause rapid and/or large-scale precipitation of milk casein, as well as in centrifugation steps, which can reduce EV yields from milk.

### Example 31

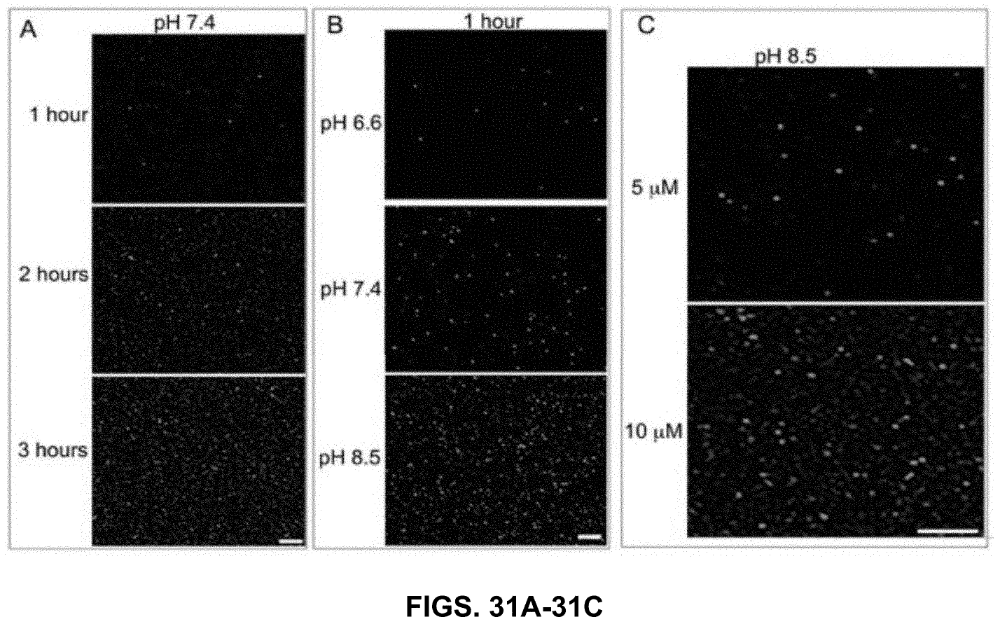

This Example can demonstrate methods developed for loading of Milk EVs with cargo molecules. An exemplar of these methods is shown in FIGS. 31A-31C wherein Milk EVs incubated with Calcein AM show time—(FIG. 31A), pH—(FIG. 31B) and concentration-dependent effects on uptake of Calcein by EVs. Important to this method in the case of Calcein AM, are the multiple chemical groups linked by ester bonds to the molecule, which shield it's negatively charged moieties. Cleavage of these groups by ester bond breaking activities within EVs results in Calcein becoming negatively charged, fluorescent and retained within the EV—as exemplified by the green spots seen in most figure panels in FIG. 31. In FIG. 31A EVs are illustrated that were incubated for 1, 2 or 3 hours in PBS at 37 C at pH 7.4 with Calcein AM (5 μM). Increasing numbers of EVs (green spots) show Calcein fluorescence with increasing time—indicating time dependent uptake. In FIG. 31B EVs were incubated at pH 6.6, 7.4 and 8.5 in PBS buffer at 37 C with Calcein AM (5 μM). Increasing numbers of EVs show Calcein fluorescence with increasing alkalinity of the buffer—indicating pH dependent uptake. Without being bound by theory, the mechanism driving EV uptake can be a pH gradient between the outside (less acidic) and inside (more acidic) that favors that accumulation of Calcein AM inside the EV. In FIG. 31C increasing numbers of EVs show Calcein fluorescence with increasing concentration of the dye—indicating concentration-dependent uptake during incubation in 37 C PBS at pH 8.5. These time-, pH- and cargo concentration-dependent effects are optimized herein to provide a novel method for highly efficient uptake of cargo molecules into milk EVs.

### Example 32

This Example can demonstrate methods for loading of Milk EVs with cargo molecules. A further exemplar of these methods is shown in FIG. 32. In this figure, milk EVs (red spots) incubated with fluorescent-tagged RhodamineB-aCT11 with multiple charge shielding allyl groups linked by ester bonds at aspartic (D) and glutamic (E) acid residues, as well as its carboxyl terminus—RhodB-aCT11-Est—are shown. EVs were incubated for 1, 2, 4 or 24 hours in PBS at 37 C with RhodB-aCT11-Est (1 mM) with the pH of PBS buffer solutions adjusted to pH 6.6, 7.4 or 8.5. FIG. 32 can demonstrate that peptide uptake into EVs occurs in a time- and pH-dependent manner, with the highest levels of uptake occurring in EVs incubated for 4 or 24 hours at pH 6.6. With its chemical groups shielding negatively charged COOH groups, RhodB-aCT11-Est has a positive charge. Fluor-tagged RhodamineB-aCT11 with no charge shielding groups showed little evidence of uptake by milk EVs. Without being bound by theory, the mechanism driving EV uptake can be a pH gradient between outside (more acidic) and inside (less acidic) of the EV that favors that accumulation of positively charged RhodB-aCT11-Est inside the EV. These time-, pH- and cargo-concentration dependent effects are optimized herein to provide a novel method for highly efficient uptake of cargo molecules milk EVs. In the case of RhodB-aCT11-Est, EVs loaded with drug cargo can be employed to provide the clinical benefits described at length herein, including as provided in the examples given in e.g. FIGS. 6, 8, 13, 20 and 21.

### Example 33

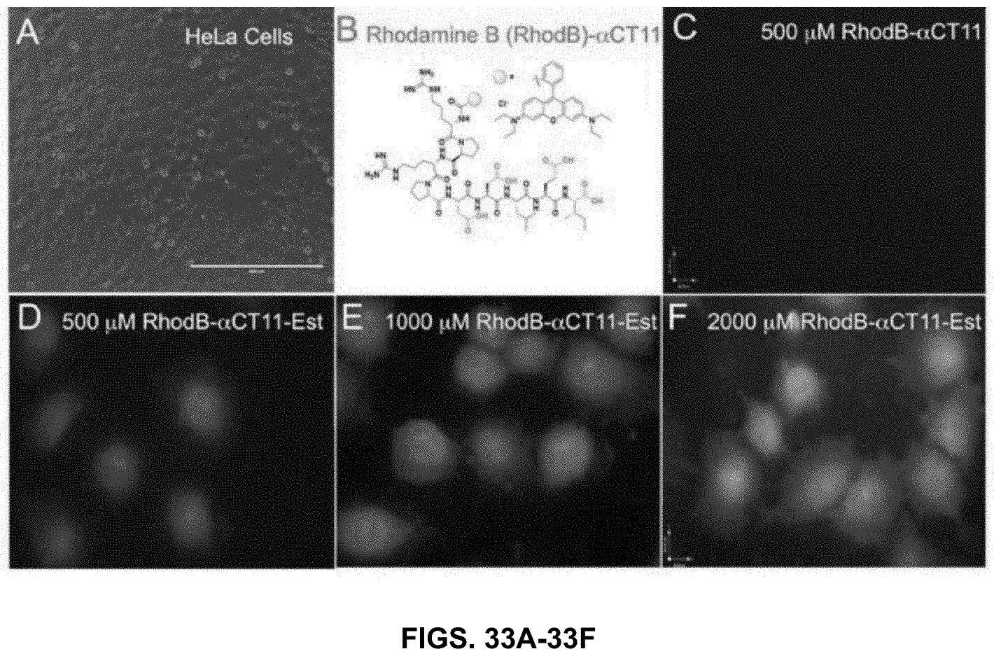

This Example can demonstrate methods for loading of Milk EVs with cargo molecules following exposure of EV-producing cells to a cargo molecule. An exemplar of these methods is shown in FIG. 33. FIG. 33A shows a monolayer of HeLa cells as imaged by Normaski optics. When a fluorescently tagged RhodamineB aCT11 peptide (RhodB-aCT11—FIG. 33B), not having allyl groups linked by ester bonds to its aspartic (D) and glutamic (E) acid residues, as well as the carboxyl terminus, is placed on HeLa cell monolayers at 500 μM in culture media for 90 minutes at 37 C little evidence for uptake of RhodB-aCT11 is observed (FIG. 33C). By contrast to RhodB-aCT11 (i.e., FIG. 33B), when RhodB-aCT11-Est (with allyl groups linked by ester bonds at its aspartic (D) and glutamic (E) acid residues, as well as its carboxyl terminus) is placed on HeLa monolayers at 500 or 2000 μM in culture media for 90 minutes at 37 C, fluorescent signals are readily observable within cells. This result indicates that RhodB-aCT11-Est is cell permeant and stably accumulates inside cells following esterase cleavage. The concentration-dependent uptake of RhodB-aCT11-Est can be used in methods wherein exosome producing cells are incubated with the peptide. Cells can take up the peptide, cytoplasmic esterases will cleave the allyl groups converting the peptide to RhodB-aCT11. RhodB-aCT11-Est, or any chemically modified drug molecule designed for cell uptake using ester bonded groups or related chemical modifications, can be packaged as cargo into EVs and exported by the cell into the media. EVs loaded with cargo molecules by this method can then be isolated using standard protocols and used in the treatment and other methods detailed herein.

### Example 34

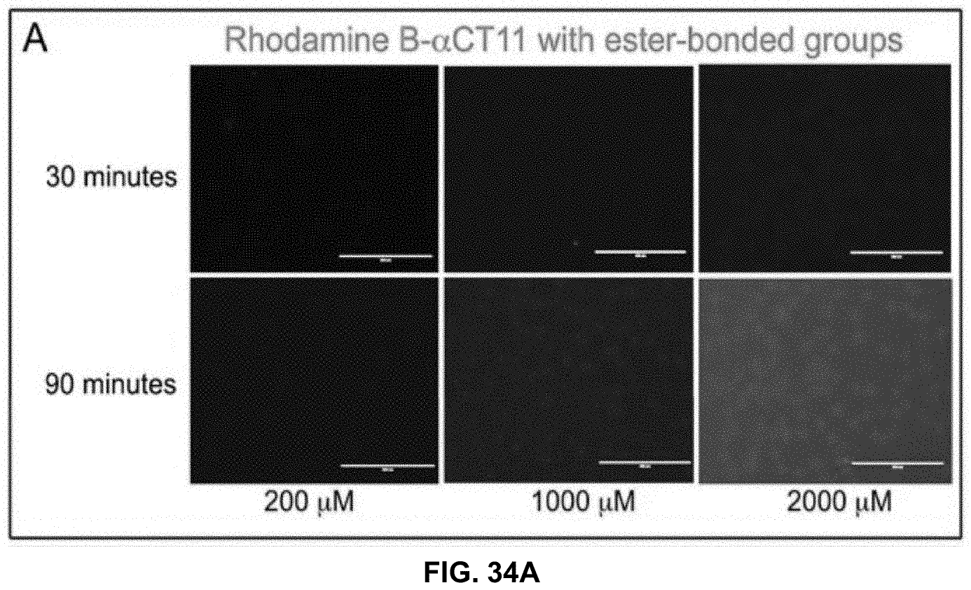

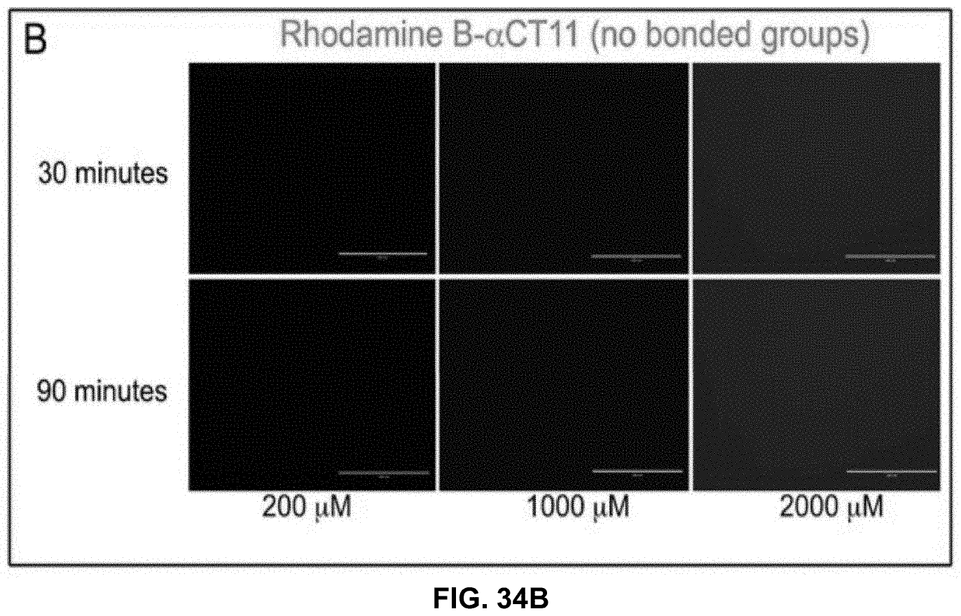

This Example can demonstrate methods of loading Milk EVs with a cargo molecule (e.g., Rhod B-aCT11-Est) following exposure of EV-producing cells to the said cargo molecule. Exemplars of the time- and drug-concentration dependent aspects of these methods are shown in FIG. 34. Monolayers of HeLa cells incubated with fluorescent-tagged RhodamineB-aCT11-Est, a cell-permeant peptide with allyl groups linked by ester bonds at aspartic (D) and glutamic (E) acid residues, as well as its carboxyl terminus are shown in FIG. 34A. The cells were incubated for 30 or 90 minutes at 37 C at pH 7.4 with different concentrations of peptides between 200 and 2000 μM. Cells similarly incubated with RhodamineB aCT11 peptide not having ester bonded groups is shown in FIG. 34B. Only those monolayers incubated with the cell-permeant peptide RhodB-aCT11-Est show uptake, which is seen to occur in a time- and concentration-dependent manner. Cellular uptake can be observed in FIG. 34A to be particularly evident following 90 minutes at higher peptide concentrations (i.e., >1000 μM). The uniform fluorescence in the 2000 mM incubations in FIG. 34B results from general fluorescence of concentrated peptide dissolved in the media i.e., it does not indicate cellular uptake. RhodB-aCT11-Est taken up in this manner by cells can be packaged as cargo into EVs and following isolation can these EVs be used in treatment and other methods detailed herein.

### Example 35

Additional Materials and Methods for Examples 21-25.

Peptides used in this project are synthesized by the American Peptide Company (Part of Thermo Fisher Scientific). This company has provided the lab reliable synthesis of purified (>98%) peptides since 2001. Peptides (with modifications) synthesized for the project include RPRPDDLEI (alpha CT11) (SEQ ID NO: 13), DRDPEIPLR (SEQ ID NO: 123) (scrambled inactive alpha CT11 control peptide), biotin-RPRPDDLEI (SEQ ID NO: 124), biotin-DRDPEIPLR (SEQ ID NO: 125), biotin-RPRPDDLAI (SEQ ID NO: 126) (Cx43 CT binding incompetent variant of alpha CT11), FAM (5,6)-RPRPDDLEI (SEQ ID NO: 127), and FAM (5,6)-DRDPEIPLR (SEQ ID NO: 128).

Antibodies

Antibodies were either purchased or generated. Antibodies against Nav1.5 and beta1 were generated against peptide epitopes from beta1 (AA 44-60) and Nav1.5 (AA 1947-1966) via a commercial custom antibody service (Thermo Fisher). Both antibodies displayed labeling of IDs in Guinea Pig ventricle, consistent with reported distribution of Nav1.5 sodium channels. Nav1.5 and beta1 each localized to a single band of expected molecule mass on Western Blots of Guinea Pig (GP) ventricle. Immunolabeling and Western signals were abolished by peptides to which the Nav1.5 and beta1 Abs were raised. Blots of lysates from parental and beta1 overexpressing (beta1 OX) 1610 cells, as well as from the hearts of Scn1b KO mice provided further confirmation of the specificity of the beta1 antibody.

The efficiency of loading cargo compounds can be improved by generating gradients between the inside and outside of EVs, appropriate to the charge of the cargo molecule (e.g., FIGS. 30-32). FIG. 31, in particular, shows that the efficiency of milk EV loading with neutral Calcein AM is increased by raising the alkalinity of the external solution to pH 8.5, causing a pH difference between the outside of the EV and interior of the EV. Decreased loading with Calcein AM is observed when the buffering solution is pH 7.0 or 6.6. The efficiency of milk EV loading with cationic RhodamineB-aCT11 with ester bonded allyl groups masking its negatively charged D and E amino acid residues, as well as it carboxyl terminus, is increased by decreasing the pH of the external solution to pH 6.6 (i.e., as illustrated in FIG. 32), whereas decreased loading with peptide is observed when the pH of the external solution is at pH 7.2 or pH 8.5. FIG. 32 further illustrates that cargo peptide uptake by EVs can be further enhanced by lengthening the time of incubation for 1 hour or more or increasing concentration of the cargo molecule in the buffer solution.

What is claimed is:

1. An engineered hemichannel comprising:
an engineered connexin 43 polypeptide comprising a non-functional c-terminus, wherein the engineered hemichannel is non-responsive to a change in pH.

2. The engineered hemichannel of claim 1, wherein the hemichannel is responsive to calcium concentration.

3. The engineered hemichannel of any one of claims 1-3, wherein the engineered connexin 43 polypeptide has a modified c-terminal region as compared to SEQ ID NO: 1.

4. The engineered hemichannel of claim 3, wherein the modification in the c-terminal region renders the engineered hemichannel non-responsive to changes in pH.

5. The engineered hemichannel of any one of claims 1-4, wherein the hemichannel is composed of 3-10 engineered connexin 43 polypeptides.

6. The engineered hem ichannel of any one of claims 1-5, wherein the change in pH is a change to an acidic pH.

7. The engineered hemichannel of any one of claims 1-5, wherein the change in pH is a change to a pH less than 8.5.

8. An engineered polypeptide comprising:
a modified connexin 43 polypeptide, wherein the modified connexin 43 polypeptide is modified as compared to SEQ ID NO: 1 and comprises one or more amino acid deletions, one or more amino acid insertions, one or more amino acid mutations, or any combination thereof in the c-terminal region of SEQ ID NO 1.

9. The engineered polypeptide of claim 8, wherein the engineered polypeptide is an amino acid sequence according to any one of SEQ ID NOs: 3-12.

10. The engineered polypeptide of claim 8, wherein the engineered polypeptide is an amino acid sequence that is about 50-100 percent identical to amino acids 1-224 of SEQ ID NO: 1 and has amino acids 225 to 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, or 382 of SEQ ID NO: 1 deleted.

11. The engineered polypeptide of claim 8, wherein the engineered polypeptide is an amino acid sequence that is about 50-100 percent identical to amino acids 1-224 of SEQ ID NO: 1 and has amino acids 382 to 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, or 381, of SEQ ID NO: 1 deleted.

12. The engineered polypeptide of claim 8, wherein the engineered polypeptide is about 50 percent to about 100% identical to amino acids 1-224 of SEQ ID NO: 1 and has one or more of amino acids 225-382 of SEQ ID NO: 1 deleted.

13. The engineered polypeptide of claim 12, wherein amino acids 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, or any combination thereof of SEQ ID NO: 1 is deleted.

14. The engineered polypeptide of claim 8, wherein the engineered polypeptide is about 50-100 percent identical to amino acids 1-224 of SEQ ID NO: 1 and has one or more amino acids inserted between any two amino acids from amino acid residues 224-382 of SEQ ID NO: 1.

15. The engineered polypeptide of claim 14, wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acids are inserted between any two amino acid residues in the c-terminus region ranging from amino acid residues 224 and 382 of SEQ ID NO: 1.

16. The engineered polypeptide of any one of claims 14-15, wherein at least two insertions are present in the engineered polypeptide.

17. The engineered polypeptide of claim 16, wherein the insertions are the same amino acid, peptide, or polypeptide.

18. The engineered polypeptide of claim 16, at least two of the insertions can be different from each other.

19. The engineered polypeptide of any one of claims 14-16, wherein the insertion is A, I, L, M, V, F, W, Y, N, C, Q, S, T, D, E, R, H, K, G, P or any combination thereof.

20. The engineered polypeptide of claim 8, wherein the engineered polypeptide can include one or more amino acid mutations in the c-terminal region as compared to SEQ ID NO: 1.

21. The engineered polypeptide of claim 20, wherein any one or more of the amino acids residues 225-382 can be substituted with any one of amino acids A, I, L, M, V, F, W, Y, N, C, Q, S, T, D, E, R, H, K, G, P that is not the same as the amino acid that it is being substituted for.

22. The engineered polypeptide of any one of claims 20-21, wherein the mutation is selected from the group consisting of: S368A, S368D, S365A, S365D, S373A, S373A D379A, E381A, S364P, C298A, E381A, D379A, D378A, S325A, S328A, S330A, and any combination thereof.

23. A polynucleotide comprising:
a polynucleotide configured to encode an engineered polypeptide as in any one of claims 8-22.

24. A vector comprising:
a polynucleotide as in claim 23 and a regulatory polynucleotide, wherein the regulatory polynucleotide is operably linked to the polynucleotide configured to encode the engineered polypeptide.

25. A cell comprising a vector as in claim 24.

26. A cell comprising a polynucleotide as in claim 23.

27. A cell comprising an engineered hemichannel as in any one of claims 1-7, one or more polypeptides as in any one of claims 8-22, or both.

28. An engineered hemichannel comprising:
an engineered polypeptide as in any one of claims 8-22.

29. The engineered hemichannel of claim 28, wherein the engineered hemichannel has 3 to 10 engineered polypeptides as in any one of claims 8-22.

30. The engineered hemichannel of any one of claims 28-30, wherein the engineered polypeptides are all the same.

31. The engineered hemichannel of any one of claim 28-30, wherein at least two of the engineered polypeptides are different.

32. The engineered hemichannel of any one of claims 28-30, wherein all of the engineered polypeptides are different.

33. An engineered vesicle comprising:
a lipid bilayer; and
an engineered hem ichannel as in any of claims 1-7, an engineered polypeptide as in any one of claims 8-22, or both, wherein the engineered polypeptide is integrated in the lipid bilayer.

34. An engineered vesicle comprising:
a lipid bilayer; and
a plurality of engineered polypeptides, wherein each engineered polypeptide of the plurality of engineered polypeptides is as in any one of claims 8-22 wherein the engineered polypeptides are integrated in the lipid bilayer.

35. The engineered vesicle of claim 34, wherein the plurality of engineered polypeptides forms a hemichannel.

36. The engineered vesicle of claim 34, further comprising a cargo compound, wherein the cargo compound is contained within the engineered vesicle.

37. An engineered vesicle comprising:
a lipid bilayer; and
an engineered hemichannel as in any one of claim 1-7 or 28-32.

38. The engineered vesicle of claim 37, further comprising a cargo compound, wherein the cargo compound is contained within the engineered vesicle.

39. The engineered vesicle of any one of claims 33-38, wherein the engineered vesicle is substantially spherical and has a diameter of about 1 nm to about 200 nm.

40. The engineered vesicle of any one of claims 33-39, wherein the engineered vesicle is a milk-based engineered vesicle.

41. An engineered vesicle comprising:
a milk exosome; and
a peptide cargo molecule contained within the milk exosome, wherein the peptide compound is selected from the group consisting of: SEQ ID NOS: 13-47, 49-114, and 133.

42. The engineered vesicle of claim 41, wherein the milk exosome is a natural milk exosome.

43. The engineered vesicle of any one of claims 33-42, wherein the engineered vesicle further comprises an esterase.

44. A cell, wherein the cell is capable of producing the engineered vesicle of any one of claims 33-43.

45. The cell of claim 44, wherein the cell is capable of secreting the engineered vesicles.

46. The cell of any one of claims 44-45, wherein the cell comprises an engineered vesicle as in any one of claims 33-43.

47. A cell comprising:
an engineered vesicle as in any one of claims 33-43.

48. A method of loading a cargo compound in an engineered vesicle of any one of claim 33-40 or 43, the method comprising:
exposing an engineered vesicle to a solution comprising a low concentration of calcium and a cargo compound, wherein the low concentration of calcium opens the engineered hem ichannel of the engineered vesicle,
allowing the cargo compound to enter the engineered vesicle through the open engineered hem ichannel,
closing the engineered hemichannel by exposing the engineered vesicle to a solution comprising a high concentration of calcium.

49. The method of claim 48, wherein the solution comprising a low concentration of calcium further comprises EDTA.

50. The method of any one of claims 48-49, wherein the low concentration of calcium ranges from 0 mM to about 0.2 mM.

51. The method of any one of claims 48-50, wherein the high concentration of calcium ranges from 0 mM to about 2 mM.

52. The method of any one of claims 48-51, wherein the cargo compound comprises a cleavable ester group.

53. The method of claim 52, wherein the cleavable ester group is cleaved by an esterase present in the engineered vesicle.

54. A method comprising:
opening an engineered hemichannel as in any one of claim 1-7 or 28-32 or as in any one of claim 33-40 or 43 by contacting the engineered hem ichannel with a solution comprising a low concentration of Ca²⁺, wherein the low concentration of Ca²⁺ is capable of stimulating opening of the engineered hemichannel.

55. The method of claim 54, wherein the solution further comprises a cargo compound, wherein the concentration of the cargo compound in solution is such that it drives movement of the agent through the engineered hemichannel.

56. The method of any one of claims 54-55, wherein the engineered hemichannel is integrated in a lipid bilayer of a vesicle.

57. The method of any one of claims 54-56, further comprising the step of closing the engineered hemichannel by removing the engineered hem ichannel from contact with the solution comprising a low concentration of calcium.

58. The method of claim 57, wherein the step of closing the engineered hem ichannel is carried out by raising the concentration of calcium in the solution.

59. The method of any one of claims 55-58, wherein the cargo compound comprises one or more cleavable ester bond-linked groups.

60. The method of claim 59, wherein the cleavable ester bond-linked group is cleaved by an esterase or via other ester bond breaking acitivty present in the engineered vesicle.

61. A method of loading a cargo compound into a vesicle, the method comprising:
exposing a vesicle or component thereof to a cargo compound,
allowing the cargo compound to enter the vesicle, be encapsulated by the vesicle, or both, wherein the vesicle comprises an esterase and wherein the cargo compound comprises one or more cleavable groups, wherein each cleavable group is linked by an ester bond to the cargo compound.

62. The method of claim 61, wherein the vesicle is an engineered vesicle as in any one of claim 33-40 or 43.

63. The method of claim 61, wherein the vesicle is a milk exosome as in any one of claims 41-43.

64. The method of any of claims 61-63, wherein the vesicle and cargo compound are exposed to a pH gradient formed between the inside of the vesicle and the outside of the vesicle during the step of exposing the vesicle or component thereof to the cargo compound, allowing the cargo compound to enter the vesicle, or both.

65. The method of claim 64, wherein the vesicle is exposed to an acidic pH.

66. The method of claim 64, wherein the vesicle is exposed to a basic pH.

67. The method of claim 66, wherein the vesicle is exposed to a pH of 8.5 or greater.

68. The method of any one of claims 61-67, wherein the steps of exposing and allowing occur for at least 1 hour.

69. The method of any one of claims 61-68, wherein the cargo compound is negatively charged.

70. The method of any one of claims 61-68, wherein the cargo compound is positively charged.

71. The method of any one of claims 61-68, wherein the cargo compound is neutrally charged.

72. The method of any one of claims 61-71, wherein the cargo compound further comprises one or more charge modifying groups capable of shielding a charged group, adding a charged group, or both to the compound and modifying the charge of the cargo compound.

73. A method comprising:
administering an amount of an engineered vesicle as in any one of claims 33-43 or a cell as in any one of claim 25-27 or 44-47 to a subject.

74. The method of claim 73, wherein the subject has a disease, disorder, or condition.

75. The method of any one of claims 73-74, wherein the subject has a chronic wound.

76. The method of any one of claims 73-75, wherein the subject has a diabetic ulcer.

77. The method of any one of claims 73-76, wherein the engineered vesicle contains a cargo compound.

78. The method of claim 77, wherein the cargo compound is a peptide compound.

79. The method of claim 78, wherein the peptide compound is selected from the group consisting of: SEQ ID NOS: 13-47, 49-114, and 133.

80. The method of any one of claims 54-79, wherein the cargo compound comprises a cleavable ester group.

81. The method of claim 80, wherein the cleavable ester group is cleaved by an esterase present in the engineered vesicle.

82. A method of treating a disease in a subject in need thereof, the method comprising:
administering an engineered vesicle containing a cargo compound as in any one of claims 36 and 38-43, wherein the cargo compound is capable of treating and/or preventing a disease or a symptom thereof in the subject.

83. The method of claim 82, wherein the disease is a skin wound, a chronic wound, myocardial infarction, heart failure, neural stroke, lung injury, macular degeneration, and radiation injury.

84. The method of any one of claims 82-83, wherein the disease is a diabetic ulcer.

85. The method of any one of claims 82-84, wherein the cargo compound comprises a cleavable ester group.

86. The method of claim 85, wherein the cleavable ester group is cleaved by an esterase present in the engineered vesicle.

87. An engineered polypeptide comprising:
a peptide, wherein the peptide consists of a plurality of amino acids having a sequence identical to SEQ ID NO: 14 or 112.

88. The engineered polypeptide of claim 87, further comprising a second polypeptide, wherein the second polypeptide is capable of performing a function different from the peptide of claim 87.

89. The engineered polypeptide of claim 88, wherein the second polypeptide is a selectable marker.

90. An engineered polypeptide comprising:
a peptide, wherein the peptide consists of a plurality of amino acids having a sequence identical to SEQ ID NO: 14 or 112.

91. An engineered peptide consisting of:
a peptide having a sequence identical to SEQ ID NO: 14 or 112.

92. A pharmaceutical formulation comprising:
an engineered polypeptide of any one of claims 87-90 or an engineered peptide of claim 91; and
a pharmaceutically acceptable carrier.

93. A method comprising:
administering an engineered polypeptide of any one of claims 87-90 or an engineered peptide of claim 91 or a pharmaceutical formulation as in claim 92 to a subject.

94. The method of claim 93, wherein the subject has or is suspected of having a disease.

95. A method of treating a subject in need thereof, the method comprising:
administering an engineered polypeptide of any one of claims 87-90 or an engineered peptide of claim 91 or a pharmaceutical formulation as in claim 92 to the subject in need thereof.

96. A pharmaceutical formulation comprising:
an engineered vesicle as in any one claims 33-43; and a pharmaceutically acceptable carrier.

97. The pharmaceutical formulation of claim 96, wherein the pharmaceutically acceptable carrier is milk or a milk product.

98. A method comprising:
administering a pharmaceutical formulation as in any one of claims 96-97 to a subject in need thereof.


SEQUENCE LISTING (133 sequences)

SEQ ID NO: 1 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDDLEI

SEQ ID NO: 2 - protein, 226 aa, Homo sapiens
MDWGTLQTILGGVNKHSTSIGKIWLTVLFIFRIMILVVAAKEVWGDEQADFVCNTLQPGCKNVCYDHYFPISHIRLWALQLIFVSTPALLVAMHVAYRRHEKKRKFIKGEIKSEFKDIEEIKTQKVRIEGSLWWTYTSSIFFRVIFEAAFMYVFYVMYDGFSMQRLVKCNAWPCPNTVDCFVSRPTEKTVFTVFMIAVSGICILLNVTELCYLLIRYCSGKSKKPV

SEQ ID NO: 3 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRAASRASSRPRPDDLEI

SEQ ID NO: 4 - protein, 382 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGATIANAHAQPFDFPDDNQNSKKLAAGHELQPLAIVDQRPSSRASSRASSRPRPDDLEI

SEQ ID NO: 5 - protein, 258 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAK

SEQ ID NO: 6 - protein, 357 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLA

SEQ ID NO: 7 - protein, 356 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPL

SEQ ID NO: 8 - protein, 379 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRPDD

SEQ ID NO: 9 - protein, 324 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAG

SEQ ID NO: 10 - protein, 325 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGS

SEQ ID NO: 11 - protein, 377 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRPSSRASSRASSRPRP

SEQ ID NO: 12 - protein, 363 aa, Homo sapiens
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVDMHLKQIEIKKFKYGIEEHGKVKMRGGLLRTYIISILFKSIFEVAFLLIQWYIYGFSLSAVYTCKRDPCPHQVDCFLSRPTEKTIFIIFMLVVSLVSLALNIIELFYVFFKGVKDRVKGKSDPYHATSGALSPAKDCGSQKYAYFNGCSSPTAPLSPMSPPGYKLVTGDRNNSSCRNYNKQASEQNWANYSAEQNRMGQAGSTISNSHAQPFDFPDDNQNSKKLAAGHELQPLAIVDQRP

SEQ ID NO: 13 - protein, 9 aa, Artificial Sequence
  Synthetic Peptide
RPRPDDLEI

SEQ ID NO: 14 - protein, 8 aa, Artificial Sequence
  Synthetic Peptide
RPRPDDLE

SEQ ID NO: 15 - protein, 6 aa, Artificial Sequence
  Synthetic Peptide
RPRPDD

SEQ ID NO: 16 - protein, 10 aa, Artificial Sequence
  Synthetic Peptide
SRPRPDDLEI

SEQ ID NO: 17 - protein, 9 aa, Artificial Sequence
  Synthetic Peptide
SRPRPDDLE

SEQ ID NO: 18 - protein, 7 aa, Artificial Sequence
  Synthetic Peptide
SRPRPDD

SEQ ID NO: 19 - protein, 22 aa, Artificial Sequence
  Synthetic Peptide
IVDQRPSSRASSRASSRPRPDD

SEQ ID NO: 20 - protein, 20 aa, Artificial Sequence
  Synthetic Peptide
PSSRASSRASSRPRPDDLEI

SEQ ID NO: 21 - protein, 9 aa, Artificial Sequence
  Synthetic Peptide
RARPDDLDV

SEQ ID NO: 22 - protein, 9 aa, Artificial Sequence
  Synthetic Peptide
GDGKNSWWI

SEQ ID NO: 23 - protein, 10 aa, Artificial Sequence
  Synthetic Peptide
GRARPEDLAI

SEQ ID NO: 24 - protein, 8 aa, Artificial Sequence
  Synthetic Peptide
RDGKTVWI

SEQ ID NO: 25 - protein, 11 aa, Artificial Sequence
  Synthetic Peptide
GRTQSSDSAYW

SEQ ID NO: 26 - protein, 12 aa, Artificial Sequence
  Synthetic Peptide
KASSKARSDDSW

SEQ ID NO: 27 - protein, 9 aa, Artificial Sequence
  Synthetic Peptide
CSGKSKKPW

SEQ ID NO: 28 - protein, 22 aa, Artificial Sequence
  Synthetic Peptide
IVDQRPSSRASSRASSRPRPDD

SEQ ID NO: 29 - protein, 20 aa, Artificial Sequence
  Synthetic Peptide
PSSRASSRASSRPRPDDLEI

SEQ ID NO: 30 - protein, 5 aa, Artificial Sequence
  Synthetic Peptide
DDLEI

SEQ ID NO: 31 - protein, 4 aa, Artificial Sequence
  Synthetic Peptide
DLEI

SEQ ID NO: 32 - protein, 3 aa, Artificial Sequence
  Synthetic Peptide
LEI

SEQ ID NO: 33 - protein, 8 aa, Artificial Sequence
  Synthetic Peptide
PRPDDLEI

SEQ ID NO: 34 - protein, 6 aa, Artificial Sequence
  Synthetic Peptide
PDDLEI

SEQ ID NO: 35 - protein, 9 aa, Artificial Sequence
  Synthetic Peptide
KPRPDDLEI

SEQ ID NO: 36 - protein, 9 aa, Artificial Sequence
  Synthetic Peptide
RPRPDDLEV

SEQ ID NO: 37 - protein, 9 aa, Artificial Sequence
  Synthetic Peptide
RPRPDDVPV

SEQ ID NO: 38 - protein, 9 aa, Artificial Sequence
  Synthetic Peptide
RPKPDDLEI

SEQ ID NO: 39 - protein, 19 aa, Artificial Sequence
  Synthetic Peptide
SSRASSRASSRPKPDDLEI

SEQ ID NO: 40 - protein, 6 aa, Artificial Sequence
  Synthetic Peptide
RPKPDD

SEQ ID NO: 41 - protein, 19 aa, Artificial Sequence
  Synthetic Peptide
SSRASSRASSRPRPDDLDI

SEQ ID NO: 42 - protein, 19 aa, Artificial Sequence
  Synthetic Peptide
SSRASTRASSRPRPDDLEI

SEQ ID NO: 43 - protein, 9 aa, Artificial Sequence
  Synthetic Peptide
RPRPEDLEI

SEQ ID NO: 44 - protein, 19 aa, Artificial Sequence
  Synthetic Peptide
SSRASSRASSRPRPEDLEI

SEQ ID NO: 45 - protein, 9 aa, Artificial Sequence
  Synthetic Peptide
GDGKNSVWV

SEQ ID NO: 46 - protein, 23 aa, Artificial Sequence
  Synthetic Peptide
SKAGSNKSTASSKSGDGKNSVWV

SEQ ID NO: 47 - protein, 18 aa, Artificial Sequence
  Synthetic Peptide
GQKPPSRPSSSASKKLYV

SEQ ID NO: 48 - protein, 43 aa, Artificial Sequence
  Synthetic Peptide
GYSAEQNRMGQAGSTISNSHAQPFDFPDDNQNAKKVAAGHEGC

SEQ ID NO: 49 - protein, 10 aa, Artificial Sequence
  Synthetic Peptide
ERPRPDDLEI

SEQ ID NO: 50 - protein, 10 aa, Artificial Sequence
  Synthetic Peptide
DRPRPDDLEI

SEQ ID NO: 51 - protein, 11 aa, Artificial Sequence
  Synthetic Peptide
EERPRPDDLEI

SEQ ID NO: 52 - protein, 9 aa, Artificial Sequence
  Synthetic Peptide
ERPRPDDEL

SEQ ID NO: 53 - protein, 11 aa, Artificial Sequence
  Synthetic Peptide
DDRPRPDDELI

SEQ ID NO: 54 - protein, 15 aa, Artificial Sequence
  Synthetic Peptide
VFFKGVKDRVKGKSD

SEQ ID NO: 55 - protein, 10 aa, Artificial Sequence
  Synthetic Peptide
VFFKGVKDRV

SEQ ID NO: 56 - protein, 20 aa, Artificial Sequence
  Synthetic Peptide
VFFKGVKDRVKGRSDPYHAT

SEQ ID NO: 57 - protein, 9 aa, Artificial Sequence
  Synthetic Peptide
FFKGVKDRV

SEQ ID NO: 58 - protein, 8 aa, Artificial Sequence
  Synthetic Peptide
FKGVKDRV

SEQ ID NO: 59 - protein, 9 aa, Artificial Sequence
  Synthetic Peptide
VFFKGVKDR

SEQ ID NO: 60 - protein, 8 aa, Artificial Sequence
  Synthetic Peptide
VFFKGVKD

SEQ ID NO: 61 - protein, 13 aa, Artificial Sequence
  Synthetic Peptide
DRVKGRSDPYHAT

SEQ ID NO: 62 - protein, 11 aa, Artificial Sequence
  Synthetic Peptide
VKGRSDPYHAT

SEQ ID NO: 63 - protein, 15 aa, Artificial Sequence
  Synthetic Peptide
VFFKGVKDRVKGQSD

SEQ ID NO: 64 - protein, 15 aa, Artificial Sequence
  Synthetic Peptide
VFFKGIKDRVKGRND

SEQ ID NO: 65 - protein, 15 aa, Artificial Sequence
  Synthetic Peptide
VFFKGVKDRVKGRID

SEQ ID NO: 66 - protein, 15 aa, Artificial Sequence
  Synthetic Peptide
VFFKGIKDRVKGKSD

SEQ ID NO: 67 - protein, 14 aa, Artificial Sequence
  Synthetic Peptide
FFKGVKDRVKGKSD

SEQ ID NO: 68 - protein, 13 aa, Artificial Sequence
  Synthetic Peptide
FKSVKDRIKGRSD

SEQ ID NO: 69 - protein, 15 aa, Artificial Sequence
  Synthetic Oligonucleotide
VFFRSVKDHVKGKSD

SEQ ID NO: 70 - protein, 12 aa, Artificial Sequence
  Synthetic Peptide
VFFKRIKDRVKG

SEQ ID NO: 71 - protein, 13 aa, Artificial Sequence
  Synthetic Peptide
VLFKQIKDRVKGR

SEQ ID NO: 72 - protein, 13 aa, Artificial Sequence
  Synthetic Peptide
VLFKRIKDRVKGR

SEQ ID NO: 73 - protein, 15 aa, Artificial Sequence
  Synthetic Peptide
VFFKGVKDRVKGKSD

SEQ ID NO: 74 - protein, 10 aa, Artificial Sequence
  Synthetic Peptide
VFFKGVKDRV

SEQ ID NO: 75 - protein, 15 aa, Artificial Sequence
  Synthetic Peptide
IFFKGVKDRVKGKSD

SEQ ID NO: 76 - protein, 10 aa, Artificial Sequence
  Synthetic Peptide
IFFKGVKDRV

SEQ ID NO: 77 - protein, 15 aa, Artificial Sequence
  Synthetic Peptide
VIFKRMKDQIRESEK

SEQ ID NO: 78 - protein, 15 aa, Artificial Sequence
  Synthetic Peptide
VFFKGVKDRVKGKTD

SEQ ID NO: 79 - protein, 15 aa, Artificial Sequence
  Synthetic Peptide
VFFKGVKDRVKGRSD

SEQ ID NO: 80 - protein, 15 aa, Artificial Sequence
  Synthetic Peptide
VFFKGVKDRVRGKSD

SEQ ID NO: 81 - protein, 15 aa, Artificial Sequence
  Synthetic Peptide
VFFKGVKDKVKGKSD

SEQ ID NO: 82 - protein, 15 aa, Artificial Sequence
  Synthetic Peptide
IIFRGVRDRVRGRSD

SEQ ID NO: 83 - protein, 15 aa, Artificial Sequence
  Synthetic Peptide
VIFKRMKDQIRESEK

SEQ ID NO: 84 - protein, 15 aa, Artificial Sequence
  Synthetic Peptide
VIFKRMKDQIREREK

SEQ ID NO: 85 - protein, 15 aa, Artificial Sequence
  Synthetic Peptide
VIFKRMKDKIREREK

SEQ ID NO: 86 - protein, 15 aa, Artificial Sequence
  Synthetic Peptide
VFFKRVKDRIRERSK

SEQ ID NO: 87 - protein, 15 aa, Artificial Sequence
  Synthetic Peptide
VFFKGVKDRVKGRSD

SEQ ID NO: 88 - protein, 39 aa, Artificial Sequence
  Synthetic Peptide
DPYHATSGALSPAKDCGSQKYAYFNGCSSPTAPLSPMSP

SEQ ID NO: 89 - protein, 18 aa, Artificial Sequence
  Synthetic Peptide
AYFNGCSSPTAPLSPMSP

SEQ ID NO: 90 - protein, 10 aa, Artificial Sequence
  Synthetic Peptide
PTAPLSPMSP

SEQ ID NO: 91 - protein, 8 aa, Artificial Sequence
  Synthetic Peptide
PTAPLSPM

SEQ ID NO: 92 - protein, 8 aa, Artificial Sequence
  Synthetic Peptide
APLSPMSP

SEQ ID NO: 93 - protein, 30 aa, Artificial Sequence
  Synthetic Peptide
HAQPFDFPDDNQNSKKLAAGHELQPLAIVD

SEQ ID NO: 94 - protein, 10 aa, Artificial Sequence
  Synthetic Peptide
NQNSKKLAAG

SEQ ID NO: 95 - protein, 8 aa, Artificial Sequence
  Synthetic Peptide
NSKKLAAG

SEQ ID NO: 96 - protein, 10 aa, Artificial Sequence
  Synthetic Peptide
HELQPLAIVD

SEQ ID NO: 97 - protein, 9 aa, Artificial Sequence
  Synthetic Peptide
KQIEIKKFK

SEQ ID NO: 98 - protein, 26 aa, Artificial Sequence
  Synthetic Peptide
DGANVDMHLKQIEIKKFKYGIEEHGK

SEQ ID NO: 99 - protein, 11 aa, Artificial Sequence
  Synthetic Peptide
KQIEIKKFKYG

SEQ ID NO: 100 - protein, 12 aa, Artificial Sequence
  Synthetic Peptide
VDCFLSRPTEKT

SEQ ID NO: 101 - protein, 11 aa, Artificial Sequence
  Synthetic Peptide
SRPTEKTIFII

SEQ ID NO: 102 - protein, 11 aa, Artificial Sequence
  Synthetic Peptide
SRPTEKTIFLL

SEQ ID NO: 103 - protein, 7 aa, Artificial Sequence
  Synthetic Peptide
SRPTEKT

SEQ ID NO: 104 - protein, 8 aa, Artificial Sequence
  Synthetic Peptide
ESRPTEKT

SEQ ID NO: 105 - protein, 12 aa, Artificial Sequence
  Synthetic Peptide
ADCFLSRPTEKT

SEQ ID NO: 106 - protein, 12 aa, Artificial Sequence
  Synthetic Peptide
VACFLSRPTEKT

SEQ ID NO: 107 - protein, 12 aa, Artificial Sequence
  Synthetic Peptide
VDCFLSRPTAKT

SEQ ID NO: 108 - protein, 12 aa, Artificial Sequence
  Synthetic Peptide
VDCFLSRPTEAT

SEQ ID NO: 109 - protein, 10 aa, Artificial Sequence
  Synthetic Peptide
CFLSRPTEKT

SEQ ID NO: 110 - protein, 8 aa, Artificial Sequence
  LSRPTEKT
LSRPTEKT

SEQ ID NO: 111 - protein, 25 aa, Artificial Sequence
  Synthetic Peptide
RQPKIWFPNRRKPWKKRPRPDDLEI

SEQ ID NO: 112 - protein, 24 aa, Artificial Sequence
  Synthetic Peptide
RQPKIWFPNRRKPWKKRPRPDDLE

SEQ ID NO: 113 - protein, 25 aa, Artificial Sequence
  Synthetic Peptide
RQPKIWFPNRRKPWKKRPRPDDLAI

SEQ ID NO: 114 - protein, 9 aa, Artificial Sequence
  Synthetic Peptide
RPRPDDLAI

SEQ ID NO: 115 - protein, 6 aa, Artificial Sequence
  Synthetic Peptide
RPDDLE

SEQ ID NO: 116 - protein, 9 aa, Artificial Sequence
  Synthetic Peptide
RPRPDDELI

SEQ ID NO: 117 - protein, 24 aa, Artificial Sequence
  Synthetic Peptide
QPKIWFPNRRKPWKKIELDDPRPR

SEQ ID NO: 118 - protein, 25 aa, Artificial Sequence
  Synthetic Peptide
RQPKIWFPNRRKPWKKRPRPAALAI

SEQ ID NO: 119 - protein, 25 aa, Artificial Sequence
  Synthetic Peptide
RQPKIWFPNRRKPWKKRPRPAALEI

SEQ ID NO: 120 - protein, 25 aa, Artificial Sequence
  Synthetic Peptide
RQPKIWFPNRRKPWKKLPAARIAPR

SEQ ID NO: 121 - protein, 9 aa, Artificial Sequence
  Synthetic Peptide
RPRPAALAI

SEQ ID NO: 122 - protein, 9 aa, Artificial Sequence
  Synthetic Peptide
RPRPAALEI

SEQ ID NO: 123 - protein, 9 aa, Artificial Sequence
  Synthetic Peptide
DRDPEIPLR

SEQ ID NO: 124 - protein, 9 aa, Artificial Sequence
  Synthetic Peptide
  MISC_FEATURE (1)..(1): N-terminal biotin modification
RPRPDDLEI

SEQ ID NO: 125 - protein, 9 aa, Artificial Sequence
  Synthetic Peptide
  MISC_FEATURE (1)..(1): N-terminal biotin modification
DRDPEIPLR

SEQ ID NO: 126 - protein, 9 aa, Artificial Sequence
  Synthetic Peptide
  MISC_FEATURE (1)..(1): N-terminal biotin modification
RPRPDDLAI

SEQ ID NO: 127 - protein, 9 aa, Artificial Sequence
  Synthetic Peptide
  MISC_FEATURE (1)..(1): N-terminal FAM 5,6 modification
RPRPDDLEI

SEQ ID NO: 128 - protein, 9 aa, Artificial Sequence
  Synthetic Peptide
  MISC_FEATURE (1)..(1): N-terminal FAM 5,6 modification
DRDPEIPLR

SEQ ID NO: 129 - protein, 16 aa, Artificial Sequence
  Synthetic Peptide
RQIKIWFQNRRMKWKK

SEQ ID NO: 130 - protein, 21 aa, Artificial Sequence
  Synthetic Peptide
KVAAGHELQPLAIVDQRPSSR

SEQ ID NO: 131 - protein, 26 aa, Artificial Sequence
  Synthetic Peptide
GQAGSTISNSHAQPFDFPDDNQNAKK

SEQ ID NO: 132 - protein, 52 aa, Artificial Sequence
  Synthetic Peptide
HAQPFDFPDDNQNSKKLAAGHELQPLAIVDQRPSSRASSRASSRPRPDDLEI

SEQ ID NO: 133 - protein, 7 aa, Artificial Sequence
  Synthetic Peptide
RPDDLEI